(19) United States
(12) Patent Application Publication
(10) Pub. No.: US 2023/0285596 A1
(43) Pub. Date: Sep. 14, 2023

(54) COMPOSITIONS AND METHODS FOR THE TREATMENT OF NIEMANN-PICK TYPE C1 DISEASE

(71) Applicant: Voyager Therapeutics, Inc, Cambridge, MA (US)

(72) Inventors: Heather Yonutas, Cambridge, MA (US); Jeffrey Brown, Medway, MA (US); Jinzhao Hou, Lexington, MA (US); Yanqun Shu, Winchester, MA (US); Elisabeth Knoll, Cambridge, MA (US); Priyantha Herath, Cambridge, MA (US); Brett Hoffman, Cambridge, MA (US)

(21) Appl. No.: 18/018,089

(22) PCT Filed: Jul. 26, 2021
(86) PCT No.: PCT/US2021/043217

§ 371 Date: Jan. 26, 2023

Related U.S. Application Data
(60) Provisional application No. 63/057,258, filed on Jul. 27, 2020.

(51) Int. Cl. A61K 48/00; C07K 14/705; A61K 45/06; A61P 3/06; C12N 15/86

(52) U.S. Cl. CPC A61K 48/0058; C07K 14/705; A61K 48/0041; A61K 45/06; A61P 3/06; C12N 15/86; C12N 2750/14143

(57) ABSTRACT

The disclosure provides compositions and methods for altering, e.g., enhancing, the expression of NPC1 and/or NPC2 proteins, whether in vitro and/or in vivo. Such compositions include delivery of an adeno-associated viral (AAV) particle. The compositions and methods of the present disclosure are useful in the treatment of subjects diagnosed with, or suspected of having NPC1 disease or related condition resulting from a deficiency in the quantity and/or function of NPC1 and/or NPC2 proteins or associated with decreased expression or protein levels of NPC1 and/or NPC2 proteins.

### RELATED APPLICATIONS

This application claims priority to U.S. provisional application No. 63/057,258, filed on Jul. 27, 2020. The entire contents of the foregoing application are hereby incorporated herein by reference.

### REFERENCE TO THE SEQUENCE LISTING

The present application is being filed along with a Sequence Listing in electronic format. The Sequence Listing file, entitled 135333-00220_SL, was created on Jul. 23, 2021, and is 6,807,032 bytes in size. The information in electronic format of the Sequence Listing is incorporated herein by reference in its entirety.

### FIELD OF THE DISCLOSURE

Described herein are compositions and methods relating to polynucleotides, e.g. polynucleotides encoding NPC proteins for use in the treatment of NPC1 and related disorders. In some embodiments, compositions may be delivered in an adeno-associated viral (AAV) vector. In other embodiments, compositions described herein, may be used to treat a subject in need thereof, such as a human subject diagnosed with NPC1 or other condition resulting from a deficiency in the quantity and/or function of NPC1 protein and/or NPC2 protein, or as a research tool in the study of diseases or conditions in cells or animal models of such disease or condition.

### BACKGROUND

Niemann-Pick Disease describes a class of lysosomal storage diseases (Types A, B, C1, and C2) wherein cellular cholesterol and lipid metabolism and/or storage is impaired, leading to progressive decline in nervous and peripheral tissue function. Niemann-Pick Disease Type C1 (NPC1 or NPC-1) is a rare, fatal neurovisceral disease with a US prevalence estimated at 1 in about 150,000 individuals, with an estimated 500 cases in the US. NPC1 affects neonatal, adolescent, and adult patients with neurological and visceral symptoms, including progressive loss of early motor skills, sudden loss of muscle tone, learning problems, seizure, slurred speech, vertical eye movement difficulties, feeding and swallowing difficulties, hypersensitivity to touch, abdominal enlargement, enlarged spleen or liver, jaundice, unusual shortness of breath, and repeated lung infections. Typically, NPC1 onset presents in school-aged children. NPC1 is currently fatal in all cases, with patients having infant onset dying before age 10 and patients with childhood onset dying before age 20. Adult-onset patients die before 40 years of age. Cause of death in NPC1 patients is often inhalation pneumonia.

NPC1 is caused by autosomal recessive loss of function mutations in one of two proteins: NPC1 (NPC intracellular cholesterol transporter 1, Ensemble gene ID: ENSG00000141458) and NPC2 (ENSG00000119655), a binding partner of NPC1. Over 300 distinct mutations have been identified in NPC1 patients; though approximately 95% of NPC1 patients have mutations in the NPC1 gene. Disease mutations have been identified in nearly all NPC1 protein domains; however, mutations within the sterol-sensing domain and the cysteine-rich loop domain appear to be most pathogenic (Millat, G., et al. Am. J. Hum. Genetics 69.5 (2001): 1013-1021; Pacheco, C. D., and Lieberman, A. P. Exp. Rev. Mol. Med. 10 (2008); the disclosures of each of which are incorporated herein by reference in their entireties). The disease presents with a high degree of heterogeneity among patients.

NPC1 protein is an integral transmembrane protein localized predominantly in late-endosomes and lysosomes. It is required for transport of LDL-derived cholesterol. In the presence of functional NPC1 protein, LDLs are taken up by cells and delivered to lysosomes where their cholesterol esters are cleaved. Free cholesterol is then exported from lysosomes for cellular needs and storage. Defective NPC1 protein leads cell autonomously to intracellular cholesterol accumulation. Thus, NPC1 is characterized as a lysosomal storage disorder since patients are not able to properly metabolize cholesterol and other lipids, leading to their intracellular accumulation. Indeed, vacuolar accumulation of cholesterol in the perinuclear region of patient cells is thought to underly eventual atrophy in frontal lobes, cerebellum, and brainstem as well as hepatomegaly and splenomegaly, all typical of NPC1 disease progression.

Treatments for NPC1 include miglustat and 2-hydroxypropyl-β-cyclodextrin. Miglustat, a glucosylceramide synthase inhibitor, is currently approved for treatment of NPC1 only in the European Union. 2-hydroxypropyl-β-cyclodextrin, a cyclic oligosaccharide that binds and enhances the water solubility of cholesterol, is also useful in treating NPC1. 2-hydroxypropyl-β-cyclodextrin is known to cross the blood-brain barrier (BBB). Both compounds require biweekly intrathecal (IT) injection, and both can effectively delay the onset of neurological signs, ameliorate cerebellar and liver dysfunction, and prolong lifespan in animal models of NPC disease. However, adverse side effects can be severe, and include osmotic diarrhea, outer hair cell death and hearing loss.

Consequently, there remains a medical-need to develop pharmaceutical compositions and methods for the treatment of NPC1 and related disorders and to ameliorate deficiencies of NPC1 and/or NPC2 protein(s) in patients afflicted with NPC-related disorders.

### SUMMARY

The present disclosure addresses the need for providing AAV-based compositions and methods for treating NPC1 deficiency in patients. Disclosed herein are compositions and methods directed to AAV-based gene delivery of NPC1 and/or NPC2 to ameliorate loss-of-function of these genes and to improve intracellular lipid trafficking. The compositions and methods are useful to improve cholesterol and lipid metabolism, and to slow, halt, or reverse NPC1 and related disease progression in afflicted patients.

Accordingly, in one aspect, the present disclosure provides an isolated, e.g., recombinant, vial genome comprising a promoter operably linked to a nucleic acid comprising a transgene encoding an NPC1 protein, wherein the NPC1 protein is encoded by a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750 or 1749.

In another aspect, the present disclosure provides an isolated, e.g., recombinant, vial genome comprising a promoter operably linked to a nucleic acid comprising a transgene encoding an NPC1 protein, wherein the promoter comprises an EF-1a promoter variant, e.g., a truncated EF-1a promoter, which comprises a nucleotide sequence that is less than the full length of the nucleotide sequence of a wild-type (WT) EF-1a promoter comprising SEQ ID NO: 1781 (e.g., at least 10, 20, 25, 30, 50, 100, 150, 200, 250, 300, 350, 400, 450, 500, 550, 600, 650, 700, 750, 800, 850, 900, or 950 nucleotides less), optionally provided that the EF-1a promoter variant does not comprise the nucleotides “GC” at positions 235-236, numbered according to SEQ ID NO: 1781.

In yet another aspect, the present disclosure provides an isolated, e.g., recombinant, vial genome comprising a promoter operably linked to a nucleic acid comprising a transgene encoding an NPC1 protein, wherein the promoter comprises an EF-1a promoter variant comprising [A]-[B]-[C]-[D]-[E], wherein: (i) [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794; or [A] is absent; (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent; (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent; (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798 or [E] is absent.

In some embodiments, the viral genome comprises an internal terminal repeat (ITR) sequence (e.g., an ITR region described herein), an enhancer (e.g., an enhancer described herein), an intron region (e.g., an intron region described herein), a Kozak sequence (e.g., a Kozak sequence described herein), an exon region (e.g., an exon region described herein), and/or a poly A signal region (e.g., a poly A signal sequence described herein). In some embodiments, the viral genome comprises the nucleotide sequence of any one of SEQ ID NOs: 1799-1802, or a nucleotide sequence at least 95% identical thereto.

In yet another aspect, the present disclosure provides an isolated, e.g., recombinant, AAV particle comprising a capsid protein and a viral genome comprising a promoter (e.g., a promoter described herein) operably linked transgene encoding an NPC1 protein described herein. In some embodiments, the capsid protein comprises an AAV capsid protein, e.g., a wild-type AAV capsid protein or a functional variant thereof. In some embodiments, the capsid protein comprises, or is chosen from, an AAV9 capsid protein (e.g., a wild-type AAV9 capsid protein), or a functional variant thereof.

In yet another aspect, the present disclosure provides a method of making a viral genome described herein The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral from the backbone region, e.g., by cleaving the nucleic acid molecule at upstream and downstream of the viral genome.

In yet another aspect, the present disclosure provides a method of making an isolated, e.g., recombinant AAV particle. The method comprising providing a host cell comprising a viral genome described herein and incubating the host cell under conditions suitable to enclose the viral genome in the AAV particle, e.g., an AAV9 capsid protein, thereby making the isolated AAV particle.

In yet another aspect, the present disclosure provides method of delivering an exogenous NPC1 protein, to a subject. The method comprising administering an effective amount of an AAV particle or a plurality of AAV particles, described herein, said AAV particle comprising a viral genome described herein.

In yet another aspect, the present disclosure provides a method of treating a subject having or being diagnosed as having a neurological disorder and/or a disease associated with NPC1 expression. The method comprising administering to the subject an effective amount of an AAV particle or a plurality of AAV particles, described herein, comprising a viral genome described herein. In some embodiments, the disease associated with NPC1 expression comprises a lysosomal storage disease or Niemann-Pick disease, type C1.

In some aspects, the present disclosure provides adeno-associated viral (AAV) vector genomes comprising: a 5′ inverted terminal repeat (ITR), a promoter, a payload region, and a 3′ ITR; wherein the payload region encodes an NPC protein.

The AAV vector genomes can have a nucleotide sequence encoding an amino acid sequence of the NPC protein having at least 80%, at least 85%, at least 90%, at least 95%, or at least 99% sequence identity to an NPC protein as provided in Table 2.

The AAV vector can have a nucleotide sequence encoding an NPC protein having an amino acid sequence of an NPC protein as provided in Table 2.

The AAV vector genomes can have an NPC protein-encoding nucleic acid sequence having at least 80%, at least 85%, at least 90%, at least 95%, or at least 99% sequence identity to a nucleic acid sequence as provided in Table 2, or a fragment thereof.

The AAV vector genomes can have a nucleic acid sequence encoding the NPC protein which comprises SEQ ID NO: 1724 or a fragment thereof.

The AAV vector genomes can encode a cynomolgus (Macaca fascicularis) NPC protein, a rhesus macaque (Macaca mulatta) NPC protein, or an at least partially humanized NPC protein.

The AAV vector genomes can have a 5′ ITR that is an AAV2 ITR.

The AAV vector genomes can have a 5′ ITR that is 130 nucleotides in length.

The AAV vector genomes can have a 3′ ITR that is an AAV2 ITR.

The AAV vector genomes can have a 3′ ITR that is 130 nucleotides in length.

The AAV vector genomes can comprise one or more of (e.g., all of) the following components: a promoter region, a Kozak region, an NPC protein region, or a polyadenylation (polyA) region.

The AAV vector genomes can comprise an ITR to ITR sequence of SEQ ID NO: 1752, SEQ ID NO: 1753, SEQ ID NO: 1754, SEQ ID NO: 1755, or SEQ ID NO: 1756, SEQ ID NO: 1757, SEQ ID NO: 1758, or SEQ ID NO: 1759.

In some aspects, the disclosure provides AAV particles comprising the AAV vector genomes described herein and a capsid.

The AAV particles can have a capsid comprising an amino acid sequence which comprises or which is encoded by a sequence selected from SEQ ID NOs: 1-1261.

Also provided are pharmaceutical compositions comprising the AAV particles described.

In some aspects, the disclosure provides methods of treating a lysosomal storage disorder, said method comprising administering to a subject the pharmaceutical compositions described.

The lysosomal storage disorder can be NPC1 disease or related disorder.

The lysosomal storage disorder can be a disorder associated with decreased NPC (i.e., NPC1 or NPC2) protein levels.

Administration of the pharmaceutical compositions described can result in a 0.5×-3.0× increase in NPC protein expression in a target cell of the subject, as compared to NPC protein expression in an equivalent target cell in a subject without a disorder associated with decreased NPC protein levels.

The methods described can further comprise administering miglustat to the subject.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. An isolated, e.g., recombinant, nucleic acid comprising a transgene encoding an NPC1 protein, wherein the nucleotide sequence encoding the NPC1 protein (e.g., a codon optimized nucleotide sequence) comprises a nucleotide sequence with at least 85% (e.g., at least about 90, 92, 95, 96, 97, 98, or 99%) sequence identity to the nucleotide sequence of SEQ ID NO: 1750 or 1749.
2. An isolated, e.g., recombinant, vial genome comprising a promoter operably linked to a nucleic acid comprising a transgene encoding an NPC1 protein, wherein the nucleotide sequence encoding the NPC1 protein (e.g., a codon optimized nucleotide sequence) comprises a nucleotide sequence with at least 85% (e.g., at least about 90, 92, 95, 96, 97, 98, or 99%) sequence identity to the nucleotide sequence of SEQ ID NO: 1750 or 1749.
3. An isolated, e.g., recombinant, vial genome comprising a promoter operably linked to a nucleic acid comprising a transgene encoding an NPC1 protein, wherein the promoter comprises:
   (a) an EF-1a promoter variant, e.g., a truncated EF-1a promoter, which comprises a nucleotide sequence that is less than the full length of the nucleotide sequence of a wild-type (WT) EF-1a promoter comprising SEQ ID NO: 1781 (e.g., at least 10, 20, 25, 30, 50, 100, 150, 200, 250, 300, 350, 400, 450, 500, 550, 600, 650, 700, 750, 800, 850, 900, or 950 nucleotides less), optionally provided that the EF-1a promoter variant does not comprise the nucleotides “GC” at positions 235-236, numbered according to SEQ ID NO: 1781; or
   (b) an EF-1a promoter variant comprising [A]-[B]-[C]-[D]-[E], wherein:
      (i) [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794; or [A] is absent;
      (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
      (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
      (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and
      (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798 or [E] is absent.
4. The isolated nucleic acid of embodiment 1 or the viral genome of embodiment 2 or 3, wherein the nucleotide sequence encoding the NPC1 protein comprises a nucleotide sequence with at least 85% (e.g., at least about 90, 92, 95, 96, 97, 98, or 99%) sequence identity to the nucleotide sequence of SEQ ID NO: 1750 or 1749.
5. The isolated nucleic acid of embodiment 1 or the viral genome any one of embodiments 2-4, wherein the nucleotide sequence encoding the NPC1 protein comprises a nucleotide sequence with at least 95% sequence identity to the nucleotide sequence of SEQ ID NO: 1750 or 1749.
6. The isolated nucleic acid of embodiment 1 or the viral genome of any one of embodiments 2-5, wherein the nucleotide sequence encoding the NPC1 protein comprises the nucleotide sequence of SEQ ID NO: 1750.
7. The isolated nucleic acid of embodiment 1 or the viral genome of any one of embodiments 2-5, wherein the nucleotide sequence encoding the NPC1 protein comprises the nucleotide sequence of SEQ ID NO: 1749.
8. The viral genome of embodiment 3, wherein the nucleotide sequence encoding the NPC1 protein comprises the nucleotide sequence of SEQ ID NO: 1747, or a nucleotide sequence at least 85% (e.g., at least about 90, 92, 95, 96, 97, 98, or 99%) sequence identity thereto.
9. The isolated nucleic acid of embodiment 1 or the viral genome of any one of embodiments 2-7, wherein the nucleotide sequence encoding the NPC1 protein sequence is codon optimized.
10. The isolated nucleic acid of embodiment 1 or the viral genome of any one of embodiments 2-9, wherein the encoded NPC1 protein comprises the amino acid sequence of SEQ ID NO: 1748, or an amino acid sequence at least 95% identical thereto.
11. The isolated nucleic acid of embodiment 1 or the viral genome of any one of embodiments 2-7 or 9, wherein the encoded NPC1 protein comprises an amino acid sequence encoded by the nucleotide sequence of SEQ ID NO: 1749 or 1750, or a nucleotide sequence at least 85% (e.g., at least about 90, 92, 95, 96, 97, 98, or 99%) identical thereto.
12. The viral genome of any one of embodiments 2-11, wherein the promoter comprises an EF-1a promoter variant, e.g., a truncated EF-1a promoter, which comprises a nucleotide sequence that is less than the full length of a wild-type (WT) EF-1a promoter comprising SEQ ID NO: 1781 (e.g., at least 10, 20, 25, 30, 50, 100, 150, 200, 250, 300, 350, 400, 450, 500, 550, 600, 650, 700, 750, 800, 850, 900, or 950 nucleotides less), optionally provided that the EF-1a promoter variant does not comprise the nucleotides “GC” at positions 235-236, numbered according to SEQ ID NO: 1781.
13. The viral genome of any one of embodiments 3-11, wherein the EF-1a promoter variant comprises a nucleotide sequence that comprises at least 10, 20, 25, 30, 50, 100, 150, 200, 250, 300, 350, 400, 450, 500, 550, 600, 650, 700, 750, 800, 850, 900, or 950 nucleotides less than the full length of a wild-type (WT) EF-1a promoter comprising SEQ ID NO: 1781.
14. The viral genome of any one of embodiments 3-13, wherein the EF-1a promoter variant comprises an intron, e.g., an intron comprising the nucleotide sequence of positions 237-1,184 of SEQ ID NO: 1781.
15. The viral genome of any one of embodiments 3-14, wherein the EF-1a promoter variant comprises a portion of an intron, wherein the portion comprises no more than 5-50 nucleotides, e.g., 5-40 nucleotides, 5-30 nucleotides, 5-20 nucleotides, 5-10 nucleotides, 10-50 nucleotides, 10-40 nucleotides, 10-30 nucleotides, 10-20 nucleotides, 20-50 nucleotides, 20-40 nucleotides, 20-30 nucleotides, 30-50 nucleotides, 30-40 nucleotides, 40-50 nucleotides, 5 nucleotides, 10 nucleotides, 15 nucleotides, 20 nucleotides, 25 nucleotides, 30 nucleotides, 40 nucleotides, or 50 nucleotides of the intron e.g., an intron comprising the nucleotide sequence of positions 237-1,184 of SEQ ID NO: 1781.
16. The viral genome of any one of embodiments 3-15, wherein the EF-1a promoter variant comprises a portion of an intron, wherein the portion comprises the nucleotide sequence of SEQ ID NO: 1798.
17. The viral genome of any one of embodiments 3-13, wherein the EF-1a promoter variant does not comprises an intron, e.g., does not comprise the nucleotide sequence of positions 237-1,184 of SEQ ID NO: 1781.
18. The viral genome of any one of embodiments 2-16, wherein the EF-1a promoter variant comprises nucleotides 1-241 of SEQ ID NO: 1781.
19. The viral genome of any one of embodiments 2-16, wherein the EF-1a promoter variant comprises nucleotides 1-236 of SEQ ID NO: 1781.
20. The viral genome of any one of embodiments 2-16, wherein the EF-1a promoter variant comprises nucleotides 7-236 of SEQ ID NO: 1781.
21. The viral genome of any one of embodiments 2-16 or 20, wherein the EF-1a promoter variant comprises nucleotides 7-241 of SEQ ID NO: 1781.
22. The viral genome of any one of embodiments 2-16, wherein the EF-1a promoter variant comprises nucleotides 13-236 or 13-241 of SEQ ID NO: 1781.
23. The viral genome of any one of embodiments 2-16, wherein the EF-1a promoter variant comprises nucleotides 15-236 or 15-241 of SEQ ID NO: 1781.
24. The viral genome of any one of embodiments 2-23, wherein the EF-1a promoter variant further comprises the nucleotide sequence of SEQ ID NO: 1792, 1793, or 1794, or a sequence having at least one, two, or three but no more than four modifications, e.g., substitutions (e.g., conservative substitutions), relative to the nucleotide sequence of SEQ ID NO: 1792, 1793, or 1794.
25. The viral genome of any one of embodiments 2-24, wherein the EF-1a promoter variant comprises from 5′ to 3′:
   (i) the nucleotide sequence of SEQ ID NO: 1792, or a sequence having at least one or two modifications, e.g., substitutions (e.g., conservative substitutions), relative to the nucleotide sequence of SEQ ID NO: 1792; and
   (ii) nucleotides 1-236 of SEQ ID NO: 1781.
26. The viral genome of any one of embodiments 2-24, wherein the EF-1a promoter variant comprises from 5′ to 3′:
   (i) the nucleotide sequence of SEQ ID NO: 1793, or a sequence having at least one, two, or three but no more than four modifications, e.g., substitutions (e.g., conservative substitutions), relative to the nucleotide sequence of SEQ ID NO: 1793; and
   (ii) nucleotides 1-236 of SEQ ID NO: 1781.
27. The viral genome of any one of embodiments 2-24, wherein the EF-1a promoter variant comprises from 5′ to 3′:
   (i) the nucleotide sequence of SEQ ID NO: 1794, or a sequence having at least one, two, or three but no more than four modifications, e.g., substitutions (e.g., conservative substitutions), relative to the nucleotide sequence of SEQ ID NO: 1794; and
   (ii) nucleotides 1-236 of SEQ ID NO: 1781.
28. The viral genome of any one of embodiments 3-24, wherein the EF-1a promoter variant comprises the nucleotide sequence of SEQ ID NO: 1785, 1787, 1789, or 1791.
29. The viral genome of any one of embodiments 2-11, wherein the promoter comprises an EF-1a promoter variant comprising [A]-[B]-[C]-[D]-[E], wherein:
   (i) [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794; or [A] is absent;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
   (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798 or [E] is absent.
30. The viral genome of any one of embodiments 3-11 or 29, wherein [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794.
31. The viral genome of any one of embodiments 3-11 or 29-30, wherein [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795.
32. The viral genome of any one of embodiments 3-11 or 29-31, wherein [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797.
33. The viral genome of any one of embodiments 3-11 or 29-32, wherein [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781.
34. The viral genome of any one of embodiments 3-11 or 29-33, wherein [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
35. The viral genome of any one of embodiments 3-11 or 29-34, wherein:
   (i) [A] is absent;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
   (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and
   (v) [E] is absent.
36. The viral genome of any one of embodiments 3-11 or 29-34, wherein:
   (i) [A] comprises SEQ ID NO: 1792, or a sequence having at least sequence comprising at least one or two modifications, e.g., substitutions (e.g., conservative substitutions), relative to the nucleotide sequence of SEQ ID NO: 1792;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
   (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and
   (v) [E] is absent.
37. The viral genome of any one of embodiments 3-11 or 29-34, wherein:
   (i) [A] comprises SEQ ID NO: 1793, or a sequence having at least sequence comprising at least one, two, or three but no more than four modifications, e.g., substitutions (e.g., conservative substitutions), relative to the nucleotide sequence of SEQ ID NO: 1793;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
   (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and
   (v) [E] is absent.
38. The viral genome of any one of embodiments 3-11 or 29-34, wherein:
   (i) [A] comprises SEQ ID NO: 1794, or a sequence having at least sequence comprising at least one, two, or three but no more than four modifications, e.g., substitutions (e.g., conservative substitutions), relative to the nucleotide sequence of SEQ ID NO: 1794;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
   (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and
   (v) [E] is absent.
39. The viral genome of any one of embodiments 3-11 or 29-34, wherein the EF-1a promoter variant comprises the nucleotide sequence of SEQ ID NO: 1785, 1787, 1789, or 1791, or a nucleotide sequence having at least 95% sequence identity thereto, provided that the EF-1a promoter variant does not comprise the nucleotides “GC” at positions 235-236, numbered according to SEQ ID NO: 1781.
40. The viral genome of embodiment 2, wherein the promoter comprises ubiquitous promoter or a tissue specific promoter.
41. The viral genome of embodiment 2 or 40, wherein the promoter comprises an EF-1a promoter, a chicken β-actin (CBA) promoter and/or its derivative CAG, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.
42. The viral genome of any one of embodiments 2 or 40-41, wherein the promoter comprises:
   (i) a CMV promoter; or
   (ii) a CMVie enhancer and a CMV promoter, optionally wherein the CMVie enhancer comprises the nucleotide sequence of SEQ ID NO: 1743, or a nucleotide sequence at least 95% identical thereto, and the CMV promoter comprises the nucleotide sequence of SEQ ID NO: 1736, or a nucleotide sequence at least 95% identical thereto.
43. The viral genome of any one of embodiments 2 or 40-42, wherein the promoter comprises the nucleotide sequence of SEQ ID NO: 1736 or 1739-1741, or a nucleotide sequence at least 95% identical thereto.
44. The viral genome of any one of embodiments 2 or 40-43, wherein the promoter comprises the nucleotide sequence of SEQ ID NO: 1736 or a nucleotide sequence at least 95% identical thereto.
45. The viral genome of embodiment 41 or 42, wherein the CMVie enhancer comprises the nucleotide sequence of SEQ ID NO: 1737, 1742, or 1743, or a nucleotide sequence at least 95% identical thereto.
46. The viral genome of any one of embodiments 2 or 40-41, wherein the promoter comprises:
   (i) a CBA promoter; or
   (ii) a CMVie enhancer and a CBA promoter, optionally wherein:
      (a) the CMVie enhancer comprises the nucleotide sequence of SEQ ID NO: 1742, or a nucleotide sequence 95% identical thereto, and the CBA promoter comprises the nucleotide sequence of SEQ ID NO: 1735, or a nucleotide sequence at least 95% identical thereto; or
      (b) the CMVie enhancer comprises the nucleotide sequence of SEQ ID NO: 1737, or a nucleotide sequence 95% identical thereto, and the CBA promoter comprises the nucleotide sequence of SEQ ID NO: 1735, or a nucleotide sequence at least 95% identical thereto.
47. The viral genome of any one of embodiments 2, 40-41, or 46, wherein the promoter comprises the nucleotide sequence of SEQ ID NO: 1735 or 1738, or a nucleotide sequence at least 95% identical thereto.
48. The viral genome of any one of embodiments 2 or 40-41, wherein the promoter comprises an EF-1a promoter or an EF-1a promoter variant.
49. The promoter of embodiment 48, wherein the EF-1a promoter comprises the nucleotide sequence of SEQ ID NO: 1781, or a nucleotide sequence at least 95% identical thereto.
50. The promoter of embodiment 48, wherein the EF-1a promoter variant comprises [A]-[B]-[C]-[D]-[E], wherein:
   (i) [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794; or [A] is absent;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
   (iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822 or 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798 or [E] is absent.
51. The viral genome of embodiment 50, wherein:
   (i) [A] is absent;
   (ii) [B] is absent;
   (iii) [C] is absent;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
52. The viral genome of embodiment 50, wherein:
   (i) [A] is absent;
   (ii) [B] is absent;
   (iii) [C] comprises the nucleotides GT;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
53. The viral genome of embodiment 50, wherein:
   (i) [A] is absent;
   (ii) [B] is absent;
   (iii) [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
54. The viral genome of embodiment 50, wherein:
   (i) [A] is absent;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
55. The viral genome of embodiment 50, wherein:
   (i) [A] is absent;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
   (iv) [D] comprises the nucleotide sequence of SEQ ID NO: 1822, or a nucleotide sequence having at least 90% (e.g., 92, 95, 96, 97, 98, or 99%) sequence identity thereto; and
   (v) [E] is absent.
56. The viral genome of embodiment 50, wherein:
   (i) [A] comprises SEQ ID NO: 1792, or a sequence having at least sequence comprising at least one or two modifications, e.g., substitutions (e.g., conservative substitutions), relative to the nucleotide sequence of SEQ ID NO: 1792;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
57. The viral genome of embodiment 50, wherein:
   (i) [A] comprises SEQ ID NO: 1792, or a sequence having at least sequence comprising at least one or two modifications, e.g., substitutions (e.g., conservative substitutions), relative to the nucleotide sequence of SEQ ID NO: 1792;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1822, or a nucleotide sequence having at least 90% (e.g., 92, 95, 96, 97, 98, or 99%) sequence identity thereto; and
   (v) [E] is absent.
58. The viral genome of embodiment 50, wherein:
   (i) [A] comprises SEQ ID NO: 1793, or a sequence having at least sequence comprising at least one, two, or three but no more than four modifications, e.g., substitutions (e.g., conservative substitutions), relative to the nucleotide sequence of SEQ ID NO: 1793; (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
59. The viral genome of embodiment 50, wherein:
   (i) [A] comprises SEQ ID NO: 1793, or a sequence having at least sequence comprising at least one, two, or three but no more than four modifications, e.g., substitutions (e.g., conservative substitutions), relative to the nucleotide sequence of SEQ ID NO: 1793;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1822, or a nucleotide sequence having at least 90% (e.g., 92, 95, 96, 97, 98, or 99%) sequence identity thereto; and
   (v) [E] is absent.
60. The viral genome of embodiment 50, wherein:
   (i) [A] comprises SEQ ID NO: 1794, or a sequence having at least sequence comprising at least one, two, or three but no more than four modifications, e.g., substitutions (e.g., conservative substitutions), relative to the nucleotide sequence of SEQ ID NO: 1794;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1823, or a nucleotide sequence having at least 90% (e.g., 92, 95, 96, 97, 98, or 99%) sequence identity thereto; and
   (v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.
61. The viral genome of embodiment 50, wherein:
   (i) [A] comprises SEQ ID NO: 1794, or a sequence having at least sequence comprising at least one, two, or three but no more than four modifications, e.g., substitutions (e.g., conservative substitutions), relative to the nucleotide sequence of SEQ ID NO: 1794;
   (ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
   (iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
   (iv) [D] comprises the nucleotide sequence of SEQ ID NO: 1822, or a nucleotide sequence having at least 90% (e.g., 92, 95, 96, 97, 98, or 99%) sequence identity thereto; and
   (v) [E] is absent.
62. The viral genome of any one of embodiments 2 or 40-41, wherein the promoter comprises an EF-1a promoter variant, e.g., a truncated EF-1a promoter, which comprises the nucleotide sequence of the WT EF-1a promoter comprising SEQ ID NO: 1781 and a deletion (e.g., a deletion of at least 10, 20, 25, 30, 50, 100, 150, 200, 250, 300, 350, 400, 450, 500, 550, 600, 650, 700, 750, 800, 850, 900, or 950 nucleotides).
63. The viral genome of embodiment 62, wherein the deletion comprises at least 10, 20, 25, 30, 50, 100, 150, 200, 250, 300, 350, 400, 450, 500, 550, 600, 650, 700, 750, 800, 850, 900, or 950 nucleotides.
64. The viral genome of embodiment 62 or 63, wherein the deletion is from the 3′ end of the WT EF-1a promoter, the 5′ end of the WT EF-1a promoter, both.
65. The viral genome of any one of embodiments 2 or 40-41, wherein the promoter comprises an EF-1a promoter variant, e.g., an EF-1a truncated promoter, comprises the nucleotide sequence of SEQ ID NO: 1781 or a sequence with at least 95% identity thereto, with a fragment of the WT EF-1a promoter deleted, optionally wherein the fragment comprises nucleotides 237-1,184 of SEQ ID NO: 1781 or nucleotides 242-1,184 of SEQ ID NO: 1781.
66. The viral genome of any one of embodiments 62-65, wherein the EF-1a promoter variant further comprises the nucleotide sequence of SEQ ID NO: 1792, 1793, or 1794, or a sequence having at least one, two, or three but no more than four modifications, e.g., substitutions (e.g., conservative substitutions), relative to the nucleotide sequence of SEQ ID NO: 1792, 1793, or 1794.
67. The viral genome of any one of embodiments 2, 40-41, or 48-66, wherein the promoter comprises the nucleotide sequence of SEQ ID NO: 1785, 1782-1784, 1786-1791, or a sequence at least 95% identical thereto.
68. The viral genome of any one of embodiments 2, 40-41, 48-50, 55, or 62-66, wherein the promoter comprises the nucleotide sequence of SEQ ID NO: 1785, or a sequence at least 95% identical thereto.
69. The viral genome of any one of embodiments 2-68, which further comprises an inverted terminal repeat (ITR) sequence.
70. The viral genome of any one of embodiments 2-69, which comprises an ITR sequence positioned 5′ relative to the transgene encoding the NPC1 protein.
71. The viral genome of any one of embodiments 2-70, which comprises an ITR sequence positioned 3′ relative to the transgene encoding the NPC1 protein.
72. The viral genome of any one of embodiments 2-71, which comprises an ITR sequence positioned 5′ relative to the transgene encoding the NPC1 protein and an ITR sequence positioned 3′ relative to the transgene encoding the NPC1 protein.
73. The viral genome of any one of embodiments 69-72, wherein the ITR comprises a nucleotide sequence of SEQ ID NO: 1733, 1734, 1837, or 1838 or a nucleotide sequence at least 95% identical thereto.
74. The viral genome of any one of embodiments 69-73, wherein:
   (i) the ITR sequence positioned 5′ relative to the transgene encoding the NPC1 protein comprises the nucleotide sequence of SEQ ID NO: 1733, or a nucleotide sequence with at least 95% sequence identity thereto; and/or the ITR sequence positioned 3′ relative to the transgene encoding the NPC1 protein comprises the nucleotide sequence of SEQ ID NO: 1734, or a nucleotide sequence with at least 95% sequence identity thereto; or
   (ii) the ITR sequence positioned 5′ relative to the transgene encoding the NPC1 protein comprises the nucleotide sequence of SEQ ID NO: 1837, or a nucleotide sequence with at least 95% sequence identity thereto; and/or the ITR sequence positioned 3′ relative to the transgene encoding the NPC1 protein comprises the nucleotide sequence of SEQ ID NO: 1838, or a nucleotide sequence with at least 95% sequence identity thereto.
75. The viral genome of any one of embodiments 2-74, which further comprises a polyadenylation (polyA) signal region.
76. The viral genome of embodiment 75, wherein the polyA signal region comprises the nucleotide sequence of SEQ ID NO: 1751, or a nucleotide sequence at least 95% identical thereto.
77. The viral genome of any one of embodiments 2-76, which further comprises an intron region.
78. The viral genome of embodiment 77, wherein the intron region comprises the nucleotide sequence of SEQ ID NO: 1780, or a nucleotide sequence at least 95% identical thereto.
79. The viral genome of any one of embodiments 2-78, which further comprises an exon region, e.g., at least one, two, or three exon regions.
80. The viral genome of any one of embodiments 2-79, which further comprises a Kozak sequence.
81. The viral genome of embodiment 80, wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746.
82. The viral genome of any one of embodiments 2-81, which further comprises a nucleotide sequence encoding a miR binding site, e.g., a miR binding site that modulates, e.g., reduces, expression of the payload encoded by the viral genome in a cell or tissue where the corresponding miRNA is expressed.
83. The viral genome of embodiment 82, wherein the encoded miRNA binding site is complementary, e.g., fully complementary or partially complementary, to a miRNA expressed in a cell or tissue of the DRG, liver, hematopoietic, or a combination thereof.
84. The viral genome of embodiment 82 or 83, which comprises at least 1-5 copies of an encoded miR binding site, e.g., at least 1, 2, 3, 4, or 5 copies.
85. The viral genome of any one of embodiments 82-84, which comprises at least 3 copies of an encoded miR binding sites, optionally wherein all three copies comprise the same miR binding site, or at least one, two, or all of the copies comprise a different miR binding site.
86. The viral genome of embodiment 85, wherein the 3 copies of the encoded miR binding sites are continuous (e.g., not separated by a spacer), or are separated by a spacer, optionally wherein the spacer comprises the nucleotide sequence of SEQ ID NO: 1846, or a nucleotide sequence having at least one, two, or three modifications, but no more than four modifications of SEQ ID NO: 1846.
87. The viral genome of any one of embodiments 82-84, which comprises at least 4 copies of an encoded miR binding site, optionally wherein all four copies comprise the same miR binding site, or at least one, two, three, or all of the copies comprise a different miR binding site.
88. The viral genome of embodiment 87, wherein the 4 copies of the encoded miR binding sites are continuous (e.g., not separated by a spacer), or are separated by a spacer, optionally wherein the spacer comprises the nucleotide sequence of SEQ ID NO: 1846, or a nucleotide sequence having at least one, two, or three modifications, but no more than four modifications of SEQ ID NO: 1846.
89. The viral genome of any one of embodiments 82-88, wherein the encoded miR binding site comprises a miR122 binding site, a miR183 binding site, a miR-142-3p, or a combination thereof, optionally wherein:
   (i) the encoded miR122 binding site comprises the nucleotide sequence of SEQ ID NO: 1840, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1840;
   (ii) the encoded miR183 binding site comprises the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843; and/or
   (iii) the encoded miR-142-3p binding site comprises the nucleotide sequence of SEQ ID NO: 1842, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1842.
90. The viral genome of any one of embodiments 2-89, wherein the viral genome comprises an encoded miR183 binding site.
91. The viral genome of any one of embodiments 2-90, wherein the viral genome comprises at least 1-5 copies, e.g., 1, 2, or 3 copies of a miR183 binding site, optionally wherein each copy is continuous (e.g., not separated by a spacer), or each copy is separated by a spacer, optionally wherein the spacer comprises the nucleotide sequence of SEQ ID NO: 1846, or a nucleotide sequence having at least one, two, or three modifications, but no more than four modifications of SEQ ID NO: 1846.
92. The viral genome of embodiment 90 or 91, wherein the encoded miR183 binding site comprises the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843.
93. The viral genome of any one of embodiments 2-93, wherein the viral genome comprises:
(A) (i) a first encoded miR183 binding site comprising the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843;
   (ii) a first spacer comprising the nucleotide sequence of SEQ ID NO: 1846, or a nucleotide sequence having at least one, two, or three modifications, but no more than four modifications of SEQ ID NO: 1846; and
   (iii) a second encoded miR183 binding site comprising the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843; or
(B) (i) a first encoded miR183 binding site comprising the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843;
   (ii) a first spacer comprising the nucleotide sequence of SEQ ID NO: 1846, or a nucleotide sequence having at least one, two, or three modifications, but no more than four modifications of SEQ ID NO: 1846;
   (iii) a second encoded miR183 binding site comprising the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843;
   (iv) a second spacer comprising the nucleotide sequence of SEQ ID NO: 1846, or a nucleotide sequence having at least one, two, or three modifications, but no more than four modifications of SEQ ID NO: 1846; and
   (v) a third encoded miR183 binding site comprising the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843.
94. The viral genome of any one of embodiments 2-93, which is single stranded.
95. A viral genome comprising in 5′ to 3′ order:
   (i) a 5′ adeno-associated (AAV) ITR, optionally wherein the 5′ AAV ITR comprises the nucleotide sequence of SEQ ID NO: 1733, or a nucleotide sequence at least 95% identical thereto;
   (ii) an EF-1a promoter variant, optionally wherein the EF-1a promoter variant comprises the nucleotide sequence of SEQ ID NO: 1785, or a nucleotide sequence at least 95% identical thereto;
   (iii) a Kozak sequence, optionally wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746;
   (iv) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
   (v) a polyA signal region, optionally wherein the polyA signal region comprises the nucleotide sequence of SEQ ID NO: 1751, or a nucleotide sequence at least 95% identical thereto; and
   (vi) a 3′ AAV ITR, optionally wherein the 3′ AAV ITR comprises the nucleotide sequence of SEQ ID NO: 1734, or a nucleotide sequence at least 95% identical thereto.
96. A viral genome comprising in 5′ to 3′ order:
   (i) a 5′ adeno-associated (AAV) ITR, optionally wherein the 5′ AAV ITR comprises the nucleotide sequence of SEQ ID NO: 1733, or a nucleotide sequence at least 95% identical thereto;
   (ii) an EF-1a promoter variant, optionally wherein the EF-1a promoter variant comprises the nucleotide sequence of SEQ ID NO: 1785, or a nucleotide sequence at least 95% identical thereto;
   (iii) an intron region, optionally wherein the intron region comprises the nucleotide sequence of SEQ ID NO: 1780, or a nucleotide sequence at least 95% identical thereto;
   (iv) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
   (v) a polyA signal region, optionally wherein the polyA signal region comprises the nucleotide sequence of SEQ ID NO: 1751, or a nucleotide sequence at least 95% identical thereto; and
   (vi) a 3′ AAV ITR, optionally wherein the 3′ AAV ITR comprises the nucleotide sequence of SEQ ID NO: 1734, or a nucleotide sequence at least 95% identical thereto.
97. A viral genome comprising in 5′ to 3′ order:
   (i) a 5′ adeno-associated (AAV) ITR, optionally wherein the 5′ AAV ITR comprises the nucleotide sequence of SEQ ID NO: 1733, or a nucleotide sequence at least 95% identical thereto;
   (ii) a CMV promoter variant, optionally wherein the CMV promoter comprises the nucleotide sequence of SEQ ID NO: 1736, or a nucleotide sequence at least 95% identical thereto;
   (iii) a Kozak sequence, optionally wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746;
   (iv) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
   (v) a polyA signal region, optionally wherein the polyA signal region comprises the nucleotide sequence of SEQ ID NO: 1751, or a nucleotide sequence at least 95% identical thereto; and
   (vi) a 3′ AAV ITR, optionally wherein the 3′ AAV ITR comprises the nucleotide sequence of SEQ ID NO: 1734, or a nucleotide sequence at least 95% identical thereto.
98. A viral genome comprising in 5′ to 3′ order:
   (i) a 5′ adeno-associated (AAV) ITR, optionally wherein the 5′ AAV ITR comprises the nucleotide sequence of SEQ ID NO: 1733, or a nucleotide sequence at least 95% identical thereto;
   (ii) a CMV promoter variant, optionally wherein the CMV promoter comprises the nucleotide sequence of SEQ ID NO: 1736, or a nucleotide sequence at least 95% identical thereto;
   (iii) an intron region, optionally wherein the intron region comprises the nucleotide sequence of SEQ ID NO: 1780, or a nucleotide sequence at least 95% identical thereto;
   (iv) a Kozak sequence, optionally wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746;
   (v) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
   (vi) a polyA signal region, optionally wherein the polyA signal region comprises the nucleotide sequence of SEQ ID NO: 1751, or a nucleotide sequence at least 95% identical thereto; and
   (vii) a 3′ AAV ITR, optionally wherein the 3′ AAV ITR comprises the nucleotide sequence of SEQ ID NO: 1734, or a nucleotide sequence at least 95% identical thereto.
99. The viral genome of any one of embodiments 2-95, which comprises the nucleotide sequence of SEQ ID NO: 1799, or a nucleotide sequence at least 95% identical thereto.
100. The viral genome of any one of embodiments 2-94 or 96, which comprises the nucleotide sequence of SEQ ID NO: 1800, or a nucleotide sequence at least 95% identical thereto.
101. The viral genome of any one of embodiments 2-94 or 97, which comprises the nucleotide sequence of SEQ ID NO: 1801, or a nucleotide sequence at least 95% identical thereto.
102. The viral genome of any one of embodiments 2-94 or 98, which comprises the nucleotide sequence of SEQ ID NO: 1802, or a nucleotide sequence at least 95% identical thereto.
103. The viral genome of any one of embodiments 2, 4-7, 9-11, 12-94, which comprises the nucleotide sequence of SEQ ID NO: 1755, 1757, 1803-1087, 1810-1812, 1815-1816, 1824, 1827, 1828, or 1830-1831, or a nucleotide sequence at least 95% identical thereto.
104. The viral genome of any one of embodiments 3-94, which comprises the nucleotide sequence of SEQ ID NO: 1814-1821, 1825-1828, or a nucleotide sequence at least 95% identical thereto.
105. An isolated, e.g., recombinant, vial genome comprising a promoter operably linked to a nucleic acid comprising a transgene encoding an NPC1 protein, wherein:
   (i) the promoter is chosen from a CMV promoter, CBA promoter, or functional variant thereof, optionally wherein the CMV promoter or functional variant thereof is chosen from any one of SEQ ID NOs: 1736 or 1739-1741, and/or the CBA promoter, or functional variant thereof is chosen from SEQ ID NO: 1735 or 1738; and
   (ii) the nucleotide sequence encoding the NPC1 protein comprises the nucleotide sequence of SEQ ID NO: 1747, or a nucleotide sequence at least 85% (e.g., 90, 92, 95, 96, 97, 98, 99%) identical thereto.
106. An isolated, e.g., recombinant, vial genome comprising a promoter operably linked to a nucleic acid comprising a transgene encoding an NPC1 protein, wherein:
   (i) the promoter is chosen from a CMV promoter, CBA promoter, or functional variant thereof, optionally wherein the CMV promoter or functional variant thereof is chosen from any one of SEQ ID NOs: 1736 or 1739-1741, and/or the CBA promoter, or functional variant thereof is chosen from SEQ ID NO: 1735 or 1738; and
   (ii) the nucleotide sequence encoding the NPC1 protein comprises the nucleotide sequence of SEQ ID NO: 1747, 1749, or 1750, or a nucleotide sequence at least 85% (e.g., 90, 92, 95, 96, 97, 98, 99%) identical thereto.
107. The viral genome of embodiment 105, wherein the viral genome comprises the nucleotide sequence of any of SEQ ID NOs: 1752, 1756, 1758-1759, 1808-1809, 1829, or 1833-1836, or a nucleotide sequence at least 95% identical thereto.
108. An isolated, e.g., recombinant, vial genome comprising the nucleotide sequence of any of SEQ ID NOs: 1753-1754, 1813, or 1832, or a nucleotide sequence at least 95% identical thereto.
109. The viral genome of any one of embodiments 2-108, which further comprises a nucleic acid encoding a capsid protein, e.g., a structural protein, wherein the capsid protein comprises a VP1 polypeptide, a VP2 polypeptide, and/or a VP3 polypeptide.
110. The viral genome of embodiment 109, wherein the VP1 polypeptide, the VP2 polypeptide, and/or the VP3 polypeptide are encoded by at least one Cap gene.
111. The viral genome of any one of embodiments 2-110, which further comprises a nucleic acid encoding a Rep protein, e.g., a non-structural protein, wherein the Rep protein comprises a Rep78 protein, a Rep68, Rep52 protein, and/or a Rep40 protein.
112. The viral genome of embodiment 111, wherein the Rep78 protein, the Rep68 protein, the Rep52 protein, and/or the Rep40 protein are encoded by at least one Rep gene.
113. An isolated, e.g., recombinant, NPC1 protein encoded by the viral genome of any one of embodiments 2-112 or the isolated nucleic acid of embodiment 1.
114. An isolated, e.g., recombinant, AAV particle comprising:
   (i) a capsid protein; and
   (ii) the viral genome of any one of embodiments 2-112.
115. The AAV particle of embodiment 114, wherein:
   (i) the capsid protein comprises the amino acid sequence of SEQ ID NO: 138, or an amino acid sequence with at least 80% (e.g., at least about 85, 90, 95, 96, 97, 98, or 99%) sequence identity thereto;
   (ii) the capsid protein comprises an amino acid sequence having at least one, two or three modifications but not more than 30, 20 or 10 modifications of the amino acid sequence of SEQ ID NO: 138;
   (iii) the capsid protein comprises the amino acid sequence of SEQ ID NO: 11, or an amino acid sequence with at least 80% (e.g., at least about 85, 90, 95, 96, 97, 98, or 99%) sequence identity thereto;
   (iv) the capsid protein comprises an amino acid sequence having at least one, two or three modifications but not more than 30, 20 or 10 modifications of the amino acid sequence of SEQ ID NO: 11;
   (v) the capsid protein comprises an amino acid sequence encoded by the nucleotide sequence of SEQ ID NO: 137, or a sequence with at least 80% (e.g., at least about 85, 90, 95, 96, 97, 98, or 99%) sequence identity thereto; and/or
   (vi) the nucleotide sequence encoding the capsid protein comprises the nucleotide sequence of SEQ ID NO: 137, or a sequence with at least 80% (e.g., at least about 85, 90, 95, 96, 97, 98, or 99%) sequence identity thereto.
116. The AAV particle of embodiment 114 or 115, wherein the capsid protein comprises:
   (i) an amino acid substitution at position K449, e.g., a K449R substitution, numbered according to SEQ ID NO:138;
   (ii) an insert comprising the amino acid sequence of TLAVPFK (SEQ ID NO: 1262), optionally wherein the insert is present immediately subsequent to position 588, relative to a reference sequence numbered according to SEQ ID NO:138;
   (iii) an amino acid other than “A” at position 587 and/or an amino acid other than “Q” at position 588, numbered according to SEQ ID NO: 138;
   (iv) the amino acid substitution of A587D and/or Q588G, numbered according to SEQ ID NO:138.
117. The AAV particle of any one of embodiments 114-116, wherein the capsid protein comprises (i) the amino acid substitution of K449R numbered according to SEQ ID NO:138; and (ii) an insert comprising the amino acid sequence of TLAVPFK (SEQ ID NO: 1262), optionally wherein the insert is present immediately subsequent to position 588 of SEQ ID NO:138.
118. The AAV particle of any one of embodiments 114-116, wherein the capsid protein comprises (i) the amino acid substitution of K449R numbered according to SEQ ID NO:138; (ii) an insert comprising the amino acid sequence of TLAVPFK (SEQ ID NO: 1262), optionally wherein the insert is present immediately subsequent to position 588, relative to a reference sequence numbered according to SEQ ID NO:138; and (iii) the amino acid substitutions of A587D and Q588G, numbered according to SEQ ID NO:138.
119. The AAV particle of any one of embodiments 114-116, wherein the capsid protein comprises (i) an insert comprising the amino acid sequence of TLAVPFK (SEQ ID NO: 1262), optionally wherein the insert is present immediately subsequent to position 588, relative to a reference sequence numbered according to SEQ ID NO:138; and (ii) the amino acid substitutions of A587D and Q588G, numbered according to SEQ ID NO:138.
120. The AAV particle of any one of embodiments 114-119, wherein the capsid protein comprises any of the capsid proteins listed in Table 1 or a functional variant thereof.
121. The AAV particle of any one of embodiments 114-120, wherein the capsid protein comprises a VOY101, VOY201, AAVPHP.N (PHP.N), AAVPHP.B (PHP.B), AAVPHP.A (PHP.A), PHP.B2, PHP.B3, G2B4, G2B5, AAV9, AAVrh10, or a functional variant thereof.
122. The AAV particle of embodiment 114-121, wherein:
   (i) the capsid protein comprises the amino acid sequence of SEQ ID NO: 1, or an amino acid sequence substantially identical (e.g., having at least 70%, 75%, 80%, 85%, 90%, 92%, 95%, 97%, 98%, or 99% sequence identity) thereto;
   (ii) the capsid protein comprises an amino acid sequence comprising at least one, two, or three modifications but no more than 30, 20, or 10 modifications, e.g., substitutions, relative to the amino acid sequence of SEQ ID NO: 1;
   (iii) the capsid protein comprises an amino acid sequence encoded by the nucleotide sequence of SEQ ID NO: 2 or a nucleotide sequence substantially identical (e.g., having at least 70%, 75%, 80%, 85%, 90%, 92%, 95%, 97%, 98%, or 99% sequence identity) thereto; and/or
   (iv) the nucleotide sequence encoding the capsid protein comprises the nucleotide sequence of SEQ ID NO: 2, or a nucleotide sequence substantially identical (e.g., having at least 70%, 75%, 80%, 85%, 90%, 92%, 95%, 97%, 98%, or 99% sequence identity) thereto.
123. The AAV particle of any one of embodiments 114-116 or 120-121, wherein the capsid protein comprises:
   (i) a VP1 polypeptide, VP2 polypeptide, VP3 polypeptide, or a combination thereof;
   (ii) the amino acid sequence corresponding to positions 138-736, e.g., a VP2, of SEQ ID NO: 138, or a sequence with at least 80% (e.g., at least about 85, 90, 92, 95, 96, 97, 98, or 99%) sequence identity thereto;
   (iii) the amino acid sequence corresponding to positions 203-736, e.g., a VP3, of SEQ ID NO: 138, or a sequence with at least 80% (e.g., at least about 85, 90, 92, 95, 96, 97, 98, or 99%) sequence identity thereto; and/or
   (iv) the amino acid sequence corresponding to positions 1-736, e.g., a VP1, of SEQ ID NO: 138, or a sequence with at least 80% (e.g., at least about 85, 90, 92, 95, 96, 97, 98, or 99%) sequence identity thereto.
124. The AAV particle of any one of embodiments 114-116, 120-121, or 123, wherein the nucleotide sequence encoding the capsid protein comprises:
   (i) a CTG initiation codon; and/or
   (ii) the nucleotide sequence of SEQ ID NO: 137 which comprises 3-20 mutations, e.g., substitutions, e.g., 3-15 mutations, 3-10 mutations, 3-5 mutations, 5-20 mutations, 5-15 mutations, 5-10 mutations, 10-20 mutations, 10-15 mutations, 15-20 mutations, 3 mutations, 5 mutations, 10 mutations, 12 mutations, 15 mutations, 18 mutations, or 20 mutations.
125. A vector comprising the viral genome of any one of embodiments 2-112 or the nucleic acid of embodiment 1.
126. A cell comprising the viral genome of any one of embodiments 2-112, the viral particle of any one of embodiments 114-124, or the vector of embodiment 125.
127. The cell of embodiment 126, which a mammalian cell, e.g., an HEK293 cell, an insect cell, e.g., an Sf9 cell, or a bacterial cell.
128. A nucleic acid comprising the viral genome of any one of embodiments 2-112 or the isolated nucleic acid of embodiment 1, and a backbone region suitable for replication of the viral genome in a cell, e.g., a bacterial cell (e.g., wherein the backbone region comprises one or both of a bacterial origin of replication and a selectable marker).
129. The nucleic acid of embodiment 128, wherein the viral genome comprises a nucleotide sequence of any one of SEQ ID NOs: 1799-1082, 1752-1759, 1803-1821, or 1824-1830.
130. A method of making a viral genome, the method comprising:
   (i) providing the nucleic acid molecule comprising the viral genome embodiment 128 or 129 or a nucleic acid encoding the viral genome of any one of embodiments 2-112; and
   (ii) excising the viral genome from the backbone region, e.g., by cleaving the nucleic acid molecule at upstream and downstream of the viral genome.
131. A method of making an isolated, e.g., recombinant, AAV particle, the method comprising
   (i) providing a host cell comprising the viral genome of any one of embodiments 2-112 or the nucleic acid encoding the viral genome of embodiment 128 or 129; and
   (ii) incubating the host cell under conditions suitable to enclose the viral genome in a capsid protein, e.g., an AAV9 capsid protein;
   thereby making the isolated AAV particle.
132. The method of embodiment 131, further comprising, prior to step (i), introducing a first nucleic acid molecule comprising the viral genome into the host cell.
133. The method of embodiment 131 or 132, wherein the host cell comprises a second nucleic acid encoding a capsid protein, e.g., an AAV9 capsid protein.
134. The method of embodiment 133, further comprising introducing the second nucleic acid into the cell.
135. The method of any of embodiments 131-134, wherein the second nucleic acid molecule is introduced into the host cell prior to, concurrently with, or after the first nucleic acid molecule.
136. The method of any one of embodiments 131-135, wherein the host cell comprises a mammalian cell, e.g., an HEK293 cell, an insect cell, e.g., an Sf9 cell, or a bacterial cell.
137. A pharmaceutical composition comprising the AAV particle of any one of embodiments 114-124, or an AAV particle comprising the viral genome of any one of embodiments 2-112, and a pharmaceutically acceptable excipient.
138. A method of delivering an exogenous NPC1 protein to a subject, comprising administering an effective amount of the pharmaceutical composition of embodiment 137, the AAV particle of any one of embodiments 114-124, or an AAV particle comprising the viral genome of any one of embodiments 2-112.
139. The method of embodiment 138, wherein the subject has, has been diagnosed with having, or is at risk of having a disease associated with expression of NPC1, e.g., aberrant or reduced NPC1 expression, e.g., expression of an NPC1 gene, NPC1 mRNA, and/or NPC1 protein.
140. The method of embodiment 138 or 139, wherein the subject has, has been diagnosed with having, or is at risk of having a lysosomal storage disease or Niemann-Pick disease, type C1.
141. A method of treating a subject having or diagnosed with having a disease associated with NPC1 expression comprising administering to the subject an effective amount of the pharmaceutical composition of embodiment 137, the AAV particle of any one of embodiments 114-124, or an AAV particle comprising the viral genome of any one of embodiments 2-112, thereby treating the disease associated with NPC1 expression in the subject.
142. A method of treating a subject having or diagnosed with having a lysosomal storage disease, comprising administering to the subject an effective amount of the pharmaceutical composition of embodiment 137, the AAV particle of any one of embodiments 114-124, or an AAV particle comprising the viral genome of any one of embodiments 2-112, thereby treating the lysosomal storage disease in the subject.
143. The method of embodiment 141 or 142, wherein the disease associated with NPC1 expression or the lysosomal storage disease is Niemann-Pick disease, type C1.
144. The method of embodiment 143, wherein the Niemann-Pick disease, type C1 is neonate onset Niemann-Pick disease, type C1 or juvenile onset Niemann-Pick disease, type C1.
145. A method of treating a subject having or diagnosed with having a Niemann-Pick disease, type C1 comprising administering to the subject an effective amount of the pharmaceutical composition of embodiment 137, the AAV particle of any one of embodiments 114-124, or an AAV particle comprising the viral genome of any one of embodiments 2-112, thereby treating the Niemann-Pick disease, type C1 in the subject.
146. The method of any one of embodiments 141-145, wherein treating comprises prevention of progression of the disease in the subject.
147. The method of any one of embodiments 141-146, wherein treating results in amelioration of at least one symptom of the disease associated with NPC1 expression, the lysosomal storage disease, and/or Niemann-Pick disease, type C1 in the subject.
148. The method of embodiment 147, wherein the symptom of the disease associated with NPC1 expression, the lysosomal storage disease, and/or Niemann-Pick disease, type C1 in the subject comprises accumulation of unesterified cholesterol, impaired cholesterol metabolism, cognitive impairment, muscular impairment, physical impairment, sensory impairment, delayed development (e.g., delayed onset of developmental milestones), impaired brain function and/or resting brain function, reduced body max, aberrant levels of neurodegeneration biomarkers (e.g., biofluid (plasma/CSF) markers of neurodegeneration (e.g., NfL)), seizures, and/or reduced brain volume.
149. The method of any one of embodiments 138-124, wherein the subject is a human.
150. The method of any one of embodiments 138-125, wherein the subject comprises a mutation in the NPC1 gene, NPC1 mRNA, and/or NPC1 protein.
151. The method of any one of embodiments 138-150, wherein the subject is a perinatal or neonatal subject, e.g., between 0 to 3 months of age.
152. The method of any one of embodiments 138-150, wherein the subject is between 3 months to 2 years of age, e.g., an early infantile subject.
153. The method of any one of embodiments 138-150, wherein the subject is between 2 years of age to 6 years of age, e.g., a late infantile subject.
154. The method of any one of embodiments 138-150, wherein the subject is between 6 years of age to 15 years of age, e.g., a juvenile.
155. The method of any one of embodiments 138-150, wherein the subject is above 15 years of age, e.g., an adult.
156. The method of any one of embodiments 138-155, wherein the AAV particle is administered to the subject intramuscularly, intravenously, intracerebrally, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM).
157. The method of any one of embodiments 138-156, wherein the AAV particle is administered to the subject via intravenous administration, optionally wherein the intravenous administration is via focused ultrasound (FUS), e.g., coupled with the intravenous administration of microbubbles (FUS-MB), or MRI-guided FUS coupled with intravenous administration.
158. The method of any one of embodiments 138-157, wherein the AAV particle is administered to a cell, tissue, or region of the CNS, e.g., a region of the brain, e.g., the parenchyma, the cortex, cerebellum, corpus callosum, brain stem caudate-putamen, thalamus, superior colliculus, or a combination thereof.
159. The method of any one of embodiments 138-158, further comprising performing a blood test, an imaging test, a CNS biopsy sample, or an aqueous cerebral spinal fluid biopsy.
160. The method of any one of embodiments 138-159, which further comprises evaluating, e.g., measuring, the level of NPC1 expression, e.g., NPC1 gene, NPC1 mRNA, and/or NPC1 protein expression in a subject, e.g., in a cell or tissue of the subject, optionally wherein the level of NPC1 protein is measured by an assay described herein, e.g., an ELISA, a Western blot, or an immunohistochemistry assay.
161. The method of embodiment 160, wherein measuring the level of NPC1 expression is performed prior to, during, or subsequent to treatment with the AAV particle.
162. The method of embodiment 160 or 161, wherein the cell is a cell of the central nervous system (e.g., parenchyma).
163. The method of any one of embodiments 138-162, wherein the administration results in increased level of NPC1 protein expression (e.g., 2-fold, 3-fold, 4-fold, 5-fold, 6-fold, 7-fold, 8-fold, 9-fold, 10-fold, 15-fold, 20-fold more NPC protein expression) in a cell of the subject (e.g., a cell of the CNS, e.g., a cell of the cortex, hippocampus, cerebellum, or brainstem), relative to reference level, e.g., a subject that has not received treatment, e.g., has not been administered the AAV particle.
164. The method of any one of embodiments 138-163, wherein administration results in a reduction of cholesterol accumulation in CNS cells (e.g. as measured by filipin staining, HP-β-Calbindin D staining, mass spectrometry, and quantification), as compared to a reference level, e.g., a subject that has not received treatment, e.g., has not been administered the AAV particle.
165. The method of any one of embodiments 138-164, wherein administration results in improved results in percentage of Motor Milestones Responders on Hammersmith Infant Neurological Examination (HINE) assessment, improved rate of slowing or normalization of age appropriate development milestones, improved Child Brain Function including Resting Brain Function (Alpha, Gamma and Theta power, Decreased Seizure count), improved Body Mass Index, improved readouts of neurodegeneration biomarkers including biofluid (plasma/CSF) markers of neurodegeneration (e.g., NfL), improved neurologic and/or functional readouts such as Inventory of Non-Ataxia Signs (INAS), SCA Functional Index or SARA, or a combination thereof in the subject, as compared to a reference level, e.g., a subject that has not received treatment, e.g., has not been administered the AAV particle.
166. The method of any one of embodiments 138-165, further comprising administration of an additional therapeutic agent and/or therapy suitable for treatment or prevention of the disease associated with NPC1 expression, the lysosomal storage disease, and/or Niemann-Pick disease, type C1.
167. The method of embodiment 166, wherein the additional therapeutic agent and/or therapy comprises TRAPPSOL CYCLO, VTS-270 (e.g., a 2-hydroxypropyl-β-cyclodextrin (HPβCD) mixture), arimoclomol (e.g., arimoclomol citrate), or a combination thereof.
168. The isolated viral genome of any one of embodiments 2-112, the AAV particle of any one of embodiments 114-124, or the pharmaceutical composition of embodiment 137, for use in the manufacture of a medicament.
169. The isolated viral genome of any one of embodiments 2-112, the AAV particle of any one of embodiments 114-124, or the pharmaceutical composition of embodiment 137, for use in the treatment of a disease associated with NPC1 expression, the lysosomal storage disease, and/or Niemann-Pick disease, type C1.
170. Use of an effective amount of an AAV particle comprising the genome of any one of embodiments 2-112, the AAV particle of any one of embodiments 114-124, or the pharmaceutical composition of embodiment 137, in the manufacture of a medicament for the treatment of a disease associated with NPC1 expression, the lysosomal storage disease, and/or Niemann-Pick disease, type C1.
171. An adeno-associated viral (AAV) vector genome comprising: a 5′ inverted terminal repeat (ITR), a promoter, a payload region, and a 3′ ITR; wherein the payload region encodes an NPC protein.
172. The AAV vector genome of embodiment 171, wherein the NPC protein is a human (Homo sapiens) NPC protein.
173. The AAV vector genome of embodiment 171, wherein the amino acid sequence of the NPC protein has at least 80%, at least 85%, at least 90%, at least 95%, or at least 99% sequence identity to an NPC protein as provided in Table 2.
174. The AAV vector genome of embodiment 171, wherein the NPC protein has an amino acid sequence of an NPC protein as provided in Table 2.
175. The AAV vector genome of embodiment 171, wherein the nucleic acid sequence encoding the NPC protein has at least 80%, at least 85%, at least 90%, at least 95%, or at least 99% sequence identity to a nucleic acid sequence as provided in Table 2, or a fragment thereof.
176. The AAV vector genome of embodiment 171, wherein the nucleic acid sequence encoding the NPC protein comprises SEQ ID NO: 1724 or a fragment thereof.
177. The AAV vector genome of embodiment 171, wherein the NPC protein is a cynomolgus (Macaca fascicularis) NPC protein.
178. The AAV vector genome of embodiment 171, wherein the NPC protein is a rhesus macaque (Macaca mulatta) NPC protein.
179. The AAV vector genome of any one of embodiments 177-178, wherein the NPC protein is at least partially humanized.
180. The AAV vector genome of any one of embodiments 171-179, wherein the 5′ ITR is an AAV2 ITR.
181. The AAV vector genome of any one of embodiments 171-180, wherein the 5′ ITR is 130 nucleotides in length.
182. The AAV vector genome of any one of embodiments 171-181, wherein the 3′ ITR is an AAV2 ITR.
183. The AAV vector genome of any one of embodiments 171-182, wherein the 3′ ITR is 130 nucleotides in length.
184. The AAV vector genome of any one of embodiments 171-183, wherein the AAV vector genome comprises one or more of (e.g. all of) the following components: a promoter region, a Kozak region, an NPC protein region, or a polyadenylation (polyA) region.
185. The AAV vector genome of any one of embodiments 171-184, comprising an ITR to ITR sequence of SEQ ID NO: 1752, SEQ ID NO: 1753, SEQ ID NO: 1754, SEQ ID NO: 1755, or SEQ ID NO: 1756, SEQ ID NO: 1757, SEQ ID NO: 1758, or SEQ ID NO: 1759.
186. An AAV particle comprising the AAV vector genome of any one of embodiments 171-185 and a capsid.
187. The AAV particle of embodiment 186, wherein the capsid comprises an amino acid sequence which comprises or which is encoded by a sequence selected from SEQ ID NOs: 1-1261.
188. A pharmaceutical composition comprising the AAV particle of any one of embodiments 186-187.
189. A method of treating a lysosomal storage disorder, said method comprising administering to a subject the pharmaceutical composition of embodiment 188.
190. The method of embodiment 189, wherein the lysosomal storage disorder is NPC1 disease or related disorder.
191. The method of embodiment 189 or 190, wherein the lysosomal storage disorder is a disorder associated with decreased NPC protein levels.
192. The method of any one of embodiments 189-191, wherein the administration of the pharmaceutical composition results in a 0.5×-3.0× increase in NPC protein expression in a target cell of the subject, as compared to NPC protein expression in an equivalent target cell in a subject without a disorder associated with decreased NPC protein levels.
193. The method of any one of embodiments 189-192, further comprising administering miglustat to the subject.

The details of various aspects or embodiments of the present disclosure are set forth below. Other features, objects, and advantages of the disclosure will be apparent from the description and the claims. In the description, the singular forms also include the plural unless the context clearly dictates otherwise. Unless defined otherwise, all technical and scientific terms used herein have the same meaning as commonly understood by one of ordinary skill in the art in the field of this disclosure. In the case of conflict, the present description will control.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the fold over reference expression of NPC1 in HEK293 cells upon transfection with the indicated viral genome construct encoding an NPC1 protein on the X-axis, which are from left to right: no transfection control, reference construct control (ITR_ITR 2; SEQ ID NO: 1753), ITR_ITR 1 (SEQ ID NO: 1752), ITR_ITR 3 (SEQ ID NO: 1754), ITR_ITR 6 (SEQ ID NO: 1757), ITR_ITR 4 (SEQ ID NO: 1755), ITR_ITR 45, ITR_ITR 5, ITR_ITR 43 (SEQ ID NO: 1835), and ITR_ITR 44 (SEQ ID NO: 1836).

FIG. 2 depicts NPC1 expression normalized to GAPDH and transfection efficiency for the indicated ITR to ITR constructs encoding NPC1 on the X-axis comprising EF-1a promoter variants on the X-axis, which are from left to right: the reference construct control (ITR_ITR 2; SEQ ID NO: 1753), ITR_ITR 27 (promoter variant 11), ITR_ITR 28 (promoter variant 13), ITR_ITR 30 (promoter variant 15), ITR_ITR 31 (promoter variant 18), and a no transfection control.

FIG. 3 depicts NPC1 expression normalized to GAPDH and transfection efficiency for the ITR to ITR constructs comprising EF-1a promoter variants, promoter variant 8 (SEQ ID NO: 1782), promoter variant 11 (SEQ ID NO: 1785) or promoter 13 (SEQ ID NO: 1787) operably linked to the wild-type nucleotide sequence encoding NPC1 (wtNPC1) or codon-optimized NPC1 coding sequence 2 (SEQ ID NO: 1750), with an intron (SEQ ID NO: 1780) (“+intron”) or without an intron (“−intron”).

FIG. 4 depicts the percent NPC1 expression relative to healthy patients in patient fibroblasts transfected with the ITR to ITR constructs encoding NPC1 as indicated on the X-axis, which are from left to right: the healthy control, the patient control, ITR_ITR 2 (reference, SEQ ID NO: 1753), ITR_ITR 1 (SEQ ID NO: 1752), ITR_ITR 3 (SEQ ID NO: 1754), ITR_ITR 4 (SEQ ID NO: 1755), or ITR_ITR 5 (SEQ ID NO: 1756).

FIG. 5 depicts in the leftmost graph, the NPC1 fold expression over treatment in hepatocytes transduced with the ITR to ITR constructs encoding NPC1 as indicated on the X-axis, which are from left to right: no treatment, ITR_ITR 2 (reference, SEQ ID NO: 1753), ITR_ITR 1 (SEQ ID NO: 1752), ITR_ITR 3 (SEQ ID NO: 1754), ITR_ITR 4 (SEQ ID NO: 1755), or ITR_ITR 5 (SEQ ID NO: 1756). FIG. 5 depicts in the rightmost graph, the NPC1 fold expression over treatment in a human neuronal line transfected with the ITR to ITR constructs encoding NPC1 as indicated on the X-axis, which are from left to right: no treatment, ITR_ITR 2 (reference, SEQ ID NO: 1753), ITR_ITR 1 (SEQ ID NO: 1752), ITR_ITR 3 (SEQ ID NO: 1754), ITR_ITR 4 (SEQ ID NO: 1755), ITR_ITR 5 (SEQ ID NO: 1756), ITR_ITR 6 (SEQ ID NO: 1757), or ITR_ITR 7 (SEQ ID NO: 1758).

FIG. 6 depicts the cholesterol levels (ug/mL) in healthy fibroblasts, NPC1 patient fibroblasts, and NPC1 patient fibroblasts following transduction with the ITR_ITR 2 construct (SEQ ID NO: 1753) encoding an NPC1 protein at increasing MOI as indicated on the X-axis (1e6, 1e5, 1e4, or 1e3).

FIG. 7 depicts the cholesterol levels (ug/mL) in healthy fibroblasts, NPC1 patient fibroblasts, and NPC1 patient fibroblasts following transduction with the ITR_ITR 3 construct (SEQ ID NO: 1754) (left portion of graph), ITR_ITR 4 construct (SEQ ID NO: 1755) (center portion of the graph), or ITR_ITR 6 construct (SEQ ID NO: 1757) as the doses indicated on the X-axis (1e5, 1e4, or 1e3).

FIG. 8 depicts the cholesterol levels (relative to a BCA control) in healthy fibroblasts, NPC1 patient fibroblasts, and NPC1 patient fibroblasts following transduction with constructs ITR_ITR 9, ITR_ITR 10, ITR_ITR 11, or ITR_ITR 12 (SEQ ID NOs: 1799-1802, respectively).

FIG. 9A depicts performance by age of NPC1^{-/-} mice compared to wild-type mice in beam walk test (null n=7, wild-type n=8). Mice were tested for how long it took for them to cross a narrow beam (top panel) and how many times they slipped while walking across (bottom panel). FIG. 9B depicts performance by age of NPC1^{-/-} mice compared to wild-type mice in rotarod test (null n=7, wild-type n=8). Mice were tested for how long they could walk on an accelerating rotating rod before falling for up to 180 seconds.

FIGS. 10A-10B depict NPC1 expression (NPC1/B-actin) in human neurons (FIG. 10A) and patient fibroblasts (FIG. 10B), transduced with the ITR to ITR constructs encoding NPC1 as indicated on the X-axis, which are from left to right: untreated, ITR_ITR 2 (reference, SEQ ID NO: 1753), ITR_ITR 9, ITR_ITR 10, ITR_ITR 11, or ITR_ITR 12 (SEQ ID NOs: 1799-1802, respectively). Promoter A is Promoter Variant 11 (SEQ ID NO: 1785) and Promoter B is a short CMV promoter (SEQ ID NO: 1736).

FIGS. 11A-11E depict human NPC1 relative to mouse NPC1 expression in the cortex (FIG. 11A), hippocampus (FIG. 11B), cerebellum (FIG. 11C), brainstem (FIG. 11D), and liver (FIG. 11E) in mice post-IV injection of the constructs ITR_ITR 2 (reference, SEQ ID NO: 1753), ITR_ITR 9, ITR_ITR 10, ITR_ITR 11, or ITR_ITR 12 (SEQ ID NOs: 1799-1802, respectively) in an AAV9 vector at a dose of 1e14 Vg/kg.

### DETAILED DESCRIPTION

### Overview

Described herein, inter alia, are compositions comprising isolated, e.g., recombinant, viral particles, e.g., AAV particles, for delivery, e.g., vectorized delivery, of a protein, e.g., an NPC protein, e.g., an NPC1 protein, and methods of making and using the same. Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety as related to parvoviruses.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide, e.g., NPC1 and/or NPC2, which may be delivered to a target cell, tissue, or organism. In some embodiments, the target cell is a CNS cell. In some embodiments, the target tissue is a CNS tissue. The target CNS tissue may be brain tissue. In some embodiments, the brain target tissues comprise a caudate-putamen, thalamus, superior colliculus, cortex, brain stem, corpus collosum, or combination thereof.

Gene therapy presents an alternative treatment approach for NPC1 and related diseases. AAV vectors and particles are commonly used in gene therapy approaches as a result of a number of advantageous features. Without wishing to be bound by theory, it is believed in some embodiments, that expression vectors, e.g., an adeno-associated viral vector (AAVs) or AAV particle, e.g., an AAV particle described herein, can be used to administer and/or deliver NPC1 and/or NPC2 and related proteins, in order to achieve sustained, high concentrations, allowing for longer lasting efficacy, fewer dose treatments, and/or more consistent levels of the NPC1 and/or NPC2 protein, relative to a non-AAV therapy.

As demonstrated in the Examples herein below, the compositions and methods described herein provides improved features compared to prior enzyme replacement approaches, including increased NPC1 expression and biodistribution. In some embodiments, an AAV viral genome encoding an NPC1 protein described herein which comprise an optimized nucleotide sequence encoding the NPC1 protein (e.g., SEQ ID NO: 1750 or 1749) result in high biodistribution in the CNS and increased NPC1 expression. The compositions and methods described herein can be used in the treatment of disorders associated with a lack of an NPC1 protein, such as a lysosomal storage disease or Niemann-Pick disease type C1.

### I. Compositions

### Adeno-Associated Viral (AAV) Vectors

AAV have a genome of about 5,000 nucleotides in length which contains two open reading frames encoding the proteins responsible for replication (Rep) and the structural protein of the capsid (Cap). In some embodiments, the open reading frames are flanked by two Inverted Terminal Repeat (ITR) sequences, which serve as the origin of replication of the viral genome. The wild-type AAV viral genome comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are used for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

The AAV vector typically requires a co-helper (e.g., adenovirus) to undergo productive infection in cells. In the absence of such helper functions, the AAV virions essentially enter host cells but do not integrate into the cells' genome.

AAV vectors have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector, and (v) the non-integrative nature in a host chromosome thereby reducing potential for long-term genetic alterations. Moreover, infection with AAV vectors has minimal influence on changing the pattern of cellular gene expression (Stilwell and Samulski et al., Biotechniques, 2003, 34, 148, the contents of which are herein incorporated by reference in their entirety).

Typically, AAV vectors for NPC protein delivery may be recombinant viral vectors which are replication defective as they lack sequences encoding functional Rep and Cap proteins within the viral genome. In some cases, the defective AAV vectors may lack most or all coding sequences and essentially only contain one or two AAV ITR sequences and a payload sequence. In certain embodiments, the viral genome encodes NPC protein. For example, the viral genome encodes human NPC protein(s).

In some embodiments, the AAV particles of the present disclosure may be introduced into mammalian cells.

AAV vectors may be modified to enhance the efficiency of delivery. Such modified AAV vectors of the present disclosure can be packaged efficiently and can be used to successfully infect the target cells at high frequency and with minimal toxicity.

In other embodiments, AAV particles of the present disclosure may be used to deliver NPC protein to the central nervous system (see, e.g., U.S. Pat. No. 6,180,613; the contents of which are herein incorporated by reference in their entirety) or to specific tissues of the CNS.

As used herein, the term “AAV vector” or “AAV particle” comprises a capsid and a viral genome comprising a polynucleotide payload. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, e.g., NPC protein.

It is understood that the compositions described herein may have additional conservative or non-essential amino acid substitutions, which do not have a substantial effect on their functions.

### AAV Serotypes

AAV particles of the present disclosure may comprise or be derived from any natural or recombinant AAV serotype. According to the present disclosure, the AAV particles may utilize or be based on a serotype or include a peptide selected from any of the following VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LKO1, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the AAV serotype may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LKO1 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV serotype may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in their entirety.

In some embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVF3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U.S. Pat. No. 8,734,809), AAV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ ID NO: 29 and 103 of U.S. Pat. No. 8,734,809), AAV CLv-E1 (SEQ ID NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: X and X of U.S. Pat. No. 8,734,809), AAV CLv-R9 (SEQ ID NO: X and X of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ ID NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), AAV CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLv-3 (SEQ ID NO: 69 and 143 of U.S. Pat. No. 8,734,809), AAV CLv-4 (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809), AAV CLv1-1 (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV particle may have, or may be a serotype selected from any of those found in Table 1.

In some embodiments, the AAV capsid may comprise a sequence, fragment or variant thereof, of any of the sequences in Table 1.

In some embodiments, the AAV capsid may be encoded by a sequence, fragment or variant as described in Table 1.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; 0 (Pyl) for Pyrrolysine; U (Sec) for Selenocysteine; X (Xaa) for any amino acid; and Z (Glx) for Glutamine or Glutamic acid.

TABLE 1
AAV Serotypes

| Serotype | SEQ ID NO: | Reference Information |
|---|---|---|
| VOY101 | 1 | — |
| VOY101 | 2 | — |
| VOY201 | 3 | — |
| PHP.N/PHP.B-DGT | 4 | WO2017100671 SEQ ID NO: 46 |
| AAVPHP.B or G2B-26 | 5 | WO2015038958 SEQ ID NO: 8 and 13 |
| AAVPHP.B | 6 | WO2015038958 SEQ ID NO: 9 |
| AAVG2B-13 | 7 | WO2015038958 SEQ ID NO: 12 |
| AAVTH1.1-32 | 8 | WO2015038958 SEQ ID NO: 14 |
| AAVTH1.1-35 | 9 | WO2015038958 SEQ ID NO: 15 |
| PHP.S/G2A12 | 10 | WO2017100671 SEQ ID NO: 47 |
| AAV9/hu.14 K449R | 11 | WO2017100671 SEQ ID NO: 45 |
| AAV1 | 12 | US20150159173 SEQ ID NO: 11, |
| | | US20150315612 SEQ ID NO: 202 |
| AAV1 | 13 | US20160017295 SEQ ID NO: 1, |
| | | US20030138772 SEQ ID NO: 64, |
| | | US20150159173 SEQ ID NO: 27, |
| | | US20150315612 SEQ ID NO: 219, |
| | | U.S. Pat. No. 7,198,951 SEQ ID NO: 5 |
| AAV1 | 14 | US20030138772 SEQ ID NO: 6 |
| AAV1.3 | 15 | US20030138772 SEQ ID NO: 14 |
| AAV10 | 16 | US20030138772 SEQ ID NO: 117 |
| AAV10 | 17 | WO2015121501 SEQ ID NO: 9 |
| AAV10 | 18 | WO2015121501 SEQ ID NO: 8 |
| AAV11 | 19 | US20030138772 SEQ ID NO: 118 |
| AAV12 | 20 | US20030138772 SEQ ID NO: 119 |
| AAV2 | 21 | US20150159173 SEQ ID NO: 7, |
| | | US20150315612 SEQ ID NO: 211 |
| AAV2 | 22 | US20030138772 SEQ ID NO: 70, |
| | | US20150159173 SEQ ID NO: 23, |
| | | US20150315612 SEQ ID NO: 221, |
| | | US20160017295 SEQ ID NO: 2, |
| | | U.S. Pat. No. 6,156,303 SEQ ID NO: 4, |
| | | U.S. Pat. No. 7,198,951 SEQ ID NO: 4, |
| | | WO2015121501 SEQ ID NO: 1 |
| AAV2 | 23 | U.S. Pat. No. 6,156,303 SEQ ID NO: 8 |
| AAV2 | 24 | US20030138772 SEQ ID NO: 7 |
| AAV2 | 25 | U.S. Pat. No. 6,156,303 SEQ ID NO: 3 |
| AAV2.5T | 26 | U.S. Pat. No. 9,233,131 SEQ ID NO: 42 |
| AAV223.10 | 27 | US20030138772 SEQ ID NO: 75 |
| AAV223.2 | 28 | US20030138772 SEQ ID NO: 49 |
| AAV223.2 | 29 | US20030138772 SEQ ID NO: 76 |
| AAV223.4 | 30 | US20030138772 SEQ ID NO: 50 |
| AAV223.4 | 31 | US20030138772 SEQ ID NO: 73 |
| AAV223.5 | 32 | US20030138772 SEQ ID NO: 51 |
| AAV223.5 | 33 | US20030138772 SEQ ID NO: 74 |
| AAV223.6 | 34 | US20030138772 SEQ ID NO: 52 |
| AAV223.6 | 35 | US20030138772 SEQ ID NO: 78 |
| AAV223.7 | 36 | US20030138772 SEQ ID NO: 53 |
| AAV223.7 | 37 | US20030138772 SEQ ID NO: 77 |
| AAV29.3 | 38 | US20030138772 SEQ ID NO: 82 |
| AAV29.4 | 39 | US20030138772 SEQ ID NO: 12 |
| AAV29.5 | 40 | US20030138772 SEQ ID NO: 83 |
| AAV29.5 (AAVbb.2) | 41 | US20030138772 SEQ ID NO: 13 |
| AAV3 | 42 | US20150159173 SEQ ID NO: 12 |
| AAV3 | 43 | US20030138772 SEQ ID NO: 71, |
| | | US20150159173 SEQ ID NO: 28, |
| | | US20160017295 SEQ ID NO: 3, |
| | | U.S. Pat. No. 7,198,951 SEQ ID NO: 6 |
| AAV3 | 44 | US20030138772 SEQ ID NO: 8 |
| AAV3.3b | 45 | US20030138772 SEQ ID NO: 72 |
| AAV3-3 | 46 | US20150315612 SEQ ID NO: 200 |
| AAV3-3 | 47 | US20150315612 SEQ ID NO: 217 |
| AAV3a | 48 | U.S. Pat. No. 6,156,303 SEQ ID NO: 5 |
| AAV3a | 49 | U.S. Pat. No. 6,156,303 SEQ ID NO: 9 |
| AAV3b | 50 | U.S. Pat. No. 6,156,303 SEQ ID NO: 6 |
| AAV3b | 51 | U.S. Pat. No. 6,156,303 SEQ ID NO: 10 |
| AAV3b | 52 | U.S. Pat. No. 6,156,303 SEQ ID NO: 1 |
| AAV4 | 53 | US20140348794 SEQ ID NO: 17 |
| AAV4 | 54 | US20140348794 SEQ ID NO: 5 |
| AAV4 | 55 | US20140348794 SEQ ID NO: 3 |
| AAV4 | 56 | US20140348794 SEQ ID NO: 14 |
| AAV4 | 57 | US20140348794 SEQ ID NO: 15 |
| AAV4 | 58 | US20140348794 SEQ ID NO: 19 |
| AAV4 | 59 | US20140348794 SEQ ID NO: 12 |
| AAV4 | 60 | US20140348794 SEQ ID NO: 13 |
| AAV4 | 61 | US20140348794 SEQ ID NO: 7 |
| AAV4 | 62 | US20140348794 SEQ ID NO: 8 |
| AAV4 | 63 | US20140348794 SEQ ID NO: 9 |
| AAV4 | 64 | US20140348794 SEQ ID NO: 2 |
| AAV4 | 65 | US20140348794 SEQ ID NO: 10 |
| AAV4 | 66 | US20140348794 SEQ ID NO: 11 |
| AAV4 | 67 | US20140348794 SEQ ID NO: 18 |
| AAV4 | 68 | US20030138772 SEQ ID NO: 63, |
| | | US20160017295 SEQ ID NO: 4, |
| | | US20140348794 SEQ ID NO: 4 |
| AAV4 | 69 | US20140348794 SEQ ID NO: 16 |
| AAV4 | 70 | US20140348794 SEQ ID NO: 20 |
| AAV4 | 71 | US20140348794 SEQ ID NO: 6 |
| AAV4 | 72 | US20140348794 SEQ ID NO: 1 |
| AAV42.2 | 73 | US20030138772 SEQ ID NO: 9 |
| AAV42.2 | 74 | US20030138772 SEQ ID NO: 102 |
| AAV42.3b | 75 | US20030138772 SEQ ID NO: 36 |
| AAV42.3B | 76 | US20030138772 SEQ ID NO: 107 |
| AAV42.4 | 77 | US20030138772 SEQ ID NO: 33 |
| AAV42.4 | 78 | US20030138772 SEQ ID NO: 88 |
| AAV42.8 | 79 | US20030138772 SEQ ID NO: 27 |
| AAV42.8 | 80 | US20030138772 SEQ ID NO: 85 |
| AAV43.1 | 81 | US20030138772 SEQ ID NO: 39 |
| AAV43.1 | 82 | US20030138772 SEQ ID NO: 92 |
| AAV43.12 | 83 | US20030138772 SEQ ID NO: 41 |
| AAV43.12 | 84 | US20030138772 SEQ ID NO: 93 |
| AAV43.20 | 85 | US20030138772 SEQ ID NO: 42 |
| AAV43.20 | 86 | US20030138772 SEQ ID NO: 99 |
| AAV43.21 | 87 | US20030138772 SEQ ID NO: 43 |
| AAV43.21 | 88 | US20030138772 SEQ ID NO: 96 |
| AAV43.23 | 89 | US20030138772 SEQ ID NO: 44 |
| AAV43.23 | 90 | US20030138772 SEQ ID NO: 98 |
| AAV43.25 | 91 | US20030138772 SEQ ID NO: 45 |
| AAV43.25 | 92 | US20030138772 SEQ ID NO: 97 |
| AAV43.5 | 93 | US20030138772 SEQ ID NO: 40 |
| AAV43.5 | 94 | US20030138772 SEQ ID NO: 94 |
| AAV4-4 | 95 | US20150315612 SEQ ID NO: 201 |
| AAV4-4 | 96 | US20150315612 SEQ ID NO: 218 |
| AAV44.1 | 97 | US20030138772 SEQ ID NO: 46 |
| AAV44.1 | 98 | US20030138772 SEQ ID NO: 79 |
| AAV44.5 | 99 | US20030138772 SEQ ID NO: 47 |
| AAV44.5 | 100 | US20030138772 SEQ ID NO: 80 |
| AAV4407 | 101 | US20150315612 SEQ ID NO: 90 |
| AAV5 | 102 | U.S. Pat. No. 7,427,396 SEQ ID NO: 1 |
| AAV5 | 103 | US20030138772 SEQ ID NO: 114 |
| AAV5 | 104 | US20160017295 SEQ ID NO: 5, |
| | | U.S. Pat. No. 7,427,396 SEQ ID NO: 2, |
| | | US20150315612 SEQ ID NO: 216 |
| AAV5 | 105 | US20150315612 SEQ ID NO: 199 |
| AAV6 | 106 | US20150159173 SEQ ID NO: 13 |
| AAV6 | 107 | US20030138772 SEQ ID NO: 65, |
| | | US20150159173 SEQ ID NO: 29, |
| | | US20160017295 SEQ ID NO: 6, |
| | | U.S. Pat. No. 6,156,303 SEQ ID NO: 7 |
| AAV6 | 108 | U.S. Pat. No. 6,156,303 SEQ ID NO: 11 |
| AAV6 | 109 | U.S. Pat. No. 6,156,303 SEQ ID NO: 2 |
| AAV6 | 110 | US20150315612 SEQ ID NO: 203 |
| AAV6 | 111 | US20150315612 SEQ ID NO: 220 |
| AAV6.1 | 112 | US20150159173 |
| AAV6.12 | 113 | US20150159173 |
| AAV6.2 | 114 | US20150159173 |
| AAV7 | 115 | US20150159173 SEQ ID NO: 14 |
| AAV7 | 116 | US20150315612 SEQ ID NO: 183 |
| AAV7 | 117 | US20030138772 SEQ ID NO: 2, |
| | | US20150159173 SEQ ID NO: 30, |
| | | US20150315612 SEQ ID NO: 181, |
| | | US20160017295 SEQ ID NO: 7 |
| AAV7 | 118 | US20030138772 SEQ ID NO: 3 |
| AAV7 | 119 | US20030138772 SEQ ID NO: 1, |
| | | US20150315612 SEQ ID NO: 180 |
| AAV7 | 120 | US20150315612 SEQ ID NO: 213 |
| AAV7 | 121 | US20150315612 SEQ ID NO: 222 |
| AAV8 | 122 | US20150159173 SEQ ID NO: 15 |
| AAV8 | 123 | US20150376240 SEQ ID NO: 7 |
| AAV8 | 124 | US20030138772 SEQ ID NO: 4, |
| | | US20150315612 SEQ ID NO: 182 |
| AAV8 | 125 | US20030138772 SEQ ID NO: 95, |
| | | US20140359799 SEQ ID NO: 1, |
| | | US20150159173 SEQ ID NO: 31, |
| | | US20160017295 SEQ ID NO: 8, |
| | | U.S. Pat. No. 7,198,951 SEQ ID NO: 7, |
| | | US20150315612 SEQ ID NO: 223 |
| AAV8 | 126 | US20150376240 SEQ ID NO: 8 |
| AAV8 | 127 | US20150315612 SEQ ID NO: 214 |
| AAV-8b | 128 | US20150376240 SEQ ID NO: 5 |
| AAV-8b | 129 | US20150376240 SEQ ID NO: 3 |
| AAV-8h | 130 | US20150376240 SEQ ID NO: 6 |
| AAV-8h | 131 | US20150376240 SEQ ID NO: 4 |
| AAV9 | 132 | US20030138772 SEQ ID NO: 5 |
| AAV9 | 133 | U.S. Pat. No. 7,198,951 SEQ ID NO: 1 |
| AAV9 | 134 | US20160017295 SEQ ID NO: 9 |
| AAV9 | 135 | US20030138772 SEQ ID NO: 100, |
| | | U.S. Pat. No. 7,198,951 SEQ ID NO: 2 |
| AAV9 | 136 | U.S. Pat. No. 7,198,951 SEQ ID NO: 3 |
| AAV9 (AAVhu.14) | 137 | U.S. Pat. No. 7,906,111 SEQ ID NO: 3; |
| | | WO2015038958 SEQ ID NO: 11 |
| AAV9 (AAVhu.l4) | 138 | U.S. Pat. No. 7,906,111 SEQ ID NO: 123; |
| | | WO2015038958 SEQ ID NO: 2 |
| AAVA3.1 | 139 | US20030138772 SEQ ID NO: 120 |
| AAVA3.3 | 140 | US20030138772 SEQ ID NO: 57 |
| AAVA3.3 | 141 | US20030138772 SEQ ID NO: 66 |
| AAVA3.4 | 142 | US20030138772 SEQ ID NO: 54 |
| AAVA3.4 | 143 | US20030138772 SEQ ID NO: 68 |
| AAVA3.5 | 144 | US20030138772 SEQ ID NO: 55 |
| AAVA3.5 | 145 | US20030138772 SEQ ID NO: 69 |
| AAVA3.7 | 146 | US20030138772 SEQ ID NO: 56 |
| AAVA3.7 | 147 | US20030138772 SEQ ID NO: 67 |
| AAV29.3 (AAVbb.1) | 148 | US20030138772 SEQ ID NO: 11 |
| AAVC2 | 149 | US20030138772 SEQ ID NO: 61 |
| AAVCh.5 | 150 | US20150159173 SEQ ID NO: 46, |
| | | US20150315612 SEQ ID NO: 234 |
| AAVcy.2 (AAV13.3) | 151 | US20030138772 SEQ ID NO: 15 |
| AAV24.1 | 152 | US20030138772 SEQ ID NO: 101 |
| AAVcy.3 (AAV24.1) | 153 | US20030138772 SEQ ID NO: 16 |
| AAV27.3 | 154 | US20030138772 SEQ ID NO: 104 |
| AAVcy.4 (AAV27.3) | 155 | US20030138772 SEQ ID NO: 17 |
| AAVcy.5 | 156 | US20150315612 SEQ ID NO: 227 |
| AAV7.2 | 157 | US20030138772 SEQ ID NO: 103 |
| AAVcy.5 (AAV7.2) | 158 | US20030138772 SEQ ID NO: 18 |
| AAV16.3 | 159 | US20030138772 SEQ ID NO: 105 |
| AAVcy.6 (AAV16.3) | 160 | US20030138772 SEQ ID NO: 10 |
| AAVcy.5 | 161 | US20150159173 SEQ ID NO: 8 |
| AAVcy.5 | 162 | US20150159173 SEQ ID NO: 24 |
| AAVCy.5R1 | 163 | US20150159173 |
| AAVCy.5R2 | 164 | US20150159173 |
| AAVCy.5R3 | 165 | US20150159173 |
| AAVCy.5R4 | 166 | US20150159173 |
| AAVDJ | 167 | US20140359799 SEQ ID NO: 3, |
| | | U.S. Pat. No. 7,588,772 SEQ ID NO: 2 |
| AAVDJ | 168 | US20140359799 SEQ ID NO: 2, |
| | | U.S. Pat. No. 7,588,772 SEQ ID NO: 1 |
| AAVDJ-8 | 169 | U.S. Pat. No. 7,588,772; Grimm et al 2008 |
| AAVDJ-8 | 170 | U.S. Pat. No. 7,588,772; Grimm et al 2008 |
| AAVF5 | 171 | US20030138772 SEQ ID NO: 110 |
| AAVH2 | 172 | US20030138772 SEQ ID NO: 26 |
| AAVH6 | 173 | US20030138772 SEQ ID NO: 25 |
| AAVhE1.1 | 174 | U.S. Pat. No. 9,233,131 SEQ ID NO: 44 |
| AAVhEr1.14 | 175 | U.S. Pat. No. 9,233,131 SEQ ID NO: 46 |
| AAVhEr1.16 | 176 | U.S. Pat. No. 9,233,131 SEQ ID NO: 48 |
| AAVhEr1.18 | 177 | U.S. Pat. No. 9,233,131 SEQ ID NO: 49 |
| AAVhEr1.23 (AAVhEr2.29) | 178 | U.S. Pat. No. 9,233,131 SEQ ID NO: 53 |
| AAVhEr1.35 | 179 | U.S. Pat. No. 9,233,131 SEQ ID NO: 50 |
| AAVhEr1.36 | 180 | U.S. Pat. No. 9,233,131 SEQ ID NO: 52 |
| AAVhEr1.5 | 181 | U.S. Pat. No. 9,233,131 SEQ ID NO: 45 |
| AAVhEr1.7 | 182 | U.S. Pat. No. 9,233,131 SEQ ID NO: 51 |
| AAVhEr1.8 | 183 | U.S. Pat. No. 9,233,131 SEQ ID NO: 47 |
| AAVhEr2.16 | 184 | U.S. Pat. No. 9,233,131 SEQ ID NO: 55 |
| AAVhEr2.30 | 185 | U.S. Pat. No. 9,233,131 SEQ ID NO: 56 |
| AAVhEr2.31 | 186 | U.S. Pat. No. 9,233,131 SEQ ID NO: 58 |
| AAVhEr2.36 | 187 | U.S. Pat. No. 9,233,131 SEQ ID NO: 57 |
| AAVhEr2.4 | 188 | U.S. Pat. No. 9,233,131 SEQ ID NO: 54 |
| AAVhEr3.1 | 189 | U.S. Pat. No. 9,233,131 SEQ ID NO: 59 |
| AAVhu.1 | 190 | US20150315612 SEQ ID NO: 46 |
| AAVhu.1 | 191 | US20150315612 SEQ ID NO: 144 |
| AAVhu.10 (AAV16.8) | 192 | US20150315612 SEQ ID NO: 56 |
| AAVhu.10 (AAV16.8) | 193 | US20150315612 SEQ ID NO: 156 |
| AAVhu.11 (AAV16.12) | 194 | US20150315612 SEQ ID NO: 57 |
| AAVhu.11 (AAV16.12) | 195 | US20150315612 SEQ ID NO: 153 |
| AAVhu.12 | 196 | US20150315612 SEQ ID NO: 59 |
| AAVhu.12 | 197 | US20150315612 SEQ ID NO: 154 |
| AAVhu.13 | 198 | US20150159173 SEQ ID NO: 16, |
| | | US20150315612 SEQ ID NO: 71 |
| AAVhu.13 | 199 | US20150159173 SEQ ID NO: 32, |
| | | US20150315612 SEQ ID NO: 129 |
| AAVhu.136.1 | 200 | US20150315612 SEQ ID NO: 165 |
| AAVhu.140.1 | 201 | US20150315612 SEQ ID NO: 166 |
| AAVhu.140.2 | 202 | US20150315612 SEQ ID NO: 167 |
| AAVhu.145.6 | 203 | US20150315612 SEQ ID No: 178 |
| AAVhu.15 | 204 | US20150315612 SEQ ID NO: 147 |
| AAVhu.15 (AAV33.4) | 205 | US20150315612 SEQ ID NO: 50 |
| AAVhu.156.1 | 206 | US20150315612 SEQ ID No: 179 |
| AAVhu.16 | 207 | US20150315612 SEQ ID NO: 148 |
| AAVhu.16 (AAV33.8) | 208 | US20150315612 SEQ ID NO: 51 |
| AAVhu.17 | 209 | US20150315612 SEQ ID NO: 83 |
| AAVhu.17 (AAV33.12) | 210 | US20150315612 SEQ ID NO: 4 |
| AAVhu.172.1 | 211 | US20150315612 SEQ ID NO: 171 |
| AAVhu.172.2 | 212 | US20150315612 SEQ ID NO: 172 |
| AAVhu.173.4 | 213 | US20150315612 SEQ ID NO: 173 |
| AAVhu.173.8 | 214 | US20150315612 SEQ ID NO: 175 |
| AAVhu.18 | 215 | US20150315612 SEQ ID NO: 52 |
| AAVhu.18 | 216 | US20150315612 SEQ ID NO: 149 |
| AAVhu.19 | 217 | US20150315612 SEQ ID NO: 62 |
| AAVhu.19 | 218 | US20150315612 SEQ ID NO: 133 |
| AAVhu.2 | 219 | US20150315612 SEQ ID NO: 48 |
| AAVhu.2 | 220 | US20150315612 SEQ ID NO: 143 |
| AAVhu.20 | 221 | US20150315612 SEQ ID NO: 63 |
| AAVhu.20 | 222 | US20150315612 SEQ ID NO: 134 |
| AAVhu.21 | 223 | US20150315612 SEQ ID NO: 65 |
| AAVhu.21 | 224 | US20150315612 SEQ ID NO: 135 |
| AAVhu.22 | 225 | US20150315612 SEQ ID NO: 67 |
| AAVhu.22 | 226 | US20150315612 SEQ ID NO: 138 |
| AAVhu.23 | 227 | US20150315612 SEQ ID NO: 60 |
| AAVhu.23.2 | 228 | US20150315612 SEQ ID NO: 137 |
| AAVhu.24 | 229 | US20150315612 SEQ ID NO: 66 |
| AAVhu.24 | 230 | US20150315612 SEQ ID NO: 136 |
| AAVhu.25 | 231 | US20150315612 SEQ ID NO: 49 |
| AAVhu.25 | 232 | US20150315612 SEQ ID NO: 146 |
| AAVhu.26 | 233 | US20150159173 SEQ ID NO: 17, |
| | | US20150315612 SEQ ID NO: 61 |
| AAVhu.26 | 234 | US20150159173 SEQ ID NO: 33, |
| | | US20150315612 SEQ ID NO: 139 |
| AAVhu.27 | 235 | US20150315612 SEQ ID NO: 64 |
| AAVhu.27 | 236 | US20150315612 SEQ ID NO: 140 |
| AAVhu.28 | 237 | US20150315612 SEQ ID NO: 68 |
| AAVhu.28 | 238 | US20150315612 SEQ ID NO: 130 |
| AAVhu.29 | 239 | US20150315612 SEQ ID NO: 69 |
| AAVhu.29 | 240 | US20150159173 SEQ ID NO: 42, |
| | | US20150315612 SEQ ID NO: 132 |
| AAVhu.29 | 241 | US20150315612 SEQ ID NO: 225 |
| AAVhu.29R | 242 | US20150159173 |
| AAVhu.3 | 243 | US20150315612 SEQ ID NO: 44 |
| AAVhu.3 | 244 | US20150315612 SEQ ID NO: 145 |
| AAVhu.30 | 245 | US20150315612 SEQ ID NO: 70 |
| AAVhu.30 | 246 | US20150315612 SEQ ID NO: 131 |
| AAVhu.31 | 247 | US20150315612 SEQ ID NO: 1 |
| AAVhu.31 | 248 | US20150315612 SEQ ID NO: 121 |
| AAVhu.32 | 249 | US20150315612 SEQ ID NO: 2 |
| AAVhu.32 | 250 | US20150315612 SEQ ID NO: 122 |
| AAVhu.33 | 251 | US20150315612 SEQ ID NO: 75 |
| AAVhu.33 | 252 | US20150315612 SEQ ID NO: 124 |
| AAVhu.34 | 253 | US20150315612 SEQ ID NO: 72 |
| AAVhu.34 | 254 | US20150315612 SEQ ID NO: 125 |
| AAVhu.35 | 255 | US20150315612 SEQ ID NO: 73 |
| AAVhu.35 | 256 | US20150315612 SEQ ID NO: 164 |
| AAVhu.36 | 257 | US20150315612 SEQ ID NO: 74 |
| AAVhu.36 | 258 | US20150315612 SEQ ID NO: 126 |
| AAVhu.37 | 259 | US20150159173 SEQ ID NO: 34, |
| | | US20150315612 SEQ ID NO: 88 |
| AAVhu.37 (AAV106.1) | 260 | US20150315612 SEQ ID NO: 10, |
| | | US20150159173 SEQ ID NO: 18 |
| AAVhu.38 | 261 | US20150315612 SEQ ID NO: 161 |
| AAVhu.39 | 262 | US20150315612 SEQ ID NO: 102 |
| AAVhu.39 (AAVLG-9) | 263 | US20150315612 SEQ ID NO: 24 |
| AAVhu.4 | 264 | US20150315612 SEQ ID NO: 47 |
| AAVhu.4 | 265 | US20150315612 SEQ ID NO: 141 |
| AAVhu.40 | 266 | US20150315612 SEQ ID NO: 87 |
| AAVhu.40 (AAV114.3) | 267 | US20150315612 SEQ ID No: 11 |
| AAVhu.41 | 268 | US20150315612 SEQ ID NO: 91 |
| AAVhu.41 (AAV127.2) | 269 | US20150315612 SEQ ID NO: 6 |
| AAVhu.42 | 270 | US20150315612 SEQ ID NO: 85 |
| AAVhu.42 (AAV127.5) | 271 | US20150315612 SEQ ID NO: 8 |
| AAVhu.43 | 272 | US20150315612 SEQ ID NO: 160 |
| AAVhu.43 | 273 | US20150315612 SEQ ID NO: 236 |
| AAVhu.43 (AAV128.1) | 274 | US20150315612 SEQ ID NO: 80 |
| AAVhu.44 | 275 | US20150159173 SEQ ID NO: 45, |
| | | US20150315612 SEQ ID NO: 158 |
| AAVhu.44 (AAV128.3) | 276 | US20150315612 SEQ ID NO: 81 |
| AAVhu.44R1 | 277 | US20150159173 |
| AAVhu.44R2 | 278 | US20150159173 |
| AAVhu.44R3 | 279 | US20150159173 |
| AAVhu.45 | 280 | US20150315612 SEQ ID NO: 76 |
| AAVhu.45 | 281 | US20150315612 SEQ ID NO: 127 |
| AAVhu.46 | 282 | US20150315612 SEQ ID NO: 82 |
| AAVhu.46 | 283 | US20150315612 SEQ ID NO: 159 |
| AAVhu.46 | 284 | US20150315612 SEQ ID NO: 224 |
| AAVhu.47 | 285 | US20150315612 SEQ ID NO: 77 |
| AAVhu.47 | 286 | US20150315612 SEQ ID NO: 128 |
| AAVhu.48 | 287 | US20150159173 SEQ ID NO: 38 |
| AAVhu.48 | 288 | US20150315612 SEQ ID NO: 157 |
| AAVhu.48 (AAV130.4) | 289 | US20150315612 SEQ ID NO: 78 |
| AAVhu.48R1 | 290 | US20150159173 |
| AAVhu.48R2 | 291 | US20150159173 |
| AAVhu.48R3 | 292 | US20150159173 |
| AAVhu.49 | 293 | US20150315612 SEQ ID NO: 209 |
| AAVhu.49 | 294 | US20150315612 SEQ ID NO: 189 |
| AAVhu.5 | 295 | US20150315612 SEQ ID NO: 45 |
| AAVhu.5 | 296 | US20150315612 SEQ ID NO: 142 |
| AAVhu.51 | 297 | US20150315612 SEQ ID NO: 208 |
| AAVhu.51 | 298 | US20150315612 SEQ ID NO: 190 |
| AAVhu.52 | 299 | US20150315612 SEQ ID NO: 210 |
| AAVhu.52 | 300 | US20150315612 SEQ ID NO: 191 |
| AAVhu.53 | 301 | US20150159173 SEQ ID NO: 19 |
| AAVhu.53 | 302 | US20150159173 SEQ ID NO: 35 |
| AAVhu.53 (AAV145.1) | 303 | US20150315612 SEQ ID NO: 176 |
| AAVhu.54 | 304 | US20150315612 SEQ ID NO: 188 |
| AAVhu.54 (AAV145.5) | 305 | US20150315612 SEQ ID No: 177 |
| AAVhu.55 | 306 | US20150315612 SEQ ID NO: 187 |
| AAVhu.56 | 307 | US20150315612 SEQ ID NO: 205 |
| AAVhu.56 (AAV145.6) | 308 | US20150315612 SEQ ID NO: 168 |
| AAVhu.56 (AAV145.6) | 309 | US20150315612 SEQ ID NO: 192 |
| AAVhu.57 | 310 | US20150315612 SEQ ID NO: 206 |
| AAVhu.57 | 311 | US20150315612 SEQ ID NO: 169 |
| AAVhu.57 | 312 | US20150315612 SEQ ID NO: 193 |
| AAVhu.58 | 313 | US20150315612 SEQ ID NO: 207 |
| AAVhu.58 | 314 | US20150315612 SEQ ID NO: 194 |
| AAVhu.6 (AAV3.1) | 315 | US20150315612 SEQ ID NO: 5 |
| AAVhu.6 (AAV3.1) | 316 | US20150315612 SEQ ID NO: 84 |
| AAVhu.60 | 317 | US20150315612 SEQ ID NO: 184 |
| AAVhu.60 (AAV161.10) | 318 | US20150315612 SEQ ID NO: 170 |
| AAVhu.61 | 319 | US20150315612 SEQ ID NO: 185 |
| AAVhu.61 (AAV161.6) | 320 | US20150315612 SEQ ID NO: 174 |
| AAVhu.63 | 321 | US20150315612 SEQ ID NO: 204 |
| AAVhu.63 | 322 | US20150315612 SEQ ID NO: 195 |
| AAVhu.64 | 323 | US20150315612 SEQ ID NO: 212 |
| AAVhu.64 | 324 | US20150315612 SEQ ID NO: 196 |
| AAVhu.66 | 325 | US20150315612 SEQ ID NO: 197 |
| AAVhu.67 | 326 | US20150315612 SEQ ID NO: 215 |
| AAVhu.67 | 327 | US20150315612 SEQ ID NO: 198 |
| AAVhu.7 | 328 | US20150315612 SEQ ID NO: 226 |
| AAVhu.7 | 329 | US20150315612 SEQ ID NO: 150 |
| AAVhu.7 (AAV7.3) | 330 | US20150315612 SEQ ID NO: 55 |
| AAVhu.71 | 331 | US20150315612 SEQ ID NO: 79 |
| AAVhu.8 | 332 | US20150315612 SEQ ID NO: 53 |
| AAVhu.8 | 333 | US20150315612 SEQ ID NO: 12 |
| AAVhu.8 | 334 | US20150315612 SEQ ID NO: 151 |
| AAVhu.9 (AAV3.1) | 335 | US20150315612 SEQ ID NO: 58 |
| AAVhu.9 (AAV3.1) | 336 | US20150315612 SEQ ID NO: 155 |
| AAV-LK01 | 337 | US20150376607 SEQ ID NO: 2 |
| AAV-LK01 | 338 | US20150376607 SEQ ID NO: 29 |
| AAV-LK02 | 339 | US20150376607 SEQ ID NO: 3 |
| AAV-LK02 | 340 | US20150376607 SEQ ID NO: 30 |
| AAV-LK03 | 341 | US20150376607 SEQ ID NO: 4 |
| AAV-LK03 | 342 | WO2015121501 SEQ ID NO: 12, |
| | | US20150376607 SEQ ID NO: 31 |
| AAV-LK04 | 343 | US20150376607 SEQ ID NO: 5 |
| AAV-LK04 | 344 | US20150376607 SEQ ID NO: 32 |
| AAV-LK05 | 345 | US20150376607 SEQ ID NO: 6 |
| AAV-LK05 | 346 | US20150376607 SEQ ID NO: 33 |
| AAV-LK06 | 347 | US20150376607 SEQ ID NO: 7 |
| AAV-LK06 | 348 | US20150376607 SEQ ID NO: 34 |
| AAV-LK07 | 349 | US20150376607 SEQ ID NO: 8 |
| AAV-LK07 | 350 | US20150376607 SEQ ID NO: 35 |
| AAV-LK08 | 351 | US20150376607 SEQ ID NO: 9 |
| AAV-LK08 | 352 | US20150376607 SEQ ID NO: 36 |
| AAV-LK09 | 353 | US20150376607 SEQ ID NO: 10 |
| AAV-LK09 | 354 | US20150376607 SEQ ID NO: 37 |
| AAV-LK10 | 355 | US20150376607 SEQ ID NO: 11 |
| AAV-LK10 | 356 | US20150376607 SEQ ID NO: 38 |
| AAV-LK11 | 357 | US20150376607 SEQ ID NO: 12 |
| AAV-LK11 | 358 | US20150376607 SEQ ID NO: 39 |
| AAV-LK12 | 359 | US20150376607 SEQ ID NO: 13 |
| AAV-LK12 | 360 | US20150376607 SEQ ID NO: 40 |
| AAV-LK13 | 361 | US20150376607 SEQ ID NO: 14 |
| AAV-LK13 | 362 | US20150376607 SEQ ID NO: 41 |
| AAV-LK14 | 363 | US20150376607 SEQ ID NO: 15 |
| AAV-LK14 | 364 | US20150376607 SEQ ID NO: 42 |
| AAV-LK15 | 365 | US20150376607 SEQ ID NO: 16 |
| AAV-LK15 | 366 | US20150376607 SEQ ID NO: 43 |
| AAV-LK16 | 367 | US20150376607 SEQ ID NO: 17 |
| AAV-LK16 | 368 | US20150376607 SEQ ID NO: 44 |
| AAV-LK17 | 369 | US20150376607 SEQ ID NO: 18 |
| AAV-LK17 | 370 | US20150376607 SEQ ID NO: 45 |
| AAV-LK18 | 371 | US20150376607 SEQ ID NO: 19 |
| AAV-LK18 | 372 | US20150376607 SEQ ID NO: 46 |
| AAV-LK19 | 373 | US20150376607 SEQ ID NO: 20 |
| AAV-LK19 | 374 | US20150376607 SEQ ID NO: 47 |
| AAV-PAEC | 375 | US20150376607 SEQ ID NO: 1 |
| AAV-PAEC | 376 | US20150376607 SEQ ID NO: 48 |
| AAV-PAEC11 | 377 | US20150376607 SEQ ID NO: 26 |
| AAV-PAEC11 | 378 | US20150376607 SEQ ID NO: 54 |
| AAV-PAEC12 | 379 | US20150376607 SEQ ID NO: 27 |
| AAV-PAEC12 | 380 | US20150376607 SEQ ID NO: 51 |
| AAV-PAEC13 | 381 | US20150376607 SEQ ID NO: 28 |
| AAV-PAEC13 | 382 | US20150376607 SEQ ID NO: 49 |
| AAV-PAEC2 | 383 | US20150376607 SEQ ID NO: 21 |
| AAV-PAEC2 | 384 | US20150376607 SEQ ID NO: 56 |
| AAV-PAEC4 | 385 | US20150376607 SEQ ID NO: 22 |
| AAV-PAEC4 | 386 | US20150376607 SEQ ID NO: 55 |
| AAV-PAEC6 | 387 | US20150376607 SEQ ID NO: 23 |
| AAV-PAEC6 | 388 | US20150376607 SEQ ID NO: 52 |
| AAV-PAEC7 | 389 | US20150376607 SEQ ID NO: 24 |
| AAV-PAEC7 | 390 | US20150376607 SEQ ID NO: 53 |
| AAV-PAEC8 | 391 | US20150376607 SEQ ID NO: 25 |
| AAV-PAEC8 | 392 | US20150376607 SEQ ID NO: 50 |
| AAVpi.1 | 393 | US20150315612 SEQ ID NO: 28 |
| AAVpi.1 | 394 | US20150315612 SEQ ID NO: 93 |
| AAVpi.2 | 395 | US20150315612 SEQ ID NO: 30 |
| AAVpi.2 | 396 | US20150315612 SEQ ID NO: 95 |
| AAVpi.3 | 397 | US20150315612 SEQ ID NO: 29 |
| AAVpi.3 | 398 | US20150315612 SEQ ID NO: 94 |
| AAVrh.10 | 399 | US20150159173 SEQ ID NO: 9 |
| AAVrh.10 | 400 | US20150159173 SEQ ID NO: 25 |
| AAV44.2 | 401 | US20030138772 SEQ ID NO: 59 |
| AAVrh.10 (AAV44.2) | 402 | US20030138772 SEQ ID NO: 81 |
| AAV42.1B | 403 | US20030138772 SEQ ID NO: 90 |
| AAVrh.12 (AAV42.1b) | 404 | US20030138772 SEQ ID NO: 30 |
| AAVrh.13 | 405 | US20150159173 SEQ ID NO: 10 |
| AAVrh.13 | 406 | US20150159173 SEQ ID NO: 26 |
| AAVrh.13 | 407 | US20150315612 SEQ ID NO: 228 |
| AAVrh.13R | 408 | US20150159173 |
| AAV42.3A | 409 | US20030138772 SEQ ID NO: 87 |
| AAVrh.14 (AAV42.3a) | 410 | US20030138772 SEQ ID NO: 32 |
| AAV42.5A | 411 | US20030138772 SEQ ID NO: 89 |
| AAVrh.17 (AAV42.5a) | 412 | US20030138772 SEQ ID NO: 34 |
| AAV42.5B | 413 | US20030138772 SEQ ID NO: 91 |
| AAVrh.18 (AAV42.5b) | 414 | US20030138772 SEQ ID NO: 29 |
| AAV42.6B | 415 | US20030138772 SEQ ID NO: 112 |
| AAVrh.19 (AAV42.6b) | 416 | US20030138772 SEQ ID NO: 38 |
| AAVrh.2 | 417 | US20150159173 SEQ ID NO: 39 |
| AAVrh.2 | 418 | US20150315612 SEQ ID NO: 231 |
| AAVrh.20 | 419 | US20150159173 SEQ ID NO: 1 |
| AAV42.10 | 420 | US20030138772 SEQ ID NO: 106 |
| AAVrh.21 (AAV42.10) | 421 | US20030138772 SEQ ID NO: 35 |
| AAV42.11 | 422 | US20030138772 SEQ ID NO: 108 |
| AAVrh.22 (AAV42.11) | 423 | US20030138772 SEQ ID NO: 37 |
| AAV42.12 | 424 | US20030138772 SEQ ID NO: 113 |
| AAVrh.23 (AAV42.12) | 425 | US20030138772 SEQ ID NO: 58 |
| AAV42.13 | 426 | US20030138772 SEQ ID NO: 86 |
| AAVrh.24 (AAV42.13) | 427 | US20030138772 SEQ ID NO: 31 |
| AAV42.15 | 428 | US20030138772 SEQ ID NO: 84 |
| AAVrh.25 (AAV42.15) | 429 | US20030138772 SEQ ID NO: 28 |
| AAVrh.2R | 430 | US20150159173 |
| AAVrh.31 (AAV223.1) | 431 | US20030138772 SEQ ID NO: 48 |
| AAVC1 | 432 | US20030138772 SEQ ID NO: 60 |
| AAVrh.32 (AAVC1) | 433 | US20030138772 SEQ ID NO: 19 |
| AAVrh.32/33 | 434 | US20150159173 SEQ ID NO: 2 |
| AAVrh.33 (AAVC3) | 435 | US20030138772 SEQ ID NO: 20 |
| AAVC5 | 436 | US20030138772 SEQ ID NO: 62 |
| AAVrh.34 (AAVC5) | 437 | US20030138772 SEQ ID NO: 21 |
| AAVF1 | 438 | US20030138772 SEQ ID NO: 109 |
| AAVrh.35 (AAVF1) | 439 | US20030138772 SEQ ID NO: 22 |
| AAVF3 | 440 | US20030138772 SEQ ID NO: 111 |
| AAVrh.36 (AAVF3) | 441 | US20030138772 SEQ ID NO: 23 |
| AAVrh.37 | 442 | US20030138772 SEQ ID NO: 24 |
| AAVrh.37 | 443 | US20150159173 SEQ ID NO: 40 |
| AAVrh.37 | 444 | US20150315612 SEQ ID NO: 229 |
| AAVrh.37R2 | 445 | US20150159173 |
| AAVrh.38 (AAVLG-4) | 446 | US20150315612 SEQ ID NO: 7 |
| AAVrh.38 (AAVLG-4) | 447 | US20150315612 SEQ ID NO: 86 |
| AAVrh.39 | 448 | US20150159173 SEQ ID NO: 20, |
| | | US20150315612 SEQ ID NO: 13 |
| AAVrh.39 | 449 | US20150159173 SEQ ID NO: 3, |
| | | US20150159173 SEQ ID NO: 36, |
| | | US20150315612 SEQ ID NO: 89 |
| AAVrh.40 | 450 | US20150315612 SEQ ID NO: 92 |
| AAVrh.40 (AAVLG-10) | 451 | US20150315612 SEQ ID NO: 14 |
| AAVrh.43 (AAVN721-8) | 452 | US20150315612 SEQ ID NO: 43, |
| | | US20150159173 SEQ ID NO: 21 |
| AAVrh.43 (AAVN721-8) | 453 | US20150315612 SEQ ID NO: 163, |
| | | US20150159173 SEQ ID NO: 37 |
| AAVrh.44 | 454 | US20150315612 SEQ ID NO: 34 |
| AAVrh.44 | 455 | US20150315612 SEQ ID NO: 111 |
| AAVrh.45 | 456 | US20150315612 SEQ ID NO: 41 |
| AAVrh.45 | 457 | US20150315612 SEQ ID NO: 109 |
| AAVrh.46 | 458 | US20150159173 SEQ ID NO: 22, |
| | | US20150315612 SEQ ID NO: 19 |
| AAVrh.46 | 459 | US20150159173 SEQ ID NO: 4, |
| | | US20150315612 SEQ ID NO: 101 |
| AAVrh.47 | 460 | US20150315612 SEQ ID NO: 38 |
| AAVrh.47 | 461 | US20150315612 SEQ ID NO: 118 |
| AAVrh.48 | 462 | US20150159173 SEQ ID NO: 44, |
| | | US20150315612 SEQ ID NO: 115 |
| AAVrh.48.1 | 463 | US20150159173 |
| AAVrh.48.1.2 | 464 | US20150159173 |
| AAVrh.48.2 | 465 | US20150159173 |
| AAVrh.48 (AAV1-7) | 466 | US20150315612 SEQ ID NO: 32 |
| AAVrh.49 (AAV1-8) | 467 | US20150315612 SEQ ID NO: 25 |
| AAVrh.49 (AAV1-8) | 468 | US20150315612 SEQ ID NO: 103 |
| AAVrh.50 (AAV2-4) | 469 | US20150315612 SEQ ID NO: 23 |
| AAVrh.50 (AAV2-4) | 470 | US20150315612 SEQ ID NO: 108 |
| AAVrh.51 (AAV2-5) | 471 | US20150315612 SEQ ID No: 22 |
| AAVrh.51 (AAV2-5) | 472 | US20150315612 SEQ ID NO: 104 |
| AAVrh.52 (AAV3-9) | 473 | US20150315612 SEQ ID NO: 18 |
| AAVrh.52 (AAV3-9) | 474 | US20150315612 SEQ ID NO: 96 |
| AAVrh.53 | 475 | US20150315612 SEQ ID NO: 97 |
| AAVrh.53 (AAV3-11) | 476 | US20150315612 SEQ ID NO: 17 |
| AAVrh.53 (AAV3-11) | 477 | US20150315612 SEQ ID NO: 186 |
| AAVrh.54 | 478 | US20150315612 SEQ ID NO: 40 |
| AAVrh.54 | 479 | US20150159173 SEQ ID NO: 49, |
| | | US20150315612 SEQ ID NO: 116 |
| AAVrh.55 | 480 | US20150315612 SEQ ID NO: 37 |
| AAVrh.55 (AAV4-19) | 481 | US20150315612 SEQ ID NO: 117 |
| AAVrh.56 | 482 | US20150315612 SEQ ID NO: 54 |
| AAVrh.56 | 483 | US20150315612 SEQ ID NO: 152 |
| AAVrh.57 | 484 | US20150315612 SEQ ID NO: 26 |
| AAVrh.57 | 485 | US20150315612 SEQ ID NO: 105 |
| AAVrh.58 | 486 | US20150315612 SEQ ID NO: 27 |
| AAVrh.58 | 487 | US20150159173 SEQ ID NO: 48, |
| | | US20150315612 SEQ ID NO: 106 |
| AAVrh.58 | 488 | US20150315612 SEQ ID NO: 232 |
| AAVrh.59 | 489 | US20150315612 SEQ ID NO: 42 |
| AAVrh.59 | 490 | US20150315612 SEQ ID NO: 110 |
| AAVrh.60 | 491 | US20150315612 SEQ ID NO: 31 |
| AAVrh.60 | 492 | US20150315612 SEQ ID NO: 120 |
| AAVrh.61 | 493 | US20150315612 SEQ ID NO: 107 |
| AAVrh.61 (AAV2-3) | 494 | US20150315612 SEQ ID NO: 21 |
| AAVrh.62 (AAV2-15) | 495 | US20150315612 SEQ ID No: 33 |
| AAVrh.62 (AAV2-15) | 496 | US20150315612 SEQ ID NO: 114 |
| AAVrh.64 | 497 | US20150315612 SEQ ID No: 15 |
| AAVrh.64 | 498 | US20150159173 SEQ ID NO: 43, |
| | | US20150315612 SEQ ID NO: 99 |
| AAVrh.64 | 499 | US20150315612 SEQ ID NO: 233 |
| AAVRh.64R1 | 500 | US20150159173 |
| AAVRh.64R2 | 501 | US20150159173 |
| AAVrh.65 | 502 | US20150315612 SEQ ID NO: 35 |
| AAVrh.65 | 503 | US20150315612 SEQ ID NO: 112 |
| AAVrh.67 | 504 | US20150315612 SEQ ID NO: 36 |
| AAVrh.67 | 505 | US20150315612 SEQ ID NO: 230 |
| AAVrh.67 | 506 | US20150159173 SEQ ID NO: 47, |
| | | US20150315612 SEQ ID NO: 113 |
| AAVrh.68 | 507 | US20150315612 SEQ ID NO: 16 |
| AAVrh.68 | 508 | US20150315612 SEQ ID NO: 100 |
| AAVrh.69 | 509 | US20150315612 SEQ ID NO: 39 |
| AAVrh.69 | 510 | US20150315612 SEQ ID NO: 119 |
| AAVrh.70 | 511 | US20150315612 SEQ ID NO: 20 |
| AAVrh.70 | 512 | US20150315612 SEQ ID NO: 98 |
| AAVrh.71 | 513 | US20150315612 SEQ ID NO: 162 |
| AAVrh.72 | 514 | US20150315612 SEQ ID NO: 9 |
| AAVrh.73 | 515 | US20150159173 SEQ ID NO: 5 |
| AAVrh.74 | 516 | US20150159173 SEQ ID NO: 6 |
| AAVrh.8 | 517 | US20150159173 SEQ ID NO: 41 |
| AAVrh.8 | 518 | US20150315612 SEQ ID NO: 235 |
| AAVrh.8R | 519 | US20150159173, WO2015168666 SEQ ID NO: 9 |
| AAVrh.8R A586R mutant | 520 | WO2015168666 SEQ ID NO: 10 |
| AAVrh.8R R533A mutant | 521 | WO2015168666 SEQ ID NO: 11 |
| BAAV (bovine AAV) | 522 | U.S. Pat. No. 9,193,769 SEQ ID NO: 8 |
| BAAV (bovine AAV) | 523 | U.S. Pat. No. 9,193,769 SEQ ID NO: 10 |
| BAAV (bovine AAV) | 524 | U.S. Pat. No. 9,193,769 SEQ ID NO: 4 |
| BAAV (bovine AAV) | 525 | U.S. Pat. No. 9,193,769 SEQ ID NO: 2 |
| BAAV (bovine AAV) | 526 | U.S. Pat. No. 9,193,769 SEQ ID NO: 6 |
| BAAV (bovine AAV) | 527 | U.S. Pat. No. 9,193,769 SEQ ID NO: 1 |
| BAAV (bovine AAV) | 528 | U.S. Pat. No. 9,193,769 SEQ ID NO: 5 |
| BAAV (bovine AAV) | 529 | U.S. Pat. No. 9,193,769 SEQ ID NO: 3 |
| BAAV (bovine AAV) | 530 | U.S. Pat. No. 9,193,769 SEQ ID NO: 11 |
| BAAV (bovine AAV) | 531 | U.S. Pat. No. 7,427,396 SEQ ID NO: 5 |
| BAAV (bovine AAV) | 532 | U.S. Pat. No. 7,427,396 SEQ ID NO: 6 |
| BAAV (bovine AAV) | 533 | U.S. Pat. No. 9,193,769 SEQ ID NO: 7 |
| BAAV (bovine AAV) | 534 | U.S. Pat. No. 9,193,769 SEQ ID NO: 9 |
| BNP61 AAV | 535 | US20150238550 SEQ ID NO: 1 |
| BNP61 AAV | 536 | US20150238550 SEQ ID NO: 2 |
| BNP62 AAV | 537 | US20150238550 SEQ ID NO: 3 |
| BNP63 AAV | 538 | US20150238550 SEQ ID NO: 4 |
| caprine AAV | 539 | U.S. Pat. No. 7,427,396 SEQ ID NO: 3 |
| caprine AAV | 540 | U.S. Pat. No. 7,427,396 SEQ ID NO: 4 |
| true type AAV (ttAAV) | 541 | WO2015121501 SEQ ID NO: 2 |
| AAAV (Avian AAV) | 542 | U.S. Pat. No. 9,238,800 SEQ ID NO: 12 |
| AAAV (Avian AAV) | 543 | U.S. Pat. No. 9,238,800 SEQ ID NO: 2 |
| AAAV (Avian AAV) | 544 | U.S. Pat. No. 9,238,800 SEQ ID NO: 6 |
| AAAV (Avian AAV) | 545 | U.S. Pat. No. 9,238,800 SEQ ID NO: 4 |
| AAAV (Avian AAV) | 546 | U.S. Pat. No. 9,238,800 SEQ ID NO: 8 |
| AAAV (Avian AAV) | 547 | U.S. Pat. No. 9,238,800 SEQ ID NO: 14 |
| AAAV (Avian AAV) | 548 | U.S. Pat. No. 9,238,800 SEQ ID NO: 10 |
| AAAV (Avian AAV) | 549 | U.S. Pat. No. 9,238,800 SEQ ID NO: 15 |
| AAAV (Avian AAV) | 550 | U.S. Pat. No. 9,238,800 SEQ ID NO: 5 |
| AAAV (Avian AAV) | 551 | U.S. Pat. No. 9,238,800 SEQ ID NO: 9 |
| AAAV (Avian AAV) | 552 | U.S. Pat. No. 9,238,800 SEQ ID NO: 3 |
| AAAV (Avian AAV) | 553 | U.S. Pat. No. 9,238,800 SEQ ID NO: 7 |
| AAAV (Avian AAV) | 554 | U.S. Pat. No. 9,238,800 SEQ ID NO: 11 |
| AAAV (Avian AAV) | 555 | U.S. Pat. No. 9,238,800 SEQ ID NO: 13 |
| AAAV (Avian AAV) | 556 | U.S. Pat. No. 9,238,800 SEQ ID NO: 1 |
| AAV Shuffle 100-1 | 557 | US20160017295 SEQ ID NO: 23 |
| AAV Shuffle 100-1 | 558 | US20160017295 SEQ ID NO: 11 |
| AAV Shuffle 100-2 | 559 | US20160017295 SEQ ID NO: 37 |
| AAV Shuffle 100-2 | 560 | US20160017295 SEQ ID NO: 29 |
| AAV Shuffle 100-3 | 561 | US20160017295 SEQ ID NO: 24 |
| AAV Shuffle 100-3 | 562 | US20160017295 SEQ ID NO: 12 |
| AAV Shuffle 100-7 | 563 | US20160017295 SEQ ID NO: 25 |
| AAV Shuffle 100-7 | 564 | US20160017295 SEQ ID NO: 13 |
| AAV Shuffle 10-2 | 565 | US20160017295 SEQ ID NO: 34 |
| AAV Shuffle 10-2 | 566 | US20160017295 SEQ ID NO: 26 |
| AAV Shuffle 10-6 | 567 | US20160017295 SEQ ID NO: 35 |
| AAV Shuffle 10-6 | 568 | US20160017295 SEQ ID NO: 27 |
| AAV Shuffle 10-8 | 569 | US20160017295 SEQ ID NO: 36 |
| AAV Shuffle 10-8 | 570 | US20160017295 SEQ ID NO: 28 |
| AAV SM 100-10 | 571 | US20160017295 SEQ ID NO: 41 |
| AAV SM 100-10 | 572 | US20160017295 SEQ ID NO: 33 |
| AAV SM 100-3 | 573 | US20160017295 SEQ ID NO: 40 |
| AAV SM 100-3 | 574 | US20160017295 SEQ ID NO: 32 |
| AAV SM 10-1 | 575 | US20160017295 SEQ ID NO: 38 |
| AAV SM 10-1 | 576 | US20160017295 SEQ ID NO: 30 |
| AAV SM 10-2 | 577 | US20160017295 SEQ ID NO: 10 |
| AAV SM 10-2 | 578 | US20160017295 SEQ ID NO: 22 |
| AAV SM 10-8 | 579 | US20160017295 SEQ ID NO: 39 |
| AAV SM 10-8 | 580 | US20160017295 SEQ ID NO: 31 |
| AAVF1/HSC1 | 581 | WO2016049230 SEQ ID NO: 20 |
| AAVF2/HSC2 | 582 | WO2016049230 SEQ ID NO: 21 |
| AAVF3/HSC3 | 583 | WO2016049230 SEQ ID NO: 22 |
| AAVF4/HSC4 | 584 | WO2016049230 SEQ ID NO: 23 |
| AAVF5/HSC5 | 585 | WO2016049230 SEQ ID NO: 25 |
| AAVF6/HSC6 | 586 | WO2016049230 SEQ ID NO: 24 |
| AAVF7/HSC7 | 587 | WO2016049230 SEQ ID NO: 27 |
| AAVF8/HSC8 | 588 | WO2016049230 SEQ ID NO: 28 |
| AAVF9/HSC9 | 589 | WO2016049230 SEQ ID NO: 29 |
| AAVF11/HSC11 | 590 | WO2016049230 SEQ ID NO: 26 |
| AAVF12/HSC12 | 591 | WO2016049230 SEQ ID NO: 30 |
| AAVF13/HSC13 | 592 | WO2016049230 SEQ ID NO: 31 |
| AAVF14/HSC14 | 593 | WO2016049230 SEQ ID NO: 32 |
| AAVF15/HSC15 | 594 | WO2016049230 SEQ ID NO: 33 |
| AAVF16/HSC16 | 595 | WO2016049230 SEQ ID NO: 34 |
| AAVF17/HSC17 | 596 | WO2016049230 SEQ ID NO: 35 |
| AAVF1/HSC1 | 597 | WO2016049230 SEQ ID NO: 2 |
| AAVF2/HSC2 | 598 | WO2016049230 SEQ ID NO: 3 |
| AAVF3/HSC3 | 599 | WO2016049230 SEQ ID NO: 5 |
| AAVF4/HSC4 | 600 | WO2016049230 SEQ ID NO: 6 |
| AAVF5/HSC5 | 601 | WO2016049230 SEQ ID NO: 11 |
| AAVF6/HSC6 | 602 | WO2016049230 SEQ ID NO: 7 |
| AAVF7/HSC7 | 603 | WO2016049230 SEQ ID NO: 8 |
| AAVF8/HSC8 | 604 | WO2016049230 SEQ ID NO: 9 |
| AAVF9/HSC9 | 605 | WO2016049230 SEQ ID NO: 10 |
| AAVF11/HSC11 | 606 | WO2016049230 SEQ ID NO: 4 |
| AAVF12/HSC12 | 607 | WO2016049230 SEQ ID NO: 12 |
| AAVF13/HSC13 | 608 | WO2016049230 SEQ ID NO: 14 |
| AAVF14/HSC14 | 609 | WO2016049230 SEQ ID NO: 15 |
| AAVF15/HSC15 | 610 | WO2016049230 SEQ ID NO: 16 |
| AAVF16/HSC16 | 611 | WO2016049230 SEQ ID NO: 17 |
| AAVF17/HSC17 | 612 | WO2016049230 SEQ ID NO: 13 |
| AAV CBr-E1 | 613 | U.S. Pat. No. 8,734,809 SEQ ID NO: 13 |
| AAV CBr-E2 | 614 | U.S. Pat. No. 8,734,809 SEQ ID NO: 14 |
| AAV CBr-E3 | 615 | U.S. Pat. No. 8,734,809 SEQ ID NO: 15 |
| AAV CBr-E4 | 616 | U.S. Pat. No. 8,734,809 SEQ ID NO: 16 |
| AAV CBr-E5 | 617 | U.S. Pat. No. 8,734,809 SEQ ID NO: 17 |
| AAV CBr-e5 | 618 | U.S. Pat. No. 8,734,809 SEQ ID NO: 18 |
| AAV CBr-E6 | 619 | U.S. Pat. No. 8,734,809 SEQ ID NO: 19 |
| AAV CBr-E7 | 620 | U.S. Pat. No. 8,734,809 SEQ ID NO: 20 |
| AAV CBr-E8 | 621 | U.S. Pat. No. 8,734,809 SEQ ID NO: 21 |
| AAV CLv-D1 | 622 | U.S. Pat. No. 8,734,809 SEQ ID NO: 22 |
| AAV CLv-D2 | 623 | U.S. Pat. No. 8,734,809 SEQ ID NO: 23 |
| AAV CLv-D3 | 624 | U.S. Pat. No. 8,734,809 SEQ ID NO: 24 |
| AAV CLv-D4 | 625 | U.S. Pat. No. 8,734,809 SEQ ID NO: 25 |
| AAV CLv-D5 | 626 | U.S. Pat. No. 8,734,809 SEQ ID NO: 26 |
| AAV CLv-D6 | 627 | U.S. Pat. No. 8,734,809 SEQ ID NO: 27 |
| AAV CLv-D7 | 628 | U.S. Pat. No. 8,734,809 SEQ ID NO: 28 |
| AAV CLv-D8 | 629 | U.S. Pat. No. 8,734,809 SEQ ID NO: 29 |
| AAV CLv-E1 | 630 | U.S. Pat. No. 8,734,809 SEQ ID NO: 13 |
| AAV CLv-R1 | 631 | U.S. Pat. No. 8,734,809 SEQ ID NO: 30 |
| AAV CLv-R2 | 632 | U.S. Pat. No. 8,734,809 SEQ ID NO: 31 |
| AAV CLv-R3 | 633 | U.S. Pat. No. 8,734,809 SEQ ID NO: 32 |
| AAV CLv-R4 | 634 | U.S. Pat. No. 8,734,809 SEQ ID NO: 33 |
| AAV CLv-R5 | 635 | U.S. Pat. No. 8,734,809 SEQ ID NO: 34 |
| AAV CLv-R6 | 636 | U.S. Pat. No. 8,734,809 SEQ ID NO: 35 |
| AAV CLv-R7 | 637 | U.S. Pat. No. 8,734,809 SEQ ID NO: 36 |
| AAV CLv-R8 | 638 | U.S. Pat. No. 8,734,809 SEQ ID NO: 37 |
| AAV CLv-R9 | 639 | U.S. Pat. No. 8,734,809 SEQ ID NO: 38 |
| AAV CLg-F1 | 640 | U.S. Pat. No. 8,734,809 SEQ ID NO: 39 |
| AAV CLg-F2 | 641 | U.S. Pat. No. 8,734,809 SEQ ID NO: 40 |
| AAV CLg-F3 | 642 | U.S. Pat. No. 8,734,809 SEQ ID NO: 41 |
| AAV CLg-F4 | 643 | U.S. Pat. No. 8,734,809 SEQ ID NO: 42 |
| AAV CLg-F5 | 644 | U.S. Pat. No. 8,734,809 SEQ ID NO: 43 |
| AAV CLg-F6 | 645 | U.S. Pat. No. 8,734,809 SEQ ID NO: 43 |
| AAV CLg-F7 | 646 | U.S. Pat. No. 8,734,809 SEQ ID NO: 44 |
| AAV CLg-F8 | 647 | U.S. Pat. No. 8,734,809 SEQ ID NO: 43 |
| AAV CSp-1 | 648 | U.S. Pat. No. 8,734,809 SEQ ID NO: 45 |
| AAV CSp-10 | 649 | U.S. Pat. No. 8,734,809 SEQ ID NO: 46 |
| AAV CSp-11 | 650 | U.S. Pat. No. 8,734,809 SEQ ID NO: 47 |
| AAV CSp-2 | 651 | U.S. Pat. No. 8,734,809 SEQ ID NO: 48 |
| AAV CSp-3 | 652 | U.S. Pat. No. 8,734,809 SEQ ID NO: 49 |
| AAV CSp-4 | 653 | U.S. Pat. No. 8,734,809 SEQ ID NO: 50 |
| AAV CSp-6 | 654 | U.S. Pat. No. 8,734,809 SEQ ID NO: 51 |
| AAV CSp-7 | 655 | U.S. Pat. No. 8,734,809 SEQ ID NO: 52 |
| AAV CSp-8 | 656 | U.S. Pat. No. 8,734,809 SEQ ID NO: 53 |
| AAV CSp-9 | 657 | U.S. Pat. No. 8,734,809 SEQ ID NO: 54 |
| AAV CHt-2 | 658 | U.S. Pat. No. 8,734,809 SEQ ID NO: 55 |
| AAV CHt-3 | 659 | U.S. Pat. No. 8,734,809 SEQ ID NO: 56 |
| AAV CKd-1 | 660 | U.S. Pat. No. 8,734,809 SEQ ID NO: 57 |
| AAV CKd-10 | 661 | U.S. Pat. No. 8,734,809 SEQ ID NO: 58 |
| AAV CKd-2 | 662 | U.S. Pat. No. 8,734,809 SEQ ID NO: 59 |
| AAV CKd-3 | 663 | U.S. Pat. No. 8,734,809 SEQ ID NO: 60 |
| AAV CKd-4 | 664 | U.S. Pat. No. 8,734,809 SEQ ID NO: 61 |
| AAV CKd-6 | 665 | U.S. Pat. No. 8,734,809 SEQ ID NO: 62 |
| AAVC Kd-7 | 666 | U.S. Pat. No. 8,734,809 SEQ ID NO: 63 |
| AAVC Kd-8 | 667 | U.S. Pat. No. 8,734,809 SEQ ID NO: 64 |
| AAV CLv-1 | 668 | U.S. Pat. No. 8,734,809 SEQ ID NO: 65 |
| AAV CLv-12 | 669 | U.S. Pat. No. 8,734,809 SEQ ID NO: 66 |
| AAV CLv-13 | 670 | U.S. Pat. No. 8,734,809 SEQ ID NO: 67 |
| AAVC Lv-2 | 671 | U.S. Pat. No. 8,734,809 SEQ ID NO: 68 |
| AAVC Lv-3 | 672 | U.S. Pat. No. 8,734,809 SEQ ID NO: 69 |
| AAV CLv-4 | 673 | U.S. Pat. No. 8,734,809 SEQ ID NO: 70 |
| AAVC Lv-6 | 674 | U.S. Pat. No. 8,734,809 SEQ ID NO: 71 |
| AAVC Lv-8 | 675 | U.S. Pat. No. 8,734,809 SEQ ID NO: 72 |
| AAVC Kd-B1 | 676 | U.S. Pat. No. 8,734,809 SEQ ID NO: 73 |
| AAV CKd-B2 | 677 | U.S. Pat. No. 8,734,809 SEQ ID NO: 74 |
| AAV CKd-B3 | 678 | U.S. Pat. No. 8,734,809 SEQ ID NO: 75 |
| AAV CKd-B4 | 679 | U.S. Pat. No. 8,734,809 SEQ ID NO: 76 |
| AAVC Kd-B5 | 680 | U.S. Pat. No. 8,734,809 SEQ ID NO: 77 |
| AAV CKd-B6 | 681 | U.S. Pat. No. 8,734,809 SEQ ID NO: 78 |
| AAV CKd-B7 | 682 | U.S. Pat. No. 8,734,809 SEQ ID NO: 79 |
| AAV CKd-B8 | 683 | U.S. Pat. No. 8,734,809 SEQ ID NO: 80 |
| AAV CKd-H1 | 684 | U.S. Pat. No. 8,734,809 SEQ ID NO: 81 |
| AAV CKd-H2 | 685 | U.S. Pat. No. 8,734,809 SEQ ID NO: 82 |
| AAV CKd-H3 | 686 | U.S. Pat. No. 8,734,809 SEQ ID NO: 83 |
| AAV CKd-H4 | 687 | U.S. Pat. No. 8,734,809 SEQ ID NO: 84 |
| AAV CKd-H5 | 688 | U.S. Pat. No. 8,734,809 SEQ ID NO: 85 |
| AAV CKd-H6 | 689 | U.S. Pat. No. 8,734,809 SEQ ID NO: 77 |
| AAV CHt-1 | 690 | U.S. Pat. No. 8,734,809 SEQ ID NO: 86 |
| AAV CLv1-1 | 691 | U.S. Pat. No. 8,734,809 SEQ ID NO: 171 |
| AAV CLv1-2 | 692 | U.S. Pat. No. 8,734,809 SEQ ID NO: 172 |
| AAV CLv1-3 | 693 | U.S. Pat. No. 8,734,809 SEQ ID NO: 173 |
| AAV CLv1-4 | 694 | U.S. Pat. No. 8,734,809 SEQ ID NO: 174 |
| AAV CLv1-7 | 695 | U.S. Pat. No. 8,734,809 SEQ ID NO: 175 |
| AAV Clv1-8 | 696 | U.S. Pat. No. 8,734,809 SEQ ID NO: 176 |
| AAV Clv1-9 | 697 | U.S. Pat. No. 8,734,809 SEQ ID NO: 177 |
| AAV Clv1-10 | 698 | U.S. Pat. No. 8,734,809 SEQ ID NO: 178 |
| AAV.VR-355 | 699 | U.S. Pat. No. 8,734,809 SEQ ID NO: 181 |
| AAV.hu.48R3 | 700 | U.S. Pat. No. 8,734,809 SEQ ID NO: 183 |
| AAV CBr-E1 | 701 | U.S. Pat. No. 8,734,809 SEQ ID NO: 87 |
| AAV CBr-E2 | 702 | U.S. Pat. No. 8,734,809 SEQ ID NO: 88 |
| AAV CBr-E3 | 703 | U.S. Pat. No. 8,734,809 SEQ ID NO: 89 |
| AAV CBr-E4 | 704 | U.S. Pat. No. 8,734,809 SEQ ID NO: 90 |
| AAV CBr-E5 | 705 | U.S. Pat. No. 8,734,809 SEQ ID NO: 91 |
| AAV CBr-e5 | 706 | U.S. Pat. No. 8,734,809 SEQ ID NO: 92 |
| AAVC Br-E6 | 707 | U.S. Pat. No. 8,734,809 SEQ ID NO: 93 |
| AAVC Br-E7 | 708 | U.S. Pat. No. 8,734,809 SEQ ID NO: 94 |
| AAV CBr-E8 | 709 | U.S. Pat. No. 8,734,809 SEQ ID NO: 95 |
| AAV CLv-D1 | 710 | U.S. Pat. No. 8,734,809 SEQ ID NO: 96 |
| AAV CLv-D2 | 711 | U.S. Pat. No. 8,734,809 SEQ ID NO: 97 |
| AAV CLv-D3 | 712 | U.S. Pat. No. 8,734,809 SEQ ID NO: 98 |
| AAV CLv-D4 | 713 | U.S. Pat. No. 8,734,809 SEQ ID NO: 99 |
| AAV CLv-D5 | 714 | U.S. Pat. No. 8,734,809 SEQ ID NO: 100 |
| AAV CLv-D6 | 715 | U.S. Pat. No. 8,734,809 SEQ ID NO: 101 |
| AAV CLv-D7 | 716 | U.S. Pat. No. 8,734,809 SEQ ID NO: 102 |
| AAV CLv-D8 | 717 | U.S. Pat. No. 8,734,809 SEQ ID NO: 103 |
| AAV CLv-E1 | 718 | U.S. Pat. No. 8,734,809 SEQ ID NO: 87 |
| AAV CLv-R1 | 719 | U.S. Pat. No. 8,734,809 SEQ ID NO: 104 |
| AAV CLv-R2 | 720 | U.S. Pat. No. 8,734,809 SEQ ID NO: 105 |
| AAV CLv-R3 | 721 | U.S. Pat. No. 8,734,809 SEQ ID NO: 106 |
| AAV CLv-R4 | 722 | U.S. Pat. No. 8,734,809 SEQ ID NO: 107 |
| AAV CLv-R5 | 723 | U.S. Pat. No. 8,734,809 SEQ ID NO: 108 |
| AAV CLv-R6 | 724 | U.S. Pat. No. 8,734,809 SEQ ID NO: 109 |
| AAV CLv-R7 | 725 | U.S. Pat. No. 8,734,809 SEQ ID NO: 110 |
| AAV CLv-R8 | 726 | U.S. Pat. No. 8,734,809 SEQ ID NO: 111 |
| AAV CLv-R9 | 727 | U.S. Pat. No. 8,734,809 SEQ ID NO: 112 |
| AAV CLg-F1 | 728 | U.S. Pat. No. 8,734,809 SEQ ID NO: 113 |
| AAV CLg-F2 | 729 | U.S. Pat. No. 8,734,809 SEQ ID NO: 114 |
| AAV CLg-F3 | 730 | U.S. Pat. No. 8,734,809 SEQ ID NO: 115 |
| AAV CLg-F4 | 731 | U.S. Pat. No. 8,734,809 SEQ ID NO: 116 |
| AAV CLg-F5 | 732 | U.S. Pat. No. 8,734,809 SEQ ID NO: 117 |
| AAV CLg-F6 | 733 | U.S. Pat. No. 8,734,809 SEQ ID NO: 117 |
| AAV CLg-F7 | 734 | U.S. Pat. No. 8,734,809 SEQ ID NO: 118 |
| AAV CLg-F8 | 735 | U.S. Pat. No. 8,734,809 SEQ ID NO: 117 |
| AAV CSp-1 | 736 | U.S. Pat. No. 8,734,809 SEQ ID NO: 119 |
| AAV CSp-10 | 737 | U.S. Pat. No. 8,734,809 SEQ ID NO: 120 |
| AAV CSp-11 | 738 | U.S. Pat. No. 8,734,809 SEQ ID NO: 121 |
| AAV CSp-2 | 739 | U.S. Pat. No. 8,734,809 SEQ ID NO: 122 |
| AAV CSp-3 | 740 | U.S. Pat. No. 8,734,809 SEQ ID NO: 123 |
| AAV CSp-4 | 741 | U.S. Pat. No. 8,734,809 SEQ ID NO: 124 |
| AAV CSp-6 | 742 | U.S. Pat. No. 8,734,809 SEQ ID NO: 125 |
| AAV CSp-7 | 743 | U.S. Pat. No. 8,734,809 SEQ ID NO: 126 |
| AAV CSp-8 | 744 | U.S. Pat. No. 8,734,809 SEQ ID NO: 127 |
| AAV CSp-9 | 745 | U.S. Pat. No. 8,734,809 SEQ ID NO: 128 |
| AAV CHt-2 | 746 | U.S. Pat. No. 8,734,809 SEQ ID NO: 129 |
| AAV CHt-3 | 747 | U.S. Pat. No. 8,734,809 SEQ ID NO: 130 |
| AAV CKd-1 | 748 | U.S. Pat. No. 8,734,809 SEQ ID NO: 131 |
| AAV CKd-10 | 749 | U.S. Pat. No. 8,734,809 SEQ ID NO: 132 |
| AAV CKd-2 | 750 | U.S. Pat. No. 8,734,809 SEQ ID NO: 133 |
| AAV CKd-3 | 751 | U.S. Pat. No. 8,734,809 SEQ ID NO: 134 |
| AAV CKd-4 | 752 | U.S. Pat. No. 8,734,809 SEQ ID NO: 135 |
| AAV CKd-6 | 753 | U.S. Pat. No. 8,734,809 SEQ ID NO: 136 |
| AAV CKd-7 | 754 | U.S. Pat. No. 8,734,809 SEQ ID NO: 137 |
| AAV CKd-8 | 755 | U.S. Pat. No. 8,734,809 SEQ ID NO: 138 |
| AAV CLv-1 | 756 | U.S. Pat. No. 8,734,809 SEQ ID NO: 139 |
| AAV CLv-12 | 757 | U.S. Pat. No. 8,734,809 SEQ ID NO: 140 |
| AAV CLv-13 | 758 | U.S. Pat. No. 8,734,809 SEQ ID NO: 141 |
| AAV CLv-2 | 759 | U.S. Pat. No. 8,734,809 SEQ ID NO: 142 |
| AAV CLv-3 | 760 | U.S. Pat. No. 8,734,809 SEQ ID NO: 143 |
| AAV CLv-4 | 761 | U.S. Pat. No. 8,734,809 SEQ ID NO: 144 |
| AAV CLv-6 | 762 | U.S. Pat. No. 8,734,809 SEQ ID NO: 145 |
| AAV CLv-8 | 763 | U.S. Pat. No. 8,734,809 SEQ ID NO: 146 |
| AAV CKd-B1 | 764 | U.S. Pat. No. 8,734,809 SEQ ID NO: 147 |
| AAV CKd-B2 | 765 | U.S. Pat. No. 8,734,809 SEQ ID NO: 148 |
| AAV CKd-B3 | 766 | U.S. Pat. No. 8,734,809 SEQ ID NO: 149 |
| AAV CKd-B4 | 767 | U.S. Pat. No. 8,734,809 SEQ ID NO: 150 |
| AAV CKd-B5 | 768 | U.S. Pat. No. 8,734,809 SEQ ID NO: 151 |
| AAV CKd-B6 | 769 | U.S. Pat. No. 8,734,809 SEQ ID NO: 152 |
| AAV CKd-B7 | 770 | U.S. Pat. No. 8,734,809 SEQ ID NO: 153 |
| AAV CKd-B8 | 771 | U.S. Pat. No. 8,734,809 SEQ ID NO: 154 |
| AAV CKd-H1 | 772 | U.S. Pat. No. 8,734,809 SEQ ID NO: 155 |
| AAV CKd-H2 | 773 | U.S. Pat. No. 8,734,809 SEQ ID NO: 156 |
| AAV CKd-H3 | 774 | U.S. Pat. No. 8,734,809 SEQ ID NO: 157 |
| AAV CKd-H4 | 775 | U.S. Pat. No. 8,734,809 SEQ ID NO: 158 |
| AAV CKd-H5 | 776 | U.S. Pat. No. 8,734,809 SEQ ID NO: 159 |
| AAV CKd-H6 | 777 | U.S. Pat. No. 8,734,809 SEQ ID NO: 151 |
| AAV CHt-1 | 778 | U.S. Pat. No. 8,734,809 SEQ ID NO: 160 |
| AAV CHt-P2 | 779 | WO2016065001 SEQ ID NO: 1 |
| AAV CHt-P5 | 780 | WO2016065001 SEQ ID NO: 2 |
| AAV CHt-P9 | 781 | WO2016065001 SEQ ID NO: 3 |
| AAV CBr-7.1 | 782 | WO2016065001 SEQ ID NO: 4 |
| AAV CBr-7.2 | 783 | WO2016065001 SEQ ID NO: 5 |
| AAV CBr-7.3 | 784 | WO2016065001 SEQ ID NO: 6 |
| AAV CBr-7.4 | 785 | WO2016065001 SEQ ID NO: 7 |
| AAV CBr-7.5 | 786 | WO2016065001 SEQ ID NO: 8 |
| AAV CBr-7.7 | 787 | WO2016065001 SEQ ID NO: 9 |
| AAV CBr-7.8 | 788 | WO2016065001 SEQ ID NO: 10 |
| AAV CBr-7.10 | 789 | WO2016065001 SEQ ID NO: 11 |
| AAV CKd-N3 | 790 | WO2016065001 SEQ ID NO: 12 |
| AAV CKd-N4 | 791 | WO2016065001 SEQ ID NO: 13 |
| AAV CKd-N9 | 792 | WO2016065001 SEQ ID NO: 14 |
| AAV CLv-L4 | 793 | WO2016065001 SEQ ID NO: 15 |
| AAV CLv-L5 | 794 | WO2016065001 SEQ ID NO: 16 |
| AAV CLv-L6 | 795 | WO2016065001 SEQ ID NO: 17 |
| AAV CLv-K1 | 796 | WO2016065001 SEQ ID NO: 18 |
| AAV CLv-K3 | 797 | WO2016065001 SEQ ID NO: 19 |
| AAV CLv-K6 | 798 | WO2016065001 SEQ ID NO: 20 |
| AAV CLv-M1 | 799 | WO2016065001 SEQ ID NO: 21 |
| AAV CLv-M11 | 800 | WO2016065001 SEQ ID NO: 22 |
| AAV CLv-M2 | 801 | WO2016065001 SEQ ID NO: 23 |
| AAV CLv-M5 | 802 | WO2016065001 SEQ ID NO: 24 |
| AAV CLv-M6 | 803 | WO2016065001 SEQ ID NO: 25 |
| AAV CLv-M7 | 804 | WO2016065001 SEQ ID NO: 26 |
| AAV CLv-M8 | 805 | WO2016065001 SEQ ID NO: 27 |
| AAV CLv-M9 | 806 | WO2016065001 SEQ ID NO: 28 |
| AAV CHt-P1 | 807 | WO2016065001 SEQ ID NO: 29 |
| AAV CHt-P6 | 808 | WO2016065001 SEQ ID NO: 30 |
| AAV CHt-P8 | 809 | WO2016065001 SEQ ID NO: 31 |
| AAV CHt-6.1 | 810 | WO2016065001 SEQ ID NO: 32 |
| AAV CHt-6.10 | 811 | WO2016065001 SEQ ID NO: 33 |
| AAV CHt-6.5 | 812 | WO2016065001 SEQ ID NO: 34 |
| AAV CHt-6.6 | 813 | WO2016065001 SEQ ID NO: 35 |
| AAV CHt-6.7 | 814 | WO2016065001 SEQ ID NO: 36 |
| AAV CHt-6.8 | 815 | WO2016065001 SEQ ID NO: 37 |
| AAV CSp-8.10 | 816 | WO2016065001 SEQ ID NO: 38 |
| AAV CSp-8.2 | 817 | WO2016065001 SEQ ID NO: 39 |
| AAV CSp-8.4 | 818 | WO2016065001 SEQ ID NO: 40 |
| AAV CSp-8.5 | 819 | WO2016065001 SEQ ID NO: 41 |
| AAV CSp-8.6 | 820 | WO2016065001 SEQ ID NO: 42 |
| AAV CSp-8.7 | 821 | WO2016065001 SEQ ID NO: 43 |
| AAV CSp-8.8 | 822 | WO2016065001 SEQ ID NO: 44 |
| AAV CSp-8.9 | 823 | WO2016065001 SEQ ID NO: 45 |
| AAV CBr-B7.3 | 824 | WO2016065001 SEQ ID NO: 46 |
| AAV CBr-B7.4 | 825 | WO2016065001 SEQ ID NO: 47 |
| AAV3B | 826 | WO2016065001 SEQ ID NO: 48 |
| AAV4 | 827 | WO2016065001 SEQ ID NO: 49 |
| AAV5 | 828 | WO2016065001 SEQ ID NO: 50 |
| AAV CHt-P2 | 829 | WO2016065001 SEQ ID NO: 51 |
| AAV CHt-P5 | 830 | WO2016065001 SEQ ID NO: 52 |
| AAV CHt-P9 | 831 | WO2016065001 SEQ ID NO: 53 |
| AAV CBr-7.1 | 832 | WO2016065001 SEQ ID NO: 54 |
| AAV CBr-7.2 | 833 | WO2016065001 SEQ ID NO: 55 |
| AAV CBr-7.3 | 834 | WO2016065001 SEQ ID NO: 56 |
| AAV CBr-7.4 | 835 | WO2016065001 SEQ ID NO: 57 |
| AAV CBr-7.5 | 836 | WO2016065001 SEQ ID NO: 58 |
| AAV CBr-7.7 | 837 | WO2016065001 SEQ ID NO: 59 |
| AAV CBr-7.8 | 838 | WO2016065001 SEQ ID NO: 60 |
| AAV CBr-7.10 | 839 | WO2016065001 SEQ ID NO: 61 |
| AAV CKd-N3 | 840 | WO2016065001 SEQ ID NO: 62 |
| AAV CK4-N4 | 841 | WO2016065001 SEQ ID NO: 63 |
| AAV CKd-N9 | 842 | WO2016065001 SEQ ID NO: 64 |
| AAV CLv-L4 | 843 | WO2016065001 SEQ ID NO: 65 |
| AAV CLv-L5 | 844 | WO2016065001 SEQ ID NO: 66 |
| AAV CLv-L6 | 845 | WO2016065001 SEQ ID NO: 67 |
| AAV CLv-K1 | 846 | WO2016065001 SEQ ID NO: 68 |
| AAV CLv-K3 | 847 | WO2016065001 SEQ ID NO: 69 |
| AAV CLv-K6 | 848 | WO2016065001 SEQ ID NO: 70 |
| AAV CLv-M1 | 849 | WO2016065001 SEQ ID NO: 71 |
| AAV CLv-M11 | 850 | WO2016065001 SEQ ID NO: 72 |
| AAV CLv-M2 | 851 | WO2016065001 SEQ ID NO: 73 |
| AAV CLv-M5 | 852 | WO2016065001 SEQ ID NO: 74 |
| AAV CLv-M6 | 853 | WO2016065001 SEQ ID NO: 75 |
| AAV CLv-M7 | 854 | WO2016065001 SEQ ID NO: 76 |
| AAV CLv-M8 | 855 | WO2016065001 SEQ ID NO: 77 |
| AAV CLv-M9 | 856 | WO2016065001 SEQ ID NO: 78 |
| AAV CHt-P1 | 857 | WO2016065001 SEQ ID NO: 79 |
| AAV CHt-P6 | 858 | WO2016065001 SEQ ID NO: 80 |
| AAV CHt-P8 | 859 | WO2016065001 SEQ ID NO: 81 |
| AAV CHt-6.1 | 860 | WO2016065001 SEQ ID NO: 82 |
| AAV CHt-6.10 | 861 | WO2016065001 SEQ ID NO: 83 |
| AAV CHt-6.5 | 862 | WO2016065001 SEQ ID NO: 84 |
| AAV CHt-6.6 | 863 | WO2016065001 SEQ ID NO: 85 |
| AAV CHt-6.7 | 864 | WO2016065001 SEQ ID NO: 86 |
| AAV CHt-6.8 | 865 | WO2016065001 SEQ ID NO: 87 |
| AAV CSp-8.10 | 866 | WO2016065001 SEQ ID NO: 88 |
| AAV CSp-8.2 | 867 | WO2016065001 SEQ ID NO: 89 |
| AAV CSp-8.4 | 868 | WO2016065001 SEQ ID NO: 90 |
| AAV CSp-8.5 | 869 | WO2016065001 SEQ ID NO: 91 |
| AAV CSp-8.6 | 870 | WO2016065001 SEQ ID NO: 92 |
| AAV CSp-8.7 | 871 | WO2016065001 SEQ ID NO: 93 |
| AAV CSp-8.8 | 872 | WO2016065001 SEQ ID NO: 94 |
| AAV CSp-8.9 | 873 | WO2016065001 SEQ ID NO: 95 |
| AAV CBr-B7.3 | 874 | WO2016065001 SEQ ID NO: 96 |
| AAV CBr-B7.4 | 875 | WO2016065001 SEQ ID NO: 97 |
| AAV3B | 876 | WO2016065001 SEQ ID NO: 98 |
| AAV4 | 877 | WO2016065001 SEQ ID NO: 99 |
| AAV5 | 878 | WO2016065001 SEQ ID NO: 100 |
| GPV | 879 | U.S. Pat. No. 9,624,274B2 SEQ ID NO: 192 |
| B19 | 880 | U.S. Pat. No. 9,624,274B2 SEQ ID NO: 193 |
| MVM | 881 | U.S. Pat. No. 9,624,274B2 SEQ ID NO: 194 |
| FPV | 882 | U.S. Pat. No. 9,624,274B2 SEQ ID NO: 195 |
| CPV | 883 | U.S. Pat. No. 9,624,274B2 SEQ ID NO: 196 |
| AAV6 | 884 | U.S. Pat. No. 9,546,112B2 SEQ ID NO: 5 |
| AAV6 | 885 | U.S. Pat. No. 9,457,103B2 SEQ ID NO: 1 |
| AAV2 | 886 | U.S. Pat. No. 9,457,103B2 SEQ ID NO: 2 |
| ShH10 | 887 | U.S. Pat. No. 9,457,103B2 SEQ ID NO: 3 |
| ShH13 | 888 | U.S. Pat. No. 9,457,103B2 SEQ ID NO: 4 |
| ShH10 | 889 | U.S. Pat. No. 9,457,103B2 SEQ ID NO: 5 |
| ShH10 | 890 | U.S. Pat. No. 9,457,103B2 SEQ ID NO: 6 |
| ShH10 | 891 | U.S. Pat. No. 9,457,103B2 SEQ ID NO: 7 |
| ShH10 | 892 | U.S. Pat. No. 9,457,103B2 SEQ ID NO: 8 |
| ShH10 | 893 | U.S. Pat. No. 9,457,103B2 SEQ ID NO: 9 |
| rh74 | 894 | U.S. Pat. No. 9,434,928B2 SEQ ID NO: 1, |
| | | US2015023924A1 SEQ ID NO: 2 |
| rh74 | 895 | U.S. Pat. No. 9,434,928B2 SEQ ID NO: 2, |
| | | US2015023924A1 SEQ ID NO: 1 |
| AAV8 | 896 | U.S. Pat. No. 9,434,928B2 SEQ ID NO: 4 |
| rh74 | 897 | U.S. Pat. No. 9,434,928B2 SEQ ID NO: 5 |
| rh74 (RHM4-1) | 898 | US2015023924A1 SEQ ID NO: 5, |
| | | US20160375110A1 SEQ ID NO: 4 |
| rh74 (RHM15-1) | 899 | US2015023924A1 SEQ ID NO: 6, |
| | | US20160375110A1 SEQ ID NO: 5 |
| rh74 (RHM15-2) | 900 | US2015023924A1 SEQ ID NO: 7, |
| | | US20160375110A1 SEQ ID NO: 6 |
| rh74 (RHM15-3/RHM15-5) | 901 | US2015023924A1 SEQ ID NO: 8, |
| | | US20160375110A1 SEQ ID NO: 7 |
| rh74 (RHM15-4) | 902 | US2015023924A1 SEQ ID NO: 9, |
| | | US20160375110A1 SEQ ID NO: 8 |
| rh74 (RHM15-6) | 903 | US2015023924A1 SEQ ID NO: 10, |
| | | US20160375110A1 SEQ ID NO: 9 |
| rh74 (RHM4-1) | 904 | US2015023924A1 SEQ ID NO: 11 |
| rh74 (RHM15-1) | 905 | US2015023924A1 SEQ ID NO: 12 |
| rh74 (RHM15-2) | 906 | US2015023924A1 SEQ ID NO: 13 |
| rh74 (RHM15-3/RHM15-5) | 907 | US2015023924A1 SEQ ID NO: 14 |
| rh74 (RHM15-4) | 908 | US2015023924A1 SEQ ID NO: 15 |
| rh74 (RHM15-6) | 909 | US2015023924A1 SEQ ID NO: 16 |
| AAV2 (comprising lung | 910 | US20160175389A1 SEQ ID NO: 9 |
| specific polypeptide) | | |
| AAV2 (comprising lung | 911 | US20160175389A1 SEQ ID NO: 10 |
| specific polypeptide) | | |
| Anc80 | 912 | US20170051257A1 SEQ ID NO: 1 |
| Anc80 | 913 | US20170051257A1 SEQ ID NO: 2 |
| Anc81 | 914 | US20170051257A1 SEQ ID NO: 3 |
| Anc80 | 915 | US20170051257A1 SEQ ID NO: 4 |
| Anc82 | 916 | US20170051257A1 SEQ ID NO: 5 |
| Anc82 | 917 | US20170051257A1 SEQ ID NO: 6 |
| Anc83 | 918 | US20170051257A1 SEQ ID NO: 7 |
| Anc83 | 919 | US20170051257A1 SEQ ID NO: 8 |
| Anc84 | 920 | US20170051257A1 SEQ ID NO: 9 |
| Anc84 | 921 | US20170051257A1 SEQ ID NO: 10 |
| Anc94 | 922 | US20170051257A1 SEQ ID NO: 11 |
| Anc94 | 923 | US20170051257A1 SEQ ID NO: 12 |
| Anc113 | 924 | US20170051257A1 SEQ ID NO: 13 |
| Anc113 | 925 | US20170051257A1 SEQ ID NO: 14 |
| Anc126 | 926 | US20170051257A1 SEQ ID NO: 15 |
| Anc126 | 927 | US20170051257A1 SEQ ID NO: 16 |
| Anc127 | 928 | US20170051257A1 SEQ ID NO: 17 |
| Anc127 | 929 | US20170051257A1 SEQ ID NO: 18 |
| Anc80L27 | 930 | US20170051257A1 SEQ ID NO: 19 |
| Anc80L59 | 931 | US20170051257A1 SEQ ID NO: 20 |
| Anc80L60 | 932 | US20170051257A1 SEQ ID NO: 21 |
| Anc80L62 | 933 | US20170051257A1 SEQ ID NO: 22 |
| Anc80L65 | 934 | US20170051257A1 SEQ ID NO: 23 |
| Anc80L33 | 935 | US20170051257A1 SEQ ID NO: 24 |
| Anc80L36 | 936 | US20170051257A1 SEQ ID NO: 25 |
| Anc80L44 | 937 | US20170051257A1 SEQ ID NO: 26 |
| Anc80L1 | 938 | US20170051257A1 SEQ ID NO: 35 |
| Anc80L1 | 939 | US20170051257A1 SEQ ID NO: 36 |
| AAV-X1 | 940 | U.S. Pat. No. 8,283,151B2 SEQ ID NO: 11 |
| AAV-X1b | 941 | U.S. Pat. No. 8,283,151B2 SEQ ID NO: 12 |
| AAV-X5 | 942 | U.S. Pat. No. 8,283,151B2 SEQ ID NO: 13 |
| AAV-X19 | 943 | U.S. Pat. No. 8,283,151B2 SEQ ID NO: 14 |
| AAV-X21 | 944 | U.S. Pat. No. 8,283,151B2 SEQ ID NO: 15 |
| AAV-X22 | 945 | U.S. Pat. No. 8,283,151B2 SEQ ID NO: 16 |
| AAV-X23 | 946 | U.S. Pat. No. 8,283,151B2 SEQ ID NO: 17 |
| AAV-X24 | 947 | U.S. Pat. No. 8,283,151B2 SEQ ID NO: 18 |
| AAV-X25 | 948 | U.S. Pat. No. 8,283,151B2 SEQ ID NO: 19 |
| AAV-X26 | 949 | U.S. Pat. No. 8,283,151B2 SEQ ID NO: 20 |
| AAV-X1 | 950 | U.S. Pat. No. 8,283,151B2 SEQ ID NO: 21 |
| AAV-X1b | 951 | U.S. Pat. No. 8,283,151B2 SEQ ID NO: 22 |
| AAV-X5 | 952 | U.S. Pat. No. 8,283,151B2 SEQ ID NO: 23 |
| AAV-X19 | 953 | U.S. Pat. No. 8,283,151B2 SEQ ID NO: 24 |
| AAV-X21 | 954 | U.S. Pat. No. 8,283,151B2 SEQ ID NO: 25 |
| AAV-X22 | 955 | U.S. Pat. No. 8,283,151B2 SEQ ID NO: 26 |
| AAV-X23 | 956 | U.S. Pat. No. 8,283,151B2 SEQ ID NO: 27 |
| AAV-X24 | 957 | U.S. Pat. No. 8,283,151B2 SEQ ID NO: 28 |
| AAV-X25 | 958 | U.S. Pat. No. 8,283,151B2 SEQ ID NO: 29 |
| AAV-X26 | 959 | U.S. Pat. No. 8,283,151B2 SEQ ID NO: 30 |
| AAVrh8 | 960 | WO2016054554A1 SEQ ID NO: 8 |
| AAVrh8VP2FC5 | 961 | WO2016054554A1 SEQ ID NO: 9 |
| AAVrh8VP2FC44 | 962 | WO2016054554A1 SEQ ID NO: 10 |
| AAVrh8VP2ApoB100 | 963 | WO2016054554A1 SEQ ID NO: 11 |
| AAVrh8VP2RVG | 964 | WO2016054554A1 SEQ ID NO: 12 |
| AAVrh8VP2Angiopep-2 | 965 | WO2016054554A1 SEQ ID NO: 13 |
| VP2 | | |
| AAV9.47VP1.3 | 966 | WO2016054554A1 SEQ ID NO: 14 |
| AAV9.47VP2ICAMg3 | 967 | WO2016054554A1 SEQ ID NO: 15 |
| AAV9.47VP2RVG | 968 | WO2016054554A1 SEQ ID NO: 16 |
| AAV9.47VP2Angiopep-2 | 969 | WO2016054554A1 SEQ ID NO: 17 |
| AAV9.47VP2A-string | 970 | WO2016054554A1 SEQ ID NO: 18 |
| AAVrh8VP2FC5 VP2 | 971 | WO2016054554A1 SEQ ID NO: 19 |
| AAVrb8VP2FC44 VP2 | 972 | WO2016054554A1 SEQ ID NO: 20 |
| AAVrh8VP2ApoB100 VP2 | 973 | WO2016054554A1 SEQ ID NO: 21 |
| AAVrh8VP2RVG VP2 | 974 | WO2016054554A1 SEQ ID NO: 22 |
| AAVrh8VP2Angiopep-2 | 975 | WO2016054554A1 SEQ ID NO: 23 |
| VP2 | | |
| AAV9.47VP2ICAMg3 VP2 | 976 | WO2016054554A1 SEQ ID NO: 24 |
| AAV9.47VP2RVG VP2 | 977 | WO2016054554A1 SEQ ID NO: 25 |
| AAV9.47VP2Angiopep-2 | 978 | WO2016054554A1 SEQ ID NO: 26 |
| VP2 | | |
| AAV9.47VP2A-string VP2 | 979 | WO2016054554A1 SEQ ID NO: 27 |
| rAAV-B1 | 980 | WO2016054557A1 SEQ ID NO: 1 |
| rAAV-B2 | 981 | WO2016054557A1 SEQ ID NO: 2 |
| rAAV-B3 | 982 | WO2016054557A1 SEQ ID NO: 3 |
| rAAV-B4 | 983 | WO2016054557A1 SEQ ID NO: 4 |
| rAAV-B1 | 984 | WO2016054557A1 SEQ ID NO: 5 |
| rAAV-B2 | 985 | WO2016054557A1 SEQ ID NO: 6 |
| rAAV-B3 | 986 | WO2016054557A1 SEQ ID NO: 7 |
| rAAV-B4 | 987 | WO2016054557A1 SEQ ID NO: 8 |
| rAAV-L1 | 988 | WO2016054557A1 SEQ ID NO: 9 |
| rAAV-L2 | 989 | WO2016054557A1 SEQ ID NO: 10 |
| rAAV-L3 | 990 | WO2016054557A1 SEQ ID NO: 11 |
| rAAV-L4 | 991 | WO2016054557A1 SEQ ID NO: 12 |
| rAAV-L1 | 992 | WO2016054557A1 SEQ ID NO: 13 |
| rAAV-L2 | 993 | WO2016054557A1 SEQ ID NO: 14 |
| rAAV-L3 | 994 | WO2016054557A1 SEQ ID NO: 15 |
| rAAV-L4 | 995 | WO2016054557A1 SEQ ID NO: 16 |
| AAV9 | 996 | WO2016073739A1 SEQ ID NO: 3 |
| rAAV | 997 | WO2016081811A1 SEQ ID NO: 1 |
| rAAV | 998 | WO2016081811A1 SEQ ID NO: 2 |
| rAAV | 999 | WO2016081811A1 SEQ ID NO: 3 |
| rAAV | 1000 | WO2016081811A1 SEQ ID NO: 4 |
| rAAV | 1001 | WO2016081811A1 SEQ ID NO: 5 |
| rAAV | 1002 | WO2016081811A1 SEQ ID NO: 6 |
| rAAV | 1003 | WO2016081811A1 SEQ ID NO: 7 |
| rAAV | 1004 | WO2016081811A1 SEQ ID NO: 8 |
| rAAV | 1005 | WO2016081811A1 SEQ ID NO: 9 |
| rAAV | 1006 | WO2016081811A1 SEQ ID NO: 10 |
| rAAV | 1007 | WO2016081811A1 SEQ ID NO: 11 |
| rAAV | 1008 | WO2016081811A1 SEQ ID NO: 12 |
| rAAV | 1009 | WO2016081811A1 SEQ ID NO: 13 |
| rAAV | 1010 | WO2016081811A1 SEQ ID NO: 14 |
| rAAV | 1011 | WO2016081811A1 SEQ ID NO: 15 |
| rAAV | 1012 | WO2016081811A1 SEQ ID NO: 16 |
| rAAV | 1013 | WO2016081811A1 SEQ ID NO: 17 |
| rAAV | 1014 | WO2016081811A1 SEQ ID NO: 18 |
| rAAV | 1015 | WO2016081811A1 SEQ ID NO: 19 |
| rAAV | 1016 | WO2016081811A1 SEQ ID NO: 20 |
| rAAV | 1017 | WO2016081811A1 SEQ ID NO: 21 |
| rAAV | 1018 | WO2016081811A1 SEQ ID NO: 22 |
| rAAV | 1019 | WO2016081811A1 SEQ ID NO: 23 |
| rAAV | 1020 | WO2016081811A1 SEQ ID NO: 24 |
| rAAV | 1021 | WO2016081811A1 SEQ ID NO: 25 |
| rAAV | 1022 | WO2016081811A1 SEQ ID NO: 26 |
| rAAV | 1023 | WO2016081811A1 SEQ ID NO: 27 |
| rAAV | 1024 | WO2016081811A1 SEQ ID NO: 28 |
| rAAV | 1025 | WO2016081811A1 SEQ ID NO: 29 |
| rAAV | 1026 | WO2016081811A1 SEQ ID NO: 30 |
| rAAV | 1027 | WO2016081811A1 SEQ ID NO: 31 |
| rAAV | 1028 | WO2016081811A1 SEQ ID NO: 32 |
| rAAV | 1029 | WO2016081811A1 SEQ ID NO: 33 |
| rAAV | 1030 | WO2016081811A1 SEQ ID NO: 34 |
| rAAV | 1031 | WO2016081811A1 SEQ ID NO: 35 |
| rAAV | 1032 | WO2016081811A1 SEQ ID NO: 36 |
| rAAV | 1033 | WO2016081811A1 SEQ ID NO: 37 |
| rAAV | 1034 | WO2016081811A1 SEQ ID NO: 38 |
| rAAV | 1035 | WO2016081811A1 SEQ ID NO: 39 |
| rAAV | 1036 | WO2016081811A1 SEQ ID NO: 40 |
| rAAV | 1037 | WO2016081811A1 SEQ ID NO: 41 |
| rAAV | 1038 | WO2016081811A1 SEQ ID NO: 42 |
| rAAV | 1039 | WO2016081811A1 SEQ ID NO: 43 |
| rAAV | 1040 | WO2016081811A1 SEQ ID NO: 44 |
| rAAV | 1041 | WO2016081811A1 SEQ ID NO: 45 |
| rAAV | 1042 | WO2016081811A1 SEQ ID NO: 46 |
| rAAV | 1043 | WO2016081811A1 SEQ ID NO: 47 |
| rAAV | 1044 | WO2016081811A1 SEQ ID NO: 48 |
| rAAV | 1045 | WO2016081811A1 SEQ ID NO: 49 |
| rAAV | 1046 | WO2016081811A1 SEQ ID NO: 50 |
| rAAV | 1047 | WO2016081811A1 SEQ ID NO: 51 |
| rAAV | 1048 | WO2016081811A1 SEQ ID NO: 52 |
| rAAV | 1049 | WO2016081811A1 SEQ ID NO: 53 |
| rAAV | 1050 | WO2016081811A1 SEQ ID NO: 54 |
| rAAV | 1051 | WO2016081811A1 SEQ ID NO: 55 |
| rAAV | 1052 | WO2016081811A1 SEQ ID NO: 56 |
| rAAV | 1053 | WO2016081811A1 SEQ ID NO: 57 |
| rAAV | 1054 | WO2016081811A1 SEQ ID NO: 58 |
| rAAV | 1055 | WO2016081811A1 SEQ ID NO: 59 |
| rAAV | 1056 | WO2016081811A1 SEQ ID NO: 60 |
| rAAV | 1057 | WO2016081811A1 SEQ ID NO: 61 |
| rAAV | 1058 | WO2016081811A1 SEQ ID NO: 62 |
| rAAV | 1059 | WO2016081811A1 SEQ ID NO: 63 |
| rAAV | 1060 | WO2016081811A1 SEQ ID NO: 64 |
| rAAV | 1061 | WO2016081811A1 SEQ ID NO: 65 |
| rAAV | 1062 | WO2016081811A1 SEQ ID NO: 66 |
| rAAV | 1063 | WO2016081811A1 SEQ ID NO: 67 |
| rAAV | 1064 | WO2016081811A1 SEQ ID NO: 68 |
| rAAV | 1065 | WO2016081811A1 SEQ ID NO: 69 |
| rAAV | 1066 | WO2016081811A1 SEQ ID NO: 70 |
| rAAV | 1067 | WO2016081811A1 SEQ ID NO: 71 |
| rAAV | 1068 | WO2016081811A1 SEQ ID NO: 72 |
| rAAV | 1069 | WO2016081811A1 SEQ ID NO: 73 |
| rAAV | 1070 | WO2016081811A1 SEQ ID NO: 74 |
| rAAV | 1071 | WO2016081811A1 SEQ ID NO: 75 |
| rAAV | 1072 | WO2016081811A1 SEQ ID NO: 76 |
| rAAV | 1073 | WO2016081811A1 SEQ ID NO: 77 |
| rAAV | 1074 | WO2016081811A1 SEQ ID NO: 78 |
| rAAV | 1075 | WO2016081811A1 SEQ ID NO: 79 |
| rAAV | 1076 | WO2016081811A1 SEQ ID NO: 80 |
| rAAV | 1077 | WO2016081811A1 SEQ ID NO: 81 |
| rAAV | 1078 | WO2016081811A1 SEQ ID NO: 82 |
| rAAV | 1079 | WO2016081811A1 SEQ ID NO: 83 |
| rAAV | 1080 | WO2016081811A1 SEQ ID NO: 84 |
| rAAV | 1081 | WO2016081811A1 SEQ ID NO: 85 |
| rAAV | 1082 | WO2016081811A1 SEQ ID NO: 86 |
| rAAV | 1083 | WO2016081811A1 SEQ ID NO: 87 |
| rAAV | 1084 | WO2016081811A1 SEQ ID NO: 88 |
| rAAV | 1085 | WO2016081811A1 SEQ ID NO: 89 |
| rAAV | 1086 | WO2016081811A1 SEQ ID NO: 90 |
| rAAV | 1087 | WO2016081811A1 SEQ ID NO: 91 |
| rAAV | 1088 | WO2016081811A1 SEQ ID NO: 92 |
| rAAV | 1089 | WO2016081811A1 SEQ ID NO: 93 |
| rAAV | 1090 | WO2016081811A1 SEQ ID NO: 94 |
| rAAV | 1091 | WO2016081811A1 SEQ ID NO: 95 |
| rAAV | 1092 | WO2016081811A1 SEQ ID NO: 96 |
| rAAV | 1093 | WO2016081811A1 SEQ ID NO: 97 |
| rAAV | 1094 | WO2016081811A1 SEQ ID NO: 98 |
| rAAV | 1095 | WO2016081811A1 SEQ ID NO: 99 |
| rAAV | 1096 | WO2016081811A1 SEQ ID NO: 100 |
| rAAV | 1097 | WO2016081811A1 SEQ ID NO: 101 |
| rAAV | 1098 | WO2016081811A1 SEQ ID NO: 102 |
| rAAV | 1099 | WO2016081811A1 SEQ ID NO: 103 |
| rAAV | 1100 | WO2016081811A1 SEQ ID NO: 104 |
| rAAV | 1101 | WO2016081811A1 SEQ ID NO: 105 |
| rAAV | 1102 | WO2016081811A1 SEQ ID NO: 106 |
| rAAV | 1103 | WO2016081811A1 SEQ ID NO: 107 |
| rAAV | 1104 | WO2016081811A1 SEQ ID NO: 108 |
| rAAV | 1105 | WO2016081811A1 SEQ ID NO: 109 |
| rAAV | 1106 | WO2016081811A1 SEQ ID NO: 110 |
| rAAV | 1107 | WO2016081811A1 SEQ ID NO: 111 |
| rAAV | 1108 | WO2016081811A1 SEQ ID NO: 112 |
| rAAV | 1109 | WO2016081811A1 SEQ ID NO: 113 |
| rAAV | 1110 | WO2016081811A1 SEQ ID NO: 114 |
| rAAV | 1111 | WO2016081811A1 SEQ ID NO: 115 |
| rAAV | 1112 | WO2016081811A1 SEQ ID NO: 116 |
| rAAV | 1113 | WO2016081811A1 SEQ ID NO: 117 |
| rAAV | 1114 | WO2016081811A1 SEQ ID NO: 118 |
| rAAV | 1115 | WO2016081811A1 SEQ ID NO: 119 |
| rAAV | 1116 | WO2016081811A1 SEQ ID NO: 120 |
| rAAV | 1117 | WO2016081811A1 SEQ ID NO: 121 |
| rAAV | 1118 | WO2016081811A1 SEQ ID NO: 122 |
| rAAV | 1119 | WO2016081811A1 SEQ ID NO: 123 |
| rAAV | 1120 | WO2016081811A1 SEQ ID NO: 124 |
| rAAV | 1121 | WO2016081811A1 SEQ ID NO: 125 |
| rAAV | 1122 | WO2016081811A1 SEQ ID NO: 126 |
| rAAV | 1123 | WO2016081811A1 SEQ ID NO: 127 |
| rAAV | 1124 | WO2016081811A1 SEQ ID NO: 128 |
| AAV8 E532K | 1125 | WO2016081811A1 SEQ ID NO: 133 |
| AAV8 E532K | 1126 | WO2016081811A1 SEQ ID NO: 134 |
| rAAV4 | 1127 | WO2016115382A1 SEQ ID NO: 2 |
| rAAV4 | 1128 | WO2016115382A1 SEQ ID NO: 3 |
| rAAV4 | 1129 | WO2016115382A1 SEQ ID NO: 4 |
| rAAV4 | 1130 | WO2016115382A1 SEQ ID NO: 5 |
| rAAV4 | 1131 | WO2016115382A1 SEQ ID NO: 6 |
| rAAV4 | 1132 | WO2016115382A1 SEQ ID NO: 7 |
| rAAV4 | 1133 | WO2016115382A1 SEQ ID NO: 8 |
| rAAV4 | 1134 | WO2016115382A1 SEQ ID NO: 9 |
| rAAV4 | 1135 | WO2016115382A1 SEQ ID NO: 10 |
| rAAV4 | 1136 | WO2016115382A1 SEQ ID NO: 11 |
| rAAV4 | 1137 | WO2016115382A1 SEQ ID NO: 12 |
| rAAV4 | 1138 | WO2016115382A1 SEQ ID NO: 13 |
| rAAV4 | 1139 | WO2016115382A1 SEQ ID NO: 14 |
| rAAV4 | 1140 | WO2016115382A1 SEQ ID NO: 15 |
| rAAV4 | 1141 | WO2016115382A1 SEQ ID NO: 16 |
| rAAV4 | 1142 | WO2016115382A1 SEQ ID NO: 17 |
| rAAV4 | 1143 | WO2016115382A1 SEQ ID NO: 18 |
| rAAV4 | 1144 | WO2016115382A1 SEQ ID NO: 19 |
| rAAV4 | 1145 | WO2016115382A1 SEQ ID NO: 20 |
| rAAV4 | 1146 | WO2016115382A1 SEQ ID NO: 21 |
| AAV11 | 1147 | WO2016115382A1 SEQ ID NO: 22 |
| AAV12 | 1148 | WO2016115382A1 SEQ ID NO: 23 |
| rh32 | 1149 | WO2016115382A1 SEQ ID NO: 25 |
| rh33 | 1150 | WO2016115382A1 SEQ ID NO: 26 |
| rh34 | 1151 | WO2016115382A1 SEQ ID NO: 27 |
| rAAV4 | 1152 | WO2016115382A1 SEQ ID NO: 28 |
| rAAV4 | 1153 | WO2016115382A1 SEQ ID NO: 29 |
| rAAV4 | 1154 | WO2016115382A1 SEQ ID NO: 30 |
| rAAV4 | 1155 | WO2016115382A1 SEQ ID NO: 31 |
| rAAV4 | 1156 | WO2016115382A1 SEQ ID NO: 32 |
| rAAV4 | 1157 | WO2016115382A1 SEQ ID NO: 33 |
| AAV2/8 | 1158 | WO2016131981A1 SEQ ID NO: 47 |
| AAV2/8 | 1159 | WO2016131981A1 SEQ ID NO: 48 |
| ancestral AAV | 1160 | WO2016154344A1 SEQ ID NO: 7 |
| ancestral AAV variant C4 | 1161 | WO2016154344A1 SEQ ID NO: 13 |
| ancestral AAV variant C7 | 1162 | WO2016154344A1 SEQ ID NO: 14 |
| ancestral AAV variant G4 | 1163 | WO2016154344A1 SEQ ID NO: 15 |
| consensus amino acid | 1164 | WO2016154344A1 SEQ ID NO: 16 |
| sequence of ancestral AAV | | |
| variants, C4, C7 and G4 | | |
| consensus amino acid | 1165 | WO2016154344A1 SEQ ID NO: 17 |
| sequence of ancestral AAV | | |
| variants, C4 and C7 | | |
| AAV8 (with a AAV2 | 1166 | WO2016150403A1 SEQ ID NO: 13 |
| phospholipase domain) | | |
| AAV VR-942n | 1167 | US20160289275A1 SEQ ID NO: 10 |
| AAV5-A (M569V) | 1168 | US20160289275A1 SEQ ID NO: 13 |
| AAV5-A (M569V) | 1169 | US20160289275A1 SEQ ID NO: 14 |
| AAV5-A (Y585V) | 1170 | US20160289275A1 SEQ ID NO: 16 |
| AAV5-A (Y585V) | 1171 | US20160289275A1 SEQ ID NO: 17 |
| AAV5-A (L587T) | 1172 | US20160289275A1 SEQ ID NO: 19 |
| AAV5-A (L587T) | 1173 | US20160289275A1 SEQ ID NO: 20 |
| AAV5-A (Y585V/L587T) | 1174 | US20160289275A1 SEQ ID NO: 22 |
| AAV5-A (Y585V/L587T) | 1175 | US20160289275A1 SEQ ID NO: 23 |
| AAV5-B (D652A) | 1176 | US20160289275A1 SEQ ID NO: 25 |
| AAV5-B (D652A) | 1177 | US20160289275A1 SEQ ID NO: 26 |
| AAV5-B (T362M) | 1178 | US20160289275A1 SEQ ID NO: 28 |
| AAV5-B (T362M) | 1179 | US20160289275A1 SEQ ID NO: 29 |
| AAV5-B (Q359D) | 1180 | US20160289275A1 SEQ ID NO: 31 |
| AAV5-B (Q359D) | 1181 | US20160289275A1 SEQ ID NO: 32 |
| AAV5-B (E350Q) | 1182 | US20160289275A1 SEQ ID NO: 34 |
| AAV5-B (E350Q) | 1183 | US20160289275A1 SEQ ID NO: 35 |
| AAV5-B (P533S) | 1184 | US20160289275A1 SEQ ID NO: 37 |
| AAV5-B (P533S) | 1185 | US20160289275A1 SEQ ID NO: 38 |
| AAV5-B (P533G) | 1186 | US20160289275A1 SEQ ID NO: 40 |
| AAV5-B (P533G) | 1187 | US20160289275A1 SEQ ID NO: 41 |
| AAV5-mutation in loop VII | 1188 | US20160289275A1 SEQ ID NO: 43 |
| AAV5-mutation in loop VII | 1189 | US20160289275A1 SEQ ID NO: 44 |
| AAV8 | 1190 | US20160289275A1 SEQ ID NO: 47 |
| Mut A (LK03/AAV8) | 1191 | WO2016181123A1 SEQ ID NO: 1 |
| Mut B (LK03/AAV5) | 1192 | WO2016181123A1 SEQ ID NO: 2 |
| Mut C (AAV8/AAV3B) | 1193 | WO2016181123A1 SEQ ID NO: 3 |
| Mut D (AAV5/AAV3B) | 1194 | WO2016181123A1 SEQ ID NO: 4 |
| Mut E (AAV8/AAV3B) | 1195 | WO2016181123A1 SEQ ID NO: 5 |
| Mut F (AAV3B/AAV8) | 1196 | WO2016181123A1 SEQ ID NO: 6 |
| AAV44.9 | 1197 | WO2016183297A1 SEQ ID NO: 4 |
| AAV44.9 | 1198 | WO2016183297A1 SEQ ID NO: 5 |
| AAVrh8 | 1199 | WO2016183297A1 SEQ ID NO: 6 |
| AAV44.9 (S470N) | 1200 | WO2016183297A1 SEQ ID NO: 9 |
| rh74 VP1 | 1201 | US20160375110A1 SEQ ID NO: 1 |
| AAV-LK03 (L125I) | 1202 | WO2017015102A1 SEQ ID NO: 5 |
| AAV3B (S663V + T492V) | 1203 | WO2017015102A1 SEQ ID NO: 6 |
| Anc80 | 1204 | WO2017019994A2 SEQ ID NO: 1 |
| Anc80 | 1205 | WO2017019994A2 SEQ ID NO: 2 |
| Anc81 | 1206 | WO2017019994A2 SEQ ID NO: 3 |
| Anc81 | 1207 | WO2017019994A2 SEQ ID NO: 4 |
| Anc82 | 1208 | WO2017019994A2 SEQ ID NO: 5 |
| Anc82 | 1209 | WO2017019994A2 SEQ ID NO: 6 |
| Anc83 | 1210 | WO2017019994A2 SEQ ID NO: 7 |
| Anc83 | 1211 | WO2017019994A2 SEQ ID NO: 8 |
| Anc84 | 1212 | WO2017019994A2 SEQ ID NO: 9 |
| Anc84 | 1213 | WO2017019994A2 SEQ ID NO: 10 |
| Anc94 | 1214 | WO2017019994A2 SEQ ID NO: 11 |
| Anc94 | 1215 | WO2017019994A2 SEQ ID NO: 12 |
| Anc113 | 1216 | WO2017019994A2 SEQ ID NO: 13 |
| Anc113 | 1217 | WO2017019994A2 SEQ ID NO: 14 |
| Anc126 | 1218 | WO2017019994A2 SEQ ID NO: 15 |
| Anc126 | 1219 | WO2017019994A2 SEQ ID NO: 16 |
| Anc127 | 1220 | WO2017019994A2 SEQ ID NO: 17 |
| Anc127 | 1221 | WO2017019994A2 SEQ ID NO: 18 |
| Anc80L27 | 1222 | WO2017019994A2 SEQ ID NO: 19 |
| Anc80L59 | 1223 | WO2017019994A2 SEQ ID NO: 20 |
| Anc80L60 | 1224 | WO2017019994A2 SEQ ID NO: 21 |
| Anc80L62 | 1225 | WO2017019994A2 SEQ ID NO: 22 |
| Anc80L65 | 1226 | WO2017019994A2 SEQ ID NO: 23 |
| Anc80L33 | 1227 | WO2017019994A2 SEQ ID NO: 24 |
| Anc80L36 | 1228 | WO2017019994A2 SEQ ID NO: 25 |
| Anc80L44 | 1229 | WO2017019994A2 SEQ ID NO: 26 |
| Anc80L1 | 1230 | WO2017019994A2 SEQ ID NO: 35 |
| Anc80L1 | 1231 | WO2017019994A2 SEQ ID NO: 36 |
| AAVrh10 | 1232 | WO2017019994A2 SEQ ID NO: 41 |
| Anc110 | 1233 | WO2017019994A2 SEQ ID NO: 42 |
| Anc110 | 1234 | WO2017019994A2 SEQ ID NO: 43 |
| AAVrh32.33 | 1235 | WO2017019994A2 SEQ ID NO: 45 |
| AAVrh74 | 1236 | WO2017049031A1 SEQ ID NO: 1 |
| AAV2 | 1237 | WO2017053629A2 SEQ ID NO: 49 |
| AAV2 | 1238 | WO2017053629A2 SEQ ID NO: 50 |
| AAV2 | 1239 | WO2017053629A2 SEQ ID NO: 82 |
| Parvo-like virus | 1240 | WO2017070476A2 SEQ ID NO: 1 |
| Parvo-like virus | 1241 | WO2017070476A2 SEQ ID NO: 2 |
| Parvo-like virus | 1242 | WO2017070476A2 SEQ ID NO: 3 |
| Parvo-like virus | 1243 | WO2017070476A2 SEQ ID NO: 4 |
| Parvo-like virus | 1244 | WO2017070476A2 SEQ ID NO: 5 |
| Parvo-like virus | 1245 | WO2017070476A2 SEQ ID NO: 6 |
| AAVrh.10 | 1246 | WO2017070516A1 SEQ ID NO: 7 |
| AAVrh.10 | 1247 | WO2017070516A1 SEQ ID NO: 14 |
| AAV2tYF | 1248 | WO2017070491A1 SEQ ID NO: 1 |
| AAV-SPK | 1249 | WO2017075619A1 SEQ ID NO: 28 |
| AAV2.5 | 1250 | US20170128528A1 SEQ ID NO: 13 |
| AAV1.1 | 1251 | US20170128528A1 SEQ ID NO: 15 |
| AAV6.1 | 1252 | US20170128528A1 SEQ ID NO: 17 |
| AAV6.3.1 | 1253 | US20170128528A1 SEQ ID NO: 18 |
| AAV2i8 | 1254 | US20170128528A1 SEQ ID NO: 28 |
| AAV2i8 | 1255 | US20170128528A1 SEQ ID NO: 29 |
| ttAAV | 1256 | US20170128528A1 SEQ ID NO: 30 |
| ttAAV-S312N | 1257 | US20170128528A1 SEQ ID NO: 32 |
| ttAAV-S312N | 1258 | US20170128528A1 SEQ ID NO: 33 |
| AAV6 (Y705, Y731, | 1259 | WO2016134337A1 SEQ ID NO: 24 |
| and T492) | | |
| AAV2 | 1260 | WO2016134375A1 SEQ ID NO: 9 |
| AAV2 | 1261 | WO2016134375A1 SEQ ID NO: 10 |

In some embodiments, the AAV serotype may be, or may have a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 137 and 138 respectively herein), PHP.B (SEQ ID NO: 8 and 9 of WO2015038958, herein SEQ ID NO: 5 and 6), G2B-13 (SEQ ID NO: 12 of WO2015038958, herein SEQ ID NO: 7), G2B-26 (SEQ ID NO: 13 of WO2015038958, herein SEQ ID NO: 5), TH1.1-32 (SEQ ID NO: 14 of WO2015038958, herein SEQ ID NO: 8), TH1.1-35 (SEQ ID NO: 15 of WO2015038958, herein SEQ ID NO: 9) or variants thereof. Further, any of the targeting peptides or amino acid inserts described in WO2015038958, may be inserted into any parent AAV serotype, such as, but not limited to, AAV9 (SEQ ID NO: 137 for the DNA sequence and SEQ ID NO: 138 for the amino acid sequence). In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, TLAVPFK (herein SEQ ID NO: 1262), KFPVALT (SEQ ID NO: 1263), LAVPFK (SEQ ID NO: 1264), AVPFK (SEQ ID NO: 1265), VPFK (SEQ ID NO: 1266), TLAVPF (SEQ ID NO: 1267), TLAVP (SEQ ID NO: 1268), TLAV (SEQ ID NO: 1269), SVSKPFL (SEQ ID NO: 1270), FTLTTPK (SEQ ID NO: 1271), MNATKNV (SEQ ID NO: 1272), QSSQTPR (SEQ ID NO: 1273), ILGTGTS (SEQ ID NO: 1274), TRTNPEA (SEQ ID NO: 1275), NGGTSSS (SEQ ID NO: 1276), or YTLSQGW (SEQ ID NO: 1277). Non-limiting examples of nucleotide sequences that may encode the amino acid inserts include the following, SEQ ID NO: 1278, SEQ ID NO: 1279, SEQ ID NO: 1280, SEQ ID NO: 1281, SEQ ID NO: 1282, SEQ ID NO: 1283, SEQ ID NO: 1284, SEQ ID NO: 1285, SEQ ID NO: 1286, or SEQ ID NO: 1287.

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 11), PHP.N (SEQ ID NO: 46 of WO2017100671, herein SEQ ID NO: 4), PHP.S (SEQ ID NO: 47 of WO2017100671, herein SEQ ID NO: 10), or variants thereof. Further, any of the targeting peptides or amino acid inserts described in WO2017100671 may be inserted into any parent AAV serotype, such as, but not limited to, AAV9. In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, AQTLAVPFKAQ (SEQ ID NO: 1288), AQSVSKPFLAQ (SEQ ID NO: 1289), AQFTLTTPKAQ (SEQ ID NO: 1290), DGTLAVPFKAQ (SEQ ID NO: 1291), ESTLAVPFKAQ (SEQ ID NO: 1292), GGTLAVPFKAQ (SEQ ID NO: 1293), AQTLATPFKAQ (SEQ ID NO: 1294), ATTLATPFKAQ (SEQ ID NO: 1295), DGTLATPFKAQ (SEQ ID NO: 1296), GGTLATPFKAQ (SEQ ID NO: 1297), SGSLAVPFKAQ (SEQ ID NO: 1298), AQTLAQPFKAQ (SEQ ID NO: 1299), AQTLQQPFKAQ (SEQ ID NO: 1300), AQTLSNPFKAQ (SEQ ID NO: 1301), AQTLAVPFSNP (SEQ ID NO: 1302), QGTLAVPFKAQ (SEQ ID NO: 1303), NQTLAVPFKAQ (SEQ ID NO: 1304), EGSLAVPFKAQ (SEQ ID NO: 1305), SGNLAVPFKAQ (SEQ ID NO: 1306), EGTLAVPFKAQ (SEQ ID NO: 1307), DSTLAVPFKAQ (SEQ ID NO: 1308), AVTLAVPFKAQ (SEQ ID NO: 1309), AQTLSTPFKAQ (SEQ ID NO: 1310), AQTLPQPFKAQ (SEQ ID NO: 1311), AQTLSQPFKAQ (SEQ ID NO: 1312), AQTLQLPFKAQ (SEQ ID NO: 1313), AQTLTMPFKAQ (SEQ ID NO: 1314), AQTLTTPFKAQ (SEQ ID NO: 1315), AQYTLSQGWAQ (SEQ ID NO: 1316), AQMNATKNVAQ (SEQ ID NO: 1317), AQVSGGHHSAQ (SEQ ID NO: 1318), AQTLTAPFKAQ (SEQ ID NO: 1319), AQTLSKPFKAQ (SEQ ID NO: 1320), QAVRTSL (SEQ ID NO: 1321), YTLSQGW (SEQ ID NO: 1277), LAKERLS (SEQ ID NO: 1322), TLAVPFK (SEQ ID NO: 1262), SVSKPFL (SEQ ID NO: 1270), FTLTTPK (SEQ ID NO: 1271), MNSTKNV (SEQ ID NO: 1323), VSGGHHS (SEQ ID NO: 1324), SAQTLAVPFKAQAQ (SEQ ID NO: 1325), SXXXLAVPFKAQAQ (wherein X may be any amino acid; SEQ ID NO: 1326), SAQXXXVPFKAQAQ (wherein X may be any amino acid; SEQ ID NO: 1327), SAQTLXXXFKAQAQ (wherein X may be any amino acid; SEQ ID NO: 1328), SAQTLAVXXXAQAQ (wherein X may be any amino acid; SEQ ID NO: 1329), SAQTLAVPFXXXAQ (wherein X may be any amino acid; SEQ ID NO: 1330), TNHQSAQ (SEQ ID NO: 1331), AQAQTGW (SEQ ID NO: 1332), DGTLATPFK (SEQ ID NO: 1333), DGTLATPFKXX (wherein X may be any amino acid; SEQ ID NO: 1334), LAVPFKAQ (SEQ ID NO: 1335), VPFKAQ (SEQ ID NO: 1336), FKAQ (SEQ ID NO: 1337), AQTLAV (SEQ ID NO: 1338), AQTLAVPF (SEQ ID NO: 1339), QAVR (SEQ ID NO: 1340), AVRT (SEQ ID NO: 1341), VRTS (SEQ ID NO: 1342), RTSL (SEQ ID NO: 1343), QAVRT (SEQ ID NO: 1344), AVRTS (SEQ ID NO: 1345), VRTSL (SEQ ID NO: 1346), QAVRTS (SEQ ID NO: 1347), or AVRTSL (SEQ ID NO: 1348). Non-limiting examples of nucleotide sequences that may encode the amino acid inserts include the following, SEQ ID NO: 1349, SEQ ID NO: 1350, SEQ ID NO: 1351, SEQ ID NO: 1352, SEQ ID NO: 1353, SEQ ID NO: 1354, SEQ ID NO: 1355, SEQ ID NO: 1356, SEQ ID NO: 1357, SEQ ID NO: 1358 (wherein N may be A, C, T, or G), SEQ ID NO: 1359 (wherein N may be A, C, T, or G), SEQ ID NO: 1360 (wherein N may be A, C, T, or G), SEQ ID NO: 1361 (wherein N may be A, C, T, or G); herein SEQ ID NO: 1362 (wherein N may be A, C, T, or G), SEQ ID NO: 1279, SEQ ID NO: 1280, SEQ ID NO: 1281, SEQ ID NO: 1287, or SEQ ID NO: 1363.

In some embodiments, the AAV serotype may be, or may have a sequence as described in U.S. Pat. No. 9,624,274, the contents of which are herein incorporated by reference in their entirety, such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ ID NO: 188 of U.S. Pat. No. 9,624,274), AAV11 (SEQ ID NO: 189 of U.S. Pat. No. 9,624,274), bAAV (SEQ ID NO: 190 of U.S. Pat. No. 9,624,274), AAV5 (SEQ ID NO: 191 of U.S. Pat. No. 9,624,274), GPV (SEQ ID NO: 192 of U.S. Pat. No. 9,624,274; herein SEQ ID NO: 879), B19 (SEQ ID NO: 193 of U.S. Pat. No. 9,624,274; herein SEQ ID NO: 880), MVM (SEQ ID NO: 194 of U.S. Pat. No. 9,624,274; herein SEQ ID NO: 881), FPV (SEQ ID NO: 195 of U.S. Pat. No. 9,624,274; herein SEQ ID NO: 882), CPV (SEQ ID NO: 196 of U.S. Pat. No. 9,624,274; herein SEQ ID NO: 883) or variants thereof. Further, any of the structural protein inserts described in U.S. Pat. No. 9,624,274, may be inserted into, but not limited to, I-453 and I-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 1364), EFCINHRGYWVCGD (SEQ ID NO: 1365), EDGQVMDVDLS (SEQ ID NO: 1366), EKQRNGTLT (SEQ ID NO: 1367), TYQCRVTHPHLPRALMR (SEQ ID NO: 1368), RHSTTQPRKTKGSG (SEQ ID NO: 1369), DSNPRGVSAYLSR (SEQ ID NO: 1370), TITCLWDLAPSK (SEQ ID NO: 1371), KTKGSGFFVF (SEQ ID NO: 1372), THPHLPRALMRS (SEQ ID NO: 1373), GETYQCRVTHPHLPRALMRSTTK (SEQ ID NO: 1374), LPRALMRS (SEQ ID NO: 1375), INHRGYWV (SEQ ID NO: 1376), CDAGSVRTNAPD (SEQ ID NO: 1377), AKAVSNLTESRSESLQS (SEQ ID NO: 1378), SLTGDEFKKVLET (SEQ ID NO: 1379), REAVAYRFEED (SEQ ID NO: 1380), INPEIITLDG (SEQ ID NO: 1381), DISVTGAPVITATYL (SEQ ID NO: 1382), DISVTGAPVITA (SEQ ID NO: 1383), PKTVSNLTESSSESVQS (SEQ ID NO: 1384), SLMGDEFKAVLET (SEQ ID NO: 1385), QHSVAYTFEED (SEQ ID NO: 1386), INPEIITRDG (SEQ ID NO: 1387), DISLTGDPVITASYL (SEQ ID NO: 1388), DISLTGDPVITA (SEQ ID NO: 1389), DQSIDFEIDSA (SEQ ID NO: 1390), KNVSEDLPLPTFSPTLLGDS (SEQ ID NO: 1391), KNVSEDLPLPT (SEQ ID NO: 1392), CDSGRVRTDAPD (SEQ ID NO: 1393), FPEHLLVDFLQSLS (SEQ ID NO: 1394), DAEFRHDSG (SEQ ID NO: 1395), HYAAAQWDFGNTMCQL (SEQ ID NO: 1396), YAAQWDFGNTMCQ (SEQ ID NO: 1397), RSQKEGLHYT (SEQ ID NO: 1398), SSRTPSDKPVAHWANPQAE (SEQ ID NO: 1399), SRTPSDKPVAHWANP (SEQ ID NO: 1400), SSRTPSDKP (SEQ ID NO: 1401), NADGNVDYHMNSVP (SEQ ID NO: 1402), DGNVDYHMNSV (SEQ ID NO: 1403), RSFKEFLQSSLRALRQ (SEQ ID NO: 1404); FKEFLQSSLRA (SEQ ID NO: 1405), or QMWAPQWGPD (SEQ ID NO: 1406).

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not be limited to, the amino acid sequence RGNRQA (SEQ ID NO: 1407), SSSTDP (SEQ ID NO: 1408), SSNTAP (SEQ ID NO: 1409), SNSNLP (herein SEQ ID NO: 1410), SSTTAP (SEQ ID NO: 1411), AANTAA (SEQ ID NO: 1412), QQNTAP (SEQ ID NO: 1413), SAQAQA (SEQ ID NO: 1414), QANTGP (SEQ ID NO: 1415), NATTAP (SEQ ID NO: 1416), SSTAGP (SEQ ID NO: 1417), QQNTAA (SEQ ID NO: 1418), PSTAGP (SEQ ID NO: 1419), NQNTAP (SEQ ID NO: 1420), QAANAP (SEQ ID NO: 1421), SIVGLP (SEQ ID NO: 1422), AASTAA (SEQ ID NO: 1423), SQNTTA (SEQ ID NO: 1424), QQDTAP (SEQ ID NO: 1425), QTNTGP (SEQ ID NO: 1426), QTNGAP (SEQ ID NO: 1427), QQNAAP (SEQ ID NO: 1428), or AANTQA (SEQ ID NO: 1429). In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 1430), QPEHSST (SEQ ID NO: 1431), VNTANST (SEQ ID NO: 1432), HGPMQKS (SEQ ID NO: 1433), PHKPPLA (SEQ ID NO: 1434), IKNNEMW (SEQ ID NO: 1435), RNLDTPM (SEQ ID NO: 1436), VDSHRQS (SEQ ID NO: 1437), YDSKTKT (SEQ ID NO: 1438), SQLPHQK (SEQ ID NO: 1439), STMQQNT (SEQ ID NO: 1440), TERYMTQ (SEQ ID NO: 1441), DASLSTS (SEQ ID NO: 1442), DLPNKKT (SEQ ID NO: 1443), DLTAARL (SEQ ID NO: 1444), EPHQFNY (SEQ ID NO: 1445), EPQSNHT (SEQ ID NO: 1446), MSSWPSQ (SEQ ID NO: 1447), NPKHNAT (SEQ ID NO: 1448), PDGMRTT (SEQ ID NO: 1449), PNNNKTT (SEQ ID NO: 1450), QSTTHDS (SEQ ID NO: 1451), TGSKQKQ (SEQ ID NO: 1452), SLKHQAL (SEQ ID NO: 1453), SPIDGEQ (SEQ ID NO: 1454), WIFPWIQL (SEQ ID NO: 1455), CDCRGDCFC (SEQ ID NO: 1456), CNGRC (SEQ ID NO: 1457), CPRECES (SEQ ID NO: 1458), CTTHWGFTLC (SEQ ID NO: 1459), CGRRAGGSC (SEQ ID NO: 1460), CKGGRAKDC (SEQ ID NO: 1461), CVPELGHEC (SEQ ID NO: 1462), CRRETAWAK (SEQ ID NO: 1463), VSWFSHRYSPFAVS (SEQ ID NO: 1464), GYRDGYAGPILYN (SEQ ID NO: 1465), XXXYXXX (SEQ ID NO: 1466), YXNW (SEQ ID NO: 1467), RPLPPLP (SEQ ID NO: 1468), APPLPPR (SEQ ID NO: 1469), DVFYPYPYASGS (SEQ ID NO: 1470), MYWYPY (SEQ ID NO: 1471), DITWDQLWDLMK (SEQ ID NO: 1472), CWDDXWLC (SEQ ID NO: 1473), EWCEYLGGYLRCYA (SEQ ID NO: 1474), YXCXXGPXTWXCXP (SEQ ID NO: 1475), IEGPTLRQWLAARA (SEQ ID NO: 1476), LWXXX (SEQ ID NO: 1477), XFXXYLW (SEQ ID NO: 1478), SSIISHFRWGLCD (SEQ ID NO: 1479), MSRPACPPNDKYE (SEQ ID NO: 1480), CLRSGRGC (SEQ ID NO: 1481), CHWMFSPWC (SEQ ID NO: 1482), WXXF (SEQ ID NO: 1483), CSSRLDAC (SEQ ID NO: 1484), CLPVASC (SEQ ID NO: 1485), CGFECVRQCPERC (SEQ ID NO: 1486), CVALCREACGEGC (SEQ ID NO: 1487), SWCEPGWCR (SEQ ID NO: 1488), YSGKWGW (SEQ ID NO: 1489), GLSGGRS (SEQ ID NO: 1490), LMLPRAD (SEQ ID NO: 1491), CSCFRDVCC (SEQ ID NO: 1492), CRDVVSVIC (SEQ ID NO: 1493), MARSGL (SEQ ID NO: 1494), MARAKE (SEQ ID NO: 1495), MSRTMS (SEQ ID NO: 1496, KCCYSL (SEQ ID NO: 1497), MYWGDSHWLQYWYE (SEQ ID NO: 1498), MQLPLAT (SEQ ID NO: 1499), EWLS (SEQ ID NO: 1500), SNEW (SEQ ID NO: 1501), TNYL (SEQ ID NO: 1502), WDLAWMFRLPVG (SEQ ID NO: 1503), CTVALPGGYVRVC (SEQ ID NO: 1504), CVAYCIEHHCWTC (SEQ ID NO: 1505), CVFAHNYDYLVC (SEQ ID NO: 1506), CVFTSNYAFC (SEQ ID NO: 1507), VHSPNKK (SEQ ID NO: 1508), CRGDGWC (SEQ ID NO: 1509), XRGCDX (SEQ ID NO: 1510), PXXX (SEQ ID NO: 1511), SGKGPRQITAL (SEQ ID NO: 1512), AAAAAAAAAXXXXX (SEQ ID NO: 1513), VYMSPF (SEQ ID NO: 1514), ATWLPPR (SEQ ID NO: 1515), HTMYYHHYQHHL (SEQ ID NO: 1516), SEVGCRAGPLQWLCEKYFG (SEQ ID NO: 1517), CGLLPVGRPDRNVWRWLC (SEQ ID NO: 1518), CKGQCDRFKGLPWEC (SEQ ID NO: 1519), SGRSA (SEQ ID NO: 1520), WGFP (SEQ ID NO: 1521), AEPMPHSLNFSQYLWYT (SEQ ID NO: 1522), WAYXSP (SEQ ID NO: 1523), IELLQAR (SEQ ID NO: 1524), AYTKCSRQWRTCMTTH (SEQ ID NO: 1525), PQNSKIPGPTFLDPH (SEQ ID NO: 1526), SMEPALPDWWWKMFK (SEQ ID NO: 1527), ANTPCGPYTHDCPVKR (SEQ ID NO: 1528), TACHQHVRMVRP (SEQ ID NO: 1529), VPWMEPAYQRFL (SEQ ID NO: 1530), DPRATPGS (SEQ ID NO: 1531), FRPNRAQDYNTN (SEQ ID NO: 1532), CTKNSYLMC (SEQ ID NO: 1533), CXXTXXXGXGC (SEQ ID NO: 1534), CPIEDRPMC (SEQ ID NO: 1535), HEWSYLAPYPWF (SEQ ID NO: 1536), MCPKHPLGC (SEQ ID NO: 1537), RMWPSSTVNLSAGRR (SEQ ID NO: 1538), SAKTAVSQRVWLPSHRGGEP (SEQ ID NO: 1539), KSREHVNNSACPSKRITAAL (SEQ ID NO: 1540), EGFR (SEQ ID NO: 1541), AGLGVR (SEQ ID NO: 1542), GTRQGHTMRLGVSDG (SEQ ID NO: 1543), IAGLATPGWSHWLAL (SEQ ID NO: 1544), SMSIARL (SEQ ID NO: 1545), HTFEPGV (SEQ ID NO: 1546), NTSLKRISNKRIRRK (SEQ ID NO: 1547), LRIKRKRRKRKKTRK (SEQ ID NO: 1548), GGG, GFS, LWS, EGG, LLV, LSP, LBS, AGG, GRR, GGH and GTV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1549) or variants thereof, wherein the specific site is at least one site selected from sites R447, G453, S578, N587, N587+1, S662 of VP1 or fragment thereof.

Further, any of the mutated sequences described in US 20160369298, may be or may have, but not limited to, any of the following sequences SDSGASN (SEQ ID NO: 1550), SPSGASN (SEQ ID NO: 1551), SHSGASN (SEQ ID NO: 1552), SRSGASN (SEQ ID NO: 1553), SKSGASN (SEQ ID NO: 1554), SNSGASN (SEQ ID NO: 1555), SGSGASN (SEQ ID NO: 1556), SASGASN (SEQ ID NO: 1557), SESGTSN (SEQ ID NO: 1558), STTGGSN (SEQ ID NO: 1559), SSAGSTN (SEQ ID NO: 1560), NNDSQA (SEQ ID NO: 1561), NNRNQA (SEQ ID NO: 1562), NNNKQA (SEQ ID NO: 1563), NAKRQA (SEQ ID NO: 1564), NDEHQA (SEQ ID NO: 1565), NTSQKA (SEQ ID NO: 1566), YYLSRTNTPSGTDTQSRLVFSQAGA (SEQ ID NO: 1567), YYLSRTNTDSGTETQSGLDFSQAGA (SEQ ID NO: 1568), YYLSRTNTESGTPTQSALEFSQAGA (SEQ ID NO: 1569), YYLSRTNTHSGTHTQSPLHFSQAGA (SEQ ID NO: 1570), YYLSRTNTSSGTITISHLIFSQAGA (SEQ ID NO: 1571), YYLSRTNTRSGIMTKSSLMFSQAGA (SEQ ID NO: 1572), YYLSRTNTKSGRKTLSNLSFSQAGA (SEQ ID NO: 1573), YYLSRTNDGSGPVTPSKLRFSQRGA (SEQ ID NO: 1574), YYLSRTNAASGHATHSDLKFSQPGA (SEQ ID NO: 1575), YYLSRTNGQAGSLTMSELGFSQVGA (SEQ ID NO: 1576), YYLSRTNSTGGNQTTSQLLFSQLSA (SEQ ID NO: 1577), YFLSRTNNNTGLNTNSTLNFSQGRA (SEQ ID NO: 1578), SKTGADNNNSEYSWTG (SEQ ID NO: 1579), SKTDADNNNSEYSWTG (SEQ ID NO: 1580), SKTEADNNNSEYSWTG (SEQ ID NO: 1581), SKTPADNNNSEYSWTG (SEQ ID NO: 1582), SKTHADNNNSEYSWTG (SEQ ID NO: 1583), SKTQADNNNSEYSWTG (SEQ ID NO: 1584), SKTIADNNNSEYSWTG (SEQ ID NO: 1585), SKTMADNNNSEYSWTG (SEQ ID NO: 1586), SKTRADNNNSEYSWTG (SEQ ID NO: 1587), SKTNADNNNSEYSWTG (SEQ ID NO: 1588), SKTVGRNNNSEYSWTG (SEQ ID NO: 1589), SKTADRNNNSEYSWTG (SEQ ID NO: 1590), SKKLSQNNNSKYSWQG (SEQ ID NO: 1591), SKPTTGNNNSDYSWPG (SEQ ID NO: 1592), STQKNENNNSNYSWPG (SEQ ID NO: 1593), HKDDEGKF (SEQ ID NO: 1594), HKDDNRKF (SEQ ID NO: 1595), HKDDTNKF (SEQ ID NO: 1596), HEDSDKNF (SEQ ID NO: 1597), HRDGADSF (SEQ ID NO: 1598), HGDNKSRF (SEQ ID NO: 1599), KQGSEKTNVDFEEV (SEQ ID NO: 1600), KQGSEKTNVDSEEV (SEQ ID NO: 1601), KQGSEKTNVDVEEV (SEQ ID NO: 1602), KQGSDKTNVDDAGV (SEQ ID NO: 1603), KQGSSKTNVDPREV (SEQ ID NO: 1604), KQGSRKTNVDHKQV (SEQ ID NO: 1605), KQGSKGGNVDTNRV (SEQ ID NO: 1606), KQGSGEANVDNGDV (SEQ ID NO: 1607), KQDAAADNIDYDHV (SEQ ID NO: 1608), KQSGTRSNAAASSV (SEQ ID NO: 1609), KENTNTNDTELTNV (SEQ ID NO: 1610), QRGNNVAATADVNT (SEQ ID NO: 1611), QRGNNEAATADVNT (SEQ ID NO: 1612), QRGNNPAATADVNT (SEQ ID NO: 1613), QRGNNHAATADVNT (SEQ ID NO: 1614), QEENNIAATPGVNT (SEQ ID NO: 1615), QPPNNMAATHEVNT (SEQ ID NO: 1616), QHHNNSAATTIVNT (SEQ ID NO: 1617), QTTNNRAAFNMVET (SEQ ID NO: 1618), QKKNNNAASKKVAT (SEQ ID NO: 1619), QGGNNKAADDAVKT (SEQ ID NO: 1620), QAAKGGAADDAVKT (SEQ ID NO: 1621), QDDRAAAANESVDT (SEQ ID NO: 1622), QQQHDDAAYQRVHT (SEQ ID NO: 1623), QSSSSLAAVSTVQT (SEQ ID NO: 1624), QNNQTTAAIRNVTT (SEQ ID NO: 1625), NYNKKSDNVDFT (SEQ ID NO: 1626), NYNKKSENVDFT (SEQ ID NO: 1627), NYNKKSLNVDFT (SEQ ID NO: 1628), NYNKKSPNVDFT (SEQ ID NO: 1629), NYSKKSHCVDFT (SEQ ID NO: 1630), NYRKTIYVDFT (SEQ ID NO: 1631), NYKEKKDVHFT (SEQ ID NO: 1632), NYGHRAIVQFT (SEQ ID NO: 1633), NYANHQFVVCT (SEQ ID NO: 1634), NYDDDPTGVLLT (SEQ ID NO: 1635), NYDDPTGVLLT (SEQ ID NO: 1636), NFEQQNSVEWT (SEQ ID NO: 1637), SQSGASN (SEQ ID NO: 1638), NNGSQA (SEQ ID NO: 1639), YYLSRTNTPSGTTTWSRLQFSQAGA (SEQ ID NO: 1640), SKTSADNNNSEYSWTG (SEQ ID NO: 1641), HKDDEEKF (SEQ ID NO: 1642), KQGSEKTNVDIEEV (SEQ ID NO: 1643), QRGNNQAATADVNT (SEQ ID NO: 1644), NYNKKSVNVDFT (SEQ ID NO: 1645), SQSGASNYNTPSGTTTQSRLQFSTSADNNNSEYSWTGATKYH (SEQ ID NO: 1646), SASGASNFNSEGGSLTQSSLGFSTDGENNNSDFSWTGATKYH (SEQ ID NO: 1647), SQSGASNYNTPSGTTTQSRLQFSTDGENNNSDFSWTGATKYH (SEQ ID NO: 1648), SASGASNYNTPSGTTTQSRLQFSTSADNNNSEFSWPGATTYH (SEQ ID NO: 1649), SQSGASNFNSEGGSLTQSSLGFSTDGENNNSDFSWTGATKYH (SEQ ID NO: 1650), SASGASNYNTPSGSLTQSSLGFSTDGENNNSDFSWTGATKYH (SEQ ID NO: 1651), SQSGASNYNTPSGTTTQSRLQFSTSADNNNSDFSWTGATKYH (SEQ ID NO: 1652), SGAGASNFNSEGGSLTQSSLGFSTDGENNNSDFSWTGATKYH (SEQ ID NO: 1653), SGAGASN (SEQ ID NO: 1654), NSEGGSLTQSSLGFS (SEQ ID NO: 1655), TDGENNNSDFS (SEQ ID NO: 1656), SEFSWPGATT (SEQ ID NO: 1657), TSADNNNSDFSWT (SEQ ID NO: 1658), SQSGASNY (SEQ ID NO: 1659), NTPSGTTTQSRLQFS (SEQ ID NO: 1660), TSADNNNSEYSWTGATKYH (SEQ ID NO: 1661), SASGASNF (SEQ ID NO: 1662), TDGENNNSDFSWTGATKYH (SEQ ID NO: 1663), SASGASNY (SEQ ID NO: 1664), TSADNNNSEFSWPGATTYH (SEQ ID NO: 1665), NTPSGSLTQSSLGFS (SEQ ID NO: 1666), TSADNNNSDFSWTGATKYH (SEQ ID NO: 1667), SGAGASNF (SEQ ID NO: 1668), CTCCAGVVSVVSMRSRVCVNSGCAGCTDHCVVSRNSGTCVMSACACAA (SEQ ID NO: 1669), CTCCAGAGAGGCAACAGACAAGCAGCTACCGCAGATGTCAACACACAA (SEQ ID NO: 1670), SAAGASN (SEQ ID NO: 1671), YFLSRTNTESGSTTQSTLRFSQAG (SEQ ID NO: 1672), SKTSADNNNSDFS (SEQ ID NO: 1673), KQGSEKTDVDIDKV (SEQ ID NO: 1674), STAGASN (SEQ ID NO: 1675), YFLSRTNTTSGIETQSTLRFSQAG (SEQ ID NO: 1676), SKTDGENNNSDFS (SEQ ID NO: 1677), KQGAAADDVEIDGV (SEQ ID NO: 1678), SEAGASN (SEQ ID NO: 1679), YYLSRTNTPSGTTTQSRLQFSQAG (SEQ ID NO: 1680), SKTSADNNNSEYS (SEQ ID NO: 1681), KQGSEKTNVDIEKV (SEQ ID NO: 1682), YFLSRTNDASGSDTKSTLLFSQAG (SEQ ID NO: 1683), STTPSENNNSEYS (SEQ ID NO: 1684), SAAGATN (SEQ ID NO: 1685), YFLSRTNGEAGSATLSELRFSQAG (SEQ ID NO: 1686), HGDDADRF (SEQ ID NO: 1687), KQGAEKSDVEVDRV (SEQ ID NO: 1688), KQDSGGDNIDIDQV (SEQ ID NO: 1689), SDAGASN (SEQ ID NO: 1690), YFLSRTNTEGGHDTQSTLRFSQAG (SEQ ID NO: 1691), KEDGGGSDVAIDEV (SEQ ID NO: 1692), SNAGASN (SEQ ID NO: 1693), and YFLSRTNGEAGSATLSELRFSQPG (SEQ ID NO: 1694). Non-limiting examples of nucleotide sequences that may encode the amino acid mutated sites include the following, SEQ ID NO: 1695, SEQ ID NO: 1696, SEQ ID NO: 1697, SEQ ID NO: 1698, SEQ ID NO: 1699, SEQ ID NO: 1700, SEQ ID NO: 1701, SEQ ID NO: 1702, SEQ ID NO: 1703, SEQ ID NO: 1704, SEQ ID NO: 1705, SEQ ID NO: 1706, SEQ ID NO: 1707, SEQ ID NO: 1708, SEQ ID NO: 1709, SEQ ID NO: 1710, AGCAGGAGCTCCTTGGCCTCAGCGTGCGAG (SEQ ID NO: 264 of US20160369298; herein SEQ ID NO: 1711), SEQ ID NO: 1712, SEQ ID NO: 1713, SEQ ID NO: 1714, SEQ ID NO: 1715, SEQ ID NO: 1716, and SEQ ID NO: 1717.

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 1718), or AAV9 (SEQ ID NO: 11 of WO2016134375; herein SEQ ID NO: 1719). In some embodiments, modifications, such as insertions are made in AAV2 proteins at P34-A35, T138-A139, A139-P140, G453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ ID NO: 1720), or GETRAPL (SEQ ID NO: 4 of WO2016134375; herein SEQ ID NO: 1721).

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In some embodiments, the AAV serotype may be modified as described in the International Publication WO2017083722 the contents of which are herein incorporated by reference in their entirety. AAV serotypes may include, AAV1 (Y705+731F+T492V), AAV2 (Y444+500+730F+T491V), AAV3 (Y705+731F), AAV5, AAV 5(Y436+693+719F), AAV6 (VP3 variant Y705F/Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 1722) or NKDKLN (SEQ ID NO:2 of WO2017015102; herein SEQ ID NO: 1723). The epitope may be inserted in the region of amino acids 665 to 670 based on the numbering of the VP1 capsid of AAV8 (SEQ ID NO: 3 of WO2017015102) and/or residues 664 to 668 of AAV3B (SEQ ID NO: 3).

In some embodiments, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720L, A456T, Q457T, N458Q, K459S, T492S, K493A, S586R, S587G, S588N, T589R and/or 722T of AAV1 (SEQ ID NO: 1 of WO2017058892) in any combination, 244N, 246Q, 248R, 249E, 2501, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 5891 of AAV6 (SEQ ID NO:6 of WO2017058892) in any combination, 457T, 459N, 496G, 499N, 500N, 589Q, 590N and/or 592A of AAV8 (SEQ ID NO: 8 of WO2017058892) in any combination, 451I, 452N, 453G, 454S, 455G, 456Q, 457N and/or 458Q of AAV9 (SEQ ID NO: 9 of WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In some embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may include a peptide such as, but not limited to, PHP.B, PHP.B2, PHP.B3, PHP.A, PHP.S, G2A12, G2A15, G2A3, G2B4, and G2B5. In some embodiments, these AAV serotypes may be AAV9 (SEQ ID NO: 11 or 138) derivatives with a 7-amino acid insert between amino acids 588-589. Non-limiting examples of these 7-amino acid inserts include TLAVPFK (PHP.B; SEQ ID NO: 1262), SVSKPFL (PHP.B2; SEQ ID NO: 1270), FTLTTPK (PHP.B3; SEQ ID NO: 1271), YTLSQGW (PHP.A; SEQ ID NO: 1277), QAVRTSL (PHP.S; SEQ ID NO: 1321), LAKERLS (G2A3; SEQ ID NO: 1322), MNSTKNV (G2B4; SEQ ID NO: 1323), and/or VSGGHHS (G2B5; SEQ ID NO: 1324).

In some embodiments, the AAV serotype may be as described in Jackson et al. (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In some embodiments, the AAV serotype is a serotype comprising the AAVPHP.N (PHP.N) peptide, or a variant thereof. In some embodiments the AAV serotype is a serotype comprising the AAVPHP.B (PHP.B) peptide, or a variant thereof. In some embodiments, the AAV serotype is a serotype comprising the AAVPHP.A (PHP.A) peptide, or a variant thereof. In some embodiments, the AAV serotype is a serotype comprising the PHP.S peptide, or a variant thereof. In some embodiments, the AAV serotype is a serotype comprising the PHP.B2 peptide, or a variant thereof. In some embodiments, the AAV serotype is a serotype comprising the PHP.B3 peptide, or a variant thereof. In some embodiments, the AAV serotype is a serotype comprising the G2B4 peptide, or a variant thereof. In some embodiments, the AAV serotype is a serotype comprising the G2B5 peptide, or a variant thereof. In some embodiments the AAV serotype is VOY101, or a variant thereof. In some embodiments, the AAV serotype is VOY201, or a variant thereof.

In some embodiments the AAV serotype of an AAV particle, e.g., an AAV particle for the vectorized delivery of an NPC1 protein described herein, is AAV9, or a variant thereof. In some embodiments, the AAV particle, e.g., a recombinant AAV particle described herein, comprises an AAV9 capsid protein. In some embodiments, the AAV9 capsid protein comprises the amino acid sequence of SEQ ID NO: 138. In some embodiments, the nucleic acid sequence encoding the AAV9 capsid protein comprises the nucleotide sequence of SEQ ID NO: 137. In some embodiments, the AAV9 capsid protein comprises an amino acid sequence at least 70% identical to SEQ ID NO: 138, such as, 70%, 75%, 80%, 85%, 90%, 91%, 92%, 93%, 94%, 95%, 96%, 97%, 98%, 99%, or greater than 99%. In some embodiments, the nucleic acid sequence encoding the AAV9 capsid protein comprises a nucleotide sequence at least 70% identical to SEQ ID NO: 137, such as, 70%, 75%, 80%, 85%, 90%, 91%, 92%, 93%, 94%, 95%, 96%, 97%, 98%, 99%, or greater than 99%.

In some embodiments, the capsid protein comprises the amino acid sequence of SEQ ID NO: 11 or an amino acid sequence substantially identical (e.g., having at least 70%, 75%, 80%, 85%, 90%, 92%, 95%, 97%, 98%, or 99% sequence identity) thereto. In some embodiments the capsid protein comprises an amino acid sequence comprising at least one, two, or three modifications but no more than 30, 20, or 10 modifications, e.g., substitutions, relative to the amino acid sequence of SEQ ID NO: 11, optionally provided that position 449 does not comprise K, e.g., is R.

In some embodiments, the capsid protein, comprises the amino acid sequence of SEQ ID NO: 1 or an amino acid sequence substantially identical (e.g., having at least 70%, 75%, 80%, 85%, 90%, 92%, 95%, 97%, 98%, or 99% sequence identity) thereto. In some embodiments the capsid protein comprises an amino acid sequence comprising at least one, two, or three modifications but no more than 30, 20, or 10 modifications, e.g., substitutions, relative to the amino acid sequence of SEQ ID NO: 1. In some embodiments, the capsid protein comprises an amino acid sequence encoded by the nucleotide sequence of SEQ ID NO: 2 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the capsid protein comprises the nucleotide sequence of SEQ ID NO: 2 or a sequence substantially identical (e.g., having at least 70%, 75%, 80%, 85%, 90%, 92%, 95%, 97%, 98%, or 99% sequence identity) thereto.

In some embodiments, the capsid protein, e.g., an AAV9 capsid protein, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the capsid protein comprises an amino acid sequence comprising at least one, two, or three modifications but no more than 30, 20, or 10 modifications, e.g., substitutions, relative to the amino acid sequence of SEQ ID NO: 138. In some embodiments, the capsid protein comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the capsid protein comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the capsid protein comprises substitution at position K449, e.g., a K449R substitution, numbered according to SEQ ID NO: 138.

In some embodiments, the capsid protein comprises an insert comprising the amino acid sequence of TLAVPFK (SEQ ID NO: 1262). In some embodiments, the insert is present immediately subsequent to position 588, relative to a reference sequence numbered according to SEQ ID NO: 138. In some embodiments, the capsid protein comprises the amino acid substitutions of A587D and Q588G, numbered according to SEQ ID NO: 138.

In some embodiments, the capsid protein comprises the amino acid substitution of K449R, numbered according to SEQ ID NO: 138; and an insert comprising the amino acid sequence of TLAVPFK (SEQ ID NO: 1262), wherein the insert is present immediately subsequent to position 588, relative to a reference sequence numbered according to SEQ ID NO: 138.

In some embodiments, the capsid protein comprises the amino acid substitution of K449R, numbered according to SEQ ID NO: 138; an insert comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the capsid protein comprises an insert comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV serotype of the AAV particle, e.g., an AAV particle for the vectorized delivery of antibody molecule described herein (e.g., an anti-beta-amyloid antibody molecule), is an AAV9 K449R, or a variant thereof. In some embodiments, the AAV particle comprises an AAV9 K449 capsid protein. In some embodiments, the AAV9 K449R capsid protein comprises the amino acid sequence of SEQ ID NO: 11. In some embodiments, the AAV9 K449R capsid protein comprises an amino acid sequence at least 70% identical to SEQ ID NO: 11, such as, 70%, 75%, 80%, 85%, 90%, 91%, 92%, 93%, 94%, 95%, 96%, 97%, 98%, 99%, or greater than 99%.

In some embodiments, the AAV capsid of an AAV particle, e.g., an AAV particle for the vectorized delivery of an NPC1 protein described herein, allows for blood brain barrier penetration following intravenous administration. Non-limiting examples of such AAV capsids include AAV9, AAV9 K449R, VOY101, VOY201, or AAV capsids comprising a peptide insert such as, but not limited to, AAVPHP.N (PHP.N), AAVPHP.B (PHP.B), PHP.S, G2A3, G2B4, G2B5, G2A12, G2A15, PHP.B2, PHP.B3, AAV2.BR1, or AAVPHP.A (PHP.A).

In some embodiments, the AAV capsid has increased tropism for cells of the central nervous system. In some embodiments, the cells of the central nervous system are neurons. In another embodiment, the cells of the central nervous system are astrocytes.

In some embodiments, the AAV serotype has increased tropism for cells (e.g., neurons) in brain regions such as cortex, cerebellum, corpus callosum, and/or brain stem.

In some embodiments, the AAV capsid has increased tropism for cells in the liver (hepatic cells).

In some embodiments, the AAV capsid has increased tropism for cells of the muscle(s).

In certain embodiments, an AAV particle described herein comprises an AAV capsid from a first AAV serotype (e.g., an AAV9 serotype) and the viral genome of said AAV particle comprises an inverted terminal repeat from a second AAV serotype (e.g., an AAV2 serotype), wherein the first AAV serotype is different from the second AAV serotype.

In some embodiments, the initiation codon for translation of the AAV VP1 capsid protein may be CTG, TTG, or GTG as described in U.S. Pat. No. 8,163,543, the contents of which are herein incorporated by reference in their entirety. In some embodiments, the nucleotide sequence encoding the capsid protein, e.g., a VP1 capsid protein, comprises 3-20 mutations (e.g., substitutions), e.g., 3-15 mutations, 3-10 mutations, 3-5 mutations, 5-20 mutations, 5-15 mutations, 5-10 mutations, 10-20 mutations, 10-15 mutations, 15-20 mutations, 3 mutations, 5 mutations, 10 mutations, 12 mutations, 15 mutations, 18 mutations, or 20 mutations, relative to the nucleotide sequence of SEQ ID NO: 137.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific “SEQ ID NO:” (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### AAV Viral Genome

In some aspects, the AAV particle of the present disclosure serves as an expression vector comprising a viral genome which encodes NPC protein. Expression vectors are not limited to AAV and may be adenovirus, retrovirus, lentivirus, plasmid, vector, or any variant thereof.

In some embodiments, an AAV particle, e.g., an AAV particle for the vectorized delivery of an NPC1 protein described herein, comprises a viral genome, e.g., an AAV viral genome (e.g., a vector genome or AAV vector genome). In some embodiments, the viral genome, e.g., the AAV viral genome, further comprises an inverted terminal repeat (ITR) region, a promoter, an enhancer, an intron region, a Kozak sequence, an exon region, a nucleic acid encoding a transgene encoding a payload (e.g., an NPC1 protein described herein), a poly A signal region, or a combination thereof. In some embodiments, an AAV particle viral genome described herein may comprise, from ITR to ITR recited 5′ to 3′, an ITR, a promoter, an intron, a nucleic acid sequence encoding NPC protein, a polyA sequence, and an ITR.

### Viral Genome Component: Inverted Terminal Repeats (ITRs)

In some embodiments, the viral genome may comprise at least one inverted terminal repeat (ITR) region. In some embodiments, the AAV particles of the present disclosure comprise a viral genome with at least one ITR region and a payload region. In some embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes may be comprised of naturally occurring polynucleotide sequences or recombinantly derived polynucleotide sequences.

The ITRs may be derived from the same serotype as the capsid, selected from any of the serotypes listed in Table 1, or a derivative thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. In some embodiments, the ITR comprises 100-180 nucleotides in length, e.g., about 100-115, about 100-120, about 100-130, about 100-140, about 100-150, about 100-160, about 100-170, about 100-180, about 110-120, about 110-130, about 110-140, about 110-150, about 110-160, about 110-170, about 110-180, about 120-130, about 120-140, about 120-150, about 120-160, about 120-170, about 120-180, about 130-140, about 130-150, about 130-160, about 130-170, about 130-180, about 140-150, about 140-160, about 140-170, about 140-180, about 150-160, about 150-170, about 150-180, about 160-170, about 160-180, or about 170-180 nucleotides in length. In some embodiments, the ITR comprises about 120-140 nucleotides in length, e.g., about 130 nucleotides in length. In some embodiments, the ITRs are 140-142 nucleotides in length, e.g., 141 nucleotides in length. In some embodiments, the ITR comprises 1205-135 nucleotides in length, e.g., 130 nucleotides in length. Non-limiting examples of ITR length are 102, 130, 140, 141, 142, 145 nucleotides in length, and those having at least 95% identity thereto.

In some embodiments, each ITR may be 141 nucleotides in length.

In some embodiments, each ITR may be 130 nucleotides in length.

In some embodiments, the viral genome of an AAV particle described herein comprises two ITRs, wherein one ITR is 141 nucleotides in length and the other ITR is 130 nucleotides in length.

### Viral Genome Component: Promoters and Expression Enhancers

In some embodiments, the payload region of the viral genome comprises at least one element to enhance the transgene target specificity and expression. See, e.g., Powell et al. Viral Expression Cassette Elements to Enhance Transgene Target Specificity and Expression in Gene Therapy, 2015; the contents of which are herein incorporated by reference in their entirety. Non-limiting examples of elements to enhance the transgene target specificity and expression include promoters, endogenous miRNAs, post-transcriptional regulatory elements (PREs), polyadenylation (PolyA) signal sequences, upstream enhancers (USEs), CMV enhancers, and introns. In some embodiments, the viral genome comprises a promoter operably linked to a transgene encoded by a nucleic acid molecule encoding a payload, e.g., an NPC1 protein. In some embodiments, the viral genome comprises an enhancer, e.g., a CMVie enhancer.

In some embodiments, expression of the polypeptides in a target cell may be driven by a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In some embodiments, the viral genome comprises a promoter that is sufficient for expression, e.g., in a target cell, of a payload (e.g., an NPC1 protein) encoded by a transgene. In some embodiments, the promoter is deemed to be efficient when it drives expression of the polypeptide(s) encoded in the payload region of the viral genome of the AAV particle.

In some embodiments, the promoter is a promoter deemed to be efficient when it drives expression in the cell or tissue being targeted.

In some embodiments, the promoter drives expression of the NPC protein(s) for a period of time in targeted cells, tissues, and/or organs. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of the polypeptides (e.g., NPC protein) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be a truncated promoter.

In some embodiments, the viral genome comprises a promoter that results in expression in one or more, e.g., multiple, cells and/or tissues, e.g., a ubiquitous promoter. In some embodiments, promoters which drive or promote expression in most mammalian tissues include, but are not limited to, human elongation factor 1α-subunit (EF1α), cytomegalovirus (CMV) immediate-early enhancer and/or promoter, chicken j-actin (CBA) and its derivative CAG, β glucuronidase (GUSB), and ubiquitin C (UBC). In some embodiments, tissue-specific expression elements can be used to restrict expression to certain cell types such as, but not limited to, CNS-specific promoters, B cell promoters, monocyte promoters, leukocyte promoters, macrophage promoters, pancreatic acinar cell promoters, endothelial cell promoters, lung tissue promoters, astrocyte promoters, or various specific nervous system cell- or tissue-type promoters which can be used to restrict expression to neurons, astrocytes, or oligodendrocytes, for example.

In some embodiments, the viral genome comprises a nervous system specific promoter, e.g., a promoter that results in expression of a payload in a neuron, an astrocyte, and/or an oligodendrocyte. Non-limiting examples of tissue-specific expression elements for neurons include neuron-specific enolase (NSE), platelet-derived growth factor (PDGF), platelet-derived growth factor B-chain (PDGF-β), synapsin (Syn), synapsin 1 (Syn1), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), 0-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter. Prion promoter represents an additional tissue specific promoter useful for driving protein expression in CNS tissue (see Loftus, Stacie K., et al. “Rescue of neurodegeneration in Niemann-Pick C mice by a prion-promoter-driven Npc1 cDNA transgene.” Human molecular genetics 11.24 (2002): 3107-3114, the disclosure of which is incorporated by reference in its entirety).

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800 nucleotides.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CB6, CBh, etc.), EF-1a, PGK, UBC, GUSB (hGBp), and UCOE (promoter of HNRPA2B1-CBX3). In some embodiments, the viral genome comprises an EF-1α promoter or EF-1α promoter variant, e.g., a truncated EF-1α promoter.

In some embodiments, the promoter is a ubiquitous promoter as described in Yu et al. (Molecular Pain 2011, 7:63), Soderblom et al. (E. Neuro 2015), Gill et al., (Gene Therapy 2001, Vol. 8, 1539-1546), and Husain et al. (Gene Therapy 2009), each of which are incorporated by reference in their entirety.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides.

In some embodiments, the promoter is a 0-glucuronidase (GUSB) promoter. The GUSB promoter may have a size of 350-400 nucleotides. As a non-limiting example, the GUSB promoter is 378 nucleotides.

In some embodiments, the promoter is a neurofilament light (NFL) promoter. The NFL promoter may have a size of 600-700 nucleotides. As a non-limiting example, the NFL promoter is 650 nucleotides.

In some embodiments, the promoter is a neurofilament heavy (NFH) promoter. The NFH promoter may have a size of 900-950 nucleotides. As a non-limiting example, the NFH promoter is 920 nucleotides.

In some embodiments, the promoter is a scn8a promoter. The scn8a promoter may have a size of 450-500 nucleotides. As a non-limiting example, the scn8a promoter is 470 nucleotides.

In some embodiments, the promoter is a phosphoglycerate kinase 1 (PGK) promoter.

In some embodiments, the promoter is a chicken j-actin (CBA) promoter, or a variant thereof.

In some embodiments, the promoter is a CB6 promoter.

In some embodiments, the promoter is a minimal CB promoter.

In some embodiments, the promoter is a cytomegalovirus (CMV) promoter.

In some embodiments, the promoter is a CAG promoter.

In some embodiments, the promoter is a GFAP promoter to drive NPC protein expression in astrocytes, as described, for example, in Zhang, Min, et al. “Astrocyte-only Npc1 reduces neuronal cholesterol and triples life span of Npc1−/− mice.” Journal of neuroscience research 86.13 (2008): 2848-2856, the disclosure of which is incorporated by reference in its entirety.

In some embodiments, the promoter is a synapsin promoter.

In some embodiments, the promoter is an RNA pol III promoter. As a non-limiting example, the RNA pol III promoter is U6. As a non-limiting example, the RNA pol III promoter is H1.

In some embodiments, the viral genome comprises two promoters. As a non-limiting example, the promoters are an EF1α promoter and a CMV promoter.

In another embodiment, the viral genome comprises a promoter from a naturally expressed protein.

In some embodiments, the NPC1 and/or NPC2 promoter is used in the viral genomes of the AAV particles encoding NPC protein or a variant thereof. In some embodiments the NPC1 and/or NPC2 promoter is engineered for optimal NPC protein expression.

In some embodiments, the promoter promotes widespread NPC1 distribution throughout the periphery and CNS tissues (e.g., neurons) to provide robust efficacy.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

### Viral Genome Component: Introns

In some embodiments, the viral genome comprises at least one intron or a fragment or derivative thereof. In some embodiments, the at least one intron may enhance expression of NPC protein (see e.g., Powell et al. Viral Expression Cassette Elements to Enhance Transgene Target Specificity and Expression in Gene Therapy, 2015; the contents of which are herein incorporated by reference in their entirety). Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps), and hybrid adenovirus splice donor/IgG splice acceptor (230 bps).

In some embodiments, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

In some embodiments, the AAV viral genome may comprise an SV40 intron or fragment or variant thereof.

In some embodiments, the AAV viral genome may comprise one or more beta-globin introns or a fragment or variant thereof. In some embodiments, the intron comprises one or more human beta-globin sequences (e.g., including fragments/variants thereof).

In some embodiments, the encoded NPC protein may be located downstream of an intron (e.g., 3′ relative to the intron) in an expression vector or a viral genome described herein, such as, but not limited to, SV40 intron or beta globin intron or others known in the art. Further, the encoded NPC protein may also be located upstream of the polyadenylation sequence (e.g., 5′ relative to the polyadenylation sequence) in an expression vector or a viral genome described herein. In some embodiments, the encoded NPC protein may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides downstream from the promoter with an intron (e.g., 3′ relative to the promoter) and/or upstream of the polyadenylation sequence (e.g., 5′ relative to the polyadenylation sequence) in an expression vector or a viral genome described herein. In some embodiments, the encoded NPC protein may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30, or 25-30 nucleotides downstream from the intron (e.g., 3′ relative to the intron) and/or upstream of the polyadenylation sequence (e.g., 5′ relative to the polyadenylation sequence) in an expression vector or a viral genome described herein. In some embodiments, the encoded NPC protein may be located within the first 1%, 2%, 3%, 4%, 5%, 6%, 7%, 8%, 9%, 10%, 15%, 20%, 25%, or more than 25% of the nucleotides downstream from the intron and/or upstream of the polyadenylation sequence in an expression vector or a viral genome described herein. In some embodiments, the encoded NPC protein may be located within the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% of the sequence downstream from the intron and/or upstream of the polyadenylation sequence in an expression vector or a viral genome described herein.

In some embodiments, the viral genome encoding an NPC1 protein described herein comprises a chimeric intron (e.g., a pClneo-intron). In some embodiments, the intron comprises the nucleotide sequence of:
gtaagtatcaaggttacaagacaggtttaaggagaccaatagaaactgggcttgtcgagacagagaagactcttgcgtttctgataggcacc tattggtcttactgacatccactttgcctttctctccacag (SEQ ID NO: 1780).

In some embodiments, the intron, e.g., the chimeric intron (e.g., a pClneo-intron), is located between the promoter and the transgene encoding the NPC1 protein. Without wishing to be bound by theory, it is believed in some embodiments, that the presence of an intron, e.g., a pClneo-intron, in a viral genome described herein enhances the expression of the encoded NPC1 protein.

In some embodiments, the intron sequence is not an enhancer sequence. In certain embodiments, the intron sequence is not a sub-component of a promoter sequence.

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the disclosure to enhance expression in hepatic cell lines or liver.

In some embodiments, the viral genome encoding a transgene described herein (e.g., a transgene encoding an NPC1 protein) comprises a Kozak sequence. While not wishing to be bound by theory, wild-type 5′ untranslated regions (UTRs) include features that play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

While not wishing to be bound by theory, wild-type 3′ UTRs are known to have stretches of adenosines and uridines embedded therein. These AU rich signatures are particularly prevalent in genes with high rates of turnover. Based on their sequence features and functional properties, the AU rich elements (AREs) can be separated into three classes (Chen et al., 1995, the contents of which are herein incorporated by reference in their entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

Any UTR from any gene known in the art may be incorporated into the viral genome of the AAV particle. These UTRs, or portions thereof, may be placed in the same orientation as in the gene from which they were selected or they may be altered in orientation or location. In some embodiments, the UTR used in the viral genome of the AAV particle may be inverted, shortened, lengthened, or made with one or more other 5′ UTRs or 3′ UTRs known in the art. As used herein, the term “altered,” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTR, which is not a variant of a wild type UTR.

In some embodiments, the viral genome of the AAV particle comprises UTRs which have been selected from a family of transcripts whose proteins share a common function, structure, feature, or property.

### Viral Genome Component: miR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleic acid sequence encoding a payload), e.g., before the polyA sequence, 5′-UTR region of the viral genome (e.g., 5′ relative to the nucleic acid sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% identical), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 1840), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1840, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCA CACTCCA (SEQ ID NO: 1841), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1841, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 1842), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1842, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 1843), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1843, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g. an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site or encoded miR binding site series comprises a miR182 binding site sequence. In some embodiments, the encoded miR182 binding site comprises, the nucleotide sequence of AGTGTGAGTTCTACCATTGCCAAA (SEQ ID NO: 1844), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1844, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site or encoded miR binding site series comprises a miR96 binding site sequence. In some embodiments, the encoded miR96 binding site comprises the nucleotide sequence of AGCAAAAATGTGCTAGTGCCAAA (SEQ ID NO: 1845), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1845, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, the encoded miR binding site series comprises a miR122 binding site, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

In some embodiments, an encoded miR binding site series comprises at least 2-5 copies (e.g., 2 or 3 copies) of a combination of at least two, three, four, five, or all of a miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1846), or a nucleotide sequence having at least one, two, or three modifications, but no more than four modifications of GATAGTTA (SEQ ID NO: 1846).

### Viral Genome Component: Polyadenylation Sequence

In some embodiments, the viral genome of the AAV particles of the present disclosure comprises at least one polyadenylation sequence. The viral genome of the AAV particle may comprise a polyadenylation sequence between the 3′ end of the payload coding sequence and the 5′ end of the 3′UTR.

In some embodiments, the polyA signal region comprises a length of about 100-600 nucleotides, e.g., about 100-500 nucleotides, about 100-400 nucleotides, about 100-300 nucleotides, about 100-200 nucleotides, about 200-600 nucleotides, about 200-500 nucleotides, about 200-400 nucleotides, about 200-300 nucleotides, about 300-600 nucleotides, about 300-500 nucleotides, about 300-400 nucleotides, about 400-600 nucleotides, about 400-500 nucleotides, or about 500-600 nucleotides. In some embodiments, the polyA signal region comprises a length of about 100 to 150 nucleotides, e.g., about 127 nucleotides. In some embodiments, the polyA signal region comprises a length of about 450 to 500 nucleotides, e.g., about 477 nucleotides. In some embodiments, the polyA signal region comprises a length of about 520 to about 560 nucleotides, e.g., about 552 nucleotides. In some embodiments, the polyA signal region comprises a length of about 127 nucleotides.

In some embodiments, the encoded NPC protein may be located upstream of the polyadenylation sequence (e.g., 5′ relative to the polyadenylation sequence) in an expression vector or a viral genome described herein. Further, the encoded NPC protein may be located downstream of a promoter (e.g., 3′ relative to a promoter) such as, but not limited to, CMV, U6, CBA, or a CBA promoter with a SV40 intron in an expression vector, viral genome, or a fragment thereof (e.g., one disclosed herein). In some embodiments, the encoded NPC protein may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides downstream from the promoter (e.g., 3′ relative to the promoter) and/or upstream of the polyadenylation sequence (e.g., 5′ relative to the polyadenylation sequence) in an expression vector or a viral genome described herein. In some embodiments, the encoded NPC protein may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30, or 25-30 nucleotides downstream from the promoter (e.g., 3′ relative to the promoter) and/or upstream of the polyadenylation sequence (e.g., 5′ relative to the polyadenylation sequence) in an expression vector or a viral genome described herein. In some embodiments, the encoded NPC protein may be located within the first 1%, 2%, 3%, 4%, 5%, 6%, 7%, 8%, 9%, 10%, 15%, 20%, 25%, or more than 25% of the nucleotides downstream from the promoter (e.g., 3′ relative to the promoter) and/or upstream of the polyadenylation sequence (e.g., 5′ relative to the polyadenylation sequence) in an expression vector or a viral genome described herein. In some embodiments, the encoded NPC protein may be located within the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter (e.g., 3′ relative to the promoter) and/or upstream of the polyadenylation sequence (e.g., 5′ relative to the polyadenylation sequence) in an expression vector or viral genome described herein.

In some embodiments, the viral genome comprises a rabbit globin polyA signal region. In some embodiments, the viral genome comprises a human growth hormone (hGH) polyA sequence. In some embodiments, the viral genome comprises an hGH polyA as described above and a payload region encoding NPC protein e.g., encoding a sequence as provided in Table 2 or fragment or variant thereof.

### Viral Genome Component: Filler Sequence

In some embodiments, the viral genome comprises one or more filler sequences. The filler sequence may be a wild-type sequence or an engineered sequence. A filler sequence may be a variant of a wild-type sequence. In some embodiments, a filler sequence is a derivative of human albumin.

In some embodiments, the viral genome comprises one or more filler sequences in order to have the length of the viral genome be the optimal size for packaging. In some embodiments, the viral genome comprises at least one filler sequence in order to have the length of the viral genome be about 2.3 kb. In some embodiments, the viral genome comprises at least one filler sequence in order to have the length of the viral genome be about 4.6 kb.

In some embodiments, the viral genome is a single stranded (ss) viral genome and comprises one or more filler sequences that, independently or together, have a length about between 0.1 kb-3.8 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, or 3.8 kb. In some embodiments, the total length filler sequence in the viral genome is 3.1 kb. In some embodiments, the total length filler sequence in the viral genome is 2.7 kb. In some embodiments, the total length filler sequence in the viral genome is 0.8 kb. In some embodiments, the total length filler sequence in the viral genome is 0.4 kb. In some embodiments, the length of each filler sequence in the viral genome is 0.8 kb. In some embodiments, the length of each filler sequence in the viral genome is 0.4 kb.

In some embodiments, the viral genome is a self-complementary (sc) viral genome and comprises one or more filler sequences that, independently or together, have a length about between 0.1 kb-1.5 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, or 1.5 kb. In some embodiments, the total length filler sequence in the viral genome is 0.8 kb. In some embodiments, the total length filler sequence in the viral genome is 0.4 kb. In some embodiments, the length of each filler sequence in the viral genome is 0.8 kb. In some embodiments, the length of each filler sequence in the viral genome is 0.4 kb.

In some embodiments, the viral genome comprises any portion of a filler sequence. The viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of a filler sequence.

In some embodiments, the viral genome is a single stranded (ss) viral genome and comprises one or more filler sequences in order to have the length of the viral genome be about 4.6 kb or 2.3kb. In some embodiments, the viral genome comprises at least one filler sequence and the filler sequence is located 3′ to the 5′ ITR sequence. In some embodiments, the viral genome comprises at least one filler sequence and the filler sequence is located 5′ to a promoter sequence. In some embodiments, the viral genome comprises at least one filler sequence and the filler sequence is located 3′ to the polyadenylation signal sequence. In some embodiments, the viral genome comprises at least one filler sequence and the filler sequence is located 5′ to the 3′ ITR sequence. In some embodiments, the viral genome comprises at least one filler sequence, and the filler sequence is located between two intron sequences. In some embodiments, the viral genome comprises at least one filler sequence, and the filler sequence is located within an intron sequence. In some embodiments, the viral genome comprises two filler sequences, and the first filler sequence is located 3′ to the 5′ ITR sequence and the second filler sequence is located 3′ to the polyadenylation signal sequence. In some embodiments, the viral genome comprises two filler sequences, and the first filler sequence is located 5′ to a promoter sequence and the second filler sequence is located 3′ to the polyadenylation signal sequence. In some embodiments, the viral genome comprises two filler sequences, and the first filler sequence is located 3′ to the 5′ ITR sequence and the second filler sequence is located 5′ to the 5′ ITR sequence.

In some embodiments, the viral genome may comprise one or more filler sequences between one of more regions of the viral genome. In some embodiments, the filler region may be located before a region such as, but not limited to, a payload region, an inverted terminal repeat (ITR), a promoter region, an intron region, an enhancer region, a polyadenylation signal sequence region, and/or an exon region. In some embodiments, the filler region may be located after a region such as, but not limited to, a payload region, an inverted terminal repeat (ITR), a promoter region, an intron region, an enhancer region, a polyadenylation signal sequence region, and/or an exon region. In some embodiments, the filler region may be located before and after a region such as, but not limited to, a payload region, an inverted terminal repeat (ITR), a promoter region, an intron region, an enhancer region, a polyadenylation signal sequence region, and/or an exon region.

### Viral Genome Component: Payloads

In some embodiments, an AAV particle, e.g., an AAV particle for the vectorized delivery of an NPC protein, e.g., an NPC1 protein described herein, comprises a payload. In some embodiments, an AAV particle, e.g., an AAV particle for the vectorized delivery of an NPC protein described herein (e.g., an NPC1 protein), comprises a promoter operably linked to a nucleic acid comprising a transgene encoding a payload. In some embodiments, the payload comprises an NPC protein, e.g., an NPC1 protein.

In some embodiments, the disclosure herein provides constructs that allow for improved expression of NPC protein delivered by gene therapy vectors.

In some embodiments, the disclosure provides constructs that allow for improved biodistribution of NPC protein delivered by gene therapy vectors.

In some embodiments, the disclosure provides constructs that allow for improved sub-cellular distribution or trafficking of NPC protein delivered by gene therapy vectors.

In some embodiments, the disclosure provides constructs that allow for improved trafficking of NPC protein to lysosomal membranes delivered by gene therapy vectors.

In some aspects, the present disclosure relates to compositions containing or comprising nucleic acid sequence(s) encoding NPC protein or functional fragment(s) or variants thereof and methods of administering these compositions in vitro or in vivo in humans and/or animal models of disease.

AAV particles of the present disclosure may comprise a nucleic acid sequence encoding at least one “payload.” As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, e.g., NPC protein or fragment or variant thereof. The payload may comprise any nucleic acid known in the art that is useful for the expression (by supplementation of the protein product or gene replacement using a modulatory nucleic acid) of NPC protein in a target cell transduced or contacted with the AAV particle carrying the payload.

The payload construct may comprise a combination of coding and non-coding nucleic acid sequences.

Any segment, fragment, or the entirety of the viral genome and therein, the payload construct, may be codon optimized.

In some embodiments, the nucleic acid sequence, e.g., the viral genome, of the AAV particle may be a payload construct comprising at least one portion encoding NPC protein. Exemplary nucleic acid sequences useful for expressing an NPC protein payload as described herein are provided in Table 2, along with encoded NPC protein amino acid sequences. In some embodiments, the at least one portion of a payload construct encoding NPC protein is derived from a nucleic acid sequence useful for expressing an NPC protein payload provided in Table 2, or a sequence substantially identical, (e.g., having at least about 70%, 75%, 80%, 85%, 90%, 92%, 95%, 97%, 98%, or 99% sequence identity) to the aforesaid sequences.

In some embodiments, the payload construct encodes more than one payload. As a non-limiting example, a payload construct encoding more than one payload may be replicated and packaged into a viral particle. A target cell transduced with a viral particle comprising more than one payload may express each of the payloads in a single cell.

In some embodiments, the payload construct may encode a coding or non-coding RNA. In certain embodiments, the adeno-associated viral vector particle further comprises at least one cis-element selected from the group consisting of a Kozak sequence, a backbone sequence, and an intron sequence.

In some embodiments, the payload is a polypeptide which may be a peptide or protein. A protein encoded by the payload construct may comprise a secreted protein, an intracellular protein, an extracellular protein, and/or a membrane protein. The encoded proteins may be structural or functional. Proteins encoded by the payload construct include, but are not limited to, mammalian proteins. In certain embodiments, the AAV particle contains a viral genome that encodes NPC protein or fragment or variant thereof. The AAV particles encoding a payload may be useful in the fields of human disease, veterinary applications, and a variety of in vivo and in vitro settings.

In some embodiments, a payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. In some embodiments, a payload may comprise nucleotide sequences that provide a desired effect or regulatory function (e.g., transposons, transcription factors).

The encoded payload may comprise a gene therapy product. A gene therapy product may include, but is not limited to, a polypeptide, RNA molecule, or other gene product that, when expressed in a target cell, provides a desired therapeutic effect. In some embodiments, a gene therapy product may comprise a substitute for a non-functional gene or a gene that is absent, expressed in insufficient amounts, or mutated. In some embodiments, a gene therapy product may comprise a substitute for a non-functional protein or polypeptide or a protein or polypeptide that is absent, expressed in insufficient amounts, misfolded, degraded too rapidly, or mutated. For example, a gene therapy product may comprise an NPC protein or a polynucleotide encoding an NPC protein to treat NPC protein deficiency or NPC1 or related disorders.

In some embodiments, the payload encodes a messenger RNA (mRNA). As used herein, the term “messenger RNA” (mRNA) refers to any polynucleotide that encodes a polypeptide of interest and that is capable of being translated to produce the encoded polypeptide of interest in vitro, in vivo, in situ, or ex vivo. Certain embodiments provide the mRNA as encoding NPC protein or a variant thereof.

The components of an mRNA include, but are not limited to, a coding region, a 5′-UTR (untranslated region), a 3′-UTR, a 5′-cap and a poly-A tail. In some embodiments, the encoded mRNA or any portion of the AAV genome may be codon optimized.

In some embodiments, the protein or polypeptide encoded by the payload construct encoding NPC protein or a variant thereof is between about 50 and about 4500 amino acid residues in length (hereinafter in this context, “X amino acids in length” refers to X amino acid residues). In some embodiments, the protein or polypeptide encoded is between 50-2000 amino acids in length. In some embodiments, the protein or polypeptide encoded is between 50-1000 amino acids in length. In some embodiments, the protein or polypeptide encoded is between 50-1500 amino acids in length. In some embodiments, the protein or polypeptide encoded is between 50-1000 amino acids in length. In some embodiments, the protein or polypeptide encoded is between 50-800 amino acids in length. In some embodiments, the protein or polypeptide encoded is between 50-600 amino acids in length. In some embodiments, the protein or polypeptide encoded is between 50-400 amino acids in length. In some embodiments, the protein or polypeptide encoded is between 50-200 amino acids in length. In some embodiments, the protein or polypeptide encoded is between 50-100 amino acids in length.

A payload construct encoding a payload may comprise or encode a selectable marker. A selectable marker may comprise a gene sequence or a protein or polypeptide encoded by a gene sequence expressed in a host cell that allows for the identification, selection, and/or purification of the host cell from a population of cells that may or may not express the selectable marker. In some embodiments, the selectable marker provides resistance to survive a selection process that would otherwise kill the host cell, such as treatment with an antibiotic. In some embodiments, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload construct encoding a payload may comprise a selectable marker including, but not limited to, β-lactamase, luciferase, 0-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540); the contents of each of which are herein incorporated by reference in their entirety.

In some embodiments, a payload construct encoding a selectable marker may comprise a fluorescent protein. A fluorescent protein as herein described may comprise any fluorescent marker including but not limited to green, yellow, and/or red fluorescent protein (GFP, YFP, and/or RFP). In some embodiments, a payload construct encoding a selectable marker may comprise a human influenza hemagglutinin (HA) tag.

In certain embodiments, a nucleic acid for expression of a payload in a target cell will be incorporated into the viral genome and located between two ITR sequences.

In some embodiments, a payload construct further comprises a nucleic acid sequence encoding a peptide that binds to the cation-independent mannose 6-phosphate (M6P) receptor (CI-MPR) with high affinity, as described in Int'l Pat. App. Pub. No. WO2019213180A1, the disclosure of which is incorporated herein by reference in its entirety. The peptide that binds CI-MPR can be, e.g., an IGF2 peptide or variant thereof. Binding of CI-MPR can facilitate cellular uptake or delivery and intracellular or sub-cellular targeting of therapeutic proteins provided by gene therapy vectors.

Exemplary NPC protein Payloads

In some embodiments, the payload, e.g., of a viral genome described herein, is an NPC protein, including NPC1 protein, wild-type NPC1, and functional variants thereof, NPC2 protein, wild-type NPC2, and functional variants thereof. In some embodiments, a functional variant is a variant that retains some or all of the activity of its wild-type counterpart, so as to achieve a desired therapeutic effect. For example, in some embodiments, a functional variant is effective to be used in gene therapy to treat a disorder or condition, for example, a disease related to expression of NPC (e.g., NPC1), an NPC1 deficiency or NPC1 disease or related disorders. Unless indicated otherwise, a variant of an NPC protein as described herein (e.g., in the context of the constructs, vectors, genomes, methods, kits, compositions, etc. of the disclosure) is a functional variant.

As used herein, “associated with decreased NPC1/NPC2 protein levels” or “associated with decreased expression” means that one or more symptoms of a disease are caused by lower-than-normal NPC1/NPC2 protein levels in a target tissue or in a biofluid such as blood. A disease or condition associated with decreased NPC1 protein levels or expression may be a disorder of the central nervous system. Also specifically contemplated herein are NPC disease and related disorders arising from expression of defective NPC1/NPC2 protein. Such a disease or condition may be a neuromuscular or a neurological disorder or condition. For example, a disease associated with decreased NPC1 protein levels may be NPC1 disease or related disorder, or may be another neurological or neuromuscular disorder described herein.

The present disclosure addresses the need for new technologies by providing NPC protein related treatment deliverable by AAV-based compositions and complexes for the treatment of NPC1 disease and related disorders.

While delivery is exemplified in the AAV context, other viral vectors, non-viral vectors, nanoparticles, or liposomes may be similarly used to deliver the therapeutic NPC protein(s) and include, but are not limited to, viral genomes of any of the AAV serotypes or other viral delivery vehicles or lentivirus, etc. The observations and teachings extend to any macromolecular structure, including modified cells, introduced into the CNS in the manner as described herein.

Given in Table 2 are the sequence identifiers of exemplary polynucleotide and polypeptide sequences for NPC proteins that may be used in the viral genomes disclosed herein and which may constitute an NPC protein payload. Functional variants, e.g., those retaining at least about 90% or at least 95% sequence identity to a sequence shown in Table 2, may also be used. Codon-optimized and other variants that encode the same or essentially the same NPC protein amino acid sequence (e.g., those having at least about 90% amino acid sequence identity) may also be used.

In some embodiments, the viral genome comprises a nucleic acid comprising a transgene encoding an NPC protein, e.g., an NPC1 protein, an NPC2 protein, or functional variant thereof. In some embodiments, the encoded NPC protein (e.g., NPC1 protein, NPC2 protein, or functional variant thereof) comprises an amino acid sequence from an NPC protein described herein, e.g., as described in Table 2, or an amino acid sequence substantially identical (e.g., having at least about 70%, 75%, 80%, 85%, 90%, 92%, 95%, 97%, 98%, or 99% sequence identity) to any of the aforesaid sequences. In some embodiments, the encoded NPC protein (e.g., NPC1 protein, NPC2 protein, or functional variant thereof) comprises an amino acid sequence from an NPC protein described herein, e.g., as described in Table 2, or an amino acid sequence having at least one, two or three modifications but not more than 30, 20 or 10 modifications relative to any of the aforesaid amino acid sequences. In some embodiments, the encoded NPC protein (e.g., NPC1 NPC2 protein, or functional variant thereof) comprises an amino acid sequence encoded by a nucleotide sequence encoding an NPC protein described herein, e.g., as described in Table 2, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding the NPC protein (e.g., NPC1 protein, NPC2 protein, or functional variant thereof) comprises a nucleotide sequence encoding an NPC protein described herein, e.g., as described in Table 2, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the NPC protein (e.g., NPC1 protein, NPC2 protein, or functional variant thereof) comprises a nucleotide sequence encoding an NPC protein described herein, e.g., as described in Table 2, or a nucleotide sequence having at least one, two or three modifications but not more than 30, 20 or 10 modifications relative to any of the aforesaid nucleotide sequences. In some embodiments, the nucleotide sequence encoding an NPC protein (e.g., NPC1 protein, NPC2 protein, or functional variant thereof) is a codon optimized nucleotide sequence.

TABLE 2
Exemplary Nucleotide Sequences Encoding NPC
proteins and NPC Protein Amino Acid Sequences

| SEQ ID NO: | Type | Species | Description |
|---|---|---|---|
| 1724 | Protein | Homo sapiens | NPC1 protein NP_000262.2 |
| 1725 | DNA | Homo sapiens | NPC1 mRNA NM_000271.5 |
| 1726 | Protein | Homo sapiens | NPC2 protein NP_006423.1 |
| 1727 | DNA | Homo sapiens | NPC2 mRNA NM_006432.5 |
| 1728 | Protein | Homo sapiens | NPC2 protein NP_001350617.1 |
| 1729 | DNA | Homo sapiens | NPC2 mRNA NM_001363688.1 |
| 1730 | Protein | Homo sapiens | NPC2 protein NP_001362369.1 |
| 1731 | DNA | Homo sapiens | NPC2 mRNA NM_001375440.1 |
| 1732 | DNA | Macaca mulatta | NPC2 mRNA NM_001266760.1 |
| 1747 | DNA | Homo sapiens | Wild type Human NPC1 |
| | | | Coding Sequence |
| 1748 | Protein | Homo sapiens | Wild type Human NPC1 |
| | | | Protein Sequence |
| 1749 | DNA | Artificial sequence | Codon optimized Human |
| | | | NPC1 Coding Sequence 1 |
| 1750 | DNA | Artificial sequence | Codon optimized Human |
| | | | NPC1 Coding Sequence 2 |

In some embodiments, the encoded NPC protein (e.g., NPC1 protein, NPC2 protein, or functional variant thereof) comprises the amino acid sequence of any one of SEQ ID NOs: 1724, 1726, 1728, 1730, or 1748, or an amino acid sequence substantially identical (e.g., having at least about 70%, 75%, 80%, 85%, 90%, 92%, 95%, 97%, 98%, or 99% sequence identity) to any of the aforesaid sequences. In some embodiments, the encoded NPC protein (e.g., NPC1 protein, NPC2 protein, or functional variant thereof) comprises the amino acid sequence of any one of SEQ ID NOs: 1724, 1726, 1728, 1730, or 1748, or an amino acid sequence having at least one, two or three modifications but not more than 30, 20 or 10 modifications relative to any of the aforesaid amino acid sequences. In some embodiments, the encoded NPC protein (e.g., NPC1 protein, NPC2 protein, or functional variant thereof) comprises an amino acid sequence encoded by the nucleotide sequence of any one of SEQ ID NOs: 1725, 1727, 1729, 1731, 1732, 1747, 1749, or 1750, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding the NPC protein (e.g., NPC1 protein, NPC2 protein, or functional variant thereof) comprises the nucleotide sequence of any one of SEQ ID NOs: 1725, 1727, 1729, 1731, 1732, 1747, 1749, or 1750, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the nucleic acid sequence encoding the NPC protein (e.g., NPC1 protein, NPC2 protein, or functional variant thereof) comprises the nucleotide sequence of any one of SEQ ID NOs: 1725, 1727, 1729, 1731, 1732, 1747, 1749, or 1750, or a nucleotide sequence having at least one, two or three modifications but not more than 30, 20 or 10 modifications relative to any of the aforesaid nucleotide sequences. In some embodiments, the nucleotide sequence encoding the NPC protein (e.g., NPC1 protein, NPC2 protein, or functional variant thereof) does not comprise a stop codon. In some embodiments, the nucleotide sequence encoding the NPC protein (e.g., NPC1 protein, NPC2 protein, or functional variant thereof) is a codon optimized nucleotide sequence.

In some embodiments, the viral genome comprises a payload region encoding an NPC protein. The encoded NPC protein may be derived from any species, such as, but not limited to human, non-human primate, or rodent.

In some embodiments, the viral genome comprises a payload region encoding a human (Homo sapiens) NPC protein, or a variant thereof.

Various embodiments of the disclosure herein provide an adeno-associated viral (AAV) particle comprising a viral genome, the viral genome comprising at least one inverted terminal repeat region and a nucleic acid sequence encoding a polypeptide having at least 90% sequence identity to a human NPC protein sequence as provided in Table 2.

In some embodiments, the AAV viral genome comprises at least one inverted terminal repeat region and a nucleic acid sequence encoding a polypeptide having at least 90% sequence identity to an NPC protein sequence as provided in Table 2. In some embodiments, the AAV viral genome comprises at least one inverted terminal repeat region and a nucleic acid sequence encoding a polypeptide having at least 95% sequence identity to an NPC protein sequence as provided in Table 2. In some embodiments, the AAV viral genome comprises at least one inverted terminal repeat region and a nucleic acid sequence encoding a polypeptide having at least 98% sequence identity to an NPC protein sequence as provided in Table 2. In some embodiments, the AAV viral genome comprises at least one inverted terminal repeat region and a nucleic acid sequence encoding a polypeptide having at least 99% sequence identity to an NPC protein sequence as provided in Table 2. In some embodiments, the AAV viral genome comprises at least one inverted terminal repeat region and a nucleic acid sequence encoding an NPC protein sequence provided in Table 2.

In some embodiments, the NPC protein is derived from an NPC1/NPC2 sequence of a non-human primate, such as the cynomolgus monkey, Macaca fascicularis. Certain embodiments provide the NPC protein as a humanized version of a Macaca fascicularis sequence.

In some embodiments, the viral genome comprises a payload region encoding a cynomolgus or crab-eating (long-tailed) macaque (Macaca fascicularis) NPC protein, or a variant thereof.

In some embodiments, the viral genome comprises a payload region encoding a rhesus macaque (Macaca mulatta) NPC protein, or a variant thereof.

In some embodiments, the NPC protein may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 2.

In some embodiments, the NPC protein may be encoded by a nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above and provided in Table 2.

### Exemplary NPC1 Proteins and NPC1 Protein Coding Sequences

In some embodiments, a viral genome described herein encoding an NPC1 protein, for example as a payload, comprises a nucleotide sequence encoding a wild type human NPC1 amino acid sequence.

In some embodiments, the viral genome comprises a nucleic acid encoding a transgene encoding an NPC1 protein, e.g., an NPC1 protein described herein. In some embodiments, the encoded NPC1 protein comprises the amino acid sequence of SEQ ID NO: 1748, an amino acid sequence substantially identical (e.g., having at least about 70%, 75%, 80%, 85%, 90%, 92%, 95%, 97%, 98%, or 99% sequence identity) to SEQ ID NO: 1748, or an amino acid sequence having at least one, two or three modifications, but not more than 30, 20 or 10 modifications of the amino acid sequence of SEQ ID NO: 1748. In some embodiments, the NPC1 protein comprises an amino acid sequence encoded by the nucleotide sequence of any of SEQ ID NOs: 1747, 1749, or 1750, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded NPC1 protein comprises the amino acid sequence of SEQ ID NO: 1724, an amino acid substantially identical (e.g., having at least about 70%, 75%, 80%, 85%, 90%, 92%, 95%, 97%, 98%, or 99% sequence identity) to SEQ ID NO: 1724, or an amino acid sequence having at least one, two or three modifications, but not more than 30, 20 or 10 modifications of the amino acid sequence of SEQ ID NO: 1724. In some embodiments, the NPC1 protein comprises an amino acid sequence encoded by the nucleotide sequence of SEQ ID NO: 1725, or a nucleotide sequence substantially identical (e.g., having at least about 70%, 75%, 80%, 85%, 90%, 92%, 95%, 97%, 98%, or 99% sequence identity) thereto. In some embodiments, the encoded NPC1 protein comprises a modification, e.g., a substitution. In some embodiments, the encoded NPC1 protein comprises an amino acid substitution at position M642, e.g., an M642I substitution, numbered according to SEQ ID NO: 1724.

In some embodiments, the nucleotide sequence encoding an NPC1 protein comprises the nucleotide sequence of SEQ ID NO: 1747, or a nucleotide sequence substantially identical (e.g., having at least about 70%, 75%, 80%, 85%, 90%, 92%, 95%, 97%, 98%, or 99% sequence identity) to SEQ ID NO: 1747. In some embodiments, the nucleotide sequence encoding an NPC1 protein comprises the nucleotide sequence of SEQ ID NO: 1747, or a nucleotide sequence having at least one, two or three modifications but not more than 30, 20 or 10 modifications of the amino acid sequence of SEQ ID NO: 1747. In some embodiments, the nucleotide sequence encoding the NPC1 protein does not comprise a stop codon, e.g., nucleotides 3835-3837 numbered relative to SEQ ID NO: 1747.

In some embodiments, the nucleotide sequence encoding an NPC1 protein comprises the nucleotide sequence of SEQ ID NO: 1749, or a nucleotide sequence substantially identical (e.g., having at least about 70%, 75%, 80%, 85%, 90%, 92%, 95%, 97%, 98%, or 99% sequence identity) to SEQ ID NO: 1749. In some embodiments, the nucleotide sequence encoding an NPC1 protein comprises the nucleotide sequence of SEQ ID NO: 1749, or a nucleotide sequence having at least one, two or three modifications but not more than 30, 20 or 10 modifications of the amino acid sequence of SEQ ID NO: 1749. In some embodiments, the nucleotide sequence encoding the NPC1 protein is a codon optimized nucleotide sequence. In some embodiments, the nucleotide sequence encoding the NPC1 protein does not comprise a stop codon, e.g., nucleotides 3835-3837 numbered relative to SEQ ID NO: 1749.

In some embodiments, the nucleotide sequence encoding an NPC1 protein comprises the nucleotide sequence of SEQ ID NO: 1750, or a nucleotide sequence substantially identical (e.g., having at least about 70%, 75%, 80%, 85%, 90%, 92%, 95%, 97%, 98%, or 99% sequence identity) to SEQ ID NO: 1750. In some embodiments, the nucleotide sequence encoding an NPC1 protein comprises the nucleotide sequence of SEQ ID NO: 1750, or a nucleotide sequence having at least one, two or three modifications but not more than 30, 20 or 10 modifications of the amino acid sequence of SEQ ID NO: 1750. In some embodiments, the nucleotide sequence encoding the NPC1 protein is a codon optimized nucleotide sequence. In some embodiments, the nucleotide sequence encoding the NPC1 protein does not comprise a stop codon, e.g., nucleotides 3835-3837 numbered relative to SEQ ID NO: 1750.

One example of a nucleic acid sequence encoding a wild type human NPC1 amino acid sequence is provided as SEQ ID NO: 1747. Thus, in some embodiments, the viral genomes may comprise a nucleic acid sequence of SEQ ID NO: 1747. Some embodiments provide for viral genomes comprising a nucleic acid sequence having 75% or more, 80% or more, 85% or more, 90% or more, or 95% or more sequence identity compared to SEQ ID NO: 1747. Some embodiments provide for viral genomes comprising a nucleic acid sequence having 95%, 96%, 97%, 98%, 99%, or 100% sequence identity compared to SEQ ID NO: 1747.

Some embodiments of the viral genomes described herein encoding an NPC1 protein, for example as a payload, comprise a codon optimized nucleotide sequence encoding an NPC1 amino acid sequence. One example of a codon optimized nucleic acid sequence encoding an NPC1 amino acid sequence is provided as SEQ ID NO: 1749. Thus, in some embodiments, the viral genomes may comprise a nucleic acid sequence of SEQ ID NO: 1749. Some embodiments provide for viral genomes comprising a nucleic acid sequence having 75% or more, 80% or more, 85% or more, 90% or more, or 95% or more sequence identity compared to SEQ ID NO: 1749. Some embodiments provide for viral genomes comprising a nucleic acid sequence having 95%, 96%, 97%, 98%, 99%, or 100% sequence identity compared to SEQ ID NO: 1749. Another example of a codon optimized nucleic acid sequence encoding an NPC1 amino acid sequence is provided as SEQ ID NO: 1750. Thus, in some embodiments, the viral genomes may comprise a nucleic acid sequence of SEQ ID NO: 1750. Some embodiments provide for viral genomes comprising a nucleic acid sequence having 75% or more, 80% or more, 85% or more, 90% or more, or 95% or more sequence identity compared to SEQ ID NO: 1750. Some embodiments provide for viral genomes comprising a nucleic acid sequence having 95%, 96%, 97%, 98%, 99%, or 100% sequence identity compared to SEQ ID NO: 1750.

NPC1 protein-encoding viral genomes provided herein may encode an NPC1 protein having an amino acid sequence as provided in SEQ ID NO: 1724. Some embodiments provide for viral genomes comprising an NPC1 protein-encoding sequence encoding an NPC1 protein having 75% or more, 80% or more, 85% or more, 90% or more, or 95% or more sequence identity compared to SEQ ID NO: 1724. Some embodiments provide for viral genomes comprising an NPC1 protein-encoding sequence encoding an NPC1 protein having 95%, 96%, 97%, 98%, 99%, or 100% sequence identity compared to SEQ ID NO: 1724. In some embodiments, one or more amino acid substitutions are introduced in the NPC1 protein. For example, the NPC1 protein may comprise an M642I substitution. Accordingly, some embodiments, of the NPC1 protein-encoding viral genomes may encode an NPC1 protein having an amino acid sequence as provided in SEQ ID NO: 1748. Some embodiments provide for viral genomes comprising an NPC1 protein-encoding sequence encoding an NPC1 protein having 75% or more, 80% or more, 85% or more, 90% or more, or 95% or more sequence identity compared to SEQ ID NO: 1748. Some embodiments provide for viral genomes comprising an NPC1 protein-encoding sequence encoding an NPC1 protein having 95%, 96%, 97%, 98%, 99%, or 100% sequence identity compared to SEQ ID NO: 1748.

Additional examples of AAV vectors for use in the compositions and methods described herein include the AAV9-EF1α-NPC1 vector, e.g., as described in Chandler, Randy J., et al. “Systemic AAV9 gene therapy improves the lifespan of mice with Niemann-Pick disease, type C1.” Human molecular genetics 26.1 (2017): 52-64; Venditti, Charles P., Randy Chandler, and William J. Pavan. “Viral gene therapy as treatment for cholesterol storage disease or disorder.” U.S. patent application Ser. No. 15/565,065 (Pub. No. US2018/0104289); and Hammond, Natalie, Andrew B. Munkacsi, and Stephen L. Sturley. “The complexity of a monogenic neurodegenerative disease: More than two decades of therapeutic driven research into Niemann-Pick type C disease.” Biochimica et Biophysica Acta (BBA)—Molecular and Cell Biology of Lipids (2019); the disclosures of each of which are incorporated herein by reference in their entireties. In some embodiments, the AAV vector for use in the methods described herein is as described in Int'l Pat. App. Pub. No. WO2018/237066A1 or European Pat. No. 3280451B1, the disclosures of each of which are incorporated by reference in their entireties.

### Self-Complementary and Single Strand Vectors

In some embodiments, the AAV vector used in the present disclosure is a single strand vector (ssAAV).

In some embodiments, the AAV vectors may be self-complementary AAV vectors (scAAVs). See, e.g., U.S. Pat. No. 7,465,583. scAAV vectors contain both DNA strands that anneal together to form double stranded DNA. By skipping second strand synthesis, scAAVs allow for rapid expression in the cell.

In some embodiments, the AAV vector used in the present disclosure is a scAAV.

Methods for producing and/or modifying AAV vectors are disclosed in the art such as pseudotyped AAV vectors (International Patent Publication Nos. WO200028004; WO200123001; WO2004112727; WO 2005005610 and WO 2005072364, the content of each of which are incorporated herein by reference in their entirety).

### Viral Genome Size

In some embodiments, the viral genome of the AAV particles of the present disclosure may be single or double stranded. The size of the viral genome may be small, medium, large or the maximum size.

In some embodiments, the viral genome, which comprises a nucleic acid sequence encoding NPC protein described herein, may be a small single stranded viral genome. A small single stranded viral genome may be about 2.7 kb to about 3.5 kb in size such as about 2.7, about 2.8, about 2.9, about 3.0, about 3.1, about 3.2, about 3.3, about 3.4, or about 3.5 kb in size. In some embodiments, the small single stranded viral genome may be 3.2 kb in size.

In some embodiments, the viral genome, which comprises a nucleic acid sequence encoding NPC protein described herein, may be a small double stranded viral genome. A small double stranded viral genome may be about 1.3 to about 1.7 kb in size such as about 1.3, about 1.4, about 1.5, about 1.6, or about 1.7 kb in size. In some embodiments, the small double stranded viral genome may be 1.6 kb in size.

In some embodiments, the viral genome, which comprises a nucleic acid sequence encoding NPC protein described herein, may be a medium single stranded viral genome. A medium single stranded viral genome may be about 3.6 to about 4.3 kb in size such as about 3.6, about 3.7, about 3.8, about 3.9, about 4.0, about 4.1, about 4.2, or about 4.3 kb in size. In some embodiments, the medium single stranded viral genome may be 4.0 kb in size.

In some embodiments, the viral genome, which comprises a nucleic acid sequence encoding NPC protein described herein, may be a medium double stranded viral genome. A medium double stranded viral genome may be about 1.8 to about 2.1 kb in size such as about 1.8, about 1.9, about 2.0, or about 2.1 kb in size. In some embodiments, the medium double stranded viral genome may be 2.0 kb in size. Additionally, the viral genome may comprise a promoter and a polyA tail.

In some embodiments, the viral genome which comprises a nucleic acid sequence encoding NPC protein described herein may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size.

In some embodiments, the viral genome which comprises a nucleic acid sequence encoding NPC protein described herein may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size.

### Exemplary NPC1 AAV Viral Genome Sequence Regions and ITR to ITR Sequences

In some embodiments, a viral genome, e.g., an AAV viral genome or vector genome, described herein, comprises a promoter operably linked to a transgene encoding an NPC1 protein. In some embodiments, the viral genome further comprises an inverted terminal repeat region, an enhancer, an intron, a Kozak sequence, a polyA region, or a combination thereof.

In some embodiments, the viral genome comprises a nucleotide sequence comprising, from 5′ to 3′, a 5′ inverted terminal repeat region, a promoter region, a Kozak sequence region, an NPC protein region comprising a nucleic acid sequence encoding an NPC protein, a polyA region, and a 3′ inverted terminal repeat; that is, an ITR to ITR sequence. Exemplary sequence regions within ITR to ITR sequences for viral genomes according to the description are provided in Table 3.

TABLE 3
Exemplary NPC1 AAV Vector Sequence
Regions in ITR to ITR sequences

| | Description of | Length of | |
|---|---|---|---|
| | Sequence | Sequence | SEQ ID |
| Sequence Regions | Region | Region | NO: |
| 5′ ITR | — | 130 | 1733 |
| 3′ ITR | — | 130 | 1734 |
| 5′ ITR | — | 141 | 1837 |
| 3′ ITR | — | 141 | 1838 |
| Promoter Region | CBA Promoter | 260 | 1735 |
| Promoter Region | CMV Promoter | 205 | 1736 |
| Promoter Region | CMVie Enhancer | 380 | 1737 |
| Promoter Region | CBA-D4 Promoter | 339 | 1738 |
| Promoter Region | CMV-D3 Promoter | 361 | 1739 |
| Promoter Region | CMV-D4 Promoter | 289 | 1740 |
| Promoter Region | CMV-D6 Promoter | 163 | 1741 |
| Promoter Region | CMVie Enhancer | 66 | 1742 |
| Promoter Region | CMVie Enhancer | 156 | 1743 |
| Promoter Region | CMV-D7 | 109 | 1744 |
| Promoter Region | miniEF1a Promoter | 227 | 1745 |
| Intron Region | pClneo-intron | 133 | 1780 |
| Kozak Region | Kozak Sequence | 9 | 1746 |
| NPC Protein coding | Wild type human NPC | 3837 | 1747 |
| sequence | protein coding sequence | | |
| NPC Protein coding | Codon optimized NPC | 3837 | 1749 |
| sequence 1 | protein coding sequence 1 | | |
| NPC Protein coding | Codon optimized NPC | 3837 | 1750 |
| sequence 2 | protein coding sequence 2 | | |
| Polyadenylation | — | 127 | 1751 |
| region | | | |

In some embodiments, the viral genome comprises an inverted terminal repeat sequence region (ITR) provided in Table 3, or a nucleotide sequence with at least 70%, 75%, 80%, 85%, 90%, 95% or 99% sequence identity to any of the ITR sequences in Table 3. In some embodiments, the ITR comprises the nucleotide sequence of any one of SEQ ID NOs: 1733-1734 or 1837-1838, or a sequence with at least 70%, 75%, 80%, 85%, 90%, 95% or 99% sequence identity thereto. In some embodiments, the ITR comprises the nucleotide sequence of SEQ ID NO: 1733 or a nucleotide sequence with at least 70%, 75%, 80%, 85%, 90%, 95% or 99% sequence identity thereto. In some embodiments, the ITR comprises the nucleotide sequence of SEQ ID NO: 1734 or a nucleotide sequence with at least 70%, 75%, 80%, 85%, 90%, 95% or 99% sequence identity thereto. In some embodiments, the ITR comprises the nucleotide sequence of SEQ ID NO: 1837 or a nucleotide sequence with at least 70%, 75%, 80%, 85%, 90%, 95% or 99% sequence identity thereto. In some embodiments, the ITR comprises the nucleotide sequence of SEQ ID NO: 1838 or a nucleotide sequence with at least 70%, 75%, 80%, 85%, 90%, 95% or 99% sequence identity thereto. In some embodiments, the viral genome comprises an ITR, e.g., a 5′ ITR, comprising the nucleotide sequence of SEQ ID NO: 1733 or a sequence with at least with at least 70%, 75%, 80%, 85%, 90%, 95% or 99% sequence identity thereto; and an ITR, e.g., a 3′ ITR, comprising the nucleotide sequence of SEQ ID NO: 1734 or a nucleotide sequence at least 70%, 75%, 80%, 85%, 90%, 95% or 99% sequence identity thereto. In some embodiments, the viral genome comprises an ITR, e.g., a 5′ ITR, comprising the nucleotide sequence of SEQ ID NO: 1837 or a sequence with at least with at least 70%, 75%, 80%, 85%, 90%, 95% or 99% sequence identity thereto; and an ITR, e.g., a 3′ ITR, comprising the nucleotide sequence of SEQ ID NO: 1838 or a nucleotide sequence at least 70%, 75%, 80%, 85%, 90%, 95% or 99% sequence identity thereto.

In some embodiments, the viral genome comprises a promoter provided in Table 3 or Table 12 or a nucleotide sequence with at least 70%, 75%, 80%, 85%, 90%, 95% or 99% sequence identity to any of the promoter sequences in Table 3 or Table 12.

In some embodiments, the promoter of a viral genome described herein comprises a CMV promoter or a functional variant thereof. In some embodiments the promoter comprises the nucleotide sequence of any one of SEQ ID NOs: 1736 or 1739-1741, or a nucleotide sequence least 70%, 75%, 80%, 85%, 90%, 95% or 99% identical thereto. In some embodiments, the viral genome comprises a CMVie enhancer. In some embodiments, the CMVie enhancer comprises the nucleotide sequence of any one of SEQ ID NOs: 1737, 1742, or 1743, or a nucleotide sequence least 70%, 75%, 80%, 85%, 90%, 95% or 99% identical thereto. In some embodiments, the viral genome comprises a CMVie enhancer and a CMV promoter. In some embodiments, the CMVie enhancer comprises the nucleotide sequence of SEQ ID NO: 1743, or a nucleotide sequence at least 70%, 75%, 80%, 85%, 90%, 95% or 99% identical thereto, and the CMV promoter comprises the nucleotide sequence of SEQ ID NO: 1736, or a nucleotide sequence at least 70%, 75%, 80%, 85%, 90%, 95% or 99% identical thereto.

In some embodiments, the promoter of a viral genome described herein comprises a CBA promoter or a functional variant thereof. In some embodiments, the promoter comprises the nucleotide sequence of SEQ ID NO: 1735 or 1738, or a nucleotide sequence at least 70%, 75%, 80%, 85%, 90%, 95% or 99% identical thereto. In some embodiments, the viral genome described herein comprises a CMVie enhancer and a CBA promoter. In some embodiments, the CMVie enhancer comprises the nucleotide sequence of SEQ ID NO: 1742, or a nucleotide sequence 70%, 75%, 80%, 85%, 90%, 95% or 99% identical thereto, and the CBA promoter comprises the nucleotide sequence of SEQ ID NO: 1735, or a nucleotide sequence at least 70%, 75%, 80%, 85%, 90%, 95% or 99% identical thereto. In some embodiments, the CMVie enhancer comprises the nucleotide sequence of SEQ ID NO: 1737, or a nucleotide sequence 70%, 75%, 80%, 85%, 90%, 95% or 99% identical thereto, and the CBA promoter comprises the nucleotide sequence of SEQ ID NO: 1735, or a nucleotide sequence at least 70%, 75%, 80%, 85%, 90%, 95% or 99% identical thereto.

In some embodiments, the viral genome described herein comprises an EF-1a promoter or an EF-1a promoter variant. In some embodiments, the EF-1a promoter or the EF-1a promoter variant comprises a nucleotide sequence of a promoter or promoter variant, e.g., as described in Table 12, or a nucleotide sequence at least 70%, 75%, 80%, 85%, 90%, 95% or 99% identical thereto.

In some embodiments, the EF-1a promoter comprises the nucleotide sequence of SEQ ID NO: 1781, or a nucleotide sequence at least 70%, 75%, 80%, 85%, 90%, 95% or 99% identical thereto. In some embodiments, the EF-1a promoter comprises an intron, e.g., an intron comprising the nucleotide sequence of positions 237-1,184 of SEQ ID NO: 1781. In some embodiments, the EF-1a promoter does not comprise an intron, e.g., an intron comprising the nucleotide sequence of positions 237-1,184 of SEQ ID NO: 1781. In some embodiments, the EF-1a promoter comprises a portion of an intron, e.g., a portion comprising no more than 5-50 nucleotides, 5-40 nucleotides, 5-30 nucleotides, 5-20 nucleotides, 5-10 nucleotides, 10-50 nucleotides, 10-40 nucleotides, 10-30 nucleotides, 10-20 nucleotides, 20-50 nucleotides, 20-40 nucleotides, 20-30 nucleotides, 30-50 nucleotides, 30-40 nucleotides, 40-50 nucleotides, 5 nucleotides, 10 nucleotides, 15 nucleotides, 20 nucleotides, 25 nucleotides, 30 nucleotides, 40 nucleotides, or 50 nucleotides of the intron e.g., an intron comprising the nucleotide sequence of positions 237-1,184 of SEQ ID NO: 1781. In some embodiments, EF-1a promoter comprises a portion of an intron, wherein the portion comprises the nucleotide sequence of SEQ ID NO: 1798.

In some embodiments, the viral genome comprises an EF-1a promoter variant. In some embodiments, the EF-1a promoter variant comprises [A]-[B]-[C]-[D]-[E], wherein [A] comprises SEQ ID NO: 1792, 1793, or 1794, or a sequence having at least sequence comprising at least one, two, or three but no more than four modifications, e.g., substitutions, relative to the nucleotide sequence of SEQ ID NO: 1792, 1793, or 1794; or [A] is absent; [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795 or [B] is absent; [C] comprises the nucleotides GT, the nucleotide sequence of SEQ ID NO: 1797, or a nucleotide sequence comprising at least one, two, or three but no more than four modifications, e.g., substitutions, relative to the nucleotide sequence of SEQ ID NO: 1797, or [C] is absent; [D] comprises the nucleotide sequence of SEQ ID NO: 1822 or 1823, or a nucleotide sequence having at least 90% (e.g., 92, 95, 96, 97, 98, or 99%) sequence identity thereto; and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798 or [E] is absent.

In some embodiments, the EF-1a promoter variant comprises [A]-[B]-[C]-[D]-[E], wherein [A] comprises SEQ ID NO: 1792, 1793, or 1794, or a sequence having at least sequence comprising at least one, two, or three but no more than four modifications, e.g., substitutions, relative to the nucleotide sequence of SEQ ID NO: 1792, 1793, or 1794; or [A] is absent; [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795 or [B] is absent; [C] comprises the nucleotides GT, the nucleotide sequence of SEQ ID NO: 1797, or a nucleotide sequence comprising at least one, two, or three but no more than four modifications, e.g., substitutions, relative to the nucleotide sequence of SEQ ID NO: 1797, or [C] is absent; [D] comprises the nucleotide sequence of SEQ ID NO: 1822, or a nucleotide sequence having at least 90% (e.g., 92, 95, 96, 97, 98, or 99%) sequence identity thereto, provided that [D] does not comprise the nucleotides “GC” at positions 235-236, numbered according to SEQ ID NO: 1781; and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798 or [E] is absent.

In some embodiments, the EF-1a promoter variant comprises [A]-[B]-[C]-[D]-[E], wherein [A] is absent, [B] is absent, [C] is absent, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] is absent, [B] is absent, [C] comprises the nucleotides GT, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] is absent, [B] is absent, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] is absent, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] is absent, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1822, or a nucleotide sequence having at least 90% (e.g., 92, 95, 96, 97, 98, or 99%) sequence identity thereto, and [E] is absent.

In some embodiments, the EF-1a promoter variant comprises [A]-[B]-[C]-[D]-[E], wherein [A] comprises SEQ ID NO: 1792, or a sequence having at least sequence comprising at least one or two modifications, e.g., substitutions, relative to the nucleotide sequence of SEQ ID NO: 1792, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] comprises SEQ ID NO: 1792, or a sequence having at least sequence comprising at least one or two modifications, e.g., substitutions, relative to the nucleotide sequence of SEQ ID NO: 1792, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1822, or a nucleotide sequence having at least 90% (e.g., 92, 95, 96, 97, 98, or 99%) sequence identity thereto, and [E] is absent.

In some embodiments, the EF-1a promoter variant comprises [A]-[B]-[C]-[D]-[E], wherein [A] comprises SEQ ID NO: 1793, or a sequence having at least sequence comprising at least one, two, or three but no more than four modifications, e.g., substitutions, relative to the nucleotide sequence of SEQ ID NO: 1793, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] comprises SEQ ID NO: 1793, or a sequence having at least sequence comprising at least one, two, or three but no more than four modifications, e.g., substitutions, relative to the nucleotide sequence of SEQ ID NO: 1793, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1822, or a nucleotide sequence having at least 90% (e.g., 92, 95, 96, 97, 98, or 99%) sequence identity thereto, and [E] is absent.

In some embodiments, the EF-1a promoter variant comprises [A]-[B]-[C]-[D]-[E], wherein [A] comprises SEQ ID NO: 1794, or a sequence having at least sequence comprising at least one, two, or three but no more than four modifications, e.g., substitutions, relative to the nucleotide sequence of SEQ ID NO: 1794, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1823, or a nucleotide sequence having at least 90% (e.g., 92, 95, 96, 97, 98, or 99%) sequence identity thereto, and [E] comprises the nucleotide sequence of SEQ ID NO: 1798, or a sequence having at least sequence comprising at least one or two, but no more than 3 modifications, e.g., substitutions, relative to the nucleotide sequence of SEQ ID NO: 1798.

In some embodiments, the EF-1a promoter variant comprises [A]-[B]-[C]-[D]-[E], wherein [A] comprises SEQ ID NO: 1794, or a sequence having at least sequence comprising at least one, two, or three but no more than four modifications, e.g., substitutions, relative to the nucleotide sequence of SEQ ID NO: 1794, [B] comprises the nucleotide sequence of SEQ ID NO: 1795, or a sequence having at least sequence comprising at least one or two, but no more than 3 modifications, e.g., substitutions, relative to the nucleotide sequence of SEQ ID NO: 1795, [C] comprises the nucleotide sequence of SEQ ID NO: 1797, or a nucleotide sequence comprising at least one, two, or three but no more than four modifications, e.g., substitutions, relative to the nucleotide sequence of SEQ ID NO: 1797, [D] comprises the nucleotide sequence of SEQ ID NO: 1822, or a nucleotide sequence having at least 90% (e.g., 92, 95, 96, 97, 98, or 99%) sequence identity thereto, [E] is absent.

In some embodiments, the viral genome comprises an EF-1a promoter variant, wherein the EF-1a promoter variant is a truncated EF-1a promoter variant. In some embodiments, the EF-1a promoter variant comprises the nucleotide sequence of the WT EF-1a promoter comprising SEQ ID NO: 1781 and a deletion. In some embodiments, the deletion comprises at least 10, 20, 25, 30, 50, 100, 150, 200, 250, 300, 350, 400, 450, 500, 550, 600, 650, 700, 750, 800, 850, 900, or 950 nucleotides. In some embodiments, the deletion is from the 3′ end of the EF-1a promoter (e.g., the WT EF-1a promoter, e.g., comprising SEQ ID NO: 1781), the 5′ end of the EF-1a promoter (e.g., the WT EF-1a promoter, e.g., comprising SEQ ID NO: 1781), both. In some embodiments, the EF-1a promoter variant, e.g., an EF-1a truncated promoter, comprises the nucleotide sequence of SEQ ID NO: 1781 or a sequence with at least 95% identity thereto, with a fragment of the WT EF-1a promoter deleted, optionally wherein the fragment comprises nucleotides 237-1,184 of SEQ ID NO: 1781 or nucleotides 242-1,184 of SEQ ID NO: 1781. In some embodiments, the EF-1a promoter variant further comprises the nucleotide sequence of SEQ ID NO: 1792, 1793, or 1794, or a sequence having at least one, two, or three but no more than four modifications, e.g., substitutions, relative to the nucleotide sequence of SEQ ID NO: 1792, 1793, or 1794.

In some embodiments, the EF-1a promoter variant, e.g., truncated EF-1a promoter, comprises a nucleotide sequence that is less than the full length of the nucleotide sequence of a wild-type (WT) EF-1a promoter comprising SEQ ID NO: 1781, optionally provided that the EF-1a promoter variant does not comprise the nucleotides “GC” at positions 235-236, numbered according to SEQ ID NO: 1781. In some embodiments, the EF-1a promoter variant, e.g., truncated EF-1a promoter, comprises a nucleotide sequence that comprises at least 10, 20, 25, 30, 50, 100, 150, 200, 250, 300, 350, 400, 450, 500, 550, 600, 650, 700, 750, 800, 850, 900, or 950 nucleotides less than the full length of a wild-type (WT) EF-1a promoter comprising SEQ ID NO: 1781. In some embodiments, the EF-1a promoter variant, e.g., truncated EF-1a promoter, comprises nucleotides 1-241 of SEQ ID NO: 1781. In some embodiments, the EF-1a promoter, e.g., truncated EF-1a promoter, variant comprises nucleotides 7-236 of SEQ ID NO: 1781. In some embodiments, the EF-1a promoter variant, e.g., truncated EF-1a promoter, comprises nucleotides 7-241 of SEQ ID NO: 1781. In some embodiments, the EF-1a promoter variant, e.g., truncated EF-1a promoter, comprises nucleotides 13-236 or 13-241 of SEQ ID NO: 1781. In some embodiments, the EF-1a promoter variant, e.g., truncated EF-1a promoter, comprises nucleotides 15-236 or 15-241 of SEQ ID NO: 1781. In some embodiments, the EF-1a promoter variant, e.g., truncated EF-1a promoter, further comprises the nucleotide sequence of SEQ ID NO: 1792, 1793, or 1794, or a sequence having at least one, two, or three but no more than four modifications, e.g., substitutions, relative to the nucleotide sequence of SEQ ID NO: 1792, 1793, or 1794.

In some embodiments, the EF-1a promoter variant, e.g., truncated EF-1a promoter, comprises from 5′ to 3,′ the nucleotide sequence of SEQ ID NO: 1792, or a sequence having at least one or two modifications, e.g., substitutions, relative to the nucleotide sequence of SEQ ID NO: 1792; and nucleotides 1-236 of SEQ ID NO: 1781. In some embodiments, the EF-1a promoter variant, e.g., truncated EF-1a promoter, comprises from 5′ to 3,′ the nucleotide sequence of SEQ ID NO: 1793, or a sequence having at least one, two, or three but no more than four modifications, e.g., substitutions, relative to the nucleotide sequence of SEQ ID NO: 1793; and nucleotides 1-236 of SEQ ID NO: 1781. In some embodiments, the EF-1a promoter variant, e.g., truncated EF-1a promoter, comprises from 5′ to 3,′ the nucleotide sequence of SEQ ID NO: 1794, or a sequence having at least one, two, or three but no more than four modifications, e.g., substitutions, relative to the nucleotide sequence of SEQ ID NO: 1794; and nucleotides 1-236 of SEQ ID NO: 1781.

In some embodiments, the EF-1a promoter variant, e.g., truncated EF-1a promoter, comprises an intron, e.g., an intron comprising the nucleotide sequence of positions 237-1,184 of SEQ ID NO: 1781. In some embodiments, the EF-1a promoter variant, e.g., truncated EF-1a promoter, comprises a portion of an intron, wherein the portion comprises no more than 5-50 nucleotides, e.g., 5-40 nucleotides, 5-30 nucleotides, 5-20 nucleotides, 5-10 nucleotides, 10-50 nucleotides, 10-40 nucleotides, 10-30 nucleotides, 10-20 nucleotides, 20-50 nucleotides, 20-40 nucleotides, 20-30 nucleotides, 30-50 nucleotides, 30-40 nucleotides, 40-50 nucleotides, 5 nucleotides, 10 nucleotides, 15 nucleotides, 20 nucleotides, 25 nucleotides, 30 nucleotides, 40 nucleotides, or 50 nucleotides of the intron e.g., an intron comprising the nucleotide sequence of positions 237-1,184 of SEQ ID NO: 1781. In some embodiments, the EF-1a promoter variant, e.g., truncated EF-1a promoter, comprises a portion of an intron, wherein the portion comprises the nucleotide sequence of SEQ ID NO: 1798. In some embodiments, the EF-1a promoter variant, e.g., truncated EF-1a promoter, does not comprises an intron, e.g., does not comprise the nucleotide sequence of positions 237-1,184 of SEQ ID NO: 1781.

In some embodiments, the promoter of a viral genome described herein comprises the nucleotide sequence of any of SEQ ID NOs: 1785, 1782-1784, 1786-1791, or a nucleotide sequence having at least 90% (e.g., 92, 95, 96, 97, 98, or 99%) sequence identity to any of the aforesaid sequence. In some embodiments, the promoter of a viral genome described herein comprises the nucleotide sequence of SEQ ID NO: 1785, or a sequence having at least 90% (e.g., 92, 95, 96, 97, 98, or 99%) sequence identity thereto. In some embodiments, the promoter of a viral genome described herein comprises the nucleotide sequence of SEQ ID NO: 1787, or a sequence having at least 90% (e.g., 92, 95, 96, 97, 98, or 99%) sequence identity thereto. In some embodiments, the promoter of a viral genome described herein comprises the nucleotide sequence of SEQ ID NO: 1789, or a sequence having at least 90% (e.g., 92, 95, 96, 97, 98, or 99%) sequence identity thereto. In some embodiments, the promoter of a viral genome described herein comprises the nucleotide sequence of SEQ ID NO: 1791, or a sequence having at least 90% (e.g., 92, 95, 96, 97, 98, or 99%) sequence identity thereto.

TABLE 12
Exemplary Promoter Variants

| | | | SEQ |
|---|---|---|---|
| | | | ID |
| Description | Sequences | Length | NO: |
| EF1a | CGTGAGGCTCCGGTGCCCGTCAGTGGGCAGAGCGCACATCGCCCACAGTCCCC | 1,184 | 1781 |
| Promoter | GAGAAGTTGGGGGGAGGGGTCGGCAATTGAACCGGTGCCTAGAGAAGGTGGCG | | |
| (intron | CGGGGTAAACTGGGAAAGTGATGTCGTGTACTGGCTCCGCCTTTTTCCCGAGG | | |
| underlined) | GTGGGGGAGAACCGTATATAAGTGCAGTAGTCGCCGTGAACGTTCTTTTTCGC | | |
| | AACGGGTTTGCCGCCAGAACACAGGTAAGTGCCGTGTGTGGTTCCCGCGGGCC | | |
| | TGGCCTCTTTACGGGTTATGGCCCTTGCGTGCCTTGAATTACTTCCACCTGGC | | |
| | TGCAGTACGTGATTCTTGATCCCGAGCTTCGGGTTGGAAGTGGGTGGGAGAGT | | |
| | TCGAGGCCTTGCGCTTAAGGAGCCCCTTCGCCTCGTGCTTGAGTTGAGGCCTG | | |
| | GCCTGGGCGCTGGGGCCGCCGCGTGCGAATCTGGTGGCACCTTCGCGCCTGTC | | |
| | TCGCTGCTTTCGATAAGTCTCTAGCCATTTAAAATTTTTGATGACCTGCTGCG | | |
| | ACGCTTTTTTTCTGGCAAGATAGTCTTGTAAATGCGGGCCAAGATCTGCACAC | | |
| | TGGTATTTCGGTTTTTGGGGCCGCGGGCGGCGACGGGGCCCGTGCGTCCCAGC | | |
| | GCACATGTTCGGCGAGGCGGGGCCTGCGAGCGCGGCCACCGAGAATCGGACGG | | |
| | GGGTAGTCTCAAGCTGGCCGGCCTGCTCTGGTGCCTGGCCTCGCGCCGCCGTG | | |
| | TATCGCCCCGCCCTGGGCGGCAAGGCTGGCCCGGTCGGCACCAGTTGCGTGAG | | |
| | CGGAAAGATGGCCGCTTCCCGGCCCTGCTGCAGGGAGCTCAAAATGGAGGACG | | |
| | CGGCGCTCGGGAGAGCGGGCGGGTGAGTCACCCACACAAAGGAAAAGGGCCTT | | |
| | TCCGTCCTCAGCCGTCGCTTCATGTGACTCCACGGAGTACCGGGCGCCGTCCA | | |
| | GGCACCTCGATTAGTTCTCGAGCTTTTGGAGTACGTCGTCTTTAGGTTGGGGG | | |
| | GAGGGGTTTTATGCGATGGAGTTTCCCCACACTGAGTGGGTGGAGACTGAAGT | | |
| | TAGGCCAGCTTGGCACTTGATGTAATTCTCCTTGGAATTTGCCCTTTTTGAGT | | |
| | TTGGATCTTGGTTCATTCTCAAGCCTCAGACAGTGGTTCAAAGTTTTTTTCTT | | |
| | CCATTTCAGGTGTCGTGA | | |
| miniEF1a | GCCCGTCAGTGGGCAGAGCGCACATCGCCCACAGTCCCCGAGAAGTTGGGGGG | 227 nt | 1745 |
| | AGGGGTCGGCAATTGAACCGGTGCCTAGAGAAGGTGGCGCGGGGTAAACTGGG | | |
| | AAAGTGATGTCGTGTACTGGCTCCGCCTTTTTCCCGAGGGTGGGGGAGAACCG | | |
| | TATATAAGTGCAGTAGTCGCCGTGAACGTTCTTTTTCGCAACGGGTTTGCCGC | | |
| | CAGAACACGCGTAAG | | |
| Promoter | GCATG | 5 nt | 1792 |
| Variant 1 | | | |
| Promoter | GGTGGAGAAGAGCATG | 16 nt | 1793 |
| Variant 2 | | | |
| Promoter | GTCATCACTGAGGTGGAGAAGAGCATG | 27 nt | 1794 |
| Variant 3 | | | |
| Promoter | CGTGAG | 6 nt | 1795 |
| Variant 4 | | | |
| Promoter | GT | 2 nt | 1796 |
| Variant 5 | | | |
| Promoter | GCTCCGGT | 8 nt | 1797 |
| Variant 6 | | | |
| Promoter | GCCCGTCAGTGGGCAGAGCGCACATCGCCCACAGTCCCCGAGAAGTTGGGGGG | 222 nt | 1822 |
| Variant 19 | AGGGGTCGGCAATTGAACCGGTGCCTAGAGAAGGTGGCGCGGGGTAAACTGGG | | |
| | AAAGTGATGTCGTGTACTGGCTCCGCCTTTTTCCCGAGGGTGGGGGAGAACCG | | |
| | TATATAAGTGCAGTAGTCGCCGTGAACGTTCTTTTTCGCAACGGGTTTGCCGC | | |
| | CAGAACACAG | | |
| Promoter | GCCCGTCAGTGGGCAGAGCGCACATCGCCCACAGTCCCCGAGAAGTTGGGGGG | 222 nt | 1823 |
| Variant 20 | AGGGGTCGGCAATTGAACCGGTGCCTAGAGAAGGTGGCGCGGGGTAAACTGGG | | |
| | AAAGTGATGTCGTGTACTGGCTCCGCCTTTTTCCCGAGGGTGGGGGAGAACCG | | |
| | TATATAAGTGCAGTAGTCGCCGTGAACGTTCTTTTTCGCAACGGGTTTGCCGC | | |
| | CAGAACACGC | | |
| Promoter | GTAAG | 5 nt | 1798 |
| Variant 7 | | | |
| Promoter | GTGCCCGTCAGTGGGCAGAGCGCACATCGCCCACAGTCCCCGAGAAGTTGGGG | 229 nt | 1782 |
| Variant 8 | GGAGGGGTCGGCAATTGAACCGGTGCCTAGAGAAGGTGGCGCGGGGTAAACTG | | |
| | GGAAAGTGATGTCGTGTACTGGCTCCGCCTTTTTCCCGAGGGTGGGGGAGAAC | | |
| | CGTATATAAGTGCAGTAGTCGCCGTGAACGTTCTTTTTCGCAACGGGTTTGCC | | |
| | GCCAGAACACGCGTAAG | | |
| Promoter | GCTCCGGTGCCCGTCAGTGGGCAGAGCGCACATCGCCCACAGTCCCCGAGAAG | 235 nt | 1783 |
| Variant 9 | TTGGGGGGAGGGGTCGGCAATTGAACCGGTGCCTAGAGAAGGTGGCGCGGGGT | | |
| | AAACTGGGAAAGTGATGTCGTGTACTGGCTCCGCCTTTTTCCCGAGGGTGGGG | | |
| | GAGAACCGTATATAAGTGCAGTAGTCGCCGTGAACGTTCTTTTTCGCAACGGG | | |
| | TTTGCCGCCAGAACACGCGTAAG | | |
| Promoter | CGTGAGGCTCCGGTGCCCGTCAGTGGGCAGAGCGCACATCGCCCACAGTCCCC | 241 nt | 1784 |
| Variant 10 | GAGAAGTTGGGGGGAGGGGTCGGCAATTGAACCGGTGCCTAGAGAAGGTGGCG | | |
| | CGGGGTAAACTGGGAAAGTGATGTCGTGTACTGGCTCCGCCTTTTTCCCGAGG | | |
| | GTGGGGGAGAACCGTATATAAGTGCAGTAGTCGCCGTGAACGTTCTTTTTCGC | | |
| | AACGGGTTTGCCGCCAGAACACGCGTAAG | | |
| Promoter | CGTGAGGCTCCGGTGCCCGTCAGTGGGCAGAGCGCACATCGCCCACAGTCCCC | 236 nt | 1785 |
| Variant 11 | GAGAAGTTGGGGGGAGGGGTCGGCAATTGAACCGGTGCCTAGAGAAGGTGGCG | | |
| | CGGGGTAAACTGGGAAAGTGATGTCGTGTACTGGCTCCGCCTTTTTCCCGAGG | | |
| | GTGGGGGAGAACCGTATATAAGTGCAGTAGTCGCCGTGAACGTTCTTTTTCGC | | |
| | AACGGGTTTGCCGCCAGAACACAG | | |
| Promoter | GCATGCGTGAGGCTCCGGTGCCCGTCAGTGGGCAGAGCGCACATCGCCCACAG | 246 nt | 1786 |
| Variant 12 | TCCCCGAGAAGTTGGGGGGAGGGGTCGGCAATTGAACCGGTGCCTAGAGAAGG | | |
| | TGGCGCGGGGTAAACTGGGAAAGTGATGTCGTGTACTGGCTCCGCCTTTTTCC | | |
| | CGAGGGTGGGGGAGAACCGTATATAAGTGCAGTAGTCGCCGTGAACGTTCTTT | | |
| | TTCGCAACGGGTTTGCCGCCAGAACACGCGTAAG | | |
| Promoter | GCATGCGTGAGGCTCCGGTGCCCGTCAGTGGGCAGAGCGCACATCGCCCACAG | 241 nt | 1787 |
| Variant 13 | TCCCCGAGAAGTTGGGGGGAGGGGTCGGCAATTGAACCGGTGCCTAGAGAAGG | | |
| | TGGCGCGGGGTAAACTGGGAAAGTGATGTCGTGTACTGGCTCCGCCTTTTTCC | | |
| | CGAGGGTGGGGGAGAACCGTATATAAGTGCAGTAGTCGCCGTGAACGTTCTTT | | |
| | TTCGCAACGGGTTTGCCGCCAGAACACAG | | |
| Promoter | GGTGGAGAAGAGCATGCGTGAGGCTCCGGTGCCCGTCAGTGGGCAGAGCGCAC | 257 nt | 1788 |
| Variant 14 | ATCGCCCACAGTCCCCGAGAAGTTGGGGGGAGGGGTCGGCAATTGAACCGGTG | | |
| | CCTAGAGAAGGTGGCGCGGGGTAAACTGGGAAAGTGATGTCGTGTACTGGCTC | | |
| | CGCCTTTTTCCCGAGGGTGGGGGAGAACCGTATATAAGTGCAGTAGTCGCCGT | | |
| | GAACGTTCTTTTTCGCAACGGGTTTGCCGCCAGAACACGCGTAAG | | |
| Promoter | GGTGGAGAAGAGCATGCGTGAGGCTCCGGTGCCCGTCAGTGGGCAGAGCGCAC | 252 nt | 1789 |
| Variant 15 | ATCGCCCACAGTCCCCGAGAAGTTGGGGGGAGGGGTCGGCAATTGAACCGGTG | | |
| | CCTAGAGAAGGTGGCGCGGGGTAAACTGGGAAAGTGATGTCGTGTACTGGCTC | | |
| | CGCCTTTTTCCCGAGGGTGGGGGAGAACCGTATATAAGTGCAGTAGTCGCCGT | | |
| | GAACGTTCTTTTTCGCAACGGGTTTGCCGCCAGAACACAG | | |
| Promoter | GTCATCACTGAGGTGGAGAAGAGCATGCGTGAGGCTCCGGTGCCCGTCAGTGG | 268 nt | 1790 |
| Variant 16 | GCAGAGCGCACATCGCCCACAGTCCCCGAGAAGTTGGGGGGAGGGGTCGGCAA | | |
| | TTGAACCGGTGCCTAGAGAAGGTGGCGCGGGGTAAACTGGGAAAGTGATGTCG | | |
| | TGTACTGGCTCCGCCTTTTTCCCGAGGGTGGGGGAGAACCGTATATAAGTGCA | | |
| | GTAGTCGCCGTGAACGTTCTTTTTCGCAACGGGTTTGCCGCCAGAACACGCGT | | |
| | AAG | | |
| Promoter | GTCATCACTGAGGTGGAGAAGAGCATGCGTGAGGCTCCGGTGCCCGTCAGTGG | 263 nt | 1791 |
| Variant 18 | GCAGAGCGCACATCGCCCACAGTCCCCGAGAAGTTGGGGGGAGGGGTCGGCAA | | |
| | TTGAACCGGTGCCTAGAGAAGGTGGCGCGGGGTAAACTGGGAAAGTGATGTCG | | |
| | TGTACTGGCTCCGCCTTTTTCCCGAGGGTGGGGGAGAACCGTATATAAGTGCA | | |
| | GTAGTCGCCGTGAACGTTCTTTTTCGCAACGGGTTTGCCGCCAGAACACAG | | |

In some embodiments, the viral genome comprises an intron. In some embodiments, the viral genome comprises at least two, three, four, or more introns. In some embodiments, the intron is a chimeric intron. In some embodiments, the intron is present 5′ relative to the transgene encoding the NPC protein, e.g., the NPC1 protein. In some embodiments, the intron comprises the nucleotide sequence of SEQ ID NO: 1780, or a nucleotide sequence at least 80%, 85%, 90%, 92%, 95%, 97%, 98%, or 99% identical thereto.

In some embodiments, the viral genome comprises a Kozak sequence. In some embodiments, the Kozak sequence comprises the nucleotide sequence of SEQ ID NO: 1746, or a nucleotide sequence comprising at least one, two, or three but no more than four modifications, e.g., substitutions, relative to the nucleotide sequence of SEQ ID NO: 1746. In some embodiments, the Kozak sequence is located 5′ relative to the transgene encoding the NPC1 protein. In some embodiments, the Kozak sequence is located immediately adjacent to the 5′ end of the transgene encoding the NPC1 protein.

In some embodiments, the viral genome comprises a polyadenylation (polyA) signal region. In some embodiments, the viral genome comprises a rabbit globin polyA signal region. In some embodiments, the polyA signal region comprises the nucleotide sequence of SEQ ID NO: 1751, or a nucleotide sequence at least 80%, 85%, 90%, 92%, 95%, 97%, 98%, or 99% identical thereto.

In some embodiments, the viral genome comprises a nucleic acid comprising a transgene encoding an NPC1 protein, wherein the transgene comprises the nucleotide sequence of any of SEQ ID NO: 1747, 1749, 1750, or a nucleotide sequence substantially identical (e.g., having at least about 70%, 75%, 80%, 85%, 90%, 92%, 95%, 97%, 98%, or 99% sequence identity), to any of the aforesaid nucleotide sequences. In some the transgene comprises the nucleotide sequence of SEQ ID NO: 1749, nucleotide sequence substantially identical (e.g., having at least about 70%, 75%, 80%, 85%, 90%, 92%, 95%, 97%, 98%, or 99% sequence identity) to SEQ ID NO: 1749. In some the transgene comprises the nucleotide sequence of SEQ ID NO: 1750, nucleotide sequence substantially identical (e.g., having at least about 70%, 75%, 80%, 85%, 90%, 92%, 95%, 97%, 98%, or 99% sequence identity) to SEQ ID NO: 1750.

In some embodiments, the AAV viral genome comprises a 5′ ITR of SEQ ID NO: 1733; a promoter region of SEQ ID NO: 1735, SEQ ID NO: 1736, SEQ ID NO: 1737, SEQ ID NO: 1738, SEQ ID NO: 1739, SEQ ID NO: 1740, SEQ ID NO: 1741, SEQ ID NO: 1742, SEQ ID NO: 1743, SEQ ID NO: 1744, or SEQ ID NO: 1745; a Kozak region of SEQ ID NO: 1746; an NPC protein coding sequence of SEQ ID NO: 1747, SEQ ID NO: 1749, or SEQ ID NO: 1750; a polyA region of SEQ ID NO: 1751; and a 3′ ITR of SEQ ID NO: 1734.

Tables 4 and 13-17 provide exemplary ITR to ITR sequences for AAV viral genomes according to the present disclosure. In some embodiments, the viral genome comprises one or more additional nucleotide sequences, e.g., one or more nucleotide sequences described in Table 3 or 12, or a nucleotide sequence (e.g., having at least about 70%, 75%, 80%, 85%, 90%, 92%, 95%, 97%, 98%, or 99% sequence identity), to any of the aforesaid sequences.

In some embodiments, the viral genome of an AAV particle described herein comprises the nucleotide sequence, e.g., the nucleotide sequence from the 5′ ITR to the 3′ ITR, of the nucleotide sequences of ITR_ITR 1-ITR_ITR 44, e.g., as described in Tables 4 or 13-17, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the viral genome of an AAV particle described herein comprises the nucleotide sequence, e.g., the nucleic acid sequence from the 5′ ITR to the 3′ ITR, of any of the nucleotide sequences in Table 4 or 13-17, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the viral genome of an AAV particle described herein comprises the nucleotide sequence, e.g., the nucleic acid sequence from the 5′ ITR to the 3′ ITR, of any of the nucleotide sequences of SEQ ID NOs: 1752-1759, 1799-1821, or 1824-1836, or a nucleotide sequence substantially identical (e.g., having at least about 70%, 75%, 80%, 85%, 90%, 92%, 95%, 97%, 98%, or 99% sequence identity) to any of the aforesaid sequences.

This disclosure also provides in some embodiments, an NPC1 protein encoded by any one of SEQ ID NOs: 1752-1759, 1799-1821, or 1824-1836, or a nucleotide sequence substantially identical (e.g., having at least about 70%, 75%, 80%, 85%, 90%, 92%, 95%, 97%, 98%, or 99% sequence identity) to any of the aforesaid sequences.

TABLE 4
Exemplary NPC1 AAV viral genome ITR to ITR sequences

| | | Length of ITR | |
|---|---|---|---|
| ITR to ITR | Description of Region | to ITR | SEQ |
| Construct ID | encoding the NPC Protein | Construct (nts) | ID NO: |
| ITR_ITR 1 | short CMV promoter (SEQ ID NO: 1736); | 4629 | 1752 |
| | WT NPC1 protein coding sequence | | |
| | (SEQ ID NO: 1747) | | |
| ITR_ITR 2 | Promoter Variant 8 (SEQ ID NO: 1782); | 4639 | 1753 |
| | WT NPC1 protein coding sequence | | |
| | (SEQ ID NO: 1747) | | |
| ITR_ITR 3 | Promoter Variant 8 (SEQ ID NO: 1782); pClneo- | 4787 | 1754 |
| | intron; WT NPC1 protein coding sequence | | |
| | (SEQ ID NO: 1747) | | |
| ITR_ITR 4 | Promoter Variant 8 (SEQ ID NO: 1782); codon | 4639 | 1755 |
| | optimized NPC1 protein coding sequence 2 | | |
| | (SEQ ID NO: 1750) | | |
| ITR_ITR 5 | CMV-D3 Promoter (SEQ ID NO: 1739); | 4773 | 1756 |
| | WT NPC1 protein coding sequence | | |
| | (SEQ ID NO: 1747) | | |
| ITR_ITR 6 | Promoter Variant 8 (SEQ ID NO: 1782); codon | 4639 | 1757 |
| | optimized NPC1 protein coding sequence 1 | | |
| | (SEQ ID NO: 1749) | | |
| ITR_ITR 7 | short CBA promoter (SEQ ID NO: 1735); | 4672 | 1758 |
| | WT NPC1 protein coding sequence | | |
| | (SEQ ID NO: 1747) | | |
| ITR_ITR 8 | CBA-D4 promoter (SEQ ID NO: 1738); | 4744 | 1759 |
| | WT NPC1 protein coding sequence | | |
| | (SEQ ID NO: 1747) | | |
| ITR_ITR 9 | Promoter Variant 11 (SEQ ID NO: 1785); codon | 4648 | 1799 |
| | optimized NPC1 protein coding sequence 2 | | |
| | (SEQ ID NO: 1750) | | |
| ITR_ITR 10 | Promoter Variant 11 (SEQ ID NO: 1785); pClneo- | 4796 | 1800 |
| | intron; codon optimized NPC1 protein coding | | |
| | sequence 2 (SEQ ID NO: 1750) | | |
| ITR_ITR 11 | short CMV promoter (SEQ ID NO: 1736); codon | 4629 | 1801 |
| | optimized NPC1 protein coding sequence 2 | | |
| | (SEQ ID NO: 1750) | | |
| ITR_ITR 12 | short CMV promoter (SEQ ID NO: 1736); pClneo- | 4777 | 1802 |
| | intron; codon optimized NPC1 protein coding | | |
| | sequence 2 (SEQ ID NO: 1750) | | |
| ITR_ITR 13 | short CBA promoter (SEQ ID NO: 1735); codon | 4672 | 1803 |
| | optimized NPC1 protein coding sequence 2 | | |
| | (SEQ ID NO: 1750) | | |
| ITR_ITR 14 | Promoter Variant 8 (SEQ ID NO: 1782); pClneo- | 4787 | 1804 |
| | intron; codon optimized NPC1 protein coding | | |
| | sequence 2 (SEQ ID NO: 1750) | | |
| ITR_ITR 15 | short CBA promoter (SEQ ID NO: 1735); codon | 4672 | 1805 |
| | optimized NPC1 protein coding sequence 1 | | |
| | (SEQ ID NO: 1749) | | |
| ITR_ITR 16 | short CMV promoter (SEQ ID NO: 1736); codon | 4629 | 1806 |
| | optimized NPC1 protein coding sequence 1 | | |
| | (SEQ ID NO: 1749) | | |
| ITR_ITR 17 | Promoter Variant 8 (SEQ ID NO: 1782); pClneo- | 4787 | 1807 |
| | intron; codon optimized NPC1 protein coding | | |
| | sequence 1 (SEQ ID NO: 1749) | | |
| ITR_ITR 18 | short CBA promoter (SEQ ID NO: 1735); pClneo- | 4820 | 1808 |
| | intron; WT NPC1 protein coding sequence | | |
| | (SEQ ID NO: 1747) | | |
| ITR_ITR 19 | short CMV promoter (SEQ ID NO: 1736); pClneo- | 4777 | 1809 |
| | intron; WT NPC1 protein coding sequence | | |
| | (SEQ ID NO: 1747) | | |
| ITR_ITR 20 | short CBA promoter (SEQ ID NO: 1735); pClneo- | 4820 | 1810 |
| | intron; codon optimized NPC1 protein coding | | |
| | sequence 2 (SEQ ID NO: 1750) | | |
| ITR_ITR 21 | short CBA promoter (SEQ ID NO: 1735); pClneo- | 4820 | 1811 |
| | intron; codon optimized NPC1 protein coding | | |
| | sequence 1 (SEQ ID NO: 1749) | | |
| ITR_ITR 22 | short CMV promoter (SEQ ID NO: 1736); pClneo- | 4777 | 1812 |
| | intron; codon optimized NPC1 protein coding | | |
| | sequence 1 (SEQ ID NO: 1749) | | |
| ITR_ITR 23 | Promoter Variant 8 (SEQ ID NO: 1782); | 4684 | 1813 |
| | WT NPC1 protein coding sequence | | |
| | (SEQ ID NO: 1747) | | |
| ITR_ITR 24 | Promoter Variant 13 (SEQ ID NO: 1787); pClneo- | 4801 | 1814 |
| | intron; WT NPC1 protein coding sequence | | |
| | (SEQ ID NO: 1747) | | |
| ITR_ITR 25 | Promoter Variant 13 (SEQ ID NO: 1787); codon | 4653 | 1815 |
| | optimized NPC1 protein coding sequence 2 | | |
| | (SEQ ID NO: 1750) | | |
| ITR_ITR 26 | Promoter Variant 13 (SEQ ID NO: 1787); pClneo- | 4801 | 1816 |
| | intron; codon optimized NPC1 protein coding | | |
| | sequence 2 (SEQ ID NO: 1750) | | |
| ITR_ITR 27 | Promoter Variant 11 (SEQ ID NO: 1785); | 4648 | 1817 |
| | WT NPC1 protein coding sequence | | |
| | (SEQ ID NO: 1747) | | |
| ITR_ITR 28 | Promoter Variant 13 (SEQ ID NO: 1787); | 4653 | 1818 |
| | WT NPC1 protein coding sequence | | |
| | (SEQ ID NO: 1747) | | |
| ITR_ITR 29 | Promoter Variant 11 (SEQ ID NO: 1785); pClneo- | 4796 | 1819 |
| | intron; WT NPC1 protein coding sequence | | |
| | (SEQ ID NO: 1747) | | |
| ITR_ITR 30 | Promoter Variant 15 (SEQ ID NO: 1789); | 4664 | 1820 |
| | WT NPC1 protein coding sequence | | |
| | (SEQ ID NO: 1747) | | |
| ITR_ITR 31 | Promoter Variant 18 (SEQ ID NO: 1791); | 4675 | 1821 |
| | WT NPC1 protein coding sequence | | |
| | (SEQ ID NO: 1747) | | |
| ITR_ITR 32 | short CMV promoter (SEQ ID NO: 1736); pClneo- | 4777 | 1824 |
| | intron; codon optimized NPC1 protein coding | | |
| | sequence 2 (SEQ ID NO: 1750) | | |
| ITR_ITR 33 | Promoter Variant 11 (SEQ ID NO: 1785); | 4648 | 1825 |
| | WT NPC1 protein coding sequence | | |
| | (SEQ ID NO: 1747) | | |
| ITR_ITR 34 | Promoter Variant 11 (SEQ ID NO: 1785); pClneo- | 4796 | 1826 |
| | intron; WT NPC1 protein coding sequence | | |
| | (SEQ ID NO: 1747) | | |
| ITR_ITR 35 | Promoter Variant 11 (SEQ ID NO: 1785); codon | 4648 | 1827 |
| | optimized NPC1 protein coding sequence 2 | | |
| | (SEQ ID NO: 1750) | | |
| ITR_ITR 36 | Promoter Variant 11 (SEQ ID NO: 1785); pClneo- | 4796 | 1828 |
| | intron; codon optimized NPC1 protein coding | | |
| | sequence 2 (SEQ ID NO: 1750) | | |
| ITR_ITR 37 | short CMV promoter (SEQ ID NO: 1736); pClneo- | 4777 | 1829 |
| | intron; WT NPC1 protein coding sequence | | |
| | (SEQ ID NO: 1747) | | |
| ITR_ITR 38 | short CMV promoter (SEQ ID NO: 1736); codon | 4629 | 1830 |
| | optimized NPC1 protein coding sequence 2 | | |
| | (SEQ ID NO: 1750) | | |
| ITR_ITR 39 | short CMV promoter (SEQ ID NO: 1736); pClneo- | 4777 | 1831 |
| | intron; codon optimized NPC1 protein coding | | |
| | sequence 2 (SEQ ID NO: 1750 | | |
| ITR_ITR 40 | Promoter Variant 8 (SEQ ID NO: 1782); | 4639 | 1832 |
| | WT NPC1 protein coding sequence | | |
| | (SEQ ID NO: 1747) | | |
| ITR_ITR 41 | CMVie enhancer (SEQ ID NO: 1737); short CBA | 5059 | 1833 |
| | promoter (SEQ ID NO: 1735); WT NPC1 protein | | |
| | coding sequence (SEQ ID NO: 1747) | | |
| ITR_ITR 42 | CBA-D4 Promoter (SEQ ID NO: 1738); | 4824 | 1834 |
| | WT NPC1 protein coding sequence | | |
| | (SEQ ID NO: 1747) | | |
| ITR_ITR 43 | CMV-D4 Promoter (SEQ ID NO: 1740); | 4701 | 1835 |
| | WT NPC1 protein coding sequence | | |
| | (SEQ ID NO: 1747) | | |
| ITR_ITR 44 | CMV-D6 Promoter (SEQ ID NO: 1741); | 4575 | 1836 |
| | WT NPC1 protein coding sequence | | |
| | (SEQ ID NO: 1747) | | |

TABLE 13
Exemplary ITR to ITR sequences encoding an NPC1 protein

| ITR to ITR | | SEQ |
|---|---|---|
| Construct | | ID |
| ID | Sequence | NO: |
| ITR_ITR 9 | ctgcgcgctcgctcgctcactgaggccgcccgggcaaagcccgggcgtcgggcgacctttggtc | 1799 |
| | gcccggcctcagtgagcgagcgagcgcgcagagagggagtggccaactccatcactaggggttc | |
| | cttgtagttaatgattaacccgccatgctacttatctaccagggtaatggggatcctctagaac | |
| | tatagctaacgcgtcgtgaggctccggtgcccgtcagtgggcagagcgcacatcgcccacagtc | |
| | cccgagaagttggggggaggggtcggcaattgaaccggtgcctagagaaggtggcgcggggtaa | |
| | actgggaaagtgatgtcgtgtactggctccgcctttttcccgagggtgggggagaaccgtatat | |
| | aagtgcagtagtcgccgtgaacgttctttttcgcaacgggtttgccgccagaacacagcgggag | |
| | caagcttcgtttcgaaccggtgccgccaccatgaccgccaggggcctggccctgggactgctgc | |
| | tgctgctgctgtgcccagcccaggtgttctctcagagctgcgtgtggtatggcgagtgtggcat | |
| | cgcctacggcgacaagcggtacaactgcgagtacagcggaccacctaagccactgccaaaggac | |
| | ggctacgatctggtgcaggagctgtgccctggcttctttttcggcaacgtgtccctgtgctgtg | |
| | acgtgcggcagctgcagaccctgaaggataatctgcagctgcctctgcagtttctgtctagatg | |
| | cccaagctgtttttataacctgctgaatctgttctgcgagctgacctgttccccaaggcagtct | |
| | cagttcctgaacgtgaccgccacagaggactacgtggatcccgtgacaaaccagaccaagacaa | |
| | atgtgaaggagctgcagtactatgtgggccagagcttcgccaacgccatgtataatgcatgcag | |
| | ggacgtggaggcacccagctccaatgataaggccctgggactgctgtgcggcaaggacgcagat | |
| | gcctgtaacgccaccaattggatcgagtacatgtttaacaaggataatggccaggcccctttca | |
| | ccatcacaccagtgttttccgacttccccgtgcacggcatggagcctatgaacaatgccaccaa | |
| | gggctgcgacgagtctgtggatgaggtgacagccccttgcagctgtcaggattgctccatcgtg | |
| | tgcggacctaagccacagccaccacctccaccagcaccatggaccatcctgggactggacgcca | |
| | tgtatgtgatcatgtggatcacatacatggcctttctgctggtgtttttcggcgcctttttcgc | |
| | cgtgtggtgctatcggaagagatacttcgtgagcgagtacacccccatcgacagcaacatcgcc | |
| | ttcagcgtgaatgcctccgacaagggcgaggcatcctgctgtgatcccgtgagcgccgccttcg | |
| | agggatgcctgcggagactgtttacccggtggggctctttctgcgtgagaaacccaggctgcgt | |
| | gatctttttcagcctggtgtttatcacagcctgttctagcggcctggtgttcgtgagggtgacc | |
| | acaaatccagtggatctgtggagcgccccctcctctcaggcaaggctggagaaggagtactttg | |
| | accagcacttcggcccatttttcaggaccgagcagctgatcatcagggcaccactgacagataa | |
| | gcacatctaccagccatatcctagcggagcagacgtgcccttcggccctccactggatatccag | |
| | atcctgcaccaggtgctggacctgcagatcgccatcgagaacatcaccgcctcctatgacaatg | |
| | agaccgtgacactgcaggatatctgcctggcccctctgagcccatataacaccaattgtacaat | |
| | cctgtccgtgctgaactactttcagaattcccactctgtgctggatcacaagaagggcgacgac | |
| | ttcttcgtgtacgccgactatcacacccacttcctgtactgcgtgcgggcccctgcctctctga | |
| | acgacaccagcctgctgcacgatccatgtctgggcacatttggcggccccgtgttcccttggct | |
| | ggtgctgggcggctatgacgatcagaactacaacaatgccaccgccctggtcatcaccttccct | |
| | gtgaacaattactataatgataccgagaagctgcagagagcccaggcctgggagaaggagttta | |
| | tcaacttcgtgaagaattacaagaacccaaatctgaccatcagcttcacagccgagcggtccat | |
| | cgaggacgagctgaacagagagagcgactccgacgtgttcaccgtggtcatcagctatgccatc | |
| | atgttcctgtacatcagcctggccctgggccacatcaagtcctgcaggcgcctgctggtggata | |
| | gcaaggtgtccctgggcatcgccggcatcctgatcgtgctgagctccgtggcctgttccctggg | |
| | cgtgttctcttatatcggcctgcctctgaccctgatcgtgatcgaagtgatcccatttctggtg | |
| | ctggccgtgggcgtggacaatatcttcatcctggtgcaggcataccagagggacgagcgcctgc | |
| | agggagagacactggatcagcagctgggaagggtgctgggagaggtggcaccatccatgtttct | |
| | gtctagcttctctgagaccgtggcctttttcctgggcgccctgtccgtgatgccagcagtgcac | |
| | acattttctctgttcgccggcctggccgtgtttatcgacttcctgctgcagatcacctgcttcg | |
| | tgagcctgctgggcctggacatcaagaggcaggagaagaaccgcctggatatcttctgctgcgt | |
| | gcggggagcagaggacggcacatccgtgcaggcctctgagagctgtctgttccggtttttcaag | |
| | aattcctattctccactgctgctgaaggactggatgagacccatcgtgatcgccatctttgtgg | |
| | gcgtgctgagcttctccatcgccgtgctgaacaaggtggacatcggcctggatcagtctctgag | |
| | catgcccgacgatagctatatggtggattactttaagtccatctctcagtatctgcacgcagga | |
| | ccccccgtgtacttcgtgctggaggagggccacgactacacctcctctaagggccagaatatgg | |
| | tgtgcggcggcatgggctgtaacaatgattccctggtgcagcagatcttcaacgccgcccagct | |
| | ggacaattatacacggatcggctttgcccccagctcctggatcgacgattacttcgattgggtg | |
| | aagcctcagtctagctgctgtagagtggacaacatcaccgatcagttttgcaatgcaagcgtgg | |
| | tggaccctgcatgcgtgaggtgtaggccactgacaccagagggcaagcagaggccacagggagg | |
| | cgacttcatgcgcttcctgcctatgttcctgtccgacaacccaaatccaaagtgtggcaaggga | |
| | ggacacgcagcatactcctctgccgtgaacatcctgctgggacacggaaccagggtgggagcca | |
| | catacttcatgacctaccacacagtgctgcagacctccgccgacttcatcgatgccctgaagaa | |
| | ggccaggctgatcgcctctaacgtgaccgagacaatgggcatcaatggctctgcctatcgcgtg | |
| | tttccctacagcgtgttttacgtgttctatgagcagtacctgaccatcatcgacgatacaatct | |
| | ttaacctgggcgtgagcctgggcgccatcttcctggtgaccatggtgctgctgggatgcgagct | |
| | gtggtccgccgtgatcatgtgcgccacaatcgccatggtgctggtgaatatgttcggcgtgatg | |
| | tggctgtggggcatcagcctgaacgccgtgtccctggtgaacctggtcatgtcttgcggcatca | |
| | gcgtggagttttgtagccacatcaccagagccttcacagtgtccatgaagggctctcgggtgga | |
| | gagagcagaggaggccctggcacacatgggcagctccgtgttttctggcatcaccctgacaaag | |
| | ttcggcggcatcgtggtgctggcctttgccaagagccagatcttccagatcttttatttccgga | |
| | tgtacctggcaatggtgctgctgggagcaacccacggcctgatctttctgcccgtgctgctgtc | |
| | ttatatcggccctagcgtgaacaaggccaagtcctgcgccaccgaggagagatacaagggcaca | |
| | gagcgggagagactgctgaatttctgactcgaggacggggtgaactacgcctgaggatccgatc | |
| | tttttccctctgccaaaaattatggggacatcatgaagccccttgagcatctgacttctggcta | |
| | ataaaggaaatttattttcattgcaatagtgtgttggaattttttgtgtctctcactcggccta | |
| | ggtagataagtagcatggcgggttaatcattaactacaaggaacccctagtgatggagttggcc | |
| | actccctctctgcgcgctcgctcgctcactgaggccgggcgaccaaaggtcgcccgacgcccgg | |
| | gctttgcccgggcggcctcagtgagcgagcgagcgcgcag | |
| ITR_ITR 10 | ctgcgcgctcgctcgctcactgaggccgcccgggcaaagcccgggcgtcgggcgacctttggtc | 1800 |
| | gcccggcctcagtgagcgagcgagcgcgcagagagggagtggccaactccatcactaggggttc | |
| | cttgtagttaatgattaacccgccatgctacttatctaccagggtaatggggatcctctagaac | |
| | tatagctaacgcgtcgtgaggctccggtgcccgtcagtgggcagagcgcacatcgcccacagtc | |
| | cccgagaagttggggggaggggtcggcaattgaaccggtgcctagagaaggtggcgcggggtaa | |
| | actgggaaagtgatgtcgtgtactggctccgcctttttcccgagggtgggggagaaccgtatat | |
| | aagtgcagtagtcgccgtgaacgttctttttcgcaacgggtttgccgccagaacacagcgggag | |
| | caagcttcgtttagtgaaccggtaagtatcaaggttacaagacaggtttaaggagaccaataga | |
| | aactgggcttgtcgagacagagaagactcttgcgtttctgataggcacctattggtcttactga | |
| | catccactttgcctttctctccacaggggattcgaaccggtgccgccaccatgaccgccagggg | |
| | cctggccctgggactgctgctgctgctgctgtgcccagcccaggtgttctctcagagctgcgtg | |
| | tggtatggcgagtgtggcatcgcctacggcgacaagcggtacaactgcgagtacagcggaccac | |
| | ctaagccactgccaaaggacggctacgatctggtgcaggagctgtgccctggcttctttttcgg | |
| | caacgtgtccctgtgctgtgacgtgcggcagctgcagaccctgaaggataatctgcagctgcct | |
| | ctgcagtttctgtctagatgcccaagctgtttttataacctgctgaatctgttctgcgagctga | |
| | cctgttccccaaggcagtctcagttcctgaacgtgaccgccacagaggactacgtggatcccgt | |
| | gacaaaccagaccaagacaaatgtgaaggagctgcagtactatgtgggccagagcttcgccaac | |
| | gccatgtataatgcatgcagggacgtggaggcacccagctccaatgataaggccctgggactgc | |
| | tgtgcggcaaggacgcagatgcctgtaacgccaccaattggatcgagtacatgtttaacaagga | |
| | taatggccaggcccctttcaccatcacaccagtgttttccgacttccccgtgcacggcatggag | |
| | cctatgaacaatgccaccaagggctgcgacgagtctgtggatgaggtgacagccccttgcagct | |
| | gtcaggattgctccatcgtgtgcggacctaagccacagccaccacctccaccagcaccatggac | |
| | catcctgggactggacgccatgtatgtgatcatgtggatcacatacatggcctttctgctggtg | |
| | tttttcggcgcctttttcgccgtgtggtgctatcggaagagatacttcgtgagcgagtacaccc | |
| | ccatcgacagcaacatcgccttcagcgtgaatgcctccgacaagggcgaggcatcctgctgtga | |
| | tcccgtgagcgccgccttcgagggatgcctgcggagactgtttacccggtggggctctttctgc | |
| | gtgagaaacccaggctgcgtgatctttttcagcctggtgtttatcacagcctgttctagcggcc | |
| | tggtgttcgtgagggtgaccacaaatccagtggatctgtggagcgccccctcctctcaggcaag | |
| | gctggagaaggagtactttgaccagcacttcggcccatttttcaggaccgagcagctgatcatc | |
| | agggcaccactgacagataagcacatctaccagccatatcctagcggagcagacgtgcccttcg | |
| | gccctccactggatatccagatcctgcaccaggtgctggacctgcagatcgccatcgagaacat | |
| | caccgcctcctatgacaatgagaccgtgacactgcaggatatctgcctggcccctctgagccca | |
| | tataacaccaattgtacaatcctgtccgtgctgaactactttcagaattcccactctgtgctgg | |
| | atcacaagaagggcgacgacttcttcgtgtacgccgactatcacacccacttcctgtactgcgt | |
| | gcgggcccctgcctctctgaacgacaccagcctgctgcacgatccatgtctgggcacatttggc | |
| | ggccccgtgttcccttggctggtgctgggcggctatgacgatcagaactacaacaatgccaccg | |
| | ccctggtcatcaccttccctgtgaacaattactataatgataccgagaagctgcagagagccca | |
| | ggcctgggagaaggagtttatcaacttcgtgaagaattacaagaacccaaatctgaccatcagc | |
| | ttcacagccgagcggtccatcgaggacgagctgaacagagagagcgactccgacgtgttcaccg | |
| | tggtcatcagctatgccatcatgttcctgtacatcagcctggccctgggccacatcaagtcctg | |
| | caggcgcctgctggtggatagcaaggtgtccctgggcatcgccggcatcctgatcgtgctgagc | |
| | tccgtggcctgttccctgggcgtgttctcttatatcggcctgcctctgaccctgatcgtgatcg | |
| | aagtgatcccatttctggtgctggccgtgggcgtggacaatatcttcatcctggtgcaggcata | |
| | ccagagggacgagcgcctgcagggagagacactggatcagcagctgggaagggtgctgggagag | |
| | gtggcaccatccatgtttctgtctagcttctctgagaccgtggcctttttcctgggcgccctgt | |
| | ccgtgatgccagcagtgcacacattttctctgttcgccggcctggccgtgtttatcgacttcct | |
| | gctgcagatcacctgcttcgtgagcctgctgggcctggacatcaagaggcaggagaagaaccgc | |
| | ctggatatcttctgctgcgtgcggggagcagaggacggcacatccgtgcaggcctctgagagct | |
| | gtctgttccggtttttcaagaattcctattctccactgctgctgaaggactggatgagacccat | |
| | cgtgatcgccatctttgtgggcgtgctgagcttctccatcgccgtgctgaacaaggtggacatc | |
| | ggcctggatcagtctctgagcatgcccgacgatagctatatggtggattactttaagtccatct | |
| | ctcagtatctgcacgcaggaccccccgtgtacttcgtgctggaggagggccacgactacacctc | |
| | ctctaagggccagaatatggtgtgcggcggcatgggctgtaacaatgattccctggtgcagcag | |
| | atcttcaacgccgcccagctggacaattatacacggatcggctttgcccccagctcctggatcg | |
| | acgattacttcgattgggtgaagcctcagtctagctgctgtagagtggacaacatcaccgatca | |
| | gttttgcaatgcaagcgtggtggaccctgcatgcgtgaggtgtaggccactgacaccagagggc | |
| | aagcagaggccacagggaggcgacttcatgcgcttcctgcctatgttcctgtccgacaacccaa | |
| | atccaaagtgtggcaagggaggacacgcagcatactcctctgccgtgaacatcctgctgggaca | |
| | cggaaccagggtgggagccacatacttcatgacctaccacacagtgctgcagacctccgccgac | |
| | ttcatcgatgccctgaagaaggccaggctgatcgcctctaacgtgaccgagacaatgggcatca | |
| | atggctctgcctatcgcgtgtttccctacagcgtgttttacgtgttctatgagcagtacctgac | |
| | catcatcgacgatacaatctttaacctgggcgtgagcctgggcgccatcttcctggtgaccatg | |
| | gtgctgctgggatgcgagctgtggtccgccgtgatcatgtgcgccacaatcgccatggtgctgg | |
| | tgaatatgttcggcgtgatgtggctgtggggcatcagcctgaacgccgtgtccctggtgaacct | |
| | ggtcatgtcttgcggcatcagcgtggagttttgtagccacatcaccagagccttcacagtgtcc | |
| | atgaagggctctcgggtggagagagcagaggaggccctggcacacatgggcagctccgtgtttt | |
| | ctggcatcaccctgacaaagttcggcggcatcgtggtgctggcctttgccaagagccagatctt | |
| | ccagatcttttatttccggatgtacctggcaatggtgctgctgggagcaacccacggcctgatc | |
| | tttctgcccgtgctgctgtcttatatcggccctagcgtgaacaaggccaagtcctgcgccaccg | |
| | aggagagatacaagggcacagagcgggagagactgctgaatttctgactcgaggacggggtgaa | |
| | ctacgcctgaggatccgatctttttccctctgccaaaaattatggggacatcatgaagcccctt | |
| | gagcatctgacttctggctaataaaggaaatttattttcattgcaatagtgtgttggaattttt | |
| | tgtgtctctcactcggcctaggtagataagtagcatggcgggttaatcattaactacaaggaac | |
| | ccctagtgatggagttggccactccctctctgcgcgctcgctcgctcactgaggccgggcgacc | |
| | aaaggtcgcccgacgcccgggctttgcccgggcggcctcagtgagcgagcgagcgcgcag | |
| ITR_ITR 11 | ctgcgcgctcgctcgctcactgaggccgcccgggcaaagcccgggcgtcgggcgacctttggtc | 1801 |
| | gcccggcctcagtgagcgagcgagcgcgcagagagggagtggccaactccatcactaggggttc | |
| | cttgtagttaatgattaacccgccatgctacttatctaccagggtaatggggatcctctagaac | |
| | tatagctaacgcgtgctattaccatggtgatgcggttttggcagtacatcaatgggcgtggata | |
| | gcggtttgactcacggggatttccaagtctccaccccattgacgtcaatgggagtttgttttgg | |
| | caccaaaatcaacgggactttccaaaatgtcgtaacaactccgccccattgacgcaaatgggcg | |
| | gtaggcgtgtacggtgggaggtctatataagcagagctccgggagcaagcttcgtttcgaaccg | |
| | gtgccgccaccatgaccgccaggggcctggccctgggactgctgctgctgctgctgtgcccagc | |
| | ccaggtgttctctcagagctgcgtgtggtatggcgagtgtggcatcgcctacggcgacaagcgg | |
| | tacaactgcgagtacagcggaccacctaagccactgccaaaggacggctacgatctggtgcagg | |
| | agctgtgccctggcttctttttcggcaacgtgtccctgtgctgtgacgtgcggcagctgcagac | |
| | cctgaaggataatctgcagctgcctctgcagtttctgtctagatgcccaagctgtttttataac | |
| | ctgctgaatctgttctgcgagctgacctgttccccaaggcagtctcagttcctgaacgtgaccg | |
| | ccacagaggactacgtggatcccgtgacaaaccagaccaagacaaatgtgaaggagctgcagta | |
| | ctatgtgggccagagcttcgccaacgccatgtataatgcatgcagggacgtggaggcacccagc | |
| | tccaatgataaggccctgggactgctgtgcggcaaggacgcagatgcctgtaacgccaccaatt | |
| | ggatcgagtacatgtttaacaaggataatggccaggcccctttcaccatcacaccagtgttttc | |
| | cgacttccccgtgcacggcatggagcctatgaacaatgccaccaagggctgcgacgagtctgtg | |
| | gatgaggtgacagccccttgcagctgtcaggattgctccatcgtgtgcggacctaagccacagc | |
| | caccacctccaccagcaccatggaccatcctgggactggacgccatgtatgtgatcatgtggat | |
| | cacatacatggcctttctgctggtgtttttcggcgcctttttcgccgtgtggtgctatcggaag | |
| | agatacttcgtgagcgagtacacccccatcgacagcaacatcgccttcagcgtgaatgcctccg | |
| | acaagggcgaggcatcctgctgtgatcccgtgagcgccgccttcgagggatgcctgcggagact | |
| | gtttacccggtggggctctttctgcgtgagaaacccaggctgcgtgatctttttcagcctggtg | |
| | tttatcacagcctgttctagcggcctggtgttcgtgagggtgaccacaaatccagtggatctgt | |
| | ggagcgccccctcctctcaggcaaggctggagaaggagtactttgaccagcacttcggcccatt | |
| | tttcaggaccgagcagctgatcatcagggcaccactgacagataagcacatctaccagccatat | |
| | cctagcggagcagacgtgcccttcggccctccactggatatccagatcctgcaccaggtgctgg | |
| | acctgcagatcgccatcgagaacatcaccgcctcctatgacaatgagaccgtgacactgcagga | |
| | tatctgcctggcccctctgagcccatataacaccaattgtacaatcctgtccgtgctgaactac | |
| | tttcagaattcccactctgtgctggatcacaagaagggcgacgacttcttcgtgtacgccgact | |
| | atcacacccacttcctgtactgcgtgcgggcccctgcctctctgaacgacaccagcctgctgca | |
| | cgatccatgtctgggcacatttggcggccccgtgttcccttggctggtgctgggcggctatgac | |
| | gatcagaactacaacaatgccaccgccctggtcatcaccttccctgtgaacaattactataatg | |
| | ataccgagaagctgcagagagcccaggcctgggagaaggagtttatcaacttcgtgaagaatta | |
| | caagaacccaaatctgaccatcagcttcacagccgagcggtccatcgaggacgagctgaacaga | |
| | gagagcgactccgacgtgttcaccgtggtcatcagctatgccatcatgttcctgtacatcagcc | |
| | tggccctgggccacatcaagtcctgcaggcgcctgctggtggatagcaaggtgtccctgggcat | |
| | cgccggcatcctgatcgtgctgagctccgtggcctgttccctgggcgtgttctcttatatcggc | |
| | ctgcctctgaccctgatcgtgatcgaagtgatcccatttctggtgctggccgtgggcgtggaca | |
| | atatcttcatcctggtgcaggcataccagagggacgagcgcctgcagggagagacactggatca | |
| | gcagctgggaagggtgctgggagaggtggcaccatccatgtttctgtctagcttctctgagacc | |
| | gtggcctttttcctgggcgccctgtccgtgatgccagcagtgcacacattttctctgttcgccg | |
| | gcctggccgtgtttatcgacttcctgctgcagatcacctgcttcgtgagcctgctgggcctgga | |
| | catcaagaggcaggagaagaaccgcctggatatcttctgctgcgtgcggggagcagaggacggc | |
| | acatccgtgcaggcctctgagagctgtctgttccggtttttcaagaattcctattctccactgc | |
| | tgctgaaggactggatgagacccatcgtgatcgccatctttgtgggcgtgctgagcttctccat | |
| | cgccgtgctgaacaaggtggacatcggcctggatcagtctctgagcatgcccgacgatagctat | |
| | atggtggattactttaagtccatctctcagtatctgcacgcaggaccccccgtgtacttcgtgc | |
| | tggaggagggccacgactacacctcctctaagggccagaatatggtgtgcggcggcatgggctg | |
| | taacaatgattccctggtgcagcagatcttcaacgccgcccagctggacaattatacacggatc | |
| | ggctttgcccccagctcctggatcgacgattacttcgattgggtgaagcctcagtctagctgct | |
| | gtagagtggacaacatcaccgatcagttttgcaatgcaagcgtggtggaccctgcatgcgtgag | |
| | gtgtaggccactgacaccagagggcaagcagaggccacagggaggcgacttcatgcgcttcctg | |
| | cctatgttcctgtccgacaacccaaatccaaagtgtggcaagggaggacacgcagcatactcct | |
| | ctgccgtgaacatcctgctgggacacggaaccagggtgggagccacatacttcatgacctacca | |
| | cacagtgctgcagacctccgccgacttcatcgatgccctgaagaaggccaggctgatcgcctct | |
| | aacgtgaccgagacaatgggcatcaatggctctgcctatcgcgtgtttccctacagcgtgtttt | |
| | acgtgttctatgagcagtacctgaccatcatcgacgatacaatctttaacctgggcgtgagcct | |
| | gggcgccatcttcctggtgaccatggtgctgctgggatgcgagctgtggtccgccgtgatcatg | |
| | tgcgccacaatcgccatggtgctggtgaatatgttcggcgtgatgtggctgtggggcatcagcc | |
| | tgaacgccgtgtccctggtgaacctggtcatgtcttgcggcatcagcgtggagttttgtagcca | |
| | catcaccagagccttcacagtgtccatgaagggctctcgggtggagagagcagaggaggccctg | |
| | gcacacatgggcagctccgtgttttctggcatcaccctgacaaagttcggcggcatcgtggtgc | |
| | tggcctttgccaagagccagatcttccagatcttttatttccggatgtacctggcaatggtgct | |
| | gctgggagcaacccacggcctgatctttctgcccgtgctgctgtcttatatcggccctagcgtg | |
| | aacaaggccaagtcctgcgccaccgaggagagatacaagggcacagagcgggagagactgctga | |
| | atttctgactcgaggacggggtgaactacgcctgaggatccgatctttttccctctgccaaaaa | |
| | ttatggggacatcatgaagccccttgagcatctgacttctggctaataaaggaaatttattttc | |
| | attgcaatagtgtgttggaattttttgtgtctctcactcggcctaggtagataagtagcatggc | |
| | gggttaatcattaactacaaggaacccctagtgatggagttggccactccctctctgcgcgctc | |
| | gctcgctcactgaggccgggcgaccaaaggtcgcccgacgcccgggctttgcccgggcggcctc | |
| | agtgagcgagcgagcgcgcag | |
| ITR_ITR 12 | ctgcgcgctcgctcgctcactgaggccgcccgggcaaagcccgggcgtcgggcgacctttggtc | 1802 |
| | gcccggcctcagtgagcgagcgagcgcgcagagagggagtggccaactccatcactaggggttc | |
| | cttgtagttaatgattaacccgccatgctacttatctaccagggtaatggggatcctctagaac | |
| | tatagctaacgcgtgctattaccatggtgatgcggttttggcagtacatcaatgggcgtggata | |
| | gcggtttgactcacggggatttccaagtctccaccccattgacgtcaatgggagtttgttttgg | |
| | caccaaaatcaacgggactttccaaaatgtcgtaacaactccgccccattgacgcaaatgggcg | |
| | gtaggcgtgtacggtgggaggtctatataagcagagctccgggagcaagcttcgtttagtgaac | |
| | ccgtaagtatcaaggttacaagacaggtttaaggagaccaatagaaactgggcttgtcgagaca | |
| | gagaagactcttgcgtttctgataggcacctattggtcttactgacatccactttgcctttctc | |
| | tccacaggggattcgaaccggtgccgccaccatgaccgccaggggcctggccctgggactgctg | |
| | ctgctgctgctgtgcccagcccaggtgttctctcagagctgcgtgtggtatggcgagtgtggca | |
| | tcgcctacggcgacaagcggtacaactgcgagtacagcggaccacctaagccactgccaaagga | |
| | cggctacgatctggtgcaggagctgtgccctggcttctttttcggcaacgtgtccctgtgctgt | |
| | gacgtgcggcagctgcagaccctgaaggataatctgcagctgcctctgcagtttctgtctagat | |
| | gcccaagctgtttttataacctgctgaatctgttctgcgagctgacctgttccccaaggcagtc | |
| | tcagttcctgaacgtgaccgccacagaggactacgtggatcccgtgacaaaccagaccaagaca | |
| | aatgtgaaggagctgcagtactatgtgggccagagcttcgccaacgccatgtataatgcatgca | |
| | gggacgtggaggcacccagctccaatgataaggccctgggactgctgtgcggcaaggacgcaga | |
| | tgcctgtaacgccaccaattggatcgagtacatgtttaacaaggataatggccaggcccctttc | |
| | accatcacaccagtgttttccgacttccccgtgcacggcatggagcctatgaacaatgccacca | |
| | agggctgcgacgagtctgtggatgaggtgacagccccttgcagctgtcaggattgctccatcgt | |
| | gtgcggacctaagccacagccaccacctccaccagcaccatggaccatcctgggactggacgcc | |
| | atgtatgtgatcatgtggatcacatacatggcctttctgctggtgtttttcggcgcctttttcg | |
| | ccgtgtggtgctatcggaagagatacttcgtgagcgagtacacccccatcgacagcaacatcgc | |
| | cttcagcgtgaatgcctccgacaagggcgaggcatcctgctgtgatcccgtgagcgccgccttc | |
| | gagggatgcctgcggagactgtttacccggtggggctctttctgcgtgagaaacccaggctgcg | |
| | tgatctttttcagcctggtgtttatcacagcctgttctagcggcctggtgttcgtgagggtgac | |
| | cacaaatccagtggatctgtggagcgccccctcctctcaggcaaggctggagaaggagtacttt | |
| | gaccagcacttcggcccatttttcaggaccgagcagctgatcatcagggcaccactgacagata | |
| | agcacatctaccagccatatcctagcggagcagacgtgcccttcggccctccactggatatcca | |
| | gatcctgcaccaggtgctggacctgcagatcgccatcgagaacatcaccgcctcctatgacaat | |
| | gagaccgtgacactgcaggatatctgcctggcccctctgagcccatataacaccaattgtacaa | |
| | tcctgtccgtgctgaactactttcagaattcccactctgtgctggatcacaagaagggcgacga | |
| | cttcttcgtgtacgccgactatcacacccacttcctgtactgcgtgcgggcccctgcctctctg | |
| | aacgacaccagcctgctgcacgatccatgtctgggcacatttggcggccccgtgttcccttggc | |
| | tggtgctgggcggctatgacgatcagaactacaacaatgccaccgccctggtcatcaccttccc | |
| | tgtgaacaattactataatgataccgagaagctgcagagagcccaggcctgggagaaggagttt | |
| | atcaacttcgtgaagaattacaagaacccaaatctgaccatcagcttcacagccgagcggtcca | |
| | tcgaggacgagctgaacagagagagcgactccgacgtgttcaccgtggtcatcagctatgccat | |
| | catgttcctgtacatcagcctggccctgggccacatcaagtcctgcaggcgcctgctggtggat | |
| | agcaaggtgtccctgggcatcgccggcatcctgatcgtgctgagctccgtggcctgttccctgg | |
| | gcgtgttctcttatatcggcctgcctctgaccctgatcgtgatcgaagtgatcccatttctggt | |
| | gctggccgtgggcgtggacaatatcttcatcctggtgcaggcataccagagggacgagcgcctg | |
| | cagggagagacactggatcagcagctgggaagggtgctgggagaggtggcaccatccatgtttc | |
| | tgtctagcttctctgagaccgtggcctttttcctgggcgccctgtccgtgatgccagcagtgca | |
| | cacattttctctgttcgccggcctggccgtgtttatcgacttcctgctgcagatcacctgcttc | |
| | gtgagcctgctgggcctggacatcaagaggcaggagaagaaccgcctggatatcttctgctgcg | |
| | tgcggggagcagaggacggcacatccgtgcaggcctctgagagctgtctgttccggtttttcaa | |
| | gaattcctattctccactgctgctgaaggactggatgagacccatcgtgatcgccatctttgtg | |
| | ggcgtgctgagcttctccatcgccgtgctgaacaaggtggacatcggcctggatcagtctctga | |
| | gcatgcccgacgatagctatatggtggattactttaagtccatctctcagtatctgcacgcagg | |
| | accccccgtgtacttcgtgctggaggagggccacgactacacctcctctaagggccagaatatg | |
| | gtgtgcggcggcatgggctgtaacaatgattccctggtgcagcagatcttcaacgccgcccagc | |
| | tggacaattatacacggatcggctttgcccccagctcctggatcgacgattacttcgattgggt | |
| | gaagcctcagtctagctgctgtagagtggacaacatcaccgatcagttttgcaatgcaagcgtg | |
| | gtggaccctgcatgcgtgaggtgtaggccactgacaccagagggcaagcagaggccacagggag | |
| | gcgacttcatgcgcttcctgcctatgttcctgtccgacaacccaaatccaaagtgtggcaaggg | |
| | aggacacgcagcatactcctctgccgtgaacatcctgctgggacacggaaccagggtgggagcc | |
| | acatacttcatgacctaccacacagtgctgcagacctccgccgacttcatcgatgccctgaaga | |
| | aggccaggctgatcgcctctaacgtgaccgagacaatgggcatcaatggctctgcctatcgcgt | |
| | gtttccctacagcgtgttttacgtgttctatgagcagtacctgaccatcatcgacgatacaatc | |
| | tttaacctgggcgtgagcctgggcgccatcttcctggtgaccatggtgctgctgggatgcgagc | |
| | tgtggtccgccgtgatcatgtgcgccacaatcgccatggtgctggtgaatatgttcggcgtgat | |
| | gtggctgtggggcatcagcctgaacgccgtgtccctggtgaacctggtcatgtcttgcggcatc | |
| | agcgtggagttttgtagccacatcaccagagccttcacagtgtccatgaagggctctcgggtgg | |
| | agagagcagaggaggccctggcacacatgggcagctccgtgttttctggcatcaccctgacaaa | |
| | gttcggcggcatcgtggtgctggcctttgccaagagccagatcttccagatcttttatttccgg | |
| | atgtacctggcaatggtgctgctgggagcaacccacggcctgatctttctgcccgtgctgctgt | |
| | cttatatcggccctagcgtgaacaaggccaagtcctgcgccaccgaggagagatacaagggcac | |
| | agagcgggagagactgctgaatttctgactcgaggacggggtgaactacgcctgaggatccgat | |
| | ctttttccctctgccaaaaattatggggacatcatgaagccccttgagcatctgacttctggct | |
| | aataaaggaaatttattttcattgcaatagtgtgttggaattttttgtgtctctcactcggcct | |
| | aggtagataagtagcatggcgggttaatcattaactacaaggaacccctagtgatggagttggc | |
| | cactccctctctgcgcgctcgctcgctcactgaggccgggcgaccaaaggtcgcccgacgcccg | |
| | ggctttgcccgggcggcctcagtgagcgagcgagcgcgcag | |

In some embodiments, the viral genome of an AAV particle described herein comprises a nucleotide sequence comprising the all of the components or a combination of the components as described, e.g., in Tables 13-17, or a sequence having at least 70%, 75%, 80%, 85%, 90%, 95%, or 99% sequence identity to any of the aforesaid sequences.

TABLE 14
Sequence Regions in ITR to ITR Sequences

| | ITR_ITR 9 (SEQ ID NO: 1799) | | |
|---|---|---|---|
| | Region SEQ | Region | Position in |
| Sequence Regions | ID NO | length | SEQ ID NO: 1799 |
| 5′ ITR | 1733 | 130 | 1-130 |
| Promoter Variant 11 | 1785 | 236 | 207-442 |
| Kozak | 1746 | 9 | 470-478 |
| NPC protein coding | 1750 | 3837 | 479-4315 |
| sequence 2 | | | |
| PolyA | 1751 | 127 | 4349-4475 |
| 3′ ITR | 1734 | 130 | 4519-4648 |

In some embodiments, the AAV particle comprises a viral genome comprising the nucleotide sequence of SEQ ID NO: 1799 (ITR_ITR 9), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1799, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1799, comprises in 5′ to 3′ order: a 5′ inverted terminal repeat (ITR) sequence region comprising the nucleotide sequence of SEQ ID NO: 1733 or a nucleotide sequence at least 95% identical thereto; promoter variant 11 comprising the nucleotide sequence of SEQ ID NO: 1785, or a nucleotide sequence at least 95% identical thereto; a Kozak sequence comprising the nucleotide sequence of SEQ ID NO: 1746, or a nucleotide sequence comprising at least one, two, or three but no more than four modifications, e.g., substitutions, relative to the nucleotide sequence of SEQ ID NO: 1746; a nucleotide sequence encoding an NPC1 protein comprising the nucleotide sequence of SEQ ID NO: 1750, or a nucleotide sequence at least 85% (e.g., at least about 90, 92, 95, 96, 97, 98, or 99%) identical thereto; a polyadenylation sequence comprising the nucleotide sequence of SEQ ID NO: 1751, or a nucleotide sequence at least 95% identical thereto; and a 3′ ITR sequence region comprising the nucleotide sequence of SEQ ID NO: 1734, or a nucleotide sequence at least 95% identical thereto.

In some embodiments, the viral genome comprising the nucleotide sequence of SEQ ID NO: 1799, or a nucleotide sequence substantially identical (e.g., having at least 70%, 75%, 80%, 85%, 90%, 95%, or 99% sequence identity) thereto, encodes an NPC1 protein comprising an amino acid sequence of SEQ ID NO: 1748, or an amino acid sequence substantially identical (e.g., having at least 70%, 75%, 80%, 85%, 90%, 95%, or 99% sequence identity) thereto.

TABLE 15
Sequence Regions in ITR to ITR Sequences

| | ITR_ITR 10 (SEQ ID NO: 1800) | | |
|---|---|---|---|
| | Region SEQ | Region | Position in |
| Sequence Regions | ID NO | length | SEQ ID NO: 1800 |
| 5′ ITR | 1733 | 130 | 1-130 |
| Promoter Variant 11 | 1785 | 236 | 207-442 |
| Intron | 1780 | 133 | 470-602 |
| NPC protein coding | 1750 | 3837 | 627-4463 |
| sequence 2 | | | |
| PolyA | 1751 | 127 | 4497-4623 |
| 3′ ITR | 1734 | 130 | 4667-4796 |

In some embodiments, the AAV particle comprises a viral genome comprising the nucleotide sequence of SEQ ID NO: 1800 (ITR_ITR 10), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1800, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1800, comprises in 5′ to 3′ order: a 5′ inverted terminal repeat (ITR) sequence region comprising the nucleotide sequence of SEQ ID NO: 1733 or a nucleotide sequence at least 95% identical thereto; promoter variant 11 comprising the nucleotide sequence of SEQ ID NO: 1785, or a nucleotide sequence at least 95% identical thereto; an intron comprising the nucleotide sequence of SEQ ID NO: 1780, or nucleotide sequence at least 95% identical thereto; a nucleotide sequence encoding an NPC1 protein comprising the nucleotide sequence of SEQ ID NO: 1750, or a nucleotide sequence at least 85% (e.g., at least about 90, 92, 95, 96, 97, 98, or 99%) identical thereto; a polyadenylation sequence comprising the nucleotide sequence of SEQ ID NO: 1751, or a nucleotide sequence at least 95% identical thereto; and a 3′ ITR sequence region comprising the nucleotide sequence of SEQ ID NO: 1734, or a nucleotide sequence at least 95% identical thereto.

In some embodiments, the viral genome comprising the nucleotide sequence of SEQ ID NO: 1800, or a nucleotide sequence substantially identical (e.g., having at least 70%, 75%, 80%, 85%, 90%, 95%, or 99% sequence identity) thereto, encodes an NPC1 protein comprising an amino acid sequence of SEQ ID NO: 1748, or an amino acid sequence substantially identical (e.g., having at least 70%, 75%, 80%, 85%, 90%, 95%, or 99% sequence identity) thereto.

TABLE 16
Sequence Regions in ITR to ITR Sequences

| | ITR_ITR 11 (SEQ ID NO: 1801) | | |
|---|---|---|---|
| | Region SEQ | Region | Position in |
| Sequence Regions | ID NO | length | SEQ ID NO: 1801 |
| 5′ ITR | 1733 | 130 | 1-130 |
| Short CMV Promoter | 1736 | 205 | 219-423 |
| Kozak | 1746 | 9 | 451-459 |
| NPC protein coding | 1750 | 3837 | 460-4296 |
| sequence 2 | | | |
| PolyA | 1751 | 127 | 4330-4456 |
| 3′ ITR | 1734 | 130 | 4500-4629 |

In some embodiments, the AAV particle comprises a viral genome comprising the nucleotide sequence of SEQ ID NO: 1801 (ITR_ITR 11), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1801, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1801, comprises in 5′ to 3′ order: a 5′ inverted terminal repeat (ITR) sequence region comprising the nucleotide sequence of SEQ ID NO: 1733 or a nucleotide sequence at least 95% identical thereto; a CMV promoter comprising the nucleotide sequence of SEQ ID NO: 1736, or a nucleotide sequence at least 95% identical thereto; a Kozak sequence comprising the nucleotide sequence of SEQ ID NO: 1746, or a nucleotide sequence comprising at least one, two, or three but no more than four modifications, e.g., substitutions, relative to the nucleotide sequence of SEQ ID NO: 1746; a nucleotide sequence encoding an NPC1 protein comprising the nucleotide sequence of SEQ ID NO: 1750, or a nucleotide sequence at least 85% (e.g., at least about 90, 92, 95, 96, 97, 98, or 99%) identical thereto; a polyadenylation sequence comprising the nucleotide sequence of SEQ ID NO: 1751, or a nucleotide sequence at least 95% identical thereto; and a 3′ ITR sequence region comprising the nucleotide sequence of SEQ ID NO: 1734, or a nucleotide sequence at least 95% identical thereto.

In some embodiments, the viral genome comprising the nucleotide sequence of SEQ ID NO: 1801, or a nucleotide sequence substantially identical (e.g., having at least 70%, 75%, 80%, 85%, 90%, 95%, or 99% sequence identity) thereto, encodes an NPC1 protein comprising an amino acid sequence of SEQ ID NO: 1748, or an amino acid sequence substantially identical (e.g., having at least 70%, 75%, 80%, 85%, 90%, 95%, or 99% sequence identity) thereto.

TABLE 17
Sequence Regions in ITR to ITR Sequences

| | ITR_ITR 12 (SEQ ID NO: 1802) | | |
|---|---|---|---|
| | Region SEQ | Region | Position in |
| Sequence Regions | ID NO | length | SEQ ID NO: 1802 |
| 5′ ITR | 1733 | 130 | 1-130 |
| Short CMV Promoter | 1736 | 205 | 219-423 |
| Intron | 1780 | 133 | 451-583 |
| Kozak | 1746 | 9 | 599-607 |
| NPC protein coding | 1750 | 3837 | 608-4444 |
| sequence 2 | | | |
| PolyA | 1751 | 127 | 4478-4604 |
| 3′ ITR | 1734 | 130 | 4648-4777 |

In some embodiments, the AAV particle comprises a viral genome comprising the nucleotide sequence of SEQ ID NO: 1802 (ITR_ITR 12), or a nucleotide sequence substantially identical (e.g., having at least 70%, 75%, 80%, 85%, 90%, 95%, 99% or 100%, sequence identity) thereto. In some embodiments, the viral genome comprises the nucleotide sequence of SEQ ID NO: 1802, or a sequence substantially identical (e.g., having at least 70%, 75%, 80%, 85%, 90%, 95%, or 99% sequence identity) thereto. In some embodiments, the viral genome comprising the nucleotide sequence of SEQ ID NO: 1802, comprises in 5′ to 3′ order: a 5′ inverted terminal repeat (ITR) sequence region comprising the nucleotide sequence of SEQ ID NO: 1733 or a nucleotide sequence at least 95% identical thereto; a CMV promoter comprising the nucleotide sequence of SEQ ID NO: 1736, or a nucleotide sequence at least 95% identical thereto; an intron comprising the nucleotide sequence of SEQ ID NO: 1780, or nucleotide sequence at least 95% identical thereto; a Kozak sequence comprising the nucleotide sequence of SEQ ID NO: 1746, or a nucleotide sequence comprising at least one, two, or three but no more than four modifications, e.g., substitutions, relative to the nucleotide sequence of SEQ ID NO: 1746; a nucleotide sequence encoding an NPC1 protein comprising the nucleotide sequence of SEQ ID NO: 1750, or a nucleotide sequence at least 85% (e.g., at least about 90, 92, 95, 96, 97, 98, or 99%) identical thereto; a polyadenylation sequence comprising the nucleotide sequence of SEQ ID NO: 1751, or a nucleotide sequence at least 95% identical thereto; and a 3′ ITR sequence region comprising the nucleotide sequence of SEQ ID NO: 1734, or a nucleotide sequence at least 95% identical thereto.

In some embodiments, the viral genome comprising the nucleotide sequence of SEQ ID NO: 1802, or a nucleotide sequence substantially identical (e.g., having at least 70%, 75%, 80%, 85%, 90%, 95%, or 99% sequence identity) thereto, encodes an NPC1 protein comprising an amino acid sequence of SEQ ID NO: 1748, or an amino acid sequence substantially identical (e.g., having at least 70%, 75%, 80%, 85%, 90%, 95%, or 99% sequence identity) thereto.

In some embodiments, the AAV particle comprises an AAV viral genome comprising the nucleotide sequence of any of the viral genomes described herein, e.g., as described in Tables 4 or 13-17, or a nucleotide sequence substantially identical (e.g., having at least about 70%, 75%, 80%, 85%, 90%, 92%, 95%, 97%, 98%, or 99% sequence identity) to any of the aforesaid sequences. In some embodiments, the AAV viral genome further comprises a nucleic acid encoding a capsid protein, e.g., a structural protein. In some embodiments, the capsid protein comprises a VP1 polypeptide, a VP2 polypeptide, and/or a VP3 polypeptide. In some embodiments, the VP1 polypeptide, the VP2 polypeptide, and/or the VP3 polypeptide are encoded by at least one Cap gene. In some embodiments, the AAV viral genome further comprises a nucleic acid encoding a Rep protein, e.g., a non-structural protein. In some embodiments, the Rep protein comprises a Rep78 protein, a Rep68, Rep52 protein, and/or a Rep40 protein. In some embodiments, the Rep78 protein, the Rep68 protein, the Rep52 protein, and/or the Rep40 protein are encoded by at least one Rep gene.

In some embodiment, the AAV particle comprising a viral comprising the nucleotide sequence of any of the viral genomes described herein, e.g., as described in Tables 4 or 13-17, or a nucleotide sequence substantially identical (e.g., having at least about 70%, 75%, 80%, 85%, 90%, 92%, 95%, 97%, 98%, or 99% sequence identity) to any of the aforesaid sequences comprises, e.g., is packaged in, a capsid protein having a serotype or a functional variant thereof selected from Table 1. In some embodiments, the capsid protein comprise a VOY101, VOY201, AAVPHP.N (PHP.N), AAVPHP.B (PHP.B), AAVPHP.A (PHP.A), PHP.B2, PHP.B3, G2B4, G2B5, AAV9, AAVrh10, or a functional variant thereof. In some embodiments, the capsid protein comprises an AAV9 capsid protein, or functional variant thereof.

In some embodiments, the AAV particle comprising a viral genome comprising the nucleotide sequence of any of the viral genomes described herein, e.g., as described in Tables 4 or 13-17, or a nucleotide sequence substantially identical (e.g., having at least about 70%, 75%, 80%, 85%, 90%, 92%, 95%, 97%, 98%, or 99% sequence identity) to any of the aforesaid sequences comprises a capsid protein comprising the amino acid sequence of SEQ ID NO: 138, or a sequence substantially identical (e.g., having at least about 70%, 75%, 80%, 85%, 90%, 92%, 95%, 97%, 98%, or 99% sequence identity) thereto. In some embodiments, the capsid protein comprises an amino acid sequence having at least one, two or three modifications, but not more than 30, 20 or 10 modifications of the amino acid sequence of SEQ ID NO: 138. In some embodiments, the capsid protein is encoded by the nucleotide sequence of SEQ ID NO: 137, or a nucleotide sequence substantially identical (e.g., having at least 70%, 75%, 80%, 85%, 90%, 95% or 99%) thereto. In some embodiments, the capsid protein comprises an amino acid substitution at position K449, e.g., a K449R substitution, numbered according to SEQ ID NO:138. In some embodiments, the capsid protein comprises an insert comprising the amino acid sequence of TLAVPFK (SEQ ID NO: 1262), wherein the insert is present immediately subsequent to position 588, relative to a reference sequence numbered according to SEQ ID NO:138. In some embodiments, the capsid protein comprises an amino acid other than “A” at position 587 and/or an amino acid other than “Q” at position 588, numbered according to SEQ ID NO:138. In some embodiments, the capsid protein comprises the amino acid substitution of A587D and/or Q588G, numbered according to SEQ ID NO:138.

The present disclosure provides in some embodiments, vectors, cells, and/or AAV particles comprising the above identified viral genomes.

### Backbone

In some embodiments, a cis-element such as a vector backbone is incorporated into the viral particle encoding NPC protein. In some embodiments, the backbone sequence may regulate transcription during viral production. Without wishing to be bound by theory, it is believed, in some embodiments, the backbone sequence may contribute to the stability of NPC protein expression, and/or the level of expression of the NPC protein.

The present disclosure also provides in some embodiments, a nucleic acid encoding a viral genome, e.g., a viral genome comprising the nucleotide sequence of any of the viral genomes in Table 4 or 13-17, or a nucleotide sequence substantially identical (e.g., having at least 70%, 75%, 80%, 85%, 90%, 95%, 99% or 100%, sequence identity) thereto, an a backbone region suitable for replication of the viral genome in a cell, e.g., a bacterial cell (e.g., wherein the backbone region comprises one or both of a bacterial origin of replication and a selectable marker).

### II. Viral Production

### General Viral Production Process

Cells for the production of AAV, e.g., rAAV, particles may comprise, in some embodiments, mammalian cells (such as HEK293 cells) and/or insect cells (such as Sf9 cells).

In various embodiments, AAV production includes processes and methods for producing AAV particles and vectors which can contact a target cell to deliver a payload, e.g. a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, disclosed herein is a vector comprising a viral genome of the present disclosure. In some embodiments, disclosed herein is a cell comprising a viral genome of the present disclosure. In some embodiments, the cell is a bacterial cell, a mammalian cell (e.g., a HEK293 cell), or an insect cell (e.g., an Sf9 cell).

In some embodiments, disclosed herein is a method of making a viral genome. The method comprising providing a nucleic acid encoding a viral genome described herein and a backbone region suitable for replication of the viral genome in a cell, e.g., a bacterial cell (e.g., wherein the backbone region comprises one or both of a bacterial origin of replication and a selectable marker), and excising the viral genome from the backbone region, e.g., by cleaving the nucleic acid molecule at upstream and downstream of the viral genome. In some embodiments, the viral genome comprising a promoter operably linked to nucleic acid comprising a transgene encoding an NPC protein (e.g., an NPC1 protein described herein), will be incorporated into an AAV particle produced in the cell. In some embodiments, the cell is a bacterial cell, a mammalian cell (e.g., a HEK293 cell), or an insect cell (e.g., an Sf9 cell).

In some embodiments, disclosed herein is a method of making a recombinant AAV particle of the present disclosure, the method comprising (i) providing a host cell comprising a viral genome described herein and incubating the host cell under conditions suitable to enclose the viral genome in a capsid protein, e.g., a capsid protein described herein (e.g., a capsid protein listed in Table 1, e.g., an AAV9 capsid protein or functional variant thereof), thereby making the recombinant AAV particle. In some embodiments, the method comprises prior to step (i), introducing a first nucleic acid comprising the viral genome into a cell. In some embodiments, the host cell comprises a second nucleic acid encoding the capsid protein. In some embodiments, the second nucleic acid is introduced into the host cell prior to, concurrently with, or after the first nucleic acid molecule. In some embodiments, the host cell is a bacterial cell, a mammalian cell (e.g., a HEK293 cell), or an insect cell (e.g., an Sf9 cell).

In some embodiments, methods are provided herein of producing AAV particles or vectors by (a) contacting a viral production cell with one or more viral expression constructs encoding at least one AAV capsid protein, and one or more payload constructs encoding a payload molecule, which can be selected from: a transgene, a polynucleotide encoding protein, and a modulatory nucleic acid; (b) culturing the viral production cell under conditions such that at least one AAV particle or vector is produced, and (c) isolating the AAV particle or vector from the production stream.

In these methods, a viral expression construct may encode at least one structural protein and/or at least one non-structural protein. The structural protein may include any of the native or wild type capsid proteins VP1, VP2, and/or VP3, or a chimeric protein thereof. The non-structural protein may include any of the native or wild type Rep78, Rep68, Rep52, and/or Rep40 proteins or a chimeric protein thereof.

In some embodiments, contacting occurs via transient transfection, viral transduction, and/or electroporation.

In some embodiments, the viral production cell is selected from a mammalian cell and an insect cell. In certain embodiments, the insect cell includes a Spodoptera frugiperda insect cell. In certain embodiments, the insect cell includes a Sf9 insect cell. In certain embodiments, the insect cell includes a Sf21 insect cell.

The payload construct vector of the present disclosure may include, in some embodiments, at least one inverted terminal repeat (ITR) and may include mammalian DNA.

Also provided are AAV particles and viral vectors produced according to the methods described herein.

In some embodiments, the AAV particles of the present disclosure may be formulated as a pharmaceutical composition with one or more acceptable excipients.

In certain embodiments, an AAV particle or viral vector may be produced by a method described herein.

In some embodiments, the AAV particles may be produced by contacting a viral production cell (e.g., an insect cell or a mammalian cell) with at least one viral expression construct encoding at least one capsid protein and at least one payload construct vector. The viral production cell may be contacted by transient transfection, viral transduction, and/or electroporation. The payload construct vector may include a payload construct encoding a payload molecule such as, but not limited to, a transgene, a polynucleotide encoding protein, and a modulatory nucleic acid. The viral production cell can be cultured under conditions such that at least one AAV particle or vector is produced, isolated (e.g., using temperature-induced lysis, mechanical lysis and/or chemical lysis) and/or purified (e.g., using filtration, chromatography, and/or immunoaffinity purification). As a non-limiting example, the payload construct vector may include mammalian DNA.

In some embodiments, the AAV particles are produced in an insect cell (e.g., Spodoptera frugiperda (Sf9) cell) using a method described herein. As a non-limiting example, the insect cell is contacted using viral transduction which may include baculoviral transduction.

In some embodiments, the AAV particles are produced in an mammalian cell (e.g., HEK293 cell) using a method described herein. As a non-limiting example, the mammalian cell is contacted using viral transduction which may include multiplasmid transient transfection (such as triple plasmid transient transfection).

In some embodiments, the AAV particle production method described herein produces greater than 10¹, greater than 10², greater than 10³, greater than 10⁴, or greater than 10⁵ AAV particles in a viral production cell.

In some embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

In various embodiments, once administered, an AAV particle disclosed herein may, without being bound by theory, contact a target cell and enter the cell, e.g., in an endosome. The AAV particles, e.g., those released from the endosome, may subsequently contact the nucleus of the target cell to deliver the payload construct. The payload construct, e.g. recombinant viral construct, may be delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

In some embodiments, large scale production of AAV particles utilizes a bioreactor. Without being bound by theory, the use of a bioreactor may allow for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In some embodiments, the bioreactor is used for continuous production in which a portion of the culture is harvested at an experimentally determined time point for purification of AAV particles, and the remaining culture in the bioreactor is refreshed with additional growth media components.

In various embodiments, AAV viral particles can be extracted from viral production cells in a process which includes cell lysis, clarification, sterilization and purification. Cell lysis includes any process that disrupts the structure of the viral production cell, thereby releasing AAV particles. In certain embodiments, cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

In various embodiments, the end result of viral production is a purified collection of AAV particles which include two components: (1) a payload construct (e.g. a recombinant AAV viral genome, e.g., vector genome, construct) and (2) a viral capsid.

In some embodiments, a viral production system or process of the present disclosure includes steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In some embodiments, a viral production system or process of the present disclosure includes steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

### Viral Expression Constructs

In various embodiments, the viral production system of the present disclosure includes one or more viral expression constructs that can be transfected/transduced into a viral production cell. In certain embodiments, a viral expression construct or a payload construct of the present disclosure can be a bacmid, also known as a baculovirus plasmid or recombinant baculovirus genome. In certain embodiments, the viral expression includes a protein-coding nucleotide sequence and at least one expression control sequence for expression in a viral production cell. In certain embodiments, the viral expression includes a protein-coding nucleotide sequence operably linked to least one expression control sequence for expression in a viral production cell. In certain embodiments, the viral expression construct contains parvoviral genes under control of one or more promoters. Parvoviral genes can include nucleotide sequences encoding non-structural AAV replication proteins, such as Rep genes which encode Rep52, Rep40, Rep68, or Rep78 proteins. Parvoviral genes can include nucleotide sequences encoding structural AAV proteins, such as Cap genes which encode VP1, VP2, and VP3 proteins.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of each of which are herein incorporated by reference in their entirety as related to viral expression constructs and uses thereof.

In some embodiments, the viral expression construct is an AAV expression construct which includes one or more nucleotide sequences encoding non-structural AAV replication proteins, structural AAV capsid proteins, or a combination thereof.

In some embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In some embodiments, the viral expression construct may contain a nucleotide sequence which includes start codon region, such as a sequence encoding AAV capsid proteins which include one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG).

In some embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in their entirety as related to AAV capsid proteins and the production thereof.

In some embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG, and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may promote high vector yields.

In some embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In some embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In some embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art.

In some embodiments, the polynucleotide incorporated into the bacmid (i.e. polynucleotide insert) can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g. VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g. Rep78, Rep52, or a combination thereof).

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In some embodiments, AAV particles are produced in viral production cells (such as mammalian or insect cells) wherein all three VP proteins are expressed at a stoichiometry approaching, about or which is: 1:1:10 (VP1:VP2:VP3); 2:2:10 (VP1:VP2:VP3); 2:0:10 (VP1:VP2:VP3); 1-2:0-2:10 (VP1:VP2:VP3); 1-2:1-2:10 (VP1:VP2:VP3); 2-3:0-3:10 (VP1:VP2:VP3); 2-3:2-3:10 (VP1:VP2:VP3); 3:3:10 (VP1:VP2:VP3); 3-5:0-5:10 (VP1:VP2:VP3); or 3-5:3-5:10 (VP1:VP2:VP3).

In some embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In some embodiments of the present disclosure, Rep52 or Rep78 is transcribed from the baculoviral derived polyhedron promoter (polh). Rep52 or Rep78 can also be transcribed from a weaker promoter, for example a deletion mutant of the ie-1 promoter, the Aie-1 promoter, has about 20% of the transcriptional activity of that ie-1 promoter. A promoter substantially homologous to the Δie-1 promoter may be used. In respect to promoters, a homology of at least 50%, 60%, 70%, 80%, 90% or more, is considered to be a substantially homologous promoter.

### Mammalian Cells

Viral production of the present disclosure disclosed herein describes processes and methods for producing AAV particles or viral vector that contacts a target cell to deliver a payload construct, e.g. a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO, W138, HeLa, HEK293, HEK293T (293T), Saos, C2C12, L cells, HT1080, Huh7, HepG2, C127, 3T3, CHO, HeLa cells, KB cells, BHK and primary fibroblast, hepatocyte, and myoblast cells derived from mammals. Viral production cells can include cells derived from any mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of each of which are herein incorporated by reference in their entireties insofar as they do no conflict with the present disclosure. In certain embodiments, the AAV viral production cells are trans-complementing packaging cell lines that provide functions deleted from a replication-defective helper virus, e.g., HEK293 cells or other Ea trans-complementing cells.

In some embodiments, the packaging cell line 293-10-3 (ATCC Accession No. PTA-2361) may be used to produce the AAV particles, as described in U.S. Pat. No. 6,281,010, the contents of which are herein incorporated by reference in their entirety as related to the 293-10-3 packaging cell line and uses thereof.

In some embodiments, of the present disclosure a cell line, such as a HeLA cell line, for trans-complementing E1 deleted adenoviral vectors, which encoding adenovirus Ela and adenovirus E1b under the control of a phosphoglycerate kinase (PGK) promoter can be used for AAV particle production as described in U.S. Pat. No. 6,365,394, the contents of which are incorporated herein by reference in their entirety as related to the HeLa cell line and uses thereof.

In some embodiments, AAV particles are produced in mammalian cells using a multiplasmid transient transfection method (such as triple plasmid transient transfection). In certain embodiments, the multiplasmid transient transfection method includes transfection of the following three different constructs: (i) a payload construct, (ii) a Rep/Cap construct (parvoviral Rep and parvoviral Cap), and (iii) a helper construct. In certain embodiments, the triple transfection method of the three components of AAV particle production may be utilized to produce small lots of virus for assays including transduction efficiency, target tissue (tropism) evaluation, and stability. In certain embodiments, the triple transfection method of the three components of AAV particle production may be utilized to produce large lots of materials for clinical or commercial applications.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other Ela trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference in their entirety as related to the production and processing of recombinant AAV virus particles. Production may also involve methods known in the art including those using 293T cells, triple transfection or any suitable production method.

In some embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g. medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80C to 37C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated by reference in their entireties as related to the measurement of particle concentrations).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety as related to the growth and use of insect cells in viral production.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety as related to the use of insect cells in viral production.

In some embodiments, the AAV particles are made using the methods described in WO2015/191508, the contents of which are herein incorporated by reference in their entirety insofar as they do not conflict with the present disclosure.

In some embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety as related to the production of viral particles.

Expansion, culturing, transfection, infection and storage of insect cells can be carried out in any cell culture media, cell transfection media or storage media known in the art, including Hyclone™ SFX-Insect™ Cell Culture Media, Expression System ESF AF™ Insect Cell Culture Medium, ThermoFisher Sf-900II™ media, ThermoFisher Sf-900III™ media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production Systems

In some embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In some embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can include the viral expression construct (Expression BEV), and one or more group which can include the payload construct (Payload BEV). The baculoviruses may be used to infect a viral production cell for production of AAV particles or viral vector.

In some embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In some embodiments, BEVs are produced using a Bacmid Transfection agent, such as Promega FuGENE® HD, WFI water, or ThermoFisher Cellfectin® II Reagent. In certain embodiments, BEVs are produced and expanded in viral production cells, such as an insect cell.

In some embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN2 vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection (M.O.I.), see Urabe, M. et al. J Virol. 2006 February; 80(4):1874-85, the contents of which are herein incorporated by reference in their entirety as related to the production and use of BEVs and viral particles.

Production of AAV particles with baculovirus in an insect cell system may address known baculovirus genetic and physical instability.

In some embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture. Wasilko D J et al. Protein Expr Purif. 2009 June; 65(2):122-32, the contents of which are herein incorporated by reference in their entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such embodiments, the corresponding bacmid vector is engineered with replication control elements, including but not limited to promoters, enhancers, and/or cell-cycle regulated replication elements.

In some embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, the AAV particle of the present disclosure may be produced in insect cells (e.g., Sf9 cells).

In some embodiments, the AAV particle of the present disclosure may be produced using triple transfection.

In some embodiments, the AAV particle of the present disclosure may be produced in mammalian cells.

In some embodiments, the AAV particle of the present disclosure may be produced by triple transfection in mammalian cells.

In some embodiments, the AAV particle of the present disclosure may be produced by triple transfection in HEK293 cells.

The AAV viral genomes encoding NPC protein described herein may be useful in the fields of human disease, veterinary applications and a variety of in vivo and in vitro settings. The AAV particles of the present disclosure may be useful in the field of medicine for the treatment, prophylaxis, palliation, or amelioration of neurological or neuromuscular diseases and/or disorders. In some embodiments, the AAV particles of the disclosure are used for the prevention and/or treatment of NPC1.

Various embodiments of the disclosure herein provide a pharmaceutical composition comprising the AAV particle described herein and a pharmaceutically acceptable excipient.

Various embodiments of the disclosure herein provide a method of treating a subject in need thereof comprising administering to the subject a therapeutically effective amount of the pharmaceutical composition described herein.

Some embodiments of the method provide that the subject is treated by a route of administration of the pharmaceutical composition selected from the group consisting of: intravenous, intracerebroventricular, intraparenchymal, intrathecal, subpial, and intramuscular, or a combination thereof. Certain embodiments of the method provide that the subject is treated for NPC1 and/or other neurological disorder arising from a deficiency in the quantity or function of NPC1 and/or NPC2 proteins. In one aspect of the method, a pathological feature of the NPC1 or the other neurological disorder is alleviated and/or the progression of the NPC1 or the other neurological disorder is halted, slowed, ameliorated, or reversed.

Various embodiments of the disclosure herein describe a method of increasing the level of NPC protein in the central nervous system of a subject in need thereof comprising administering to said subject via infusion, an effective amount of the pharmaceutical composition described herein.

Also described herein are compositions, methods, processes, kits and devices for the design, preparation, manufacture and/or formulation of AAV particles. In some embodiments, payloads, such as but not limited to payloads comprising NPC protein, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of NPC1. Such methods may involve gene replacement or gene activation. Such outcomes are achieved by utilizing the methods and compositions taught herein.

### III. Pharmaceutical Compositions

The present disclosure additionally provides a method for treating NPC1 and disorders related to deficiencies in the function or expression of the NPC1 and/or NPC2 protein(s) in a mammalian subject, including a human subject, comprising administering to the subject any of the AAV polynucleotides or AAV genomes described herein (e.g., “vector genomes,” “viral genomes,” or “VGs”) or administering to the subject a particle comprising said AAV polynucleotide or AAV genome, or administering to the subject any of the described compositions, including pharmaceutical compositions.

As used herein the term “composition” comprises an AAV polynucleotide or AAV genome or AAV particle and at least one excipient.

As used herein the term “pharmaceutical composition” comprises an AAV polynucleotide or AAV genome or AAV particle and one or more pharmaceutically acceptable excipients.

Although the descriptions of pharmaceutical compositions, e.g., AAV comprising a payload encoding an NPC protein to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects.

In some embodiments, the AAV particle formulations described herein may contain a nucleic acid encoding at least one payload. In some embodiments, the formulations may contain a nucleic acid encoding 1, 2, 3, 4, or 5 payloads. In some embodiments, the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the formulation contains at least three payload constructs encoding proteins. Certain embodiments provide that at least one of the payloads is NPC protein or a variant thereof.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### IV. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise between 0.1% and 99% (w/w) of the active ingredient. By way of example, the composition may comprise between 0.1% and 100%, e.g., between 0.5% and 50%, between 1-30%, between 5-80%, or at least 80% (w/w) active ingredient.

The AAV particles of the disclosure can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding NPC protein may be formulated to optimize baricity and/or osmolality. In some embodiments, the baricity and/or osmolality of the formulation may be optimized to ensure optimal drug distribution in the central nervous system or a region or component of the central nervous system.

In some embodiments, the AAV particles of the disclosure may be formulated in PBS with 0.001% of pluronic acid (F-68) at a pH of about 7.0.

In some embodiments, the AAV particles of the disclosure may be formulated in PBS, in combination with an ethylene oxide/propylene oxide copolymer (also known as pluronic or poloxamer).

In some embodiments, the AAV particles of the disclosure may be formulated in PBS with 0.001% pluronic acid (F-68) (poloxamer 188) at a pH of about 7.0.

In some embodiments, the AAV particles of the disclosure may be formulated in PBS with 0.001% pluronic acid (F-68) (poloxamer 188) at a pH of about 7.3.

In some embodiments, the AAV particles of the disclosure may be formulated in PBS with 0.001% pluronic acid (F-68) (poloxamer 188) at a pH of about 7.4.

In some embodiments, the AAV particles of the disclosure may be formulated in a solution comprising sodium chloride, sodium phosphate and an ethylene oxide/propylene oxide copolymer.

In some embodiments, the AAV particles of the disclosure may be formulated in a solution comprising sodium chloride, sodium phosphate dibasic, potassium chloride, potassium phosphate monobasic, and poloxamer 188/pluronic acid (F-68).

In some embodiments, the AAV particles of the disclosure may be formulated in a solution comprising 192 mM sodium chloride, 10 mM sodium phosphate (dibasic), 2.7 mM potassium chloride, 2 mM potassium phosphate (monobasic) and 0.001% pluronic F-68 (v/v), at pH 7.4. This formulation is referred to as Formulation 1 in the present disclosure.

In some embodiments, the AAV particles of the disclosure may be formulated in a solution comprising about 192 mM sodium chloride, about 10 mM sodium phosphate dibasic and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate dibasic in the final solution may be 1 mM-50 mM. As non-limiting examples, the concentration of sodium phosphate dibasic in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the disclosure may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (pluronic acid (F-68)) may be 0.0001%-1%. As non-limiting examples, the concentration of poloxamer 188 (pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

### Excipients

The formulations of the disclosure can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles disclosed herein may include cations or anions. In some embodiments, the formulations include metal cations such as, but not limited to, Zn²⁺, Ca²⁺, Cu²⁺, Mg⁺, or combinations thereof. In some embodiments, formulations may include polymers or polynucleotides complexed with a metal cation (see, e.g., U.S. Pat. Nos. 6,265,389 and 6,555,525, the contents of each of which are herein incorporated by reference in their entirety).

### V. Uses and Applications

The compositions of the disclosure (e.g., a viral genome or AAV particle comprising a viral genome described herein) may be administered to a subject or used in the manufacture of a medicament for the delivery of an exogenous NPC1 protein to a subject. The compositions of the disclosure (e.g., a viral genome or AAV particle described herein) may be administered to a subject or used in the manufacture of a medicament for administration to a subject having or diagnosed with having a disease associated with NPC, e.g., NPC1, expression (e.g., a lysosomal storage disease, e.g., Niemann-Pick disease, type C1), a deficiency in the quantity or function of NPC protein or having a disease or condition associated with decreased NPC protein expression. In some embodiments, the disease is NPC1. In some embodiments, the AAV particles including NPC protein may be administered to a subject to treat NPC1. In some embodiments, administration of the AAV particles comprising viral genomes that encode NPC protein may protect central nervous system pathways from degeneration. In some embodiments, the subject has, has been diagnosed with having, or is at risk of having a disease associated with expression of NPC1, e.g., aberrant or reduced NPC1 expression, e.g., expression of an NPC1 gene, NPC1 mRNA, and/or NPC1 protein. In some embodiments, the subject has, has been diagnosed with having, or is at risk of having a lysosomal storage disease or Niemann-Pick disease, type C1.

In some embodiments, the delivery of the AAV particles results in amelioration of at least one symptom of the disease associated with NPC1 expression, the lysosomal storage disease, and/or Niemann-Pick disease, type C1 in the subject.

In some embodiments, the delivery of the AAV particles may halt or slow progression of NPC1 as measured by cholesterol accumulation in CNS cells (as determined, for example, by filipin staining and quantification). In some embodiments, the delivery of the AAV particles may halt or slow accumulation of unesterified cholesterol in NPC1-patient brain and other tissues. Accumulation of unesterified cholesterol in NPC1 patients (as opposed to free cholesterol) contributes to NPC1 disease pathology. Non-enzymatic degradation of cholesterol results in accumulated unesterified cholesterol. Thus, in some embodiments, the delivery of the AAV particles may reduce the adverse effects contributed by accumulation of unesterified cholesterol in NPC1 patients.

In some embodiments, the delivery of the AAV particles improves symptoms of NPC1, including, for example, cognitive, muscular, physical, and sensory symptoms of NPC1.

In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

In some embodiments, the pharmaceutical compositions described herein are used as research tools, particularly in in vitro investigations using human cell lines such as HEK293T and in vivo testing in nonhuman primates which will occur prior to human clinical trials.

In some embodiments, the method further comprises performing a blood test, an imaging test, a CNS biopsy sample, or an aqueous cerebral spinal fluid biopsy and/or evaluating, e.g., measuring, the level of NPC1 expression, e.g., NPC1 gene, NPC1 mRNA, and/or NPC1 protein expression in a subject, e.g., in a cell or tissue of the subject, optionally wherein the level of NPC1 protein is measured by an assay described herein, e.g., an ELISA, a Western blot, or an immunohistochemistry assay. In some embodiments, measuring the level of NPC1 expression is performed prior to, during, or subsequent to treatment with the AAV particle comprising an AAV viral genome described herein. In some embodiments, the administration of the AAV viral particle comprising an AAV viral genome described herein results in increased level of NPC1 protein expression (e.g., 0.5×-20.0× more NPC protein expression, e.g., 2-fold, 3-fold, 4-fold, 5-fold, 6-fold, 7-fold, 8-fold, 9-fold, 10-fold, 15-fold, 20-fold more NPC1 protein expression) in a cell of the subject, relative to reference level, e.g., a subject that has not received treatment, e.g., has not been administered the AAV particle. In some embodiments, the administration of the AAV viral particle comprising an AAV viral genome described herein results reduction of cholesterol accumulation in CNS cells (e.g. as measured by filipin staining, HP-β-Calbindin D staining, and quantification), as compared to a reference level, e.g., a subject that has not received treatment, e.g., has not been administered the AAV particle.

### CNS Diseases

The present disclosure provides a method for treating a disease, disorder and/or condition in a mammalian subject, including a human subject, comprising administering to the subject any of the viral particles e.g., AAV, AAV particle, or AAV genome that produces NPC protein described herein (i.e., viral genomes or “VG”) or administering to the subject a particle comprising said AAV particle or AAV genome, or administering to the subject any of the described compositions, including pharmaceutical compositions.

In some embodiments, AAV particles of the present disclosure, through delivery of a functional payload that is a therapeutic product comprising an NPC protein or variant thereof that can modulate the level or function of a gene product in the CNS.

A functional payload may alleviate or reduce symptoms that result from abnormal level and/or function of a gene product (e.g., an absence or defect in a protein) in a subject in need thereof or that otherwise confers a benefit to a CNS disorder in a subject in need thereof.

As non-limiting examples, companion or combination therapeutic products delivered by AAV particles of the present disclosure may include, but are not limited to, growth and trophic factors, cytokines, hormones, neurotransmitters, enzymes, anti-apoptotic factors, angiogenic factors, NPC proteins, and any protein known to be mutated in pathological disorders such as NPC1.

In some embodiments, AAV particles of the present disclosure may be used to treat diseases that are associated with impairments of the growth and development of the CNS, i.e., neurodevelopmental disorders. In some aspects, such neurodevelopmental disorders may be caused by genetic mutations.

In some embodiments, the neurological disorders may be functional neurological disorders with motor and/or sensory symptoms which have neurological origin in the CNS. As non-limiting examples, functional neurological disorders may be chronic pain, seizures, speech problems, involuntary movements, or sleep disturbances.

In some embodiments, the neurological or neuromuscular disease, disorder, and/or condition is NPC1. In some embodiments, the delivery of the AAV particles may halt or slow the disease progression of NPC1 by 10%, 20%, 30%, 40%, 50%, 60%, 70%, 80%, 90%, 95%, or more than 95% using a known analysis method and comparator group for NPC1. As a non-limiting example, the delivery of the AAV particles may halt or slow progression of NPC1 as measured by cholesterol accumulation in CNS cells (as determined, for example, by filipin staining and quantification).

In some embodiments, the AAV particle encoding a payload may increase the amount of NPC protein in a tissue by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or more than 100%. In some embodiments, the AAV particle encoding a payload may increase the amount of NPC protein in a tissue to be comparable to (e.g., approximately the same as) the amount of NPC protein in the corresponding tissue of a healthy subject. In some embodiments, the AAV particle encoding a payload may increase the amount of NPC protein in a tissue effective to reduce one or more symptoms of a disease associated with decreased NPC protein expression or a deficiency in the quantity and/or function of NPC protein, e.g., NPC1.

Neonates with Genetically Confirmed NPC1

In some embodiments, the delivery of the AAV particles as described herein is useful as a vector-based (e.g., AAV9) gene therapy for the treatment of neonates with genetically confirmed NPC1. Early neonatal onset presents with clinical symptoms of NPC1 within 3 months to 2 years of age. Delivery of the AAV particles as described herein may halt or slow the progressive neurological and clinical decline of neonatal onset NPC1. Primary efficacy endpoints of treatment using the AAV particles described herein include improved results in percentage of Motor Milestones Responders on Hammersmith Infant Neurological Examination (HINE) assessment and improved rate of slowing or normalization of age appropriate development milestones. Secondary efficacy endpoints of treatment using the AAV particles described herein include reduced Time to Death or Permanent Ventilation, improved Child Brain Function including Resting Brain Function (Alpha, Gamma and Theta power, Decreased Seizure count), and improved Body Mass Index, Maternal Physiological Stress, and Maternal Hair Cortisol readouts. Additional efficacy endpoints may include improved readouts of neurodegeneration biomarkers including biofluid (plasma/CSF) markers of neurodegeneration (e.g., NfL), improved imaging readouts including volumetric MRII of the cerebellum and whole brain. Pharmacodynamic assessments efficacy may include quantification of markers of cholesterol metabolism (including plasma oxysterols lyso-SM-509 and others), and CSF levels of HP—O-Calbindin D. In some embodiments, safety and tolerability of treatment using the AAV particles as described herein include acceptable incidence of adverse events (AEs) attributable to the treatment, inpatient observation after dosing, and continued monitoring for antibody- and cell-mediated immune response to the treatment. Contraindications may include presence of neutralizing antibodies at a titer greater than, for example, 1:50.

### Juvenile Onset NPC1

In some embodiments, the delivery of the AAV particles as described herein is useful as a vector-based (e.g., AAV9) gene therapy for the treatment of juvenile onset NPC1. Juvenile onset NPC1 presents with clinical symptoms of NPC1 at 6 to 15 years of age, with mean life expectation of 24 years. Delivery of the AAV particles as described herein may halt or slow the progressive neurological and clinical decline of juvenile onset NPC1. Primary efficacy endpoints of treatment using the AAV particles described herein include clinically meaningful improvements in mean rate of disease progression, e.g., statistically significant improvement as compared to untreated control populations or current standard of care treatment at 18 to 24 months post treatment. Secondary efficacy endpoints of treatment using the AAV particles described herein include improved neurologic and/or functional readouts such as Inventory of Non-Ataxia Signs (INAS), SCA Functional Index or SARA, as well as improvements in cognitive impairment (school performance, language and learning), improvement of gelastic cataplexy seizure severity and frequency, improved quality of life, and others, and improved readouts of full neuropsychiatric assessment. Additional efficacy endpoints may include improved readouts of neurodegeneration biomarkers including biofluid (plasma/CSF) markers of neurodegeneration (e.g., NfL), improved imaging readouts including volumetric MRII of the cerebellum and whole brain. Pharmacodynamic assessments efficacy may include quantification of markers of cholesterol metabolism (including plasma oxysterols lyso-SM-509 and others), and CSF levels of HP-β-Calbindin D. In some embodiments, safety and tolerability of treatment using the AAV particles as described herein include acceptable incidence of adverse events (AEs) attributable to the treatment, inpatient observation after dosing, and continued monitoring for antibody- and cell-mediated immune response to the treatment. Contraindications may include presence of neutralizing antibodies at a titer greater than, for example, 1:50.

### VI. Dosing and Administration

### Administration

In some aspects, the present disclosure provides administration and/or delivery methods for vectors and viral particles, e.g., AAV particles, encoding NPC protein or a variant thereof, for the prevention, treatment, or amelioration of diseases or disorders of the CNS. For example, administration of the AAV particles prevents, treats, or ameliorates NPC1. Thus, robust widespread NPC protein distribution throughout the CNS and periphery is desired for maximal efficacy. Particular target tissues for administration or delivery include CNS tissues, brain tissue, and, more specifically, caudate-putamen, thalamus, superior colliculus, cortex, and corpus collosum.

The AAV particles of the present disclosure may be administered by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), intracranial (into the skull), picutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intraparenchymal (into the substance of), intrathecal (into the spinal canal), intraperitoneal, (infusion or injection into the peritoneum), intravesicular infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, subpial, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, AAV particles of the present disclosure are administered so as to be delivered to a target cell or tissue. Delivery to a target cell results in NPC protein expression. A target cell may be any cell in which it is considered desirable to increase NPC protein expression levels. A target cell may be a CNS cell. Non-limiting examples of such cells and/or tissues include, dorsal root ganglia and dorsal columns, proprioceptive sensory neurons, Clark's column, gracile and cuneate nuclei, cerebellar dentate nucleus, corticospinal tracts and the cells comprising the same, Betz cells, and cells of the heart.

In some embodiments, compositions may be administered in a way that allows them to cross the blood-brain barrier, vascular barrier, or other epithelial barrier.

In some embodiments, delivery of NPC protein by adeno-associated virus (AAV) particles to cells of the central nervous system (e.g., parenchyma) comprises infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In some embodiments, the AAV particles may be delivered by systemic delivery. In some embodiments, the systemic delivery may be by intravascular administration. In some embodiments, the systemic delivery may be by intravenous (IV) administration.

In some embodiments, the AAV particles may be delivered by intravenous delivery.

In some embodiments, the AAV particle is administered to the subject via focused ultrasound (FUS), e.g., coupled with the intravenous administration of microbubbles (FUS-MB), or MRI-guided FUS coupled with intravenous administration, e.g., as described in Terstappen et al. (Nat Rev Drug Discovery, https://doi.org/10.1038/s41573-021-00139-y (2021)), Burgess et al. (Expert Rev Neurother. 15(5): 477-491 (2015)), and/or Hsu et al. (PLOS One 8(2): 1-8), the contents of which are incorporated herein by reference in its entirety.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by thalamic delivery.

In some embodiments, the AAV particles may be delivered by intracerebral delivery.

In some embodiments, the AAV particles may be delivered by intracardiac delivery.

In some embodiments, the AAV particles may be delivered by intracranial delivery.

In some embodiments, the AAV particles may be delivered by intra cisterna magna (ICM) delivery.

In some embodiments, the AAV particles may be delivered by direct (intraparenchymal) injection into an organ (e.g., CNS (brain or spinal cord)). In some embodiments, the intraparenchymal delivery may be to any region of the brain or CNS.

In some embodiments, the AAV particles may be delivered by intrastriatal injection.

In some embodiments, the AAV particles may be delivered into the putamen.

In some embodiments, the AAV particles may be delivered into the spinal cord.

In some embodiments, the AAV particles of the present disclosure may be administered to the ventricles of the brain.

In some embodiments, the AAV particles of the present disclosure may be administered to the ventricles of the brain by intracerebroventricular delivery.

In some embodiments, the AAV particles of the present disclosure may be administered by intramuscular delivery.

In some embodiments, the AAV particles of the present disclosure are administered by more than one route described above. As a non-limiting example, the AAV particles may be administered by intravenous delivery and thalamic delivery.

In some embodiments, the AAV particles of the present disclosure are administered by more than one route described above. As a non-limiting example, the AAV particles may be administered by intravenous delivery and intracerebral delivery.

In some embodiments, the AAV particles of the present disclosure are administered by more than one route described above. As a non-limiting example, the AAV particles may be administered by intravenous delivery and intracranial delivery.

In some embodiments, the AAV particles of the present disclosure are administered by more than one route described above. In some embodiments, the AAV particles of the present disclosure may be delivered by intrathecal and intracerebroventricular administration.

In some embodiments, the AAV particles may be delivered to a subject to improve and/or correct mitochondrial dysfunction.

In some embodiments, the AAV particles may be delivered to a subject to preserve neurons. The neurons may be primary and/or secondary sensory neurons. In some embodiments, AAV particles are delivered to dorsal root ganglia and/or neurons thereof.

In some embodiments, administration of the AAV particles may preserve and/or correct function in the sensory pathways.

In some embodiments, the AAV particles may be delivered via intravenous (IV), intracerebroventricular (ICV), intraparenchymal, and/or intrathecal (IT) infusion and the therapeutic agent may also be delivered to a subject via intramuscular (IM) limb infusion in order to deliver the therapeutic agent to the skeletal muscle. Delivery of AAVs by intravascular limb infusion is described by Gruntman and Flotte, Human Gene Therapy Clinical Development, 2015, 26(3), 159-164, the contents of which are herein incorporated by reference in their entirety.

In some embodiments, delivery of viral vector pharmaceutical compositions in accordance with the present disclosure to cells of the central nervous system (e.g., parenchyma) comprises a rate of delivery defined by VG/hour=mL/hour*VG/mL, wherein VG is viral genomes, VG/mL is composition concentration, and mL/hour is rate of infusion.

In some embodiments, delivery of AAV particle pharmaceutical compositions in accordance with the present disclosure to cells of the central nervous system (e.g., parenchyma) comprises infusion of up to 1 mL. In some embodiments, delivery of viral vector pharmaceutical compositions in accordance with the present disclosure to cells of the central nervous system (e.g., parenchyma) may comprise infusion of 0.0001, 0.0002, 0.001, 0.002, 0.003, 0.004, 0.005, 0.008, 0.010, 0.015, 0.020, 0.025, 0.030, 0.040, 0.050, 0.060, 0.070, 0.080, 0.090, 0.1, 0.2, 0.3, 0.4, 0.5, 0.6, 0.7, 0.8, or 0.9 mL.

In some embodiments, delivery of AAV particle pharmaceutical compositions in accordance with the present disclosure to cells of the central nervous system (e.g., parenchyma) comprises infusion of between about 1 mL to about 120 mL. In some embodiments, delivery of viral vector pharmaceutical compositions in accordance with the present disclosure to cells of the central nervous system (e.g., parenchyma) may comprise an infusion of 0.1, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In some embodiments delivery of AAV particles to cells of the central nervous system (e.g., parenchyma) comprises infusion of at least 3 mL. In some embodiments, delivery of AAV particles to cells of the central nervous system (e.g., parenchyma) consists of infusion of 3 mL. In some embodiments, delivery of AAV particles to cells of the central nervous system (e.g., parenchyma) comprises infusion of at least 10 mL. In some embodiments, delivery of AAV particles to cells of the central nervous system (e.g., parenchyma) consists of infusion of 10 mL.

In some embodiments, the volume of the AAV particle pharmaceutical composition delivered to the cells of the central nervous system (e.g., parenchyma) of a subject is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl.

In some embodiments, the volume of the AAV particle pharmaceutical composition delivered to a region in both hemispheres of a subject brain is 2 μl, 20 μl, 50 μl, 80 μl, 100 μl, 200 μl, 300 μl, 400 μl, 500 μl, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, or more than 2000 μl. In some embodiments, the volume delivered to a region in both hemispheres is 200 μl. As another non-limiting example, the volume delivered to a region in both hemispheres is 900 μl. As yet another non-limiting example, the volume delivered to a region in both hemispheres is 1800 μl.

In certain embodiments, AAV particle or viral vector pharmaceutical compositions in accordance with the present disclosure may be administered at about 10 to about 600 μl/site, about 50 to about 500 μl/site, about 100 to about 400 μl/site, about 120 to about 300 μl/site, about 140 to about 200 μl/site, or about 160 μl/site.

In some embodiments, the total volume delivered to a subject may be split between one or more administration sites e.g., 1, 2, 3, 4, 5, or more than 5 sites. In some embodiments, the total volume is split between administration to the left and right hemisphere.

### Delivery of AAV Particles

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particles of the present disclosure may be administered or delivered using the methods for the delivery of AAV virions described in European Patent Application No. EP1857552, the contents of which are herein incorporated by reference in their entirety.

In some embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the viral vector encoding NPC protein may be administered or delivered using the methods for delivering DNA molecules using AAV vectors described in U.S. Pat. No. 5,858,351, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the viral vector encoding NPC protein may be administered or delivered using the methods for delivering AAV virions described in U.S. Pat. No. 6,325,998, the contents of which are herein incorporated by reference in their entirety.

In some embodiments, the viral vector encoding NPC protein may be administered or delivered using the methods for delivering DNA to muscle cells described in U.S. Pat. No. 6,335,011, the contents of which are herein incorporated by reference in their entirety.

In some embodiments, the viral vector encoding NPC protein may be administered or delivered using the methods for delivering DNA to muscle cells and tissues described in U.S. Pat. No. 6,610,290, the contents of which are herein incorporated by reference in their entirety.

In some embodiments, the viral vector encoding NPC protein may be administered or delivered using the methods for delivering DNA to muscle cells described in U.S. Pat. No. 7,704,492, the contents of which are herein incorporated by reference in their entirety.

In some embodiments, the viral vector encoding NPC protein may be administered or delivered using the methods for delivering a payload to skeletal muscles described in U.S. Pat. No. 7,112,321, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload for the treatment of Alzheimer disease described in U.S. Pat. No. 8,318,687, the contents of which are herein incorporated by reference in their entirety.

In some embodiments, the AAV particle or pharmaceutical compositions of the present disclosure may be administered or delivered using the methods for delivering a payload described in International Patent Publication No. WO2012144446, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

In some embodiments, the AAV particle or pharmaceutical compositions of the present disclosure may be administered or delivered using the methods for delivering a payload described in International Patent Publication No. WO2001096587, the contents of which are herein incorporated by reference in their entirety.

In some embodiments, the AAV particle or pharmaceutical compositions of the present disclosure may be administered or delivered using the methods for delivering a payload to muscle tissue described in International Patent Publication No. WO2002014487, the contents of which are herein incorporated by reference in their entirety.

In some embodiments, a catheter may be used to administer the AAV particles. In certain embodiments, the catheter or cannula may be located at more than one site in the spine for multi-site delivery. The viral particles encoding may be delivered in a continuous and/or bolus infusion. Each site of delivery may be a different dosing regimen or the same dosing regimen may be used for each site of delivery. In some embodiments, the sites of delivery may be in the cervical and the lumbar region. In some embodiments, the sites of delivery may be in the cervical region. In some embodiments, the sites of delivery may be in the lumbar region.

In some embodiments, a subject may be analyzed for spinal anatomy and pathology prior to delivery of the AAV particles described herein. As a non-limiting example, a subject with scoliosis may have a different dosing regimen and/or catheter location compared to a subject without scoliosis.

In some embodiments, the delivery method and duration is chosen to provide broad transduction in the spinal cord. In some embodiments, intrathecal delivery is used to provide broad transduction along the rostral-caudal length of the spinal cord. In some embodiments, multi-site infusions provide a more uniform transduction along the rostral-caudal length of the spinal cord.

In some embodiments, the AAV particle or pharmaceutical compositions of the present disclosure may be administered or delivered to an individual in need thereof through intravenous injection of AAV9 viral particles encoding NPC1 or a functional equivalent thereof.

In some embodiments, the AAV particle or pharmaceutical compositions of the present disclosure may be administered or delivered to an individual (such as a neonatal patient or juvenile patient) in need thereof through intraparenchymal (IPa) delivery of AAV9 viral particles encoding NPC1 or a functional equivalent thereof.

In some embodiments, intraparenchymal delivery, also referred to as intracranial delivery, comprises injection of an AAV particle, e.g., an AAV particle comprising a viral genome described herein, into the brain parenchyma. Without wishing to be bound by theory, it is believed in some embodiments, that intraparenchymal delivery results in targeting of specific brain regions, such as the targeted cortex, cerebellum, corpus callosum, brain stem, or combination thereof.

### Delivery to Cells

In some aspects, the present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

In some aspects, the present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

In some embodiments, the AAV particles may be delivered to bypass anatomical blockages such as, but not limited to the blood brain barrier.

In some embodiments, the AAV particles may be formulated and delivered to a subject by a route which increases the speed of drug effect as compared to oral delivery.

In some embodiments, the AAV particles may be delivered by a method to provide uniform transduction of the spinal cord and dorsal root ganglion (DRG). In some embodiments, the AAV particles may be delivered using intrathecal infusion.

In some embodiments, a subject may be administered the AAV particles described herein using a bolus infusion. As used herein, a “bolus infusion” means a single and rapid infusion of a substance or composition.

In some embodiments, the AAV particles encoding NPC protein may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, the AAV particles may be delivered to a subject via a single route administration.

In some embodiments, the AAV particles may be delivered to a subject via a multi-site route of administration. For example, a subject may be administered the AAV particles at 2, 3, 4, 5, or more than 5 sites.

In some embodiments, a subject may be administered the AAV particles described herein using sustained delivery over a period of minutes, hours or days. The infusion rate may be changed depending on the subject, distribution, formulation or another delivery parameter known to those in the art.

In some embodiments, if continuous delivery (continuous infusion) of the AAV particles is used, the continuous infusion may be for 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more than 24 hours.

In some embodiments, the intracranial pressure may be evaluated prior to administration. The route, volume, AAV particle concentration, infusion duration and/or vector titer may be optimized based on the intracranial pressure of a subject.

In some embodiments, the AAV particles may be delivered by systemic delivery. In some embodiments, the systemic delivery may be by intravascular administration.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by direct (intraparenchymal) injection into the substance of an organ, e.g., one or more regions of the brain.

In some embodiments, the AAV particles may be delivered by subpial injection into the spinal cord. For example, subjects may be placed into a spinal immobilization apparatus. A dorsal laminectomy may be performed to expose the spinal cord. Guiding tubes and XYZ manipulators may be used to assist catheter placement. Subpial catheters may be placed into the subpial space by advancing the catheter from the guiding tube and AAV particles may be injected through the catheter (Miyanohara et al., Mol Ther Methods Clin Dev. 2016; 3: 16046). In some cases, the AAV particles may be injected into the cervical subpial space. In some cases, the AAV particles may be injected into the thoracic subpial space.

In some embodiments, the AAV particles may be delivered to a subject in order to increase the NPC protein protein levels in the caudate-putamen, thalamus, superior colliculus, cortex, and/or corpus callosum as compared to endogenous levels. The increase may be 0.1× to 5×, 0.5× to 5×, 1× to 5×, 2× to 5×, 3× to 5×, 4× to 5×, 0.1× to 4×, 0.5× to 4×, 1× to 4×, 2× to 4×, 3× to 4×, 0.1× to 3×, 0.5× to 3×, 1× to 3×, 2× to 3×, 0.1× to 2×, 0.5× to 2×, 0.1× to 1×, 0.5× to 1×, 0.1× to 0.5×, 1× to 2×, 0.1×, 0.2×, 0.3×, 0.4×, 0.5×, 0.6×, 0.7×, 0.8×, 0.9×, 1.0×, 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9×, 4.0×, 4.1×, 4.2×, 4.3×, 4.4×, 4.5×, 4.6×, 4.7×, 4.8×, 4.9× or more than 5× as compared to endogenous levels.

In some embodiments, the AAV particles may be delivered to a subject in order to increase the NPC protein protein levels in the caudate-putamen, thalamus, superior colliculus, cortex, and/or corpus callosum by transducing cells in these CNS regions. Transduction may also be referred to as the amount of cells that are positive for NPC protein. The transduction may be greater than or equal to 1%, 5%, 10%, 15%, 20%, 25%, 30%, 35%, 40%, 45%, 50%, 55%, 60%, 65%, 70%, 75%, 80%, 85%, 90%, 95%, or 99% of cells in these CNS regions.

In some embodiments, delivery of AAV particles comprising a viral genome encoding NPC protein described herein to neurons in the caudate-putamen, thalamus, superior colliculus, cortex, and/or corpus callosum will lead to an increased expression of NPC protein. The increased expression may lead to improved survival and function of various cell types in these CNS regions and subsequent improvement of NPC1 symptoms.

Specifically, in some embodiments, the increased expression of NPC protein may lead to improved gait, sensory capability, coordination of movement and strength, functional capacity, cognition, and/or quality of life.

### Dosing

In some aspects, the present disclosure provides methods comprising administering viral vectors and their payloads in accordance with the disclosure to a subject in need thereof. Viral vector pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition associated with decreased NPC protein expression or a deficiency in the quantity and/or function of NPC protein). In some embodiments, the disease, disorder, and/or condition is NPC1. The exact amount required will vary from subject to subject, depending on the species, age, and general condition of the subject, the severity of the disease, the particular composition, its mode of administration, its mode of activity, and the like. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific peptide(s) employed; the duration of the treatment; drugs used in combination or coincidental with the specific compound employed; and like factors well known in the medical arts.

In certain embodiments, AAV particle pharmaceutical compositions in accordance with the present disclosure may be administered at dosage levels sufficient to deliver NPC protein from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to VG or viral genomes per kg or into total viral genomes administered by one of skill in the art.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage form (e.g., a tablet, capsule, patch, loaded syringe, vial, etc.). As used herein, a “total daily dose” is an amount given or prescribed in 24-hour period. It may be administered as a single unit dose. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In some embodiments, delivery of the AAV particles described herein results in minimal serious adverse events (SAEs) as a result of the delivery of the AAV particles.

In some embodiments, delivery of AAV particle pharmaceutical compositions in accordance with the present disclosure to cells of the central nervous system (e.g., parenchyma) may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 1.8×10¹¹, 2×10¹¹, 3×10¹¹, 4×10¹¹, 5×10¹¹, 5.5×10¹¹, 6×10¹¹, 7×10¹¹, 8×10¹¹, 9×10¹¹, 0.8×10¹², 0.83×10¹², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.3×10¹³, 3×10¹³, 4×10¹³, 5×10¹³, 6×10¹³, 7×10¹³, 8×10¹³, 9×10¹³, 1×10¹⁴, 1.9×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In some embodiments, the concentration of the viral vector in the composition is 1×10¹³ VG/mL. In some embodiments, the concentration of the viral vector in the composition is 1.1×10¹² VG/mL. In some embodiments, the concentration of the viral vector in the composition is 3.7×10¹² VG/mL. In some embodiments, the concentration of the viral vector in the composition is 8×10¹¹ VG/mL. In some embodiments, the concentration of the viral vector in the composition is 2.6×10¹² VG/mL. In some embodiments, the concentration of the viral vector in the composition is 4.9×10¹² VG/mL. In some embodiments, the concentration of the viral vector in the composition is 0.8×10¹² VG/mL. In some embodiments, the concentration of the viral vector in the composition is 0.83×10¹² VG/mL. In some embodiments, the concentration of the viral vector in the composition is the maximum final dose which can be contained in a vial. In some embodiments, the concentration of the viral vector in the composition is 1.6×10¹¹ VG/mL. In some embodiments, the concentration of the viral vector in the composition is 5×10¹¹ VG/mL. In some embodiments, the concentration of the viral vector in the composition is 2.3×10¹³ VG/mL. In some embodiments, the concentration of the viral vector in the composition is 1.9×10¹⁴ VG/mL.

In some embodiments, delivery of AAV particle pharmaceutical compositions in accordance with the present disclosure to cells of the central nervous system (e.g., parenchyma) may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.6×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.3×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject. In some embodiments, the concentration of the viral vector in the composition is 2.3×10¹¹ VG/subject. In some embodiments, the concentration of the viral vector in the composition is 7.2×10¹¹ VG/subject. In some embodiments, the concentration of the viral vector in the composition is 7.5×10¹¹ VG/subject. In some embodiments, the concentration of the viral vector in the composition is 1.4×10¹² VG/subject. In some embodiments, the concentration of the viral vector in the composition is 4.8×10¹² VG/subject. In some embodiments, the concentration of the viral vector in the composition is 8.8×10¹² VG/subject. In some embodiments, the concentration of the viral vector in the composition is 2.3×10¹² VG/subject. In some embodiments, the concentration of the viral vector in the composition is 2×10¹⁰ VG/subject. In some embodiments, the concentration of the viral vector in the composition is 1.6×10¹¹ VG/subject. In some embodiments, the concentration of the viral vector in the composition is 4.6×10¹¹ VG/subject.

In some embodiments, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. In some embodiments, the total dose is 1×10¹³ VG. In some embodiments, the total dose is 3×10¹³ VG. In some embodiments, the total dose is 3.73×10¹⁰ VG. In some embodiments, the total dose is 1.9×10¹⁰ VG. In some embodiments, the total dose is 2.5×10¹¹ VG. In some embodiments, the total dose is 5×10¹¹ VG. In some embodiments, the total dose is 1×10¹² VG. In some embodiments, the total dose is 5×10¹² VG.

### Combinations

The AAV particles may be used in combination with one or more other therapeutic, prophylactic, diagnostic, or imaging agents. The phrase “in combination with,” is not intended to require that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

In some embodiments, the AAV particles of the present disclosure are administered to subjects on miglustat treatment for NPC1. The AAV particles and methods described herein can be used before, during, or after miglustat treatment. The AAV particles and methods can be administered alongside miglustat treatment. In some embodiments, the combined treatments of miglustat and AAV particle administration as described herein will provide additive or synergistic benefit in the amelioration of NPC1 disease symptoms.

In some embodiments, the AAV particles, compositions, and methods of the present disclosure can be administered to subjects in combination with other NPC1 treatments. NPC1 treatments which can be combined with the AAV particles, compositions, and methods of the present disclosure include cyclodextrin-based treatments for NPC1. Cyclodextrin-based treatments for NPC1 include, for example, those described in Yu, Daozhan, et al. “Niemann-Pick disease type C: induced pluripotent stem cell-derived neuronal cells for modeling neural disease and evaluating drug efficacy.” Journal of biomolecular screening 19.8 (2014): 1164-1173, the disclosure of which is incorporated by reference in its entirety. Other cyclodextrin-based NPC1 treatments include: TRAPPSOL CYCLO (CTD Holdings) (see ClinicalTrials.gov Identifiers NCT02939547, NCT02912793 and NCT03893071) and VTS-270 (Mallinckrodt Pharmaceuticals) (a 2-hydroxypropyl-β-cyclodextrin (HPβCD) mixture) (see ClinicalTrials.gov Identifier NCT03887533). Other treatments for NPC1 that can be combined with the compositions and methods of the present disclosure include administration of arimoclomol, e.g., arimoclomol citrate (Orphazyme A/S) (see ClinicalTrials.gov Identifier NCT02612129). The AAV particles and methods described herein can be administered before, during, or after other treatment for NPC1. The AAV particles and methods can be administered alongside other treatment for NPC1. In some embodiments, the combined treatments as described herein will provide additive or synergistic benefit in the amelioration of NPC1 disease symptoms.

### Measurement of Expression

Expression of NPC protein from viral genomes may be determined using various methods known in the art such as, but not limited to immunochemistry (e.g., IHC), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, PCR, and/or in situ hybridization (ISH). In some embodiments, transgenes encoding NPC protein delivered in different AAV capsids may have different expression levels in different CNS tissues.

In certain embodiments, the NPC protein is detectable by Western blot.

### VII. Kits and Devices

### Kits

In some aspects, the present disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or NPC protein of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present disclosure may be combined with, coated onto or embedded in a device. Devices may include, but are not limited to, dental implants, stents, bone replacements, artificial joints, valves, pacemakers and/or other implantable therapeutic device.

The present disclosure provides for devices which may incorporate viral vectors that encode one or more NPC protein molecules. These devices contain in a stable formulation the viral vectors which may be immediately delivered to a subject in need thereof, such as a human patient.

Devices for administration may be employed to deliver the viral vectors encoding NPC protein of the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure.

### VIII. Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV capsid polypeptide, e.g., a parent capsid sequence with at least one peptide, e.g., targeting peptide, insert. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprises a peptide, e.g., targeting peptide, present, e.g., inserted into, the capsid to enhance tropism for a desired target tissue. It is to be understood that reference to the AAV particle of the disclosure also includes pharmaceutical compositions thereof, even if not explicitly recited.

Active Ingredient: As used herein, the term “active ingredient” refers to a molecule or complex thereof that is biologically active and responsible for a generating a biological effect. The active ingredient in a pharmaceutical composition may be referred to as an active pharmaceutical ingredient. For the purposes of the present disclosure, the phrase “active ingredient” generally refers either to the viral particle carrying the payload or to the payload (or its gene product) delivered by the viral particle as described herein. In contrast, an “inactive ingredient” refers to a substance which is biologically inert. An excipient is an example of an inactive ingredient.

Activity: As used herein, the term “activity” refers to the condition in which things are happening or being done. Compositions of the disclosure may have activity and this activity may involve one or more biological events.

Administered in combination: As used herein, the term “administered in combination” or “delivered in combination” or “combined administration” refers to exposure of two or more agents (e.g., AAV) administered at the same time or within an interval such that the subject is at some point in time exposed to both agents and/or such that there is an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatuses in administration. In some embodiments, the administration of individual doses of one or more compounds and/or compositions of the present disclosure, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of a neurodegenerative disorder, amelioration includes the reduction or stabilization of neuron loss.

Animal: As used herein, the term “animal” refers to any member of the animal kingdom. In some embodiments, the terms subject or animal refers to humans at any stage of development. In some embodiments, animal refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.25%, 0.1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any substance (e.g., an AAV) that has activity in or on a biological system and/or organism. For instance, a substance that, when administered to an organism, has a biological effect on that organism, is considered to be biologically active. In particular embodiments, a compound, and/or a composition of the present disclosure may be considered biologically active if even a portion of it is biologically active or mimics an activity considered to be biologically relevant. In some embodiments, biological activity refers to inducing expression of NPC protein or a variant thereof. In some embodiments, biological activity refers to preventing and/or treating a disease associated with decreased NPC protein expression or a deficiency in the quantity and/or function of NPC protein. In some embodiments, biological activity refers to preventing and/or treating NPC1.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Central Nervous System or CNS: As used herein, “central nervous system” or “CNS” refers to one of the two major subdivisions of the nervous system, which in vertebrates includes the brain and spinal cord. The central nervous system coordinates the activity of the entire nervous system.

Cervical Region: As used herein, “cervical region” refers to the region of the spinal cord comprising the cervical vertebrae C1, C2, C3, C4, C5, C6, C7, and C8.

Cis-Elements: As used herein, cis-elements or the synonymous term “cis-regulatory elements” refer to regions of non-coding DNA which regulate the transcription of nearby genes. The Latin prefix “cis” translates to “on this side.” Cis-elements are found in the vicinity of the gene, or genes, they regulate. Examples of cis-elements include a Kozak sequence, SV40 introns, or a portion of the backbone.

CNS tissue: As used herein, “CNS tissue” or “CNS tissues” refers to the tissues of the central nervous system, which in vertebrates, include the brain and spinal cord and sub-structures thereof.

CNS structures: As used herein, “CNS structures” refers to structures of the central nervous system and sub-structures thereof. Non-limiting examples of structures in the spinal cord may include, ventral horn, dorsal horn, white matter, and nervous system pathways or nuclei within. Non-limiting examples of structures in the brain include, forebrain, midbrain, hindbrain, diencephalon, telencephalon, myelencephalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, amygdala, hippocampus, basal ganglia, corpus callosum, pituitary gland, putamen, striatum, ventricles and sub-structures thereof.

CNS Cells: As used herein, “CNS cells” refers to cells of the central nervous system and sub-structures thereof. Non-limiting examples of CNS cells include, neurons and sub-types thereof, glia, microglia, oligodendrocytes, ependymal cells and astrocytes. Non-limiting examples of neurons include sensory neurons, motor neurons, interneurons, unipolar cells, bipolar cells, multipolar cells, pseudounipolar cells, pyramidal cells, basket cells, stellate cells, Purkinje cells, Betz cells, amacrine cells, granule cell, ovoid cell, medium aspiny neurons and large aspiny neurons.

Codon optimization: As used herein, the term “codon optimization” refers to a process of changing codons of a given gene in such a manner that the polypeptide sequence encoded by the gene remains the same while the changed codons improve the process of expression of the polypeptide sequence. For example, if the polypeptide is of a human protein sequence and expressed in E. coli, expression will often be improved if codon optimization is performed on the DNA sequence to change the human codons to codons that are more effective for expression in E. coli.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In some embodiments, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a parvovirus e.g., AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present disclosure, e.g., viral particles or AAV vectors) to targeted cells.

Delivery route: As used herein, the term “delivery route” and the synonymous term “administration route” refers to any of the different methods for providing a therapeutic agent to a subject. Routes of administration are generally classified by the location at which the substance is applied and may also be classified based on where the target of action is. Examples include, but are not limited to: intravenous administration, subcutaneous administration, oral administration, parenteral administration, enteral administration, topical administration, sublingual administration, inhalation administration, and injection administration, or other routes of administration described herein.

Derivative: As used herein, the term “derivative” refers to a composition (e.g., sequence, compound, formulation, etc.) that is derived from, or finds its basis in, a parent composition. Non-limiting examples of a parent composition include a wild-type or original amino acid or nucleic acid sequence, or an undiluted formulation. In some embodiments, a derivative is a variant of a parent composition. A derivative may differ from the parent composition by less than about 1%, less than about 5%, less than about 10%, less than about 15%, less than about 20%, less than about 25%, less than about 30%, less than about 35%, less than about 40%, less than about 45%, or less than about 50%. In certain embodiments, a derivative may differ from a parent composition by more than about 50%. In certain embodiments, a derivative may differ from a parent composition by more than about 75%. In some embodiments, a derivative may be a fragment or truncation of a parent amino acid or nucleotide sequence. As a non-limiting example, a derivative may be a sequence with a nucleotide or peptide insert as compared to a parent nucleic acid or amino acid sequence (e.g., AAVPHP.B as compared to AAV9).

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats NPC1, an effective amount of an agent is, for example, an amount sufficient to achieve treatment, as defined herein, of NPC1 as compared to the response obtained without administration of the agent.

Engineered: As used herein, embodiments of the disclosure are “engineered” when they are designed to have a feature or property, whether structural or chemical, that varies from a starting point, wild-type or native molecule. Thus, engineered agents or entities are those whose design and/or production include an act of the hand of man.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and/or (5) post-translational modification of a polypeptide or protein.

Excipient: As used herein, the term “excipient” refers to an inactive substance that serves as the vehicle or medium for an active pharmaceutical agent or other active substance.

Formulation: As used herein, a “formulation” includes at least a compound and/or composition of the present disclosure (e.g., a vector, AAV particle, etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. A fragment may also refer to a truncation (e.g., an N-terminal and/or C-terminal truncation) of a protein or a truncation (e.g., at the 5′ and/or 3′ end) of a nucleic acid. A protein fragment may be obtained by expression of a truncated nucleic acid, such that the nucleic acid encodes a portion of the full-length protein.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides, and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% identical for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50, or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Humanized: As used herein, the term “humanized” refers to a non-human sequence of a polynucleotide or a polypeptide which has been altered to increase its similarity to its corresponding human sequence.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molecular Biol., 215, 403 (1990)).

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Lumbar Region: As used herein, the term “lumbar region” refers to the region of the spinal cord comprising the lumbar vertebrae L1, L2, L3, L4, and L5.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides. One or more mutations may result in a “mutant,” “derivative,” or “variant,” e.g., of a nucleic acid sequence or polypeptide or protein sequence.

Naturally occurring: As used herein, “naturally occurring” or “wild-type” means existing in nature without artificial aid, or involvement of the hand of man. “Naturally occurring” or “wild-type” may refer to a native form of a biomolecule, sequence, or entity.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

NPC protein: As used herein, the terms “NPC protein,” “NPC proteins,” “NPC protein,” “NPC proteins” and the like refer to one or both of proteins NPC1 (NPC intracellular cholesterol transporter 1, Ensemble gene ID: ENSG00000141458) and/or NPC2 (ENSG00000119655), homologs or variants thereof, and orthologs thereof, including non-human proteins and homologs thereof. NPC proteins include fragments, derivatives, and modifications of NPC1 and/or NPC2 proteins.

Nucleic acid: As used herein, the terms “nucleic acid,” “polynucleotide,” and “oligonucleotide” refer to any nucleic acid polymers composed of either polydeoxyribonucleotides (containing 2-deoxy-D-ribose), or polyribonucleotides (containing D-ribose), or any other type of polynucleotide that is an N glycoside of a purine or pyrimidine base, or modified purine or pyrimidine bases. There is no intended distinction in length between the term “nucleic acid,” “polynucleotide,” and “oligonucleotide,” and these terms will be used interchangeably. These terms refer only to the primary structure of the molecule. Thus, these terms include double- and single-stranded DNA, as well as double- and single-stranded RNA.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise a component for viral expression in a viral replication cell.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and/or xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of each of which are incorporated herein by reference in their entirety.

Pharmaceutical Composition: As used herein, the term “pharmaceutical composition” or pharmaceutically acceptable composition” comprises AAV polynucleotides, AAV genomes, or AAV particle and one or more pharmaceutically acceptable excipients, solvents, adjuvants, and/or the like.

Polypeptide: As used herein, the term “polypeptide” refers to an organic polymer consisting of a large number of amino-acid residues bonded together in a chain. A monomeric protein molecule is a polypeptide.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Promoter: As used herein, the term “promoter” refers to a nucleic acid site to which a polymerase enzyme will bind to initiate transcription (DNA to RNA) or reverse transcription (RNA to DNA).

Protein of interest: As used herein, the terms “proteins of interest” or “desired proteins” include those provided herein and fragments, mutants, variants, or alterations thereof.

Purified: As used herein, the term “purify” means to make substantially pure or clear from one or more unwanted components, material defilement, admixture or imperfection. “Purified” refers to the state of being pure. “Purification” refers to the process of making pure. As used herein, a substance is “pure” if it is substantially free of (substantially isolated from) one or more components, e.g., one or more components found in a native context.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may comprise the N- and/or C-termini as well as surrounding amino acids. In some embodiments, N- and/or C-terminal regions comprise from about 3 amino acids to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecules. In some embodiments, a “primary” sample is an aliquot of the source. In some embodiments, a primary sample is subjected to one or more processing (e.g., separation, purification, etc.) steps to prepare a sample for analysis or other use.

Serotype: As used herein, the term “serotype” refers to distinct variations in a capsid of an AAV based on surface antigens which allow epidemiologic classifications of the AAVs at the sub-species level.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Stable: As used herein “stable” refers to a compound or entity that is sufficiently robust to survive isolation to a useful degree of purity from a reaction mixture, and capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. Typical subjects include animals (e.g., mammals such as mice, rats, rabbits, non-human primates, and humans). In certain embodiments, a subject or patient may be susceptible to or suspected of having NPC1. In certain embodiments, a subject or patient may be diagnosed with NPC1.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “target cells” or “targeted cells” refers to any one or more cells of interest. The cells may be found in vitro, in vivo, in situ or in the tissue or organ of an organism. The organism may be an animal, a mammal, a human and/or a patient. The target cells may be CNS cells or cells in CNS tissue.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Thoracic Region: As used herein, a “thoracic region” refers to a region of the spinal cord comprising the thoracic vertebrae T1, T2, T3, T4, T5, T6, T7, T8, T9, T10, T11, and T12.

Treating: As used herein, the term “treating” refers to partially or completely alleviating, ameliorating, improving, relieving, reversing, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Variant: The term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: The term “functional variant” refers to a polypeptide variant or a polynucleotide variant that has at least one activity of the reference sequence.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence(s). Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, having a sequence that may be wild-type or modified from wild-type and which sequence may encode full-length or partial sequence of a protein, protein domain, or one or more subunits of NPC protein and variants thereof; a polynucleotide encoding NPC protein and variants thereof, having a sequence that may be wild-type or modified from wild-type; and a transgene encoding NPC protein and variants thereof that may or may not be modified from wild-type sequence.

Viral construct vector: As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” or “vector genome” is a polynucleotide comprising at least one inverted terminal repeat (ITR) and at least one encoded payload. A viral genome encodes at least one copy of the payload.

Wild-type: As used herein, “wild-type” is a native form of a biomolecule, sequence, or entity.

### IX. Equivalents and Scope

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

The present disclosure is further illustrated by the following non-limiting examples. Experiments described in the Examples establish that AAV-based NPC1 gene therapy treatments are superior to and/or additive with miglustat treatment in ameliorating NPC1 and related disorder progression in vivo. Overall, a research strategy was devised according to three stages: Stage 1: screening and identifying NPC protein candidates for overexpression and establishing animal models, including conducting Natural History Studies to establish baseline NPC1 disease-relevant phenotypes; Stage 2: identifying and optimizing NPC protein expression construct and initiating in vivo expression and efficacy studies; and Stage 3: selecting an optimized NPC protein expression construct and conducting exploratory route-of-administration and toxicity studies in large animal species (such as non-human primate).

### Cell Lines, Tissues, and Animal Models

Cell-lines and reagents: Standard cell-lines are used for initial screening. This includes lines from multiple species to align with in vivo studies. Since transduction of both neuronal and non-neuronal cell types are needed in vivo, two neuronal and two non-neuronal cell types are used for in vitro screening. These cell lines include naïve neuronal SK-N-AS cells (procured from ATCC), primary neuronal cultures from NPC1^{-/-} mice (see below), immortalized human hepatocytes (procured from Creative Bioarray or ATCC), NPC1 patient fibroblasts (procured from Coriell) and/or iPS Cells derived from NPC1 patients (Yu, Xiao-Hua, et al. “NPC1, intracellular cholesterol trafficking and atherosclerosis.”Clinica chimica acta 429 (2014): 69-75, the disclosure of which is incorporated by reference in its entirety). Example cell lines for use in assays described herein include fibroblast cell lines, which may be procured from Coriell Institute (Camden, N.J., United States). Fibroblast cell lines can be from healthy individual donors (e.g., Coriell catalog nos. GM00038, GM00302, GM00500, GM00969, and/or GM05758—referred to respectively herein as H1, H2, H3, H4, and H5 cell lines) or from diagnosed Niemann-Pick Disease, Type C1 patient donors (e.g., Coriell catalog nos. GM00110, GM03123, GM18436, and/or GM23162—referred to respectively herein as N1, N2, N3, and N4 cell lines). Additional cell lines for use in assays described herein include hepatocyte cell lines which may be procured from ATCC (e.g., Hep 3B2.1-7 ATCC HB-8064™ and/or Hep G2 P4 ATCC HB-8065™) and neuronal cells, e.g., iCell™ GlutaNeurons, which may be procured from Cellular Dynamics International (Madison, Wis., United States).

NPC1 homozygous mutant (−/−) mouse model: This model is procured from Jackson Laboratories (BALB/cNctr-Npc1m1N/J) and a breeding colony is established. This mouse strain expresses a premature stop codon within the mouse NPC1 gene leading to marked decrease of expression within the liver and brain. Homozygous mice are viable but sterile. When maintaining the colony, heterozygous mice are intercrossed or bred with wild-type siblings. Progressive functional deficits are evident as early as 40 days of age as measured by ataxia and changes in stride length. Also noted is the accumulation of free cholesterol, GlcCer, LacCer, Sphingomyelin, sphingosine and gangliosides within the CNS tissue. Histological endpoints include lysosomal storage/accumulation, axonal swelling, loss of cerebellar purkinje cells (day between 30 and 50) and general global brain atrophy. Microglial activation in the brain has been noted as early as ˜9 days, significant demyelination (CC, AC, CCx) has been documented at 10 days and hepatosplenomegaly is noted in post-mortem tissue. Males generally progress more rapidly than females.

Npc1^{-/-}, C57BL/6J mouse model: These mice have a phenotype that mimics those seen in the BALB/cNctr-Npc1m1N/J; however, these mice progress more rapidly, with death occurring close to 50 days of age.

Npc1tm(I1061T)Dso mutant mice: These mutant mice possess loxP sites flanking exons 14-20 of the Niemann-Pick type C1 (Npc1) gene, as well as the I1061T missense mutation commonly found in humans with the cholesterol-sphingolipid lysosomal storage disorder, Niemann-Pick type C1 (NPC1) disease. The phenotype within this model is less severe than both of the Npc1 null mice. However, these mice do express a disease relevant human Npc1 transcript.

Non-human primates are used to support both exploratory dose range finding (DRF) studies as well as to evaluate alternative routes of administration.

### Example 1. Vector Design and Synthesis

An AAV viral genome expressing a payload region comprising a polynucleotide encoding a human NPC1 polypeptide is generated. The viral genome will further encode an AAV capsid protein (e.g., an AAV capsid protein of Table 1, e.g., an AAV9 capsid protein or functional variant thereof), such that it can be packaged in an AAV particle. A promoter region regulates expression of the payload region. Without wishing to be bound by theory, it is believed that widespread NPC1 distribution throughout the periphery and CNS provides robust efficacy. Therefore, truncated ubiquitous promoters, such as EF1α, truncated EF1α, or tissue specific promoters (such as CaMKII promoter), are used to promote expression of NPC1 transgene. The promoter is engineered to express NPC1 at efficacious levels in CNS and peripheral tissues.

The viral genome can further comprise ITRs, introns, polyadenylation sequence, and/or linkers, as well as other viral genome components as described herein to optimize efficiency of the vector and expression of NPC1.

The AAV viral genome is a single-strand and comprises a codon optimized nucleotide sequence encoding an NPC1 protein for robust expression of the human NPC1 transgene. Without wishing to be bound by theory, it is believed that this approach allows for the widespread NPC1 expression that functions in synergy with the ubiquitous promoter. The design of this transgene provides the opportunity to use one vector in multiple current and proposed in vivo models, including those used for tolerability studies. A single-strand AAV viral genome comprising a wild-type nucleotide sequence encoding an NPC1 protein will also be generated.

An AAV viral genome expressing a payload region comprising a polynucleotide encoding a human NPC2 polypeptide is also generated. The viral genome will further encode an AAV capsid protein (e.g., an AAV capsid protein of Table 1, e.g., an AAV9 capsid protein or functional variant thereof), such that is can be packaged in an AAV particle. A promoter region regulates expression of the payload region. Without wishing to be bound by theory, it is believed that widespread NPC2 distribution throughout the periphery and CNS provides robust efficacy. Therefore, truncated ubiquitous promoters, such as EF1α, truncated EF1α, or tissue specific promoter (such as CaMKII promoter), is used to promote expression of NPC1 transgene. The promoter is engineered to express NPC2 efficacious levels in CNS and peripheral tissues. The viral genome can further comprise ITRs, introns, polyadenylation sequence, and/or linkers, as well as other viral genome components as described herein to optimize efficiency of the vector and expression of NPC2.

The AAV viral genome is a single-strand viral genome and comprises a codon optimized nucleotide sequence encoding an NPC2 protein for robust expression of the human NPC2 transgene. Without wishing to be bound by theory, it is believed that this approach will allow for the widespread NPC2 expression that will work in synergy with the ubiquitous promoter. The design of this transgene provides the opportunity to use one vector in multiple current and proposed in vivo models, including those used for tolerability studies. A single-strand AAV viral genome comprising a wild-type nucleotide sequence encoding an NPC1 protein will also be generated.

Generation of the transgene plasmid includes two rounds of screening. Within the first round, codon optimization is competed, generating two final codon optimized transgene plasmids. The second round of screening tests promoters, rendering 4-6 lead constructs with promoter and codon optimization.

First round screen (codon optimization): 10 transgene plasmids of choice are transfected into one selected cell type of choice for a dual dose screen (with quadruplicate transfection for each data point). Protein quantification of NPC1 or NPC2 protein by ELISA is the primary output. 10 concentration dose response curves are generated for the top 5 constructs (transfections also in quadruplicates) including calculation of the EC50s for the top 2 NPC1 and NPC2 constructs. Following codon optimization, a second round screen is completed to optimize the promoter design.

Second round screen (promoter optimization): transfections of 10 transgene plasmids using the top 2 codon optimized plasmids (1st round of screen) with 1 of 5 truncated ubiquitous promoters (with quadruplicate transfection for each data point) is performed. Protein quantification of NPC1 or NPC2 protein by ELISA is the primary output. 10 concentration dose-response curves are generated for the 8 constructs (transfections also in quadruplicates) including calculation of the EC50s for the top 4-6 NPC1 and NPC2 constructs. Confirmation of activity of these top NPC1 and NPC2 constructs (ubiquitous promoter with codon optimized nucleotide sequence encoding an NPC1 or NPC2 protein) is completed in 2 selected neuronal cell-lines and 2 selected non-neuronal cell lines (in a 10-point DRC).

### Example 2. Assay Development

NPC1/2 is overexpressed in HEK 293 cells using the AAV vector of Example 1 or other method of transfection. Western Blot and ELISA detection protocols are optimized using routine experimentation to establish assays for NPC1/2 protein detection. These and additional biochemical studies are used to validate assays and establish cell lines for additional in vitro analysis. For example, Western Blot and ELISA assays can be performed and optimized using fibroblast cell lines, which may be obtained from Coriell Institute (Camden, N.J., United States). Fibroblast cell lines can be from healthy individual donors (e.g., Coriell catalog nos. GM00038, GM00302, GM00500, GM00969, and/or GM05758—referred to respectively herein as H1, H2, H3, H4, and H5 cell lines) or from diagnosed Niemann-Pick Disease, Type C1 patient donors (e.g., Coriell catalog nos. GM00110, GM03123, GM18436, and/or GM23162 referred to respectively herein as N1, N2, N3, and N4 cell lines).

A filipin staining assay such as a known or commercially available assay is used to measure cholesterol accumulation in cells with or without functional NPC1/2. The filipin staining assay is optimized using routine experimentation. For example, filipin staining is conducted following transfection of each top NPC1 construct to determine its activity in patient fibroblasts. The EC50 of miglustat is used to set a lower limit of inclusion for these constructs. Specifically, a dose-response study using miglustat treatment of patient fibroblast cell lines is conducted. For example, doses of 10 μM, 30 μM, 100 μM, and higher doses of miglustat, e.g., up to 500 μM, are administered for 3 days or longer, e.g., up to 5 days, to patient fibroblast (from affected or NPC1 disease patients) and compared to control fibroblast cell lines (from healthy or normal donors). Filipin staining can be performed, for example, as described by Chandler, Randy J., et al. “Systemic AAV9 gene therapy improves the lifespan of mice with Niemann-Pick disease, type C1.” Human molecular genetics 26.1 (2017): 52-64 or by Vanier, Marie T., et al. “Diagnostic tests for Niemann-Pick disease type C (NP-C): A critical review.” Molecular genetics and metabolism 118.4 (2016): 244-254, the disclosures of both of which are incorporated by reference herein in their entireties.

Additional assays, including NPC1 staining, cholesterol quantification, and analysis of other cholesterol metabolism biomarkers are performed to quantify intracellular cholesterol. NPC1 staining is performed using anti-Niemann Pick C1 antibody (EPR5209, ab134113, Abcam (Cambridge, Mass., United States)) at 1:1000 to 1:2000 dilution. Cholesterol quantification is performed using Amplex™ Red Cholesterol Assay Kit (cat. no. A12216, Invitrogen). Briefly, healthy and NPC1 patient fibroblasts are cultured and subjected to 4-hour serum starvation prior to beginning the assay. Cells are collected in 0.1% SDS or Microplate Lysis Buffer (lacking sodium vanadate to avoid horseradish peroxidase inhibition). Cells are frozen overnight and the assay is carried out according to manufacturer instructions the following day. Constructs with activity superior or equal to miglustat in reducing intracellular cholesterol are selected as “hits” for in vivo testing. Oxysterol biomarkers may be assessed in plasma and cerebrospinal fluid (CSF) as a key fluid biomarker of disease progression and or treatment efficacy (Porter F D, Scherrer D E, Lanier M H, et al. Cholesterol oxidation products are sensitive and specific blood-based biomarkers for Niemann-Pick C1 disease. Sci Transl Med. 2010; 2(56):56ra81 doi:10.1126/scitranslmed.3001417, the disclosure of which is incorporated herein by reference in its entirety). For example, oxysterols are elevated in plasma of NPC-1 patients, a trait not observed in other lysosomal storage disorders. 7-KC and 3β,5α,6β-triol levels correlate with disease onset (years) and disease severity. In addition, NPC-1 mouse models display elevations in oxysterols compared to control animals. Levels of 3β,5α,6β-triol peak around disease onset (peak body weight and phenotypic deficits), with elevations seen in plasma, brain and liver. Accordingly, in vitro assays for quantifying oxysterols are optimized in sample cell lines.

Thus, the in vitro studies described are used to validate assays and reagents for further in vivo studies. The top 4-6 NPC protein expression constructs driven by a ubiquitous promoter with activity superior or equal to miglustat are identified and packaged into AAV capsid for in vivo studies.

### Example 3. Generation of Viral Genomes for the Expression of NPC1 and In Vitro Screening

### Design of Viral Genome Constructs for the Expression of an NPC1 Protein

Viral genomes were designed for AAV delivery of an NPC1 protein. The nucleotide sequence from 5′ ITR to 3′ ITR of the viral genome constructs that comprise a transgene encoding an NPC1 protein, are provided ITR_ITR 1-ITR_ITR 44 herein, which are SEQ ID NOs: 1752-1759, 1799-1821, or 1824-1836, respectively. These constructs are also summarized in Table 4, as well as Tables 13-17.

Each of these viral genome constructs comprise a nucleic acid comprising a transgene encoding an NPC1 protein. The transgene was designed to comprise a wild type nucleotide sequence encoding NPC1 (SEQ ID NO: 1747), or one of two different codon optimized nucleotide sequence encoding an NPC1 protein, SEQ ID NO: 1749 or 1750. In designing these viral genome constructs for expression of NPC1, several promoters were selected and tested (e.g., promoters as described in Tables 3 and 12), including a CMV promoter (SEQ ID NOs: 1736 or 1739-1741); a CMVie enhancer and a CMV promoter (SEQ ID NO: 1743 and 1736, respectively); a CBA promoter (SEQ ID NO: 1735 or 1738); a CMVie enhancer and a CBA promoter (SEQ ID NO: 1737 and 1735 respectively); or an EF-1α promoter variant (SEQ ID NOs: 1745 or 1782-1791, e.g., as described in Table 12).

Some of the viral genome constructs further comprised an intron region, of SEQ ID NO: 1780 and/or a Kozak sequence of SEQ ID NO: 1746. The viral constructs comprised a 5′ ITR of SEQ ID NO: 1733 or 1837; and a 3′ ITR of 1734 or 1738. The polyadenylation sequence (SEQ ID NO: 1751) was the same across all viral genome constructs designed.

Expression of NPC1 Protein from Viral Genome Constructs

The viral genome constructs encoding NPC1 generated, and investigated for expression of NPC1 protein in vitro. Use of different promoters (e.g., CBA promoters or variants thereof, CMV promoters and variants thereof, EF1a promoter variants) and/or codon optimized nucleotide sequences encoding the NPC1 protein (e.g., SEQ ID NO: 1750 or SEQ ID NO: 1749) (Table 6). An additional set of viral genome constructs were also investigated, testing different combinations of promoters and codon optimized sequences (Tables 7, 9 and 10).

In order to assess NPC1 expression, the viral genome constructs comprising a promoter operably linked to a transgene encoding were transfected into HEK293 cells. NPC1 protein level was determined by ELISA and Western Blot, and normalized to GAPDH control and transfection efficiency. The expression of NPC1 was calculated as percent overexpression relative to the reference construct (ITR_ITR 2 (SEQ ID NO: 1753) which comprises promoter variant 8 (SEQ ID NO: 1782) and the wild-type nucleotide sequence encoding NPC1 (SEQ ID NO: 1747)) (% over reference or % overexpression to reference).

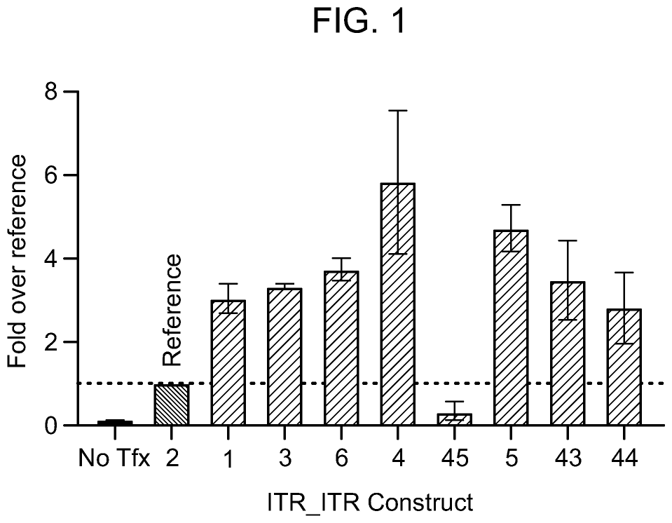

As shown in Table 6 and FIG. 1, multiple constructs resulted in increased levels of NPC1 expression compared to the reference construct (ITR_ITR 2 (SEQ ID NO: 1753)). In addition, increased expression was observed with both codon optimized sequences encoding NPC1 (SEQ ID NO: 1749 or 1750), inclusion of an intron, and/or CMV-based promoters.

TABLE 6
Expression of NPC1 protein by one set of exemplary constructs

| | ITR_ITR | % over |
|---|---|---|
| Description | Construct ID | Ref.* |
| pAAVss_CBA (SEQ ID NO:1735)_wtNPC1 | 7 | −15% |
| (SEQ ID NO: 1747) | | |
| pAAVss_short CMV (SEQ ID NO: | 1 | 203% |
| 1736)_wtNPC1 | | |
| pAAVss_CMVie enhancer (SEQ ID NO: | 41 | 165% |
| 1737)_CBA (SEQ ID NO: 1735)_wtNPC1 | | |
| (SEQ ID NO: 1747) | | |
| pAAAVss_Promoter Variant 8 (SEQ ID NO: | 2 | Reference |
| 1782)_wtNPC1 (SEQ ID NO: 1747) | | |
| pAAVss_Promoter Variant 8 (SEQ ID NO: | 3 | 231% |
| 1782)_pClneo-Intron_wtNPC1 | | |
| (SEQ ID NO: 1747) | | |
| pAAVss_Promoter Variant 8 (SEQ ID NO: | 6 | 274% |
| 1782)_codon optimized NPC1 protein | | |
| coding sequence 1 (SEQ ID NO: 1749) | | |
| pAAVss_Promoter Variant 8 (SEQ ID NO: | 4 | 483% |
| 1782)_codon optimized NPC1 protein | | |
| coding sequence 2 (SEQ ID NO: 1750) | | |
| pAAVss_CBA-D4 (SEQ ID NO: 1738)_wtNPC1 | 42 | −27% |
| (SEQ ID NO: 1747) | | |
| pAAVss_CBA-D4 (SEQ ID NO: 1738)_wtNPC1 | 8 | −22% |
| (SEQ ID NO: 1747) | | |
| pAAVss_CMV-D3 (SEQ ID NO: 1739)_wtNPC1 | 5 | 372% |
| (SEQ ID NO: 1747) | | |
| pAAVss_CMV-D4 (SEQ ID NO: 1740)_wtNPC1 | 43 | 248% |
| (SEQ ID NO: 1747) | | |
| pAAVss_CMV-D6 (SEQ ID NO: 1741)_wtNPC1 | 44 | 182% |
| (SEQ ID NO: 1747) | | |
| pAAVss_Promoter Variant 8 (SEQ ID NO: | 45 | −67% |
| 1782)_NPC1_IDT | | |

* % overexpression compared to that of the reference ITR_ITR 2 (SEQ ID NO: 1753).

An addition set of exemplary of constructs encoding an NPC1 protein were investigated as shown in Table 7 and NPC1 protein expression level in HEK293 cells was measured and compared to a reference construct, ITR_ITR 2 (% overexpression compared to reference).

TABLE 7
Expression of NPC1 protein by one set of exemplary constructs (*
% overexpression compared to that of the reference ITR_ITR 2)

| | Size | ITR_ITR | % Over |
|---|---|---|---|
| Name | (Kb) | Construct ID | Ref .* |
| pAAAVss_Promoter Variant 8 (SEQ ID NO: | 4.64 | 2 | Reference |
| 1782)_wtNPC1 (SEQ ID NO: 1747) | | | |
| pAAVss_short CBA promoter (SEQ ID NO: | 4.6 | 13 | 130% |
| 1735)_codon optimized NPC1 protein coding | | | |
| sequence 2 (SEQ ID NO: 1750) | | | |
| pAAVss_short CMV promoter (SEQ ID NO: | 4.63 | 11 | 363% |
| 1736)_codon optimized NPC1 protein coding | | | |
| sequence 2 (SEQ ID NO: 1750) | | | |
| pAAVss_Promoter Variant 8 (SEQ ID NO: | 4.79 | 14 | 434% |
| 1782)_pClneo-Intron_codon optimized NPC1 | | | |
| protein coding sequence 2 (SEQ ID NO: 1750) | | | |
| pAAVss_short CBA promoter (SEQ ID NO: | 4.67 | 15 | 49% |
| 1735)_codon optimized NPC1 protein coding | | | |
| sequence 1 (SEQ ID NO: 1749) | | | |
| pAAVss_short CMV promoter (SEQ ID NO: | 4.63 | 16 | 581% |
| 1736)_codon optimized NPC1 protein coding | | | |
| sequence 1 (SEQ ID NO: 1749) | | | |
| pAAVss_Promoter Variant 8 (SEQ ID NO: | 4.79 | 17 | 423% |
| 1782)_pClneo-Intron_codon optimized NPC1 | | | |
| protein coding sequence 1 (SEQ ID NO: 1749) | | | |
| pAAVss_short CBA promoter (SEQ ID NO: | 4.82 | 18 | 72% |
| 1735)_pClneo-Intron_wtNPC1 | | | |
| (SEQ ID NO: 1747) | | | |
| pAAVss_short CMV promoter (SEQ ID NO: | 4.78 | 19 | 356% |
| 1736)_pClneo-Intron_wtNPC1 | | | |
| (SEQ ID NO: 1747) | | | |
| pAAVss_short CBA promoter (SEQ ID NO: | 4.82 | 20 | 507% |
| 1735)_pClneo-Intron_codon optimized NPC1 | | | |
| protein coding sequence 2 (SEQ ID NO: 1750) | | | |
| pAAVss_short CMV promoter (SEQ ID NO: | 4.78 | 12 | 1014% |
| 1736)_pClneo-Intron_codon optimized NPC1 | | | |
| protein coding sequence 2 (SEQ ID NO: 1750) | | | |
| pAAVss_short CBA promoter (SEQ ID NO: | 4.82 | 21 | 340% |
| 1735)_pClneo-Intron_codon optimized NPC1 | | | |
| protein coding sequence 1 (SEQ ID NO: 1749) | | | |
| pAAVss_short CMV promoter (SEQ ID NO: | 4.78 | 22 | 1248% |
| 1736)_pClneo-Intron_codon optimized NPC1 | | | |
| protein coding sequence 1 (SEQ ID NO: 1749) | | | |

Table 8 provides a direct comparison between the constructs comprising an EF-1α promoter variant (promoter variant 8, SEQ ID NO: 1782) and wild type (WT, SEQ ID NO: 1747)) or codon optimized NPC1 sequences (SEQ ID NO: 1749 or 1750) with or without an intron (SEQ ID NO: 1780).

TABLE 8
Comparison between exemplary NPC1 constructs comprising an EF-1a
promoter variant, a CMV promoter, or a CBA promoter and a wild-type
or codon optimized nucleotide sequence encoding the NPC1 protein

| | Promoter | | | | | |
|---|---|---|---|---|---|---|
| | Promoter | | | | | |
| | Variant 8 | | | | | |
| | (SEQ ID | | | | | |
| | NO: 1782) | | CBA | | CMV | |
| | without | with | without | with | without | with |
| NPC1 | intron | intron | intron | intron | intron | intron |
| wtNPC1 | ~ | 311% | −15% | 72% | 203% | 356% |
| Codon Optimized | 192% | 434% | 130% | 507% | 363% | 1014% |
| NPC1 Protein | | | | | | |
| Coding Sequence | | | | | | |
| 2 (SEQ ID | | | | | | |
| NO: 1750) | | | | | | |
| Codon Optimized | 140% | 423% | 49% | 340% | 581% | 1248% |
| NPC1 Protein | | | | | | |
| Coding Sequence | | | | | | |
| 1 (SEQ ID | | | | | | |
| NO: 1749) | | | | | | |

As demonstrated in Tables 7-8, inclusion of an intron led to increased NPC1 expression regardless which promoter was used in the construct and the use of a CMV promoter led to increased NPC1 expression relative to the reference construct. Also, both codon optimized nucleotide sequences improved expression of NPC1 protein compared to the wild-type nucleotide sequence. The EF-1a promoter variant and CBA-promoter resulted in similar expression, but the EF-1a promoter variant demonstrated greater overall consistency

Additional viral genome constructs encoding an NPC1 protein were generated using additional EF-1a promoter variants. The EF-1a promoter variants designed are summarized in Table 12 herein. More specifically, promoter variants 11 (SEQ ID NO: 1785), 13 (SEQ ID NO: 1787), 15 (SEQ ID NO: 1789), and 18 (SEQ ID NO: 1791) were designed to incorporate additional native sequences from the EF-1α wild-type promoter (SEQ ID NO: 1781) at their 5′ end (e.g., SEQ ID NOs: 1792-1795, and/or 1797). The addition of these native 5′ sequences restored transcription factor binding sites (TFBS) (Table 23), as measured by the ALGGEN PROMO promoter analysis tool containing the TRANSFAC transcription factor binding site database (version 8.3, using “all species” or “human only” selective criteria (Farre et al. Nucleic Acids Research, 31(13):3651-3653, 2003; the contents of which are incorporated by reference herein in their entirety)). These TFBS may contribute to enhanced ubiquitous expression and likelihood of transgene longevity. Promoter variants 11, 13, 15, and 18 also increase in length which is positively associated with heightened transgene expression. The 3′ end of promoter 11, 13, 15, and 18 resembled the wild-type EF-1α promoter (SEQ ID NO: 1781, e.g., at positions 235-236), which may aid in preserving regulatory influences of the core EF-1α promoter.

TABLE 23
Transcription factor binding site analysis of promoter variants 11, 13, 15, and 18

| | | Introduced | Introduced |
|---|---|---|---|
| | 5′ Bp | TFBS* | TFBS* |
| Promoter Variant | addition | (All Species) | (Human Specific) |
| Promoter Variant 11 | +14 bp | 21 | 4 |
| (SEQ ID NO: 1785) | (addition of SEQ ID NOs: | | |
| | 1795 and 1797) | | |
| Promoter Variant 13 | +19 bp | 29 | 7 |
| (SEQ ID NO: 1787) | (addition of SEQ ID NOs: | | |
| | 1792, 1795, and 1797) | | |
| Promoter Variant 15 | +30 bp | 34 | 8 |
| (SEQ ID NO: 1789) | (addition of SEQ ID NOs: | | |
| | 1793, 1795, and 1797) | | |
| Promoter Variant 18 | +41 bp | 42 | 8 |
| (SEQ ID NO: 1791) | (addition of SEQ ID NOs: | | |
| | 1794, 1795, and 1797) | | |

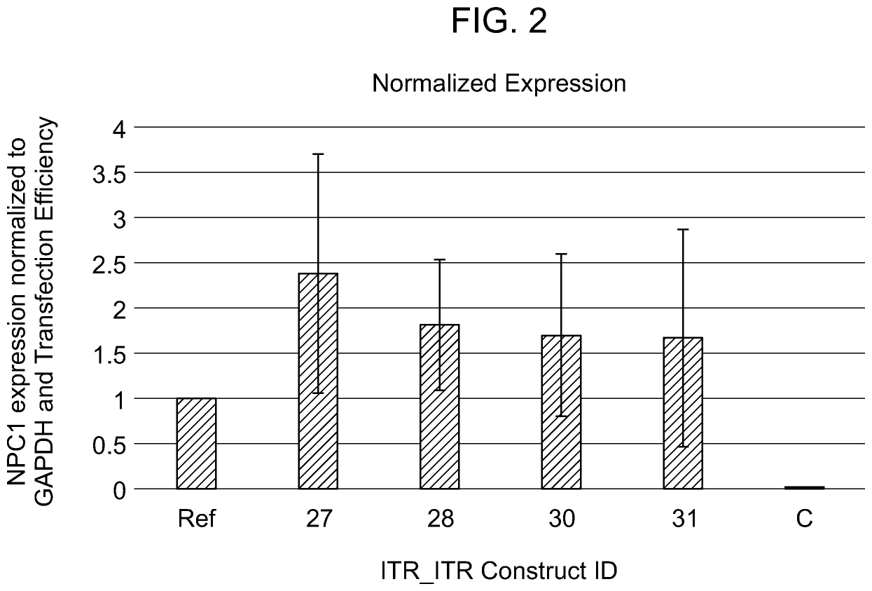

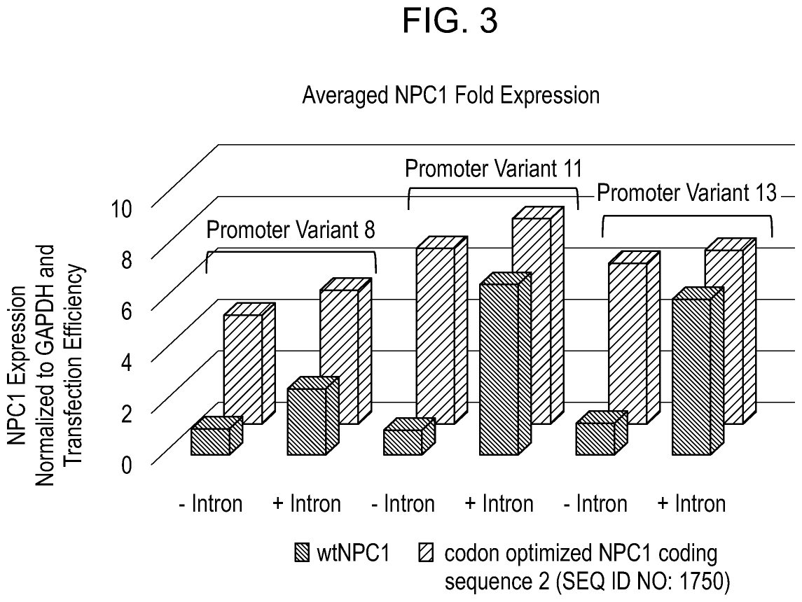

Table 9 summarizes the constructs tested that comprise promoter variant 11 (SEQ ID NO: 1785), promoter variant 13 (SEQ ID NO: 1787), promoter variant 15 (SEQ ID NO: 1789), or promoter variant 18 (SEQ ID NO: 1791), operably linked to a transgene encoding NPC1 (wild-type or codon optimized nucleotide sequences encoding NPC1). These constructs were transfected into HEK293 cells, and the NPC1 protein expression was measured by ELISA and/or western blot and normalized to expression of a GAPDH control and transfection efficiency. The percent overexpression compared to a reference construct (ITR_ITR2 (SEQ ID NO: 1753), which comprises promoter variant 8 (SEQ ID NO: 1782) and the wild-type nucleotide sequence encoding NPC1 (SEQ ID NO: 1747)) was then calculated for each construct tested (% overexpression to reference) (Table 9, FIGS. 2-3).

As demonstrated in FIG. 2, promoter variants 11, 13, 15, and 18, all resulted in increased NPC1 expression relative to the ITR_ITR 2 reference construct (SEQ ID NO: 1753). Further, as shown in Table 9 and FIG. 3, promoter variant 11 and promoter variant 13 both resulted in very strong expression of NPC1 relative to the reference, particularly in combination with an intron (SEQ ID NO: 1780) and/or a codon optimized nucleotide sequence encoding the NPC1 protein (SEQ ID NO: 1749 or SEQ ID NO: 1750).

TABLE 9
Expression of NPC1

| | | | | % |
|---|---|---|---|---|
| | | Size | ITR_ITR | Overexpression |
| Construct | Name | (Kb) | Construct | to Reference |
| 4 | pAAVss_Promoter Variant 8 | 4.64 | 2 | Reference |
| | (SEQ ID NO: 1782)_wtNPC1 | | | |
| | (SEQ ID NO: 1747) | | | |
| 2.35 | pAAVss_Promoter Variant 11 | 4.65 | 27 | −4% |
| | (SEQ ID NO: 1785)_wtNPC1 | | | |
| | (SEQ ID NO: 1747) | | | |
| 2.36 | pAAVss_Promoter Variant 13 | 4.65 | 28 | 23% |
| | (SEQ ID NO: 1787)_wtNPC1 | | | |
| | (SEQ ID NO: 1747) | | | |
| 2.37 | pAAVss_Promoter Variant 15 | 4.66 | 30 | N/A |
| | (SEQ ID NO: 1789)_wtNPC1 | | | |
| | (SEQ ID NO: 1747) | | | |
| 2.38 | pAAVss_Promoter Variant 18 | 4.67 | 31 | N/A |
| | (SEQ ID NO: 1791)_wtNPC1 | | | |
| | (SEQ ID NO: 1747) | | | |
| 2.39 | pAAVss_Promoter Variant 11 | 4.796 | 29 | 558% |
| | (SEQ ID NO: 1785)_Intron_wtNPC1 | | | |
| | (SEQ ID NO: 1747) | | | |
| 2.4 | pAAVss_Promoter Variant 11 | 4.65 | 9 | 582% |
| | (SEQ ID NO: 1785)_NPC1_codon | | | |
| | optimized NPC1 protein coding | | | |
| | sequence 2 (SEQ ID NO: 1750) | | | |
| 2.41 | pAAVss_Promoter Variant 11 | 4.796 | 10 | 696% |
| | (SEQ ID NO: 1785)_Intron_codon | | | |
| | optimized NPC1 protein coding | | | |
| | sequence 2 (SEQ ID NO: 1750) | | | |
| 2.42 | pAAVss_Promoter Variant 13 | 4.796 | 24 | 501% |
| | (SEQ ID NO: 1787)_Intron_wtNPC1 | | | |
| | (SEQ ID NO: 1747) | | | |
| 2.43 | pAAVss_Promoter Variant 13 | 4.65 | 25 | 520% |
| | (SEQ ID NO: 1787)_NPC1_codon | | | |
| | optimizedNPC1 protein coding | | | |
| | sequence 2 (SEQ ID NO: 1750) | | | |
| 2.44 | pAAVss_Promoter Variant 13 | 4.796 | 26 | 574% |
| | (SEQ ID NO: 1787)_Intron_codon | | | |
| | optimized NPC1 protein coding | | | |
| | sequence 2 (SEQ ID NO: 1750) | | | |

Lastly, the above described viral constructs have been formulated into particular expression vectors (e.g., plasmid backbones) for production in either mammalian cells (e.g., HEK193 cells) or insect cells (e.g., Sf9 cells).

### Example 4. Improved NPC1 Expression with Optimized Transgene Design in Multiple Cell Lines Including Patient Fibroblasts

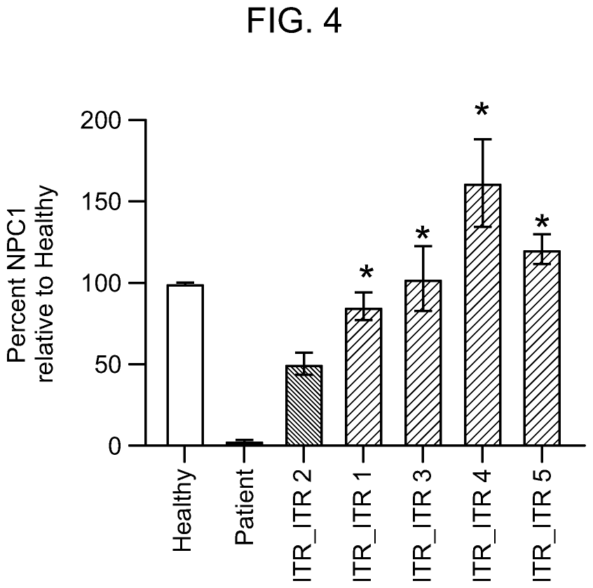

NPC1 protein expression was assessed in healthy and NPC1 patient fibroblasts according to the assays described in Example 2. Whereas patient fibroblasts had little to no NPC1 protein expression, NPC1 protein encoding transgenes of constructs ITR_ITR 2, ITR_ITR 1, ITR_ITR 3, ITR_ITR 4, and ITR_ITR 5 (SEQ ID NOs: 1753, 1752, and 1754-1756, respectively) rescued NPC1 protein expression to at or near the expression level detected in healthy fibroblasts (FIG. 4). ITR_ITR 1 rescued NPC1 protein to near 100% relative to healthy fibroblasts. ITR_ITR 3 and ITR_ITR 5 rescued NPC1 protein to 100% or greater relative to healthy fibroblasts. ITR_ITR 4, which comprises a transgene comprising a codon optimized sequence encoding NPC1 (SEQ ID NO: 1750), rescued NPC1 protein to about 150% relative to healthy fibroblasts. These results showed that codon optimized nucleotide sequence encoding an NPC1 protein as described herein resulted in strong NPC1 protein expression in NPC1-null patient fibroblasts.

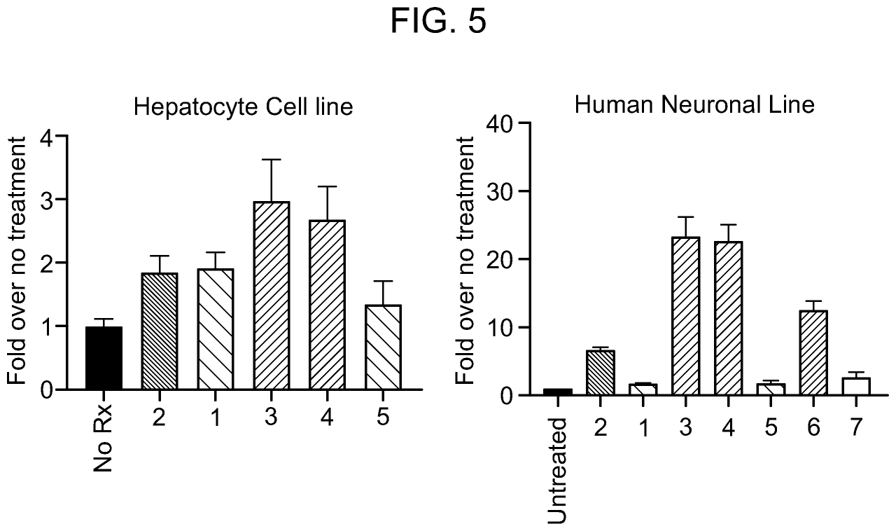

NPC1 protein expression was further assessed for constructs ITR_ITR 2, ITR_ITR 1, ITR_ITR 3, ITR_ITR 4, and ITR_ITR 5 (SEQ ID NOs: 1753, 1752, and 1754-1756, respectively) in hepatocytes (FIG. 5, left graph) and constructs ITR_ITR 2, ITR_ITR 1, ITR_ITR 3, ITR_ITR 4, ITR_ITR 5, ITR_ITR 6, and ITR_ITR 7 (SEQ ID NOs: 1753, 1752, and 1754-1758, respectively) in human neuronal cells (FIG. 5, right graph). As shown in FIG. 5, all NPC1 constructs investigated rescued NPC1 protein expression levels in both hepatocytes and neuronal cells above the expression level detected in the no treatment control. Specifically, for hepatocytes, all tested constructs rescued NPC1 protein expression to about 1.5-3 folds over no treatment control. For neuronal cells, the constructs rescued NPC1 protein expression up to 20-fold over the control group. Both ITR_ITR 4 and ITR_ITR 6 comprised transgenes comprising codon optimized nucleotide sequences encoding NPC1 (SEQ ID NO: 1750 and 1749, respectively), and therefore, these results demonstrated that codon optimized nucleotide sequence encoding an NPC1 protein as described herein resulted in strong NPC1 protein expression in one or more cell types.

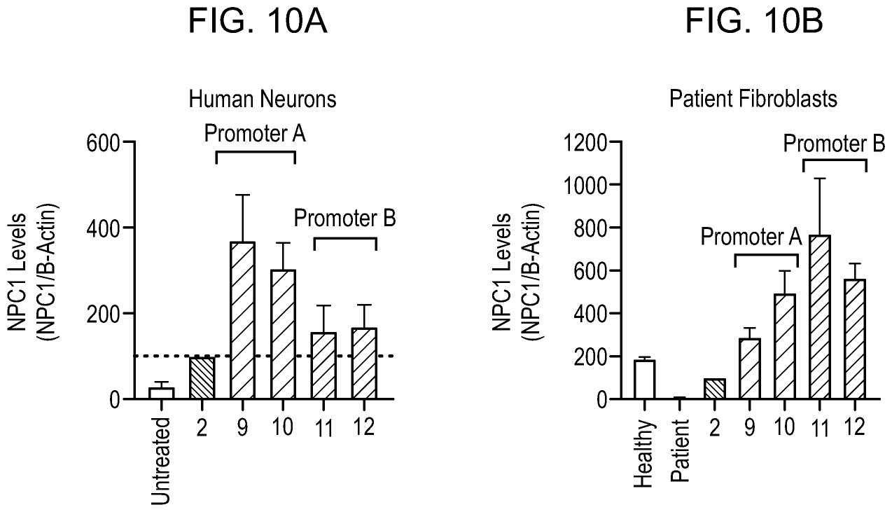

NPC1 expression was also investigated for constructs ITR_ITR 9, ITR_ITR 10, ITR_ITR 11, and ITR_ITR 12 (SEQ ID NOs: 1799-1802, respectively) in human neurons (FIG. 10A) and NPC1 patient fibroblasts (FIG. 10B), which all comprise a transgene comprising a codon optimized sequence encoding the NPC1 protein (SEQ ID NO: 1750). Constructs ITR_ITR 9 and ITR_ITR 10 comprise Promoter Variant 11 (SEQ ID NO: 1785, recited as promoter A in FIG. 10A-10B) driving expression of the transgene comprising the codon optimized sequence encoding NPC1. Constructs ITR_ITR 11 and ITR_ITR 12 comprise a short CMV promoter (SEQ ID NO: 1736; recited as Promoter B in FIG. 10A-10B) driving expression of the transgene comprising the codon optimized sequence encoding NPC1. Constructs ITR_ITR 10 and ITR_ITR 12 both also comprise an intron (SEQ ID NO: 1780).

As shown in FIG. 10A, in neuronal cells, all constructs tested led to very strong NPC1 expression and increased NPC1 expression relative to an untreated control and a reference construct (ITR_ITR 2) comprising Promoter Variant 8 (SEQ ID NO: 2) driving expression of the wild-type sequence encoding NPC1 (SEQ ID NO: 1747). Further, Promoter Variant 11 led to increased expression of NPC1 compared to the CMV promoter in neurons (FIG. 10A).

As shown in FIG. 10B, in patient fibroblasts, all constructs tested led to very strong NPC1 expression and increased NPC1 expression levels relative to a healthy control fibroblasts, patient fibroblasts, and a reference construct (ITR_ITR 2) comprising Promoter Variant 8 (SEQ ID NO: 1782) driving expression of the wild-type sequence encoding NPC1 (SEQ ID NO: 1747). Further, the CMV promoter led to increased expression of NPC1 compared to Promoter Variant 11 in patient fibroblasts (FIG. 10B).

These data demonstrate that constructs comprising Promoter Variant 11 (SEQ ID NO: 1785) driving expression of a codon optimized nucleotide sequence (SEQ ID NO: 1750) encoding the NPC1 protein or the CMV promoter (SEQ ID NO: 1736) are capable of producing robust expression of NPC1, at a level higher than constructs comprising Promoter Variant 8 (SEQ ID NO: 1782) driving expression NPC1 encoded by a wild-type nucleotide sequence.

### Example 5. Reduced Cholesterol Levels with NPC1 Construct Transduction in Patient Fibroblasts

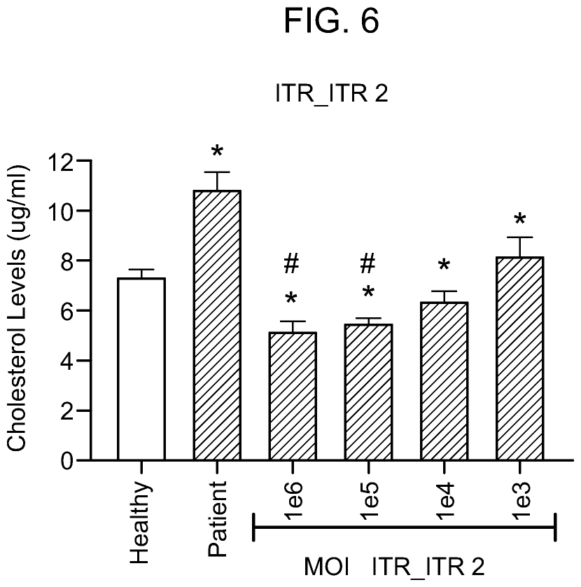

Cholesterol levels were assessed by filipin staining in healthy fibroblasts and compared to patient fibroblasts transduced with ITR_ITR 2 (SEQ ID NO: 1752) to express NPC1 protein. Transduction was performed at a range of multiplicity of infection (M.O.I.) from 0 to 1×10⁶. Patient fibroblasts receiving no NPC1 transduction showed high cholesterol levels by filipin staining, 10-12 μg/ml compared to 6-8 μg/ml for healthy fibroblasts (FIG. 6). Reduction in cholesterol levels to those seen in healthy fibroblasts was observed in a dose-dependent manner with increasing MOI.

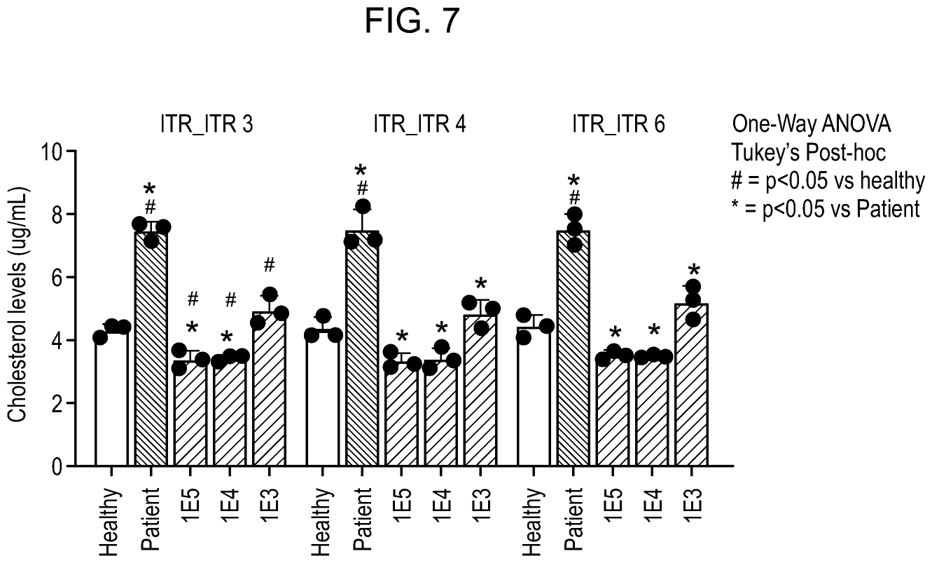

This experiment was repeated with additional NPC1 constructs: ITR_ITR 3, ITR_ITR 4 and ITR_ITR 6 (SEQ ID NOs: 1754, 1755, or 1757), respectively). Transduction was performed at a range of multiplicity of infection (MOI) from 0 to 1×105. Patient fibroblasts receiving no NPC1 transduction showed high cholesterol levels by filipin staining, about 8 μg/ml compared to about 4 μg/ml for healthy fibroblasts (FIG. 7). Similarly, reduction in cholesterol levels to those seen in healthy fibroblasts was observed in a dose-dependent manner with increasing M.O.I.

Constructs ITR_ITR 9, ITR_ITR 10, ITR_ITR 11, and ITR_ITR 12 (SEQ ID NOs: 1799-1802, respectively), which all comprise a transgene comprising a codon optimized sequence encoding the NPC1 protein (SEQ ID NO: 1750), were also tested in this assay and transduced into patient fibroblasts. Constructs ITR_ITR 9 and ITR_ITR 10 comprise Promoter Variant 11 (SEQ ID NO: 1785) driving expression of the transgene comprising the codon optimized sequence encoding NPC1. Constructs ITR_ITR 11 and ITR_ITR 12 comprise a short CMV promoter (SEQ ID NO: 1736) driving expression of the transgene comprising the codon optimized sequence encoding NPC1. Constructs ITR_ITR 10 and ITR_ITR 12 both also comprise an intron (SEQ ID NO: 1780).

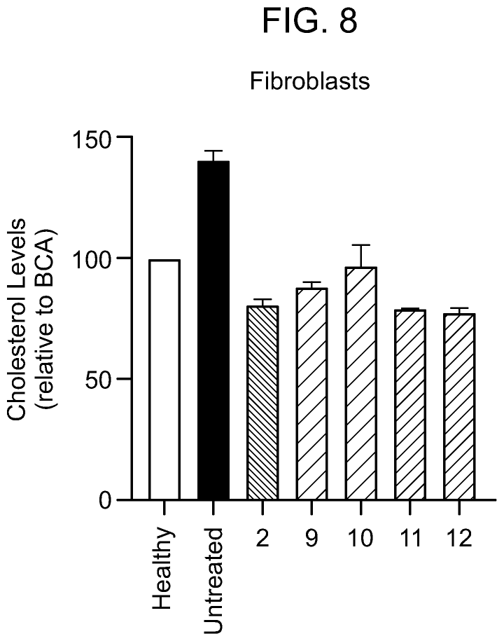

Cholesterol levels (quantified relative to a BCA control) in cells transduced by said constructs were then compared cholesterol levels in healthy control fibroblasts, untreated patient fibroblasts, or patient fibroblasts transduced with ITR_ITR 2 (SEQ ID NO: 1753; comprises Promoter Variant 8 (SEQ ID NO: 2) driving expression of the wild-type sequence encoding NPC1 (SEQ ID NO: 1747). As shown in FIG. 8, all constructs tested reduced cholesterol levels relative to untreated patient fibroblasts, and resulted in cholesterol levels similar to healthy controls.

These results show that optimized NPC1 protein transgene transduction into NPC1 patient fibroblasts effectively reduces heightened cholesterol levels to at or below levels seen in healthy fibroblasts.

### Example 6. Natural History Study

NPC1 (NPC1−/−; Jax Labs stock number 003092) mouse line was procured from Jackson laboratories. Mice were raised under controlled conditions and monitored for vital statistics and evidence of disease progression. Phenotypic and immunohistochemical analyses were performed in the NPC1 null animal model to establish a useable a relevant efficacy-based readout to support efficacy studies. Phenotypic tests included histological analysis (e.g., NPC1 staining and filipin staining) and behavioral analysis including rotarod, clasping, beam walk, gait analysis, and body weight. Benchmarks for disease progression were established. Symptomatic phenotypes for NPC1−/− mice were observable within 30 to 40 days after birth. Clinical observations of NPC1−/− mice included weight loss beginning at about 40 to 50 days after birth, tremors during tail suspension, gait ataxia, splayed limbs, urogenital prolapse, staggered gait, extended head when suspended by tail, piloerect fur, and hunched posture.

Specifically, by about 40 days of age, body weight of NPC1 null (NPC1−/−) mice started to plateau and soon decreased, while body weight of wild type mice continued to increase. Lifespan of NPC1 null mice did not exceed 12 weeks which is shorter than expected life span of healthy mice.

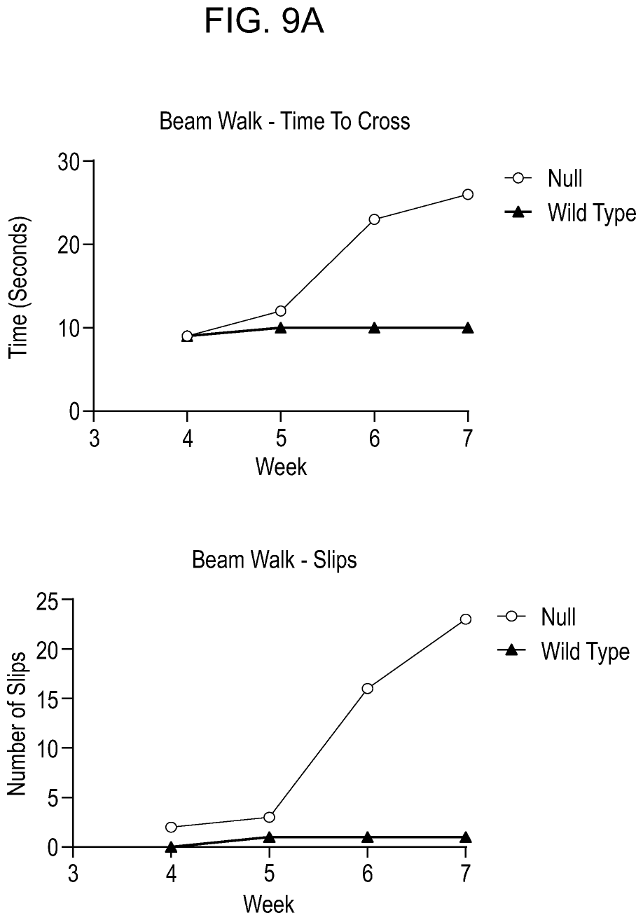

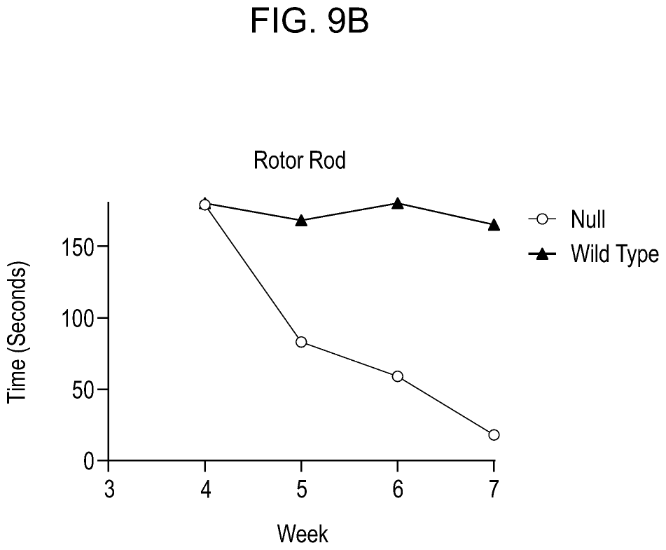

NPC1 null mice also showed severe deficits in motor coordination. For example, in balance beam test, in which mice were challenged to walk across a narrow bridge with 21 mm width after some training, NPC1 null mice at 6-7 weeks old took significant more time to walk across the beam, and slipped more times than wild-type mice (FIG. 9A). Similarly, in the rotarod test, in which mice were challenged to walk on a rotarod accelerating from 5 to 40 rpm for up to 180 seconds, NPC1 null mice at 4 weeks old could complete the task, but older mice fell of the rotating rod soon after the test began (FIG. 9B). In both motor coordination tests, NPC1 null mice were similar to wild-type mice at young age (4 weeks old) but their performance worsened as they aged. These behavioral analyses confirmed impaired motor coordination in NPC1 null mice, established a timeline for disease progression and could be used as simple yet consistent behavioral tests to examine for functional improvements when mice receive treatments.

Oxysterols in plasma and peripheral and central nervous system (CNS) tissues of NPC1 null and wild-type mice were measured by LC-MS in 7-8 weeks old mice. In CNS tissues, wild-type mice generally had higher levels of 24-hydroxycholesterol and 7-ketocholesterol, but not cholestane-3B, 5a, 6B-triol, than NPC1 null mice (Table 18). In periphery tissues, oxysterols in NPC1 null mice were much higher than wild-type mice, especially in the liver, spleen, and plasma (Table 19).

TABLE 18
Summary of oxysterols measured in the CNS in NPC1 null and wild-type mice

| | Brain Stem | | Cerebellum | | Cortex | | Hippocampus | |
|---|---|---|---|---|---|---|---|---|
| | (ng/g of tissue) | | (ng/g of tissue) | | (ng/g of tissue) | | (ng/g of tissue) | |
| | NPC1 | | NPC1 | | NPC1 | | NPC1 | |
| Oxysterol | Null | WT | Null | WT | Null | WT | Null | WT |
| 24-hydroxy- | 4466.3 | 8893.1 | 3738.9 | 2985.9 | 23053.3 | 27186.0 | 24437 | 39196.7 |
| cholesterol | | | | | | | | |
| 7-ketocholesterol | 1700.9 | 2361.3 | 2013.7 | 3800.9 | 1295.8 | 1622.0 | 1300.5 | 3725.3 |
| cholestane-3B, | 298.6 | 147.5 | 470.5 | 703.2 | 301.4 | 124.2 | 309.0 | 195.8 |
| 5a, 6B-triol | | | | | | | | |

TABLE 19
Summary of oxysterols (ng/g of tissue) measured in the periphery in NPC1 null

| | Heart | | GA muscle | | Liver | | Spleen | | Plasma | |
|---|---|---|---|---|---|---|---|---|---|---|
| | (ng/g of | | (ng/g of | | (ng/g of | | (ng/g of | | (ng/g of | |
| | tissue) | | tissue) | | tissue) | | tissue) | | tissue) | |
| | NPC1 | | NPC1 | | NPC1 | | NPC1 | | NPC1 | |
| Oxysterol | Null | WT | Null | WT | Null | WT | Null | WT | Null | WT |
| 24-hydroxy- | 50.6 | 25 | 72.6 | 45.2 | 278.9 | 32.8 | 117.9 | 25.4 | 13.0 | 2.7 |
| cholesterol | | | | | | | | | | |
| 7-keto- | 224.4 | 1497.7 | 12174.3 | 12397.5 | 2028.4 | 136.3 | 4364.5 | 480 | 14.5 | 6.8 |
| cholesterol | | | | | | | | | | |
| cholestane- | 86.5 | 137.7 | 128.3 | 100.4 | 916.1 | 81.1 | 2156.4 | 82.9 | 7.71 | 3.49 |
| 3B, 5a, | | | | | | | | | | |
| 6B-triol | | | | | | | | | | |

Immunnohistochemical staining of the cerebellum showed onset of neurodegeneration consistent with disease onset in NPC1 null mice. Specifically, cerebella of NPC1 mice of 35, 45, 55 and 63 days old were stained for calbindin and CD68 expression. Calbindin is a calcium binding protein responsible for buffering calcium in Purkinje neurons in response to stimulation of glutamate receptors. Staining of calbindin in the cerebellum reduced overtime, indicating a reduction in Purkinje neurons down to a complete loss by 63 days of age. On the contrary, staining of CD68 increased as the mice aged. CD68 is expressed on monocytes, macrophages and microglia (also known as the immune cells of the brain), and is commonly used as a marker of inflammation in the brain. In the cerebellum of 35 days old mice, CD68 was mostly absent. By day 55, CD68 staining could be observed at high frequency, indicating an increase in inflammation as the disease progressed.

In summary, the NPC1^{-/-} mice, BALB/cNctr-Npc1m1N/J, exhibit molecular and physiological phenotypes and progressive functional deficits as expected. This mouse model is useful to assess efficacy of treatments for NPC disease.

### Example 7. Miglustat Head-to-Head Comparison

The EC50 of miglustat is determined (see, for example, Yu, Daozhan, et al. “Niemann-Pick disease type C: induced pluripotent stem cell-derived neuronal cells for modeling neural disease and evaluating drug efficacy.” Journal of biomolecular screening 19.8 (2014): 1164-1173, the disclosure of which is incorporated by reference in its entirety) and used to set inclusion criteria for hits in Example 2. Miglustat is administered at about 1,200 mg/kg/day in some natural history study animals to provide an efficacious benchmark, for example, according to Hughes, Michael P., et al. “AAV9 intracerebroventricular gene therapy improves lifespan, locomotor function and pathology in a mouse model of Niemann-Pick type C1 disease.” Human molecular genetics 27.17 (2018): 3079-3098, the disclosure of which is incorporated by reference herein in its entirety. Base-line effects of miglustat on survival and other phenotypic readouts are evaluated.

To understand how lead constructs compare to miglustat treatment, a dose response study in patient fibroblasts using filipin staining (cholesterol) as the primary readout is conducted. The activity of lead constructs is compared directly to the EC50 of miglustat. Miglustat is administered to cells in vitro at about 100 μM for about 24 hours.

Following the identification of lead candidate, miglustat is run in parallel and in combination with lead candidate to provide a head-to-head comparator.

### Example 8. Biodistribution of AAV Genomes after Intravenous Injection

Biodistribution of AAV1, AAV9 and AAVrh10 genomes administered via facial intravenous (IV) injection to neonatal mice were determined. Briefly, wild-type Balb/c pups of 1-3 days old were injected via facial IV with 30 μl solution containing vehicle control or viral particles of AAV1, AAV9 and AAVrh10 vector carrying GFP as payload. 4 weeks following injection, mice were euthanized and tissues were collected and the amount of AAV genomes/cell in various CNS and peripheral tissues, including the heart, lungs, liver, spleen, muscle, cortex, hippocampus, cerebellum, and brainstem was quantified by PCR.

In one set of experiment, pups were injected with 9.72×10¹¹ viral genomes (vg) of AAV particles comprising an AAV1 capsid protein and a GFP payload. In peripheral tissues, average AAV1 genome counts per cell were found, from highest to lowest, in the heart (4.40 VG/cell), liver (2.91 VG/cell), lung (1.5 VG/cell), and muscle (0.82 VG/cell), in the spleen (0.158 VG/cell). AAV1 genome counts per cell in brainstem (5.45 VG/cell), hippocampus (5.37 VG/cell), and cortex (2.83 VG/cell) were higher than or similar to peripheral tissues, but were low in the cerebellum (0.66 VG/cell).

In another set of experiment, pups were injected with 1×10¹⁰ or 1×10¹¹ vg of AAV particles comprising an AAV1, AAV9, or AAVrh10 capsid protein and a GFP payload. There appeared to be a dose-response between dosages of AAV9 and AAVrh10 injections in all examined tissues. Overall, the AAV biodistribution detected in 4 CNS and 3 peripheral tissue samples were low (less than 5 genomes/cell) across all AAV vector treatments (Table 20).

TABLE 20
AAV genomes per cell in CNS and peripheral tissues

| | Tissue (Vg/cell) | | | | | | |
|---|---|---|---|---|---|---|---|
| | | | | | | | GA |
| AAV/dose | Cerebellum | Hippocampus | Cortex | Brainstem | Spleen | Liver | Muscle |
| AAV11e11 | 0.5 | 4.97 | 4.13 | 2.53 | 0.02 | 1.27 | 0.11 |
| AAV91e10 | 0.003 | 0.08 | 0.02 | 0.03 | 0.01 | 0.22 | 0.04 |
| AAV91e11 | 0.14 | 0.89 | 0.93 | 0.97 | 0.02 | 3.72 | 1.82 |
| AAVrh101e10 | 0.01 | 0.28 | 0.03 | 0.07 | 0.01 | 0.33 | 0.12 |
| AAVrh101e11 | 0.54 | 1.90 | 2.68 | 0.68 | 0.16 | 3.20 | 0.99 |

### Example 9. In Vivo Target Engagement

Top hits (4-6) identified in Example 2 are evaluated in vivo for target expression. Mice (NPC1 null, 6-10 per group) are injected. Initial studies assess IV and/or intrathalamic administration at P25. Starting dose include 1×10¹⁰, 1×10¹¹, or 1×10¹² vg/injection for IV. Mice are euthanized 4 weeks post-dose. Cortex, thalamus, brain stem, cerebellum, spinal cord, liver and lungs are collected and human NPC1 levels are quantified by ELISA. Constructs showing greater than or equal to 50% increase in protein expression (based on WT expression levels in surrogate mice) of NPC1 in the cortex, brainstem, cerebellum and liver are further tested in a time-response study. Briefly, NPC1−/− (n=6-10) mice receive an IV injection of top hits (dose determined based on initial dose-response studies). Cortex, thalamus, brain stem, cerebellum, spinal cord, liver and lungs are collected at various time-points (e.g. 4, 8 and 13 weeks) and human NPC1 mRNA and protein are quantified.

Based on in vivo target engagement and potency, 2-5 hits are selected for additional in vivo efficacy screening. Data obtained include dose and time-point of administration needed for optimal NPC1 expression. These data are used to drive experimental design for efficacy testing.

Constructs identified showing greater than or equal to 50% increase of human NPC1 protein in the cortex, brainstem, cerebellum and liver are tested in NPC1 null mice for efficacy. Mice (n=8-12) are injected with AAV particles carrying hNPC1 at determined doses and evaluated for multiple efficacy outcomes determined based on natural history studies. Up to 2 constructs are selected for additional safety and tolerability studies. Data obtained include a minimally efficacious dose (MED) for in vivo efficacy, data related to therapeutic index (efficacious dose) and potential tolerability findings. These data, in combination with target engagement data are used to drive experimental design for safety and tolerability studies.

### Example 10. In Vivo Evaluation of Vectorized Viral Genomes Designed to Express NPC1

This example investigates the biodistribution and NPC1 expression of the viral genome constructs ITR_ITR 9, ITR_ITR 10, ITR_ITR 11, and ITR_ITR 12 (SEQ ID NOs: 1799-1802, respectively), which all comprise a transgene comprising a codon optimized sequence encoding the NPC1 protein (SEQ ID NO: 1750). Constructs ITR_ITR 9 and ITR_ITR 10 comprise Promoter Variant 11 (SEQ ID NO: 1785) and constructs ITR_ITR 11 and ITR_ITR 12 comprise a short CMV promoter (SEQ ID NO: 1736). Constructs ITR_ITR 10 and ITR_ITR 12 both also comprise an intron (SEQ ID NO: 1780). These constructs were compared to a reference construct (ITR_ITR 2) comprising Promoter Variant 8 (SEQ ID NO: 2) driving expression of the wild-type sequence encoding NPC1 (SEQ ID NO: 1747). These constructs were vectorized in an AAV9 capsid (e.g., AAV9.ITR_ITR2, AAV9.ITR_ITR9, AAV9.ITR_ITR 10, AAV9.ITR_ITR 11, and AAV9.ITR_ITR 12).

Neonatal Balb/c wild type mice (ages P1-P3) were intravenously injected into the facial vein with 1e14, 1e13, or 1e12 Vg/kg of AAV9.ITR_ITR2, AAV9.ITR_ITR9, AAV9.ITR_ITR 10, AAV9.ITR_ITR 11, and AAV9.ITR_ITR 12, or a vehicle control. At 28-days post-IV injection various CNS (e.g., cortex, hippocampus, brainstem, and cerebellum) and peripheral tissues (e.g., liver) were harvested to measure viral genome (VG) biodistribution (VG/cell) and NPC1 expression. All animals treated remained healthy and there was no significant difference in the body weight between the AAV9.ITR_ITR2, AAV9.ITR_ITR9, AAV9.ITR_ITR 10, AAV9.ITR_ITR 11, and AAV9.ITR_ITR 12 treated mice and the mice treated with the vehicle control.

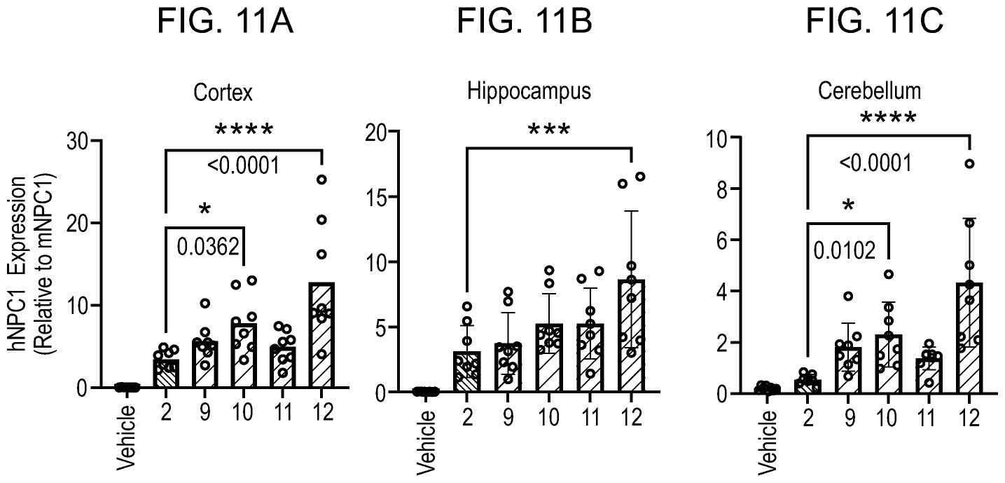

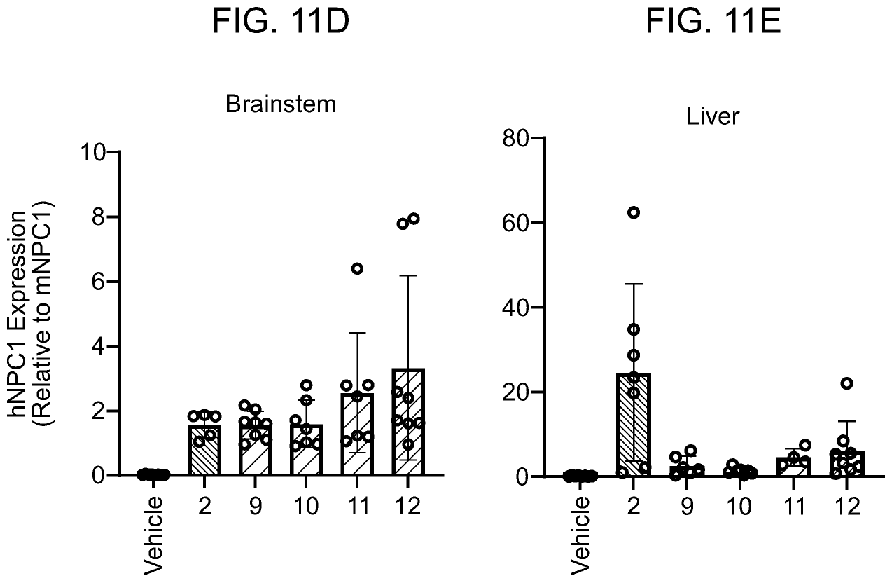

Endogenous murine NPC1 levels normalized to XPNEP1 was low and comparable across all treated and untreated mice. Expression of human NPC1 (hNPC1) (transgene specific) was quantified relative to endogenous, murine NPC1 in the cortex, hippocampus, brainstem, cerebellum, and liver as shown in Table 21 and FIGS. 11A-11E. All of the mice treated with 1e14 Vg/cell of AAV9.ITR_ITR9, AAV9.ITR_ITR 10, AAV9.ITR_ITR 11, and AAV9.ITR_ITR 12 comprising a codon-optimized nucleotide sequence encoding NPC1 (SEQ ID NO: 1750), showed increased NPC1 expression in the CNS tissues (cortex, hippocampus, brainstem, cerebellum) compared to the vehicle treated control and the mice treated with the AAV9.ITR_ITR2 construct comprising a wild-type sequence encoding NPC1 (SEQ ID NO: 1747) (Table 21 and FIGS. 11A-11D). Construct AAV9.ITR_ITR12 which comprised an intron (SEQ ID NO: 1780), CMV promoter (SEQ ID NO: 1736), as well as a codon optimized nucleotide sequence encoding the NPC1 protein (SEQ ID NO: 1750) resulted in the greatest increase in NPC1 expression relative to the AAV9.ITR_ITR2 construct comprising the wild-type sequence encoding NPC1 (SEQ ID NO: 1747), Promoter Variant 8 (SEQ ID NO: 2), and no intron, in the cortex (FIG. 11A), hippocampus (FIG. 11B), cerebellum (FIG. 11C), and brainstem (FIG. 11D) (Table 21). With respect to the liver, the mice treated with the AAV9.ITR_ITR2 construct demonstrated the highest level of NPC1 expression (FIG. 11E and Table 21). Additionally, as shown in Table 21, a dose response in NPC1 expression levels was observed following IV injection of 1e12, 1e13, or 1e14 Vg/cell of the viral constructs in the neonatal mice.

TABLE 21
Average Relative human NPC1 (hNPC1) Expression (hNPC1/mNPC1)

| Construct | Dose (vg/kg) | Cortex | Hippocampus | Brainstem | Cerebellum | Liver |
|---|---|---|---|---|---|---|
| ITR_ITR10 | 1.00E+14 | 24.84 | 13.68 | 4.89 | 3.51 | 4.32 |
| | 1.00E+13 | 1.11 | 1.30 | 0.34 | 0.83 | 0.69 |
| | 1.00E+12 | 0.07 | 0.07 | 0.03 | 0.08 | 0.11 |
| ITR_ITR12 | 1.00E+14 | 7.44 | 9.80 | 3.61 | 4.43 | 3.26 |
| | 1.00E+13 | 0.24 | 0.25 | 0.13 | 0.18 | 1.40 |
| | 1.00E+12 | 0.08 | 0.08 | 0.06 | 0.05 | 0.31 |

Biodistribution (VG/cell) was compared to hNPC1 expression (relative to mNPC1 levels). Approximately 24× hNPC1/Vg was observed in the cortex, 4× hNPC1/Vg was observed in cerebellum, and 0.07× hNPC1/Vg was observed in the liver in mice treated with AAV9.ITR_ITR10. Approximately 16× hNPC1/Vg was observed in the cortex, 8× hNPC1/Vg was observed in cerebellum, and 0.21× hNPC1/Vg was observed in the liver in mice treated with AAV9.ITR_ITR12.

These data show that inclusion of an intron as well as the use of a codon-optimized nucleotide sequence encoding the NPC1 protein (e.g., SEQ ID NO: 1750) enhance expression of NPC1 in the CNS. The ITR_ITR 9, ITR_ITR 10, ITR_ITR 11, and ITR_ITR 12 (SEQ ID NOs: 1799-1802, respectively) constructs could therefore be used in the treatment of disorders associated with a lack of NPC1 expression, such as a lysosomal storage disease or Niemann-Pick disease type C1.

### Example 11: Bioinformatics Analysis of Wild-Type and Optimized Sequences Encoding an NPC1 Protein

Bioinformatics analysis on the sequence level was performed to differentiate between viral genome constructs encoding an NPC1 protein, wherein the NPC1 protein is encoded by a wild-type nucleotide sequence of SEQ ID NO: 1747 (e.g., the nucleotide sequence encoding the NPC1 protein of ITR_ITR2, as shown in Table 4) and a optimized nucleotide sequence of SEQ ID NO: 1750 (e.g., the nucleotide sequence encoding the GBA protein of ITR_ITR9-ITR_ITR12, as shown in Table 4 and 13-17).

Briefly, sequence-level differentiation criteria, such as GC content, microRNA binding, and codon usage, were assessed using mRNA-based sequence analysis tools (e.g., Rare Codon Analyzer tool by Agarwal, Shivangi, et al., Cellular microbiology, 17(10): 1494-1509, 2015; and miRDB by Chen & Wang, 2020, Nucleic Acids Res, 48(D1): D127-D131; the contents of each herein incorporated by reference in its entirety).

Using miRDB (Chen & Wang, 2020, supra), the optimized nucleotide sequence encoding an NPC1 protein of SEQ ID NO: 1750 and the wild-type nucleotide sequence encoding an NPC1 protein of SEQ ID NO: 1747 were assessed for predicative miRNA binding sites. Predictive microRNA binding sites were unique between the WT (SEQ ID NO: 1747) and codon optimized (SEQ ID NO: 1750) NPC1 coding sequences and exhibited no overlap, demonstrating strong sequence divergence. While strictly predictive, microRNA binding can influence mRNA stability and resulting protein abundance.

TABLE 22
miRDB Summary of miRNA Binding

| | Total microRNA | High Confidence |
|---|---|---|
| 5′ Bp | Binding | Binding |
| addition | Sites | Sites |
| WT (SEQ ID | 77 | 12 |
| NO: 1747) | | |
| Optimized (SEQ | 79 | 9 |
| ID NO: 1750) | | |

The GC content was also assessed across the entire 3.7kb sequence of the optimized nucleotide sequence of SEQ ID NO: 1750 and the wild-type nucleotide sequence encoding of SEQ ID NO: 1747, using the GC Content Calculator. The wild-type nucleotide sequence of SEQ ID NO: 1747 and the optimized nucleotide sequence of SEQ ID NO: 1750 maintained a similar GC content ratio with regards to peaks and valleys. It is therefore expected that both the wild type (SEQ ID NO: 1747) and the optimized (SEQ ID NO: 1750) nucleotide sequences encoding an NPC1 protein will maintain similar translation kinetics needed for successful protein folding (changes in translational kinetics can result in misfolded protein). Additionally, the optimization that resulted in SEQ ID NO: 1750 shifted the overall GC content from 49.6% (WT) to 56.7% (optimized), and this can increase transcriptional speed and result in greater mRNA abundance.

Codon usage was then analyzed between the optimized nucleotide sequence of SEQ ID NO: 1750 and the wild-type nucleotide sequence encoding of SEQ ID NO: 1747 using the Rare Codon Analyzer tool (Agarwal, Shivangi, et al., supra). The Codon Adaptation Index (CAI) value, which measures the codon usage similarity between a gene and the host species, was calculated for each sequence. The wild-type nucleotide sequence of SEQ ID NO: 1747 had a CAI value of 0.76, and the optimized nucleotide sequence of SEQ ID NO: 1750 had a CAI value of 0.95. CAI indexes range from 0 to 1, where values closer to 1 have a higher likelihood of successful heterologous gene expression in the host species due to species-specific codon preferences. The optimization that resulted in SEQ ID NO: 1750 shifted the CAI score from 0.76 (CAI score of the wild-type nucleotide sequence of SEQ ID NO: 1747) to 0.95, which increased the theoretical expression potential in humans by incorporating more commonly used codons (versus rare codons) in the coding sequence.

What is claimed is:

1. An isolated, e.g., recombinant, nucleic acid comprising a transgene encoding an NPC1 protein, wherein the nucleotide sequence encoding the NPC1 protein comprises a nucleotide sequence with at least 90%, 92%, 95%, 96%, 97%, 98%, or 99% sequence identity to the nucleotide sequence of SEQ ID NO: 1750 or 1749.

2. An isolated, e.g., recombinant, vial genome comprising a promoter operably linked to a nucleic acid comprising a transgene encoding an NPC1 protein, wherein the nucleotide sequence encoding the NPC1 protein comprises a nucleotide sequence with at least 90%, 92%, 95%, 96%, 97%, 98%, or 99% sequence identity to the nucleotide sequence of SEQ ID NO: 1750 or 1749.

3. An isolated, e.g., recombinant, vial genome comprising a promoter operably linked to a nucleic acid comprising a transgene encoding an NPC1 protein, wherein the promoter comprises an EF-1a promoter variant comprising [A]-[B]-[C]-[D]-[E], wherein:
(i) [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794; or [A] is absent;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
(iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822, or a nucleotide sequence having at least 90%, 92%, 95%, 96%, 97%, 98%, or 99% sequence identity thereto, provided that [D] does not comprise the nucleotides “GC” at positions 235-236, numbered according to SEQ ID NO: 1781; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798 or [E] is absent.

4. The viral genome of claim 3, wherein the nucleotide sequence encoding the NPC1 protein comprises a nucleotide sequence with at least 90%, 92%, 95%, 96%, 97%, 98%, or 99% sequence identity to the nucleotide sequence of SEQ ID NO: 1750 or 1749.

5. The isolated nucleic acid of claim 1, or the viral genome of any one of claims 2-4, wherein the nucleotide sequence encoding the NPC1 protein comprises:
(i) the nucleotide sequence of SEQ ID NO: 1750 or a nucleotide sequence at least 95% identical thereto;
(ii) the nucleotide sequence of SEQ ID NO: 1749 or a sequence at least 95% identical thereto.

6. The viral genome of claim 3, wherein the nucleotide sequence encoding the NPC1 protein comprises the nucleotide sequence of SEQ ID NO: 1747, or a nucleotide sequence at least 85%, 90%, 92%, 95%, 96%, 97%, 98%, or 99% sequence identity thereto.

7. The viral genome of any one of claims 1-6, wherein the encoded NPC1 protein comprises:
(i) the amino acid sequence of SEQ ID NO: 1748, or an amino acid sequence at least 95% identical thereto;
(ii) an amino acid sequence encoded by the nucleotide sequence of SEQ ID NO: 1749 or 1750, or a nucleotide sequence at least 90%, 92%, 95%, 96%, 97%, 98%, or 99% identical thereto.

8. The viral genome of any one of claims 2-7, wherein:
(i) [A] is absent;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
(iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822, or a nucleotide sequence having at least 90%, 92%, 95%, 96%, 97%, 98%, or 99% sequence identity thereto, provided that [D] does not comprise the nucleotides “GC” at positions 235-236, numbered according to SEQ ID NO: 1781; and
(v) [E] is absent.

9. The viral genome of any one of claims 2-8, wherein:
(i) [A] comprises SEQ ID NO: 1792, or a sequence having at least sequence comprising at least one or two modifications, e.g., substitutions (e.g., conservative substitutions), relative to the nucleotide sequence of SEQ ID NO: 1792;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
(iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822, or a nucleotide sequence having at least 90%, 92%, 95%, 96%, 97%, 98%, or 99% sequence identity thereto, provided that [D] does not comprise the nucleotides “GC” at positions 235-236, numbered according to SEQ ID NO: 1781; and
(v) [E] is absent.

10. The viral genome of any one of claims 2-8, wherein:
(i) [A] comprises SEQ ID NO: 1793, or a sequence having at least sequence comprising at least one, two, or three but no more than four modifications, e.g., substitutions (e.g., conservative substitutions), relative to the nucleotide sequence of SEQ ID NO: 1793;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
(iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822, or a nucleotide sequence having at least 90%, 92%, 95%, 96%, 97%, 98%, or 99% sequence identity thereto, provided that [D] does not comprise the nucleotides “GC” at positions 235-236, numbered according to SEQ ID NO: 1781; and
(v) [E] is absent.

11. The viral genome of any one of claims 2-8, wherein:
(i) [A] comprises SEQ ID NO: 1794, or a sequence having at least sequence comprising at least one, two, or three but no more than four modifications, e.g., substitutions (e.g., conservative substitutions), relative to the nucleotide sequence of SEQ ID NO: 1794;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
(iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822, or a nucleotide sequence having at least 90%, 92%, 95%, 96%, 97%, 98%, or 99% sequence identity thereto, provided that [D] does not comprise the nucleotides “GC” at positions 235-236, numbered according to SEQ ID NO: 1781; and
(v) [E] is absent.

12. The viral genome of any one of claims 2-11, wherein the EF-1α promoter variant comprises the nucleotide sequence of SEQ ID NO: 1785, 1787, 1789, or 1791, or a nucleotide sequence having at least 95% sequence identity thereto, provided that the EF-1α promoter variant does not comprise the nucleotides “GC” at positions 235-236, numbered according to SEQ ID NO: 1781.

13. The viral genome of claim 2, wherein the promoter comprises:
(i) a ubiquitous promoter or a tissue specific promoter;
(ii) an EF-1a promoter, a chicken β-actin (CBA) promoter and/or its derivative CAG, a CMV immediate-early enhancer and/or promoter, a β glucuronidase (GUSB) promoter, a ubiquitin C (UBC) promoter, a neuron-specific enolase (NSE), a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, an enkephalin (Enk) and excitatory amino acid transporter 2 (EAAT2), a glial fibrillary acidic protein (GFAP) promoter, a myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof; and/or
(iii) an EF-1a promoter or an EF-1a promoter variant, optionally wherein the EF-1a promoter comprises the nucleotide sequence of SEQ ID NO: 1781, or a nucleotide sequence at least 95% identical thereto.

14. The viral genome of any one of claim 2 or 13, wherein the promoter comprises:
(i) a CMV promoter;
(ii) a CMVie enhancer and a CMV promoter, optionally wherein the CMVie enhancer comprises the nucleotide sequence of SEQ ID NO: 1743, or a nucleotide sequence at least 95% identical thereto, and the CMV promoter comprises the nucleotide sequence of SEQ ID NO: 1736, or a nucleotide sequence at least 95% identical thereto;
(iii) the nucleotide sequence of SEQ ID NO: 1736 or 1739-1741, or a nucleotide sequence at least 95% identical thereto; or
(iv) the nucleotide sequence of SEQ ID NO: 1736 or a nucleotide sequence at least 95% identical thereto.

15. The viral genome of claim 13 or 14, wherein the CMVie enhancer comprises the nucleotide sequence of SEQ ID NO: 1737, 1742, or 1743, or a nucleotide sequence at least 95% identical thereto.

16. The viral genome of any one of claim 2 or 13-14, wherein the promoter comprises:
(i) a CBA promoter;
(ii) a CMVie enhancer and a CBA promoter, optionally wherein:
(a) the CMVie enhancer comprises the nucleotide sequence of SEQ ID NO: 1742, or a nucleotide sequence 95% identical thereto, and the CBA promoter comprises the nucleotide sequence of SEQ ID NO: 1735, or a nucleotide sequence at least 95% identical thereto; or
(b) the CMVie enhancer comprises the nucleotide sequence of SEQ ID NO: 1737, or a nucleotide sequence 95% identical thereto, and the CBA promoter comprises the nucleotide sequence of SEQ ID NO: 1735, or a nucleotide sequence at least 95% identical thereto; or
(iii) the nucleotide sequence of SEQ ID NO: 1735 or 1738, or a nucleotide sequence at least 95% identical thereto

17. The promoter of claim 2, wherein the promoter comprises an EF-1α promoter variant comprising [A]-[B]-[C]-[D]-[E], wherein:
(i) [A] comprises SEQ ID NO: 1792, 1793, or 1794, or a sequence having at least sequence comprising at least one, two, or three but no more than four modifications, e.g., substitutions (e.g., conservative substitutions), relative to the nucleotide sequence of SEQ ID NO: 1792, 1793, or 1794; or [A] is absent;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795 or [B] is absent;
(iii) [C] comprises the nucleotides GT, the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822 or 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798 or [E] is absent.

18. The viral genome of claim 17, wherein:
(i) [A] is absent;
(ii) [B] is absent;
(iii) [C] comprises the nucleotides GT;
(iv) [D] comprises the nucleotide sequence of SEQ ID NO: 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.

19. The viral genome of claim 17, wherein:
(i) [A] is absent;
(ii) [B] is absent;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.

20. The viral genome of claim 17, wherein:
(i) [A] is absent;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.

21. The viral genome of claim 17, wherein:
(i) [A] is absent;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797, or [C] is absent;
(iv) [D] comprises the nucleotide sequence of SEQ ID NO: 1822, or a nucleotide sequence having at least 90%, 92%, 95%, 96%, 97%, 98%, or 99% sequence identity thereto; and
(v) [E] is absent.

22. The viral genome of claim 17, wherein:
(i) [A] comprises SEQ ID NO: 1792, or a sequence having at least sequence comprising at least one or two modifications, e.g., substitutions (e.g., conservative substitutions), relative to the nucleotide sequence of SEQ ID NO: 1792;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.

23. The viral genome of claim 17, wherein:
(i) [A] comprises SEQ ID NO: 1792, or a sequence having at least sequence comprising at least one or two modifications, e.g., substitutions (e.g., conservative substitutions), relative to the nucleotide sequence of SEQ ID NO: 1792;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1822, or a nucleotide sequence having at least 90%, 92%, 95%, 96%, 97%, 98%, or 99% sequence identity thereto; and
(v) [E] is absent.

24. The viral genome of claim 17, wherein:
(i) [A] comprises SEQ ID NO: 1793, or a sequence having at least sequence comprising at least one, two, or three but no more than four modifications, e.g., substitutions (e.g., conservative substitutions), relative to the nucleotide sequence of SEQ ID NO: 1793;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.

25. The viral genome of claim 43, wherein:
(i) [A] comprises SEQ ID NO: 1793, or a sequence having at least sequence comprising at least one, two, or three but no more than four modifications, e.g., substitutions (e.g., conservative substitutions), relative to the nucleotide sequence of SEQ ID NO: 1793;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1822, or a nucleotide sequence having at least 90%, 92%, 95%, 96%, 97%, 98%, or 99% sequence identity thereto; and
(v) [E] is absent.

26. The viral genome of claim 17, wherein:
(i) [A] comprises SEQ ID NO: 1794, or a sequence having at least sequence comprising at least one, two, or three but no more than four modifications, e.g., substitutions (e.g., conservative substitutions), relative to the nucleotide sequence of SEQ ID NO: 1794;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1823, or a nucleotide sequence having at least 90%, 92%, 95%, 96%, 97%, 98%, or 99% sequence identity thereto; and
(v) [E] comprises the nucleotide sequence of SEQ ID NO: 1798, or a sequence having at least sequence comprising at least one or two, but no more than 3 modifications, e.g., substitutions (e.g., conservative substitutions), relative to the nucleotide sequence of SEQ ID NO: 1798.

27. The viral genome of claim 17, wherein:
(i) [A] comprises SEQ ID NO: 1794, or a sequence having at least sequence comprising at least one, two, or three but no more than four modifications, e.g., substitutions (e.g., conservative substitutions), relative to the nucleotide sequence of SEQ ID NO: 1794;
(ii) [B] comprises the nucleotide sequence of SEQ ID NO: 1795, or a sequence having at least sequence comprising at least one or two, but no more than 3 modifications, e.g., substitutions (e.g., conservative substitutions), relative to the nucleotide sequence of SEQ ID NO: 1795;
(iii) [C] comprises the nucleotide sequence of SEQ ID NO: 1797, or a nucleotide sequence comprising at least one, two, or three but no more than four modifications, e.g., substitutions (e.g., conservative substitutions), relative to the nucleotide sequence of SEQ ID NO: 1797;
(iv) [D] comprises the nucleotide sequence of SEQ ID NO: 1822, or a nucleotide sequence having at least 90%, 92%, 95%, 96%, 97%, 98%, or 99% sequence identity thereto; and
(v) [E] is absent.

28. The viral genome of any one of claim 2 or 17-27, wherein the promoter comprises the nucleotide sequence of SEQ ID NO: 1785, 1782-1784, 1786-1791, or a sequence at least 95% identical thereto.

29. The viral genome of any one of claims 2-28, which further comprises:
(i) an inverted terminal repeat (ITR) sequence, optionally wherein the ITR sequence is positioned 5′ relative to the transgene encoding the NPC1 protein and/or the ITR sequence is positioned 3′ relative to the transgene encoding the NPC1 protein;
(ii) a polyadenylation (polyA) signal region;
(iii) an intron region;
(iv) an exon region, e.g., at least one, two, or three exon regions;
(v) a Kozak sequence; and/or
(vi) a nucleotide sequence encoding a miR binding site, e.g., a miR binding site that modulates, e.g., reduces, expression of the payload encoded by the viral genome in a cell or tissue where the corresponding miRNA is expressed.

30. The viral genome of claim 29, wherein:
(i) the ITR comprises a nucleotide sequence of SEQ ID NO: 1733, 1734, 1837, or 1838 or a nucleotide sequence at least 95% identical thereto;
(ii) the ITR sequence positioned 5′ relative to the transgene encoding the NPC1 protein comprises the nucleotide sequence of SEQ ID NO: 1733, or a nucleotide sequence with at least 95% sequence identity thereto; and/or the ITR sequence positioned 3′ relative to the transgene encoding the NPC1 protein comprises the nucleotide sequence of SEQ ID NO: 1734, or a nucleotide sequence with at least 95% sequence identity thereto; or
(iii) the ITR sequence positioned 5′ relative to the transgene encoding the NPC1 protein comprises the nucleotide sequence of SEQ ID NO: 1837, or a nucleotide sequence with at least 95% sequence identity thereto; and/or the ITR sequence positioned 3′ relative to the transgene encoding the NPC1 protein comprises the nucleotide sequence of SEQ ID NO: 1838, or a nucleotide sequence with at least 95% sequence identity thereto.

31. The viral genome of claim 29, wherein:
(i) the polyA signal region comprises the nucleotide sequence of SEQ ID NO: 1751, or a nucleotide sequence at least 95% identical thereto;
(ii) the intron region comprises the nucleotide sequence of SEQ ID NO: 1780, or a nucleotide sequence at least 95% identical thereto; and/or
(iii) the Kozak sequence comprises SEQ ID NO: 1746, or a nucleotide sequence comprising at least one, two, or three but no more than four modifications, e.g., substitutions, relative to the nucleotide sequence of SEQ ID NO: 1746

32. The viral genome of claim 29, which comprises:
(i) at least 1-5 copies of an encoded miR binding site, e.g., at least 1, 2, 3, 4, or 5 copies;
(ii) at least 1, 2 or 3 copies of an encoded miR binding sites, optionally wherein
(a) all two or three copies comprise the same miR binding site, or at least one, two, or all of the copies comprise a different miR binding site; and/or
(b) the two or three copies of the encoded miR binding sites are continuous (e.g., not separated by a spacer), or are separated by a spacer (e.g., a spacer sequence comprising the nucleotide sequence of SEQ ID NO: 1846, or a nucleotide sequence having at least one, two, or three modifications, but no more than four modifications of SEQ ID NO: 1846); and/or
(iii) at least two or three copies of an encoded miR binding site, optionally wherein
(a) all two or three copies comprise the same miR binding site, or at least one, two, or all of the copies comprise a different miR binding site; and/or
(b) the two or three copies of the encoded miR binding sites are continuous, (e.g., not separated by a spacer), or are separated by a spacer (e.g., a spacer sequence comprising the nucleotide sequence of SEQ ID NO: 1846, or a nucleotide sequence having at least one, two, or three modifications, but no more than four modifications of SEQ ID NO: 1846).

33. The viral genome of any one of claim 29 or 32, wherein the encoded miR binding site comprises a miR122 binding site, a miR183 binding site, a miR-142-3p, or a combination thereof, optionally wherein:
(i) the encoded miR122 binding site comprises the nucleotide sequence of SEQ ID NO: 1840, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1840;
(ii) the encoded miR183 binding site comprises the nucleotide sequence of SEQ ID NO: 1843, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1843; and/or
(iii) the encoded miR-142-3p binding site comprises the nucleotide sequence of SEQ ID NO: 1842, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1842.

34. The viral genome of any one of claims 2-33, which:
(i) is single stranded;
(ii) further comprises a nucleic acid encoding a capsid protein, e.g., a structural protein, wherein the capsid protein comprises a VP1 polypeptide, a VP2 polypeptide, and/or a VP3 polypeptide, optionally wherein the VP1 polypeptide, the VP2 polypeptide, and/or the VP3 polypeptide are encoded by at least one Cap gene; and/or
(iii) further comprises a nucleic acid encoding a Rep protein, e.g., a non-structural protein, wherein the Rep protein comprises a Rep78 protein, a Rep68, Rep52 protein, and/or a Rep40 protein, optionally wherein the Rep78 protein, the Rep68 protein, the Rep52 protein, and/or the Rep40 protein are encoded by at least one Rep gene.

35. A viral genome comprising in 5′ to 3′ order:
(i) a 5′ adeno-associated (AAV) ITR, optionally wherein the 5′ AAV ITR comprises the nucleotide sequence of SEQ ID NO: 1733, or a nucleotide sequence at least 95% identical thereto;
(ii) an EF-1α promoter variant, optionally wherein the EF-1α promoter variant comprises the nucleotide sequence of SEQ ID NO: 1785, or a nucleotide sequence at least 95% identical thereto;
(iii) a Kozak sequence, optionally wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746;
(iv) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
(v) a polyA signal region, optionally wherein the polyA signal region comprises the nucleotide sequence of SEQ ID NO: 1751, or a nucleotide sequence at least 95% identical thereto; and
(vi) a 3′ AAV ITR, optionally wherein the 3′ AAV ITR comprises the nucleotide sequence of SEQ ID NO: 1734, or a nucleotide sequence at least 95% identical thereto.

36. A viral genome comprising in 5′ to 3′ order:
(i) a 5′ adeno-associated (AAV) ITR, optionally wherein the 5′ AAV ITR comprises the nucleotide sequence of SEQ ID NO: 1733, or a nucleotide sequence at least 95% identical thereto;
(ii) an EF-1a promoter variant, optionally wherein the EF-1a promoter variant comprises the nucleotide sequence of SEQ ID NO: 1785, or a nucleotide sequence at least 95% identical thereto;
(iii) an intron region, optionally wherein the intron region comprises the nucleotide sequence of SEQ ID NO: 1780, or a nucleotide sequence at least 95% identical thereto;
(iv) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
(v) a polyA signal region, optionally wherein the polyA signal region comprises the nucleotide sequence of SEQ ID NO: 1751, or a nucleotide sequence at least 95% identical thereto; and
(vi) a 3′ AAV ITR, optionally wherein the 3′ AAV ITR comprises the nucleotide sequence of SEQ ID NO: 1734, or a nucleotide sequence at least 95% identical thereto.

37. A viral genome comprising in 5′ to 3′ order:
(i) a 5′ adeno-associated (AAV) ITR, optionally wherein the 5′ AAV ITR comprises the nucleotide sequence of SEQ ID NO: 1733, or a nucleotide sequence at least 95% identical thereto;
(ii) a CMV promoter variant, optionally wherein the CMV promoter comprises the nucleotide sequence of SEQ ID NO: 1736, or a nucleotide sequence at least 95% identical thereto;
(iii) a Kozak sequence, optionally wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746;
(iv) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
(v) a polyA signal region, optionally wherein the polyA signal region comprises the nucleotide sequence of SEQ ID NO: 1751, or a nucleotide sequence at least 95% identical thereto; and
(vi) a 3′ AAV ITR, optionally wherein the 3′ AAV ITR comprises the nucleotide sequence of SEQ ID NO: 1734, or a nucleotide sequence at least 95% identical thereto.

38. A viral genome comprising in 5′ to 3′ order:
(i) a 5′ adeno-associated (AAV) ITR, optionally wherein the 5′ AAV ITR comprises the nucleotide sequence of SEQ ID NO: 1733, or a nucleotide sequence at least 95% identical thereto;
(ii) a CMV promoter variant, optionally wherein the CMV promoter comprises the nucleotide sequence of SEQ ID NO: 1736, or a nucleotide sequence at least 95% identical thereto;
(iii) an intron region, optionally wherein the intron region comprises the nucleotide sequence of SEQ ID NO: 1780, or a nucleotide sequence at least 95% identical thereto;
(iv) a Kozak sequence, optionally wherein the Kozak sequence comprises SEQ ID NO: 1746, or a nucleotide sequence comprising at least one, two, or three but no more than four modifications, e.g., substitutions (e.g., conservative substitutions), relative to the nucleotide sequence of SEQ ID NO: 1746;
(v) a transgene encoding an NPC1 protein encoded by a nucleotide sequence comprising a nucleotide sequence, e.g., a codon optimized nucleotide sequence, comprising a nucleotide sequence with at least 85% (e.g., at least about 90, 92, 95, 96, 97, 98, or 99%) sequence identity to the nucleotide sequence of SEQ ID NO: 1750;
(vi) a polyA signal region, optionally wherein the polyA signal region comprises the nucleotide sequence of SEQ ID NO: 1751, or a nucleotide sequence at least 95% identical thereto; and
(vii) a 3′ AAV ITR, optionally wherein the 3′ AAV ITR comprises the nucleotide sequence of SEQ ID NO: 1734, or a nucleotide sequence at least 95% identical thereto.

39. The viral genome of any one of claims 2-38, which comprises:
(i) the nucleotide sequence of SEQ ID NO: 1799-1802, or a nucleotide sequence at least 95% identical thereto; or
(ii) the nucleotide sequence of SEQ ID NO: 1814-1821, 1825-1828, or a nucleotide sequence at least 95% identical thereto.

40. The viral genome of any one of claim 2 or 4-38, which comprises the nucleotide sequence of SEQ ID NO: 1755, 1757, 1803-1087, 1810-1812, 1815-1816, 1824, 1827, 1828, or 1830-1831, or a nucleotide sequence at least 95% identical thereto.

41. An isolated, e.g., recombinant, NPC1 protein encoded by the viral genome of any one of claims 2-40.

42. An isolated, e.g., recombinant, AAV particle comprising:
(i) a capsid protein; and
(ii) the viral genome of any one of claims 2-40.

43. The AAV particle of claim 42, wherein:
(i) the capsid protein comprises the amino acid sequence of SEQ ID NO: 138, or an amino acid sequence with at least 80% (e.g., at least about 85, 90, 95, 96, 97, 98, or 99%) sequence identity thereto;
(ii) the capsid protein comprises an amino acid sequence having at least one, two or three modifications but not more than 30, 20 or 10 modifications of the amino acid sequence of SEQ ID NO: 138;
(iii) the capsid protein comprises the amino acid sequence of SEQ ID NO: 11, or an amino acid sequence with at least 80% (e.g., at least about 85, 90, 95, 96, 97, 98, or 99%) sequence identity thereto;
(iv) the capsid protein comprises an amino acid sequence having at least one, two or three modifications but not more than 30, 20 or 10 modifications of the amino acid sequence of SEQ ID NO: 11;
(v) the capsid protein comprises an amino acid sequence encoded by the nucleotide sequence of SEQ ID NO: 137, or a sequence with at least 80% (e.g., at least about 85, 90, 95, 96, 97, 98, or 99%) sequence identity thereto; and/or
(vi) the nucleotide sequence encoding the capsid protein comprises the nucleotide sequence of SEQ ID NO: 137, or a sequence with at least 80% (e.g., at least about 85, 90, 95, 96, 97, 98, or 99%) sequence identity thereto.

44. The AAV particle of claim 42 or 43, wherein the capsid protein comprises:
(i) an amino acid substitution at position K449, e.g., a K449R substitution, numbered according to SEQ ID NO:138;
(ii) an insert comprising the amino acid sequence of TLAVPFK (SEQ ID NO: 1262), optionally wherein the insert is present immediately subsequent to position 588, relative to a reference sequence numbered according to SEQ ID NO:138;
(iii) an amino acid other than “A” at position 587 and/or an amino acid other than “Q” at position 588, numbered according to SEQ ID NO: 138;
(iv) the amino acid substitution of A587D and/or Q588G, numbered according to SEQ ID NO:138.

45. The AAV particle of any one of claims 42-93, wherein the capsid protein comprise:
(a) (i) the amino acid substitution of K449R numbered according to SEQ ID NO:138; and (ii) an insert comprising the amino acid sequence of TLAVPFK (SEQ ID NO: 1262), optionally wherein the insert is present immediately subsequent to position 588 of SEQ ID NO:138;
(b) the capsid protein comprises (i) the amino acid substitution of K449R numbered according to SEQ ID NO:138; (ii) an insert comprising the amino acid sequence of TLAVPFK (SEQ ID NO: 1262), optionally wherein the insert is present immediately subsequent to position 588, relative to a reference sequence numbered according to SEQ ID NO:138; and (iii) the amino acid substitutions of A587D and Q588G, numbered according to SEQ ID NO:138; or
(c) (i) an insert comprising the amino acid sequence of TLAVPFK (SEQ ID NO: 1262), optionally wherein the insert is present immediately subsequent to position 588, relative to a reference sequence numbered according to SEQ ID NO:138; and (ii) the amino acid substitutions of A587D and Q588G, numbered according to SEQ ID NO:138.

46. The AAV particle of claim 42-45, wherein:
(i) the capsid protein comprises the amino acid sequence of SEQ ID NO: 1, or an amino acid sequence substantially identical (e.g., having at least 70%, 75%, 80%, 85%, 90%, 92%, 95%, 97%, 98%, or 99% sequence identity) thereto;
(ii) the capsid protein comprises an amino acid sequence comprising at least one, two, or three modifications but no more than 30, 20, or 10 modifications, e.g., substitutions, relative to the amino acid sequence of SEQ ID NO: 1;
(iii) the capsid protein comprises an amino acid sequence encoded by the nucleotide sequence of SEQ ID NO: 2 or a nucleotide sequence substantially identical (e.g., having at least 70%, 75%, 80%, 85%, 90%, 92%, 95%, 97%, 98%, or 99% sequence identity) thereto; and/or
(iv) the nucleotide sequence encoding the capsid protein comprises the nucleotide sequence of SEQ ID NO: 2, or a nucleotide sequence substantially identical (e.g., having at least 70%, 75%, 80%, 85%, 90%, 92%, 95%, 97%, 98%, or 99% sequence identity) thereto.

47. A vector comprising the viral genome of any one of claims 2-40 or the isolated nucleic acid of claim 1.

48. A cell comprising the viral genome of any one of claims 2-40, the viral particle of any one of claims 42-46, or the vector of claim 47, optionally wherein the cell is a mammalian cell, e.g., an HEK293 cell, an insect cell, e.g., an Sf9 cell, or a bacterial cell.

49. A method of making an isolated, e.g., recombinant, AAV particle, the method comprising
(i) providing a host cell comprising the viral genome of any one of claims 2-40; and
(ii) incubating the host cell under conditions suitable to enclose the viral genome in a capsid protein, e.g., an AAV9 capsid protein;
thereby making the isolated AAV particle.

50. A pharmaceutical composition comprising the AAV particle of any one of claims 42-46, or an AAV particle comprising the viral genome of any one of claims 2-40, and a pharmaceutically acceptable excipient.

51. A method of delivering an exogenous NPC1 protein to a subject, comprising administering an effective amount of the pharmaceutical composition of claim 50, the AAV particle of any one of claims 42-46, or an AAV particle comprising the viral genome of any one of claims 2-40.

52. The method of claim 51, wherein the subject has, has been diagnosed with having, or is at risk of having:
(i) a disease associated with expression of NPC1, e.g., aberrant or reduced NPC1 expression, e.g., expression of an NPC1 gene, NPC1 mRNA, and/or NPC1 protein; and/or
(ii) a lysosomal storage disease or Niemann-Pick disease, type C1.

53. A method of treating a subject having or diagnosed with having a disease associated with NPC1 expression comprising administering to the subject an effective amount of the pharmaceutical composition of claim 50, the AAV particle of any one of claims 42-46, or an AAV particle comprising the viral genome of any one of claims 2-40, thereby treating the disease associated with NPC1 expression in the subject.

54. A method of treating a subject having or diagnosed with having a lysosomal storage disease, comprising administering to the subject an effective amount of the pharmaceutical composition of claim 50, the AAV particle of any one of claims 42-46, or an AAV particle comprising the viral genome of any one of claims 2-40, thereby treating the lysosomal storage disease in the subject.

55. The method of claim 53 or 54, wherein the disease associated with NPC1 expression or the lysosomal storage disease is Niemann-Pick disease, type C1, optionally wherein the Niemann-Pick disease, type C1 is neonate onset Niemann-Pick disease, type C1 or juvenile onset Niemann-Pick disease, type C1.

56. A method of treating a subject having or diagnosed with having a Niemann-Pick disease, type C1 comprising administering to the subject an effective amount of the pharmaceutical composition of claim 50, the AAV particle of any one of claims 42-46, or an AAV particle comprising the viral genome of any one of claims 2-40, thereby treating the Niemann-Pick disease, type C1 in the subject.

57. The method of any one of claims 51-56, wherein the subject:
(i) is a human;
(ii) comprises a mutation in the NPC1 gene, NPC1 mRNA, and/or NPC1 protein;
(iii) is between 0 to 3 months of age;
(iv) is between 3 months to 2 years of age;
(v) is between 2 years of age to 6 years of age;
(vi) is between 6 years of age to 15 years of age; or
(vii) is above 15 years of age.

58. The method of any one of claims 51-57, wherein the AAV particle is administered to the subject intramuscularly, intravenously, intracerebrally, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM).

59. The method of any one of embodiments 51-58, wherein the AAV particle is administered to the subject via intravenous administration, optionally wherein the intravenous administration is via focused ultrasound (FUS), e.g., coupled with the intravenous administration of microbubbles (FUS-MB), or MRI-guided FUS coupled with intravenous administration.

60. The method of any one of claims 51-59, wherein the administration results in increased level of NPC1 protein expression (e.g., 2-fold, 3-fold, 4-fold, 5-fold, 6-fold, 7-fold, 8-fold, 9-fold, 10-fold, 15-fold, 20-fold more NPC protein expression) in a cell of the subject (e.g., a cell of the CNS, e.g., a cell of the cortex, hippocampus, cerebellum, or brainstem), relative to reference level, e.g., a subject that has not received treatment, e.g., has not been administered the AAV particle.

61. The method of any one of claims 51-60, further comprising administration of an additional therapeutic agent and/or therapy suitable for treatment or prevention of the disease associated with NPC1 expression, the lysosomal storage disease, and/or Niemann-Pick disease, type C1, optionally wherein the additional therapeutic agent and/or therapy comprises TRAPPSOL CYCLO, VTS-270 (e.g., a 2-hydroxypropyl-β-cyclodextrin (HPβCD) mixture), arimoclomol (e.g., arimoclomol citrate), or a combination thereof.

62. The isolated nucleic acid of claim 1, the isolated viral genome of any one of claims 2-40, the AAV particle of any one of claims 42-46, or the pharmaceutical composition of claim 50 for use in the manufacture of a medicament.

63. The isolated nucleic acid of claim 1, the isolated viral genome of any one of claims 2-40, the AAV particle of any one of claims 42-46, or the pharmaceutical composition of claim 50 for use in the treatment of a disease associated with NPC1 expression, the lysosomal storage disease, and/or Niemann-Pick disease, type C1.

64. Use of an effective amount of an AAV particle comprising the genome of any one of claims 2-40, the AAV particle of any one of claims 42-46, or the pharmaceutical composition of claim 50 in the manufacture of a medicament for the treatment of a disease associated with NPC1 expression, the lysosomal storage disease, and/or Niemann-Pick disease, type C1.
